(19) United States
(12) Patent Application Publication
(10) Pub. No.: US 2005/0142148 A1
(43) Pub. Date: Jun. 30, 2005

(54) Recombinant parainfluenza virus expression systems and vaccines comprising heterologous antigens derived from metapneumovirus

(76) Inventors: Ronaldus Adrianus Maria Fouchier, Rotterdam (NL); Bernadetta Gerarda Van Den Hoogen, Rotterdam (NL); Albertus Dominicus Marcellinus Erasmus Osterhaus, Bunnik (NL); Aurelia Haller, Boulder, CO (US); Roderick Tang, San Mateo, CA (US)

Correspondence Address: JONES DAY, 222 EAST 41ST ST, NEW YORK, NY, 10017, US

(21) Appl. No.: 10/831,781

(22) Filed: Apr. 23, 2004

Related U.S. Application Data
(60) Provisional application No. 60/466,181, filed on Apr. 25, 2003.
(60) Provisional application No. 60/499,274, filed on Aug. 28, 2003.
(60) Provisional application No. 60/550,931, filed on Mar. 5, 2004.

(51) Int. Cl. A61K 39/12; C12N 7/00; C12N 15/86

(52) U.S. Cl. 424/199.1; 435/456; 435/235.1

(57) ABSTRACT

The present invention relates to recombinant bovine parainfluenza virus (bPIV) cDNA or RNA which may be used to express heterologous gene products in appropriate host cell systems and/or to rescue negative strand RNA recombinant viruses that express, package, and/or present the heterologous gene product. In particular, the heterologous gene products include gene product of another species of PIV or from another negative strand RNA virus, including but not limited to, influenza virus, respiratory syncytial virus, human metapneumovirus and avian pneumovirus. The chimeric viruses and expression products may advantageously be used in vaccine formulations including vaccines against a broad range of pathogens and antigens.

This application claims priority to U.S. Provisional Application No. 60/466,181, filed on Apr. 25, 2003; U.S. Provisional Application No. 60/499,274, filed on Aug. 28, 2003 and U.S. Provisional Application No. 60/550,931, filed on Mar. 5, 2004, each of which is incorporated herein by reference in its entirety.

### 1. INTRODUCTION

The present invention relates to recombinant parainfluenza virus (PIV) cDNA or RNA that may be used to express heterologous gene products in appropriate host cell systems and/or to rescue negative strand RNA recombinant viruses that express, package, and/or present the heterologous gene product. In particular, the present invention encompasses vaccine preparations comprising chimeric PIV expressing a heterologous gene product, wherein the heterologous gene product is preferably an antigenic peptide or polypeptide. In one embodiment, the PIV vector of the invention expresses one, two, or three heterologous gene products that may be encoded by the same or different viruses. In a preferred embodiment, the heterologous sequence encodes a heterologous gene product that is an antigenic polypeptide from another species of PIV or from another negative strand RNA virus, including but not limited to, influenza virus, respiratory syncytial virus (RSV), mammalian metapneumovirus, and avian pneumovirus. The vaccine preparations of the invention encompass multivalent vaccines, including bivalent and trivalent vaccine preparations. The multivalent vaccines of the invention may be administered in the form of one PIV vector expressing each heterologous antigenic sequence or two or more PIV vectors each encoding different heterologous antigenic sequences. The vaccine preparations of the invention can be administered alone or in combination with other vaccines, prophylactic agents, or therapeutic agents.

### 2. BACKGROUND OF THE INVENTION

Parainfluenza viral infection results in serious respiratory tract disease in infants and children. (Tao et al., 1999, Vaccine 17: 1100-08). Infectious parainfluenza viral infections account for approximately 20% of all hospitalizations of pediatric patients that suffer from respiratory tract infections worldwide. Id. An effective antiviral therapy is not available to treat PIV related diseases, and a vaccine to prevent PIV infection has not yet been approved.

PIV is a member of the genus respirovirus (PIV1, PIV3) or rubulavirus (PIV2, PIV4) of the paramyxoviridae family. PIV is made up of two structural modules: (1) an internal ribonucleoprotein core, or nucleocapsid, containing the viral genome, and (2) an outer, roughly spherical lipoprotein envelope. Its genome consists of a single strand of negative sense RNA, that is approximately 15,456 nucleotides in length and encodes at least eight polypeptides. These proteins include the nucleocapsid structural protein (NP, NC, or N depending on the genera), the phosphoprotein (P), the matrix protein (M), the fusion glycoprotein (F), the hemagglutinin-neuraminidase glycoprotein (HN), the large polymerase protein (L), and the C and D proteins of unknown function. Id.

The parainfluenza nucleocapsid protein (NP, NC, or N) contains two domains within each protein unit. These domains include: an amino-terminal domain, that comprises nearly two-thirds of the molecule and interacts directly with the RNA, and a carboxyl-terminal domain, that lies on the surface of the assembled nucleocapsid. A hinge is thought to exist at the junction of these two domains, thereby imparting some flexibility on this protein (see Fields et al. (ed.), 1991, FUNDAMENTAL VIROLOGY, 2^{nd} ed, Raven Press, New York, incorporated by reference herein in its entirety). The matrix protein (M) is apparently involved in viral assembly, and it interacts with both the viral membrane and the nucleocapsid proteins. The phosphoprotein (P) is subject to phosphorylation and has been implicated in transcription regulation, methylation, phosphorylation and polyadenylation. Produced initially as an inactive precursor, the fusion glycoprotein (F) is cleaved upon translation to produce two disulfide linked polypeptides. The active F protein interacts with the viral membrane where it facilitates penetration of the parainfluenza virion into host cells by promoting the fusion of the viral envelope with the host cell plasma membrane. Id. The glycoprotein, hemagglutinin-neuraminidase (HN) protrudes from the envelope and imparts hemagglutinin and neuraminidase activities on the virus. HN has a strongly hydrophobic amino terminus that functions to anchor the HN protein into the lipid bilayer. Id. Finally, the large polymerase protein (L) plays an important role in both transcription and replication. Id.

Bovine parainfluenza virus was first isolated in 1959 from calves showing signs of shipping fever. It has since been isolated from normal cattle, aborted fetuses, and cattle exhibiting signs of respiratory disease (Breker-Klassen et al., 1996, Can. J. Vet. Res. 60: 228-236. See also Shibuta, 1977, Microbiol. Immunol. 23 (7), 617-628). Human and bovine PIV3 share neutralizing epitopes but show distinct antigenic properties. Significant differences exist between the human and bovine viral strains in the HN protein. In fact, a bovine strain induces some neutralizing antibodies to hPIV infection while a human strain seems to induce a wider spectrum of neutralizing antibodies against human PIV3 (Van Wyke Coelingh et al., 1990, J. Virol. 64: 3833-3843).

The replication of all negative-strand RNA viruses, including PIV, is complicated by the absence of the cellular machinery that is required to replicate RNA. Additionally, the negative-strand genome must be transcribed into a positive-strand (mRNA) copy before translation can occur. Consequently, the genomic RNA alone cannot synthesize the required RNA-dependent RNA polymerase upon entry into the cell. The L, P and N proteins must enter the host cell along with the genomic RNA.

It is hypothesized that most or all of the viral proteins that transcribe PIV mRNA also carry out the replication of the genome. The mechanism that regulates the alternative uses (i.e., transcription or replication) of the same complement of proteins has not been clearly identified, but the process appears to involve the abundance of free forms of one or more of the nucleocapsid proteins. Directly following penetration of the virus, transcription is initiated by the L protein using the negative-sense RNA in the nucleocapsid as a template. Viral RNA synthesis is regulated such that it produces monocistronic mRNAs during transcription.

Following transcription, virus genome replication is the second essential event in infection by negative-strand RNA viruses. As with other negative-strand RNA viruses, virus genome replication in PIV is mediated by virus-specified proteins. The first products of replicative RNA synthesis are complementary copies (i.e., plus-polarity) of the PIV genomic RNA (cRNA). These plus-stranded copies (anti-genomes) differ from the plus-stranded mRNA transcripts in the structure of their termini. Unlike the mRNA transcripts, the anti-genomic cRNAs are not capped or methylated at the 5′ termini, and they are not truncated nor polyadenylated at the 3′ termini. The cRNAs are coterminal with their negative strand templates and contain all the genetic information in the complementary form. The cRNAs serve as templates for the synthesis of PIV negative-strand viral genomes (vRNAs).

The bPIV negative strand genomes (vRNAs) and antigenomes (cRNAs) are encapsidated by nucleocapsid proteins; the only unencapsidated RNA species are viral mRNAs. Replication and transcription of bPIV RNA occurs in the cytoplasm of the host cell. Assembly of the viral components appears to take place at the host cell plasma membrane where the mature virus is released by budding.

2.1. Paramyxovirus

Classically, as devastating agents of disease, paramyxoviruses account for many animal and human deaths worldwide each year. The Paramyxoviridae form a family within the order of Mononegavirales (negative-sense single stranded RNA viruses), consisting of the sub-families Paramyxovirinae and Pneumovirinae. The latter sub-family is at present taxonomically divided in the genera Pneumovirus and Metapneumovirus (Pringle, 1999, Arch. Virol. 144/2, 2065-2070). Human respiratory syncytial virus (hRSV), a species of the Pneumovirus genus, is the single most important cause of lower respiratory tract infections during infancy and early childhood worldwide (Domachowske, & Rosenberg, 1999, Clin. Microbio. Rev. 12(2): 298-309). Other members of the Pneumovirus genus include the bovine and ovine respiratory syncytial viruses and pneumonia virus of mice (PVM).

In the past decades several etiological agents of mammalian disease, in particular of respiratory tract illnesses (RTI), in particular of humans, have been identified (Evans, In: Viral Infections of Humans, Epidemiology and Control. 3th ed. (ed. Evans, A. S) 22-28 (Plenum Publishing Corporation, New York, 1989)). Classical etiological agents of RTI with mammals are respiratory syncytial viruses belonging to the genus Pneumovirus found with humans (hRSV) and ruminants such as cattle or sheep (bRSV and/or oRSV). In human RSV differences in reciprocal cross neutralization assays, reactivity of the G proteins in immunological assays and nucleotide sequences of the G gene are used to define two hRSV antigenic subgroups. Within the subgroups the amino acid sequences show 94% (subgroup A) or 98% (subgroup B) identity, while only 53% amino acid sequence identity is found between the subgroups. Additional variability is observed within subgroups based on monoclonal antibodies, RT-PCR assays and RNAse protection assays. Viruses from both subgroups have a worldwide distribution and may occur during a single season. Infection may occur in the presence of pre-existing immunity and the antigenic variation is not strictly required to allow re-infection. See, for example Sullender, 2000, Clinical Microbiology Reviews 13(1): 1-15; Collins et al. Fields Virology, ed. B. N. Knipe, Howley, P. M. 1996, Philadelphia: Lippencott-Raven. 1313-1351; Johnson et al., 1987, (Proc Natl Acad Sci USA, 84(16): 5625-9; Collins, in The Paramyxoviruses, D. W. Kingsbury, Editor. 1991, Plenum Press: New York. p. 103-153.

Another classical Pneumovirus is the pneumonia virus of mice (PVM), in general only found with laboratory mice. However, a proportion of the illnesses observed among mammals can still not be attributed to known pathogens.

2.2. RSV Infections

Respiratory syncytial virus (RSV) is the leading cause of serious lower respiratory tract disease in infants and children (Feigen et al., eds., 1987, In: Textbook of Pediatric Infectious Diseases, W B Saunders, Philadelphia at pages 1653-1675; New Vaccine Development, Establishing Priorities, Vol. 1, 1985, National Academy Press, Washington D.C. at pages 397-409; and Ruuskanen et al., 1993, Curr. Probl. Pediatr. 23:50-79). The yearly epidemic nature of RSV infection is evident worldwide, but the incidence and severity of RSV disease in a given season vary by region (Hall, 1993, Contemp. Pediatr. 10:92-110). In temperate regions of the northern hemisphere, it usually begins in late fall and ends in late spring. Primary RSV infection occurs most often in children from 6 weeks to 2 years of age and uncommonly in the first 4 weeks of life during nosocomial epidemics (Hall et al., 1979, New Engl. J. Med. 300: 393-396). Children at increased risk for RSV infection include, but are not limited to, preterm infants (Hall et al., 1979, New Engl. J. Med. 300: 393-396) and children with bronchopulmonary dysplasia (Groothuis et al., 1988, Pediatrics 82: 199-203), congenital heart disease (MacDonald et al., New Engl. J. Med. 307: 397-400), congenital or acquired immunodeficiency (Ogra et al., 1988, Pediatr. Infect. Dis. J. 7: 246-249; and Pohl et al., 1992, J. Infect. Dis. 165: 166-169), and cystic fibrosis (Abman et al., 1988, J. Pediatr. 113: 826-830). The fatality rate in infants with heart or lung disease who are hospitalized with RSV infection is 3%-4% (Navas et al., 1992, J. Pediatr. 121: 348-354).

RSV infects adults as well as infants and children. In healthy adults, RSV causes predominantly upper respiratory tract disease. It has recently become evident that some adults, especially the elderly, have symptomatic RSV infections more frequently than had been previously reported (Evans, A. S., eds., 1989, Viral Infections of Humans. Epidemiology and Control, 3rd ed., Plenum Medical Book, New York at pages 525-544). Several epidemics also have been reported among nursing home patients and institutionalized young adults (Falsey, A. R., 1991, Infect. Control Hosp. Epidemiol. 12: 602-608; and Garvie et al., 1980, Br. Med. J. 281: 1253-1254). Finally, RSV may cause serious disease in immunosuppressed persons, particularly bone marrow transplant patients (Hertz et al., 1989, Medicine 68: 269-281).

Treatment options for established RSV disease are limited. Severe RSV disease of the lower respiratory tract often requires considerable supportive care, including administration of humidified oxygen and respiratory assistance (Fields et al., eds, 1990, Fields Virology, 2nd ed., Vol. 1, Raven Press, New York at pages 1045-1072).

While a vaccine might prevent RSV infection, and/or RSV-related disease, no vaccine is yet licensed for this indication. A major obstacle to vaccine development is safety. A formalin-inactivated vaccine, though immunogenic, unexpectedly caused a higher and more severe incidence of lower respiratory tract disease due to RSV in immunized infants than in infants immunized with a similarly prepared trivalent parainfluenza vaccine (Kim et al., 1969, Am. J. Epidemiol. 89: 422-434; and Kapikian et al., 1969, Am. J. Epidemiol. 89: 405-421). Several candidate RSV vaccines have been abandoned and others are under development (Murphy et al., 1994, Virus Res. 32: 13-36), but even if safety issues are resolved, vaccine efficacy must also be improved. A number of problems remain to be solved. Immunization would be required in the immediate neonatal period since the peak incidence of lower respiratory tract disease occurs at 2-5 months of age. The immaturity of the neonatal immune response together with high titers of maternally acquired RSV antibody may be expected to reduce vaccine immunogenicity in the neonatal period (Murphy et al., 1988, J. Virol. 62: 3907-3910; and Murphy et al., 1991, Vaccine 9: 185-189). Finally, primary RSV infection and disease do not protect well against subsequent RSV disease (Henderson et al., 1979, New Engl. J. Med. 300: 530-534).

Currently, the only approved approach to prophylaxis of RSV disease is passive immunization. Initial evidence suggesting a protective role for IgG was obtained from observations involving maternal antibody in ferrets (Prince, G. A., Ph.D. diss., University of California, Los Angeles, 1975) and humans (Lambrecht et al, 1976, J. Infect. Dis. 134: 211-217; and Glezen et al., 1981, J. Pediatr. 98:708-715). Hemming et al. (Morell et al., eds., 1986, Clinical Use of Intravenous Immunoglobulins, Academic Press, London at pages 285-294) recognized the possible utility of RSV antibody in treatment or prevention of RSV infection during studies involving the pharmacokinetics of an intravenous immune globulin (IVIG) in newborns suspected of having neonatal sepsis. In this study, it was noted that one infant, whose respiratory secretions yielded RSV, recovered rapidly after IVIG infusion. Subsequent analysis of the IVIG lot revealed an unusually high titer of RSV neutralizing antibody. This same group of investigators then examined the ability of hyperimmune serum or immune globulin, enriched for RSV neutralizing antibody, to protect cotton rats and primates against RSV infection (Prince et al., 1985, Virus Res. 3: 193-206; Prince et al., 1990, J. Virol. 64: 3091-3092; Hemming et al., 1985, J. Infect. Dis. 152: 1083-1087; Prince et al., 1983, Infect. Immun. 42: 81-87; and Prince et al., 1985, J. Virol. 55: 517-520). Results of these studies indicate that IVIG may be used to prevent RSV infection, in addition to treating or preventing RSV-related disorders.

Recent clinical studies have demonstrated the ability of this passively administered RSV hyperimmune globulin (RSV IVIG) to protect at-risk children from severe lower respiratory infection by RSV (Groothius et al., 1993, New Engl. J. Med. 329: 1524-1530; and The PREVENT Study Group, 1997, Pediatrics 99: 93-99). While this is a major advance in preventing RSV infection, this treatment poses certain limitations in its widespread use. First, RSV IVIG must be infused intravenously over several hours to achieve an effective dose. Second, the concentrations of active material in hyperimmune globulins are insufficient to treat adults at risk or most children with comprised cardiopulmonary function. Third, intravenous infusion necessitates monthly hospital visits during the RSV season. Finally, it may prove difficult to select sufficient donors to produce a hyperimmune globulin for RSV to meet the demand for this product. Currently, only approximately 8% of normal donors have RSV neutralizing antibody titers high enough to qualify for the production of hyperimmune globulin.

One way to improve the specific activity of the immunoglobulin would be to develop one or more highly potent RSV neutralizing monoclonal antibodies (MAbs). Such MAbs should be human or humanized in order to retain favorable pharmacokinetics and to avoid generating a human anti-mouse antibody response, as repeat dosing would be required throughout the RSV season. Two glycoproteins, F and G, on the surface of RSV have been shown to be targets of neutralizing antibodies (Fields et al., 1990, supra; and Murphy et al., 1994, supra).

A humanized antibody directed to an epitope in the A antigenic site of the F protein of RSV, SYNAGIS®, is approved for intramuscular administration to pediatric patients for prevention of serious lower respiratory tract disease caused by RSV at recommended monthly doses of 15 mg/kg of body weight throughout the RSV season (November through April in the northern hemisphere). SYNAGIS® is a composite of human (95%) and murine (5%) antibody sequences. See, Johnson et al., 1997, J. Infect. Diseases 176: 1215-1224 and U.S. Pat. No. 5,824,307, the entire contents of which are incorporated herein by reference. The human heavy chain sequence was derived from the constant domains of human IgG1 and the variable framework regions of the VH genes of Cor (Press et al., 1970, Biochem. J. 117: 641-660) and Cess (Takashi et al., 1984, Proc. Natl. Acad. Sci. USA 81: 194-198). The human light chain sequence was derived from the constant domain of C and the variable framework regions of the VL gene K104 with J-4 (Bentley et al., 1980, Nature 288: 5194-5198). The murine sequences derived from a murine monoclonal antibody, Mab 1129 (Beeler et al., 1989, J. Virology 63: 2941-2950), in a process which involved the grafting of the murine complementarity determining regions into the human antibody frameworks.

2.3. Avian Pneumoviruses

Respiratory disease caused by an avian pneumovirus (APV) was first described in South Africa in the late 1970s (Buys et al., 1980, Turkey 28: 36-46) where it had a devastating effect on the turkey industry. The disease in turkeys was characterized by sinusitis and rhinitis and was called turkey rhinotracheitis (TRT). The European isolates of APV have also been strongly implicated as factors in swollen head syndrome (SHS) in chickens (O'Brien, 1985, Vet. Rec. 117: 619-620). Originally, the disease appeared in broiler chicken flocks infected with Newcastle disease virus (NDV) and was assumed to be a secondary problem associated with Newcastle disease (ND). Antibody against European APV was detected in affected chickens after the onset of SHS (Cook et al., 1988, Avian Pathol. 17: 403-410), thus implicating APV as the cause.

Avian pneumovirus (APV) also known as turkey rhinotracheitis virus (TRTV), the aetiological agent of avian rhinotracheitis, an upper respiratory tract infection of turkeys (Giraud et al., 1986, Vet. Res. 119: 606-607), is the sole member of the recently assigned Metapneumovirus genus, which, as said was until now not associated with infections, or what is more, with disease of mammals. Serological subgroups of APV can be differentiated on the basis of nucleotide or amino acid sequences of the G glycoprotein and neutralization tests using monoclonal antibodies that also recognize the G glycoprotein. However, other differences in the nucleotide and amino acid sequences can be used to distinguish serological subgroups of APV. Within subgroups A, B and D, the G protein shows 98.5 to 99.7% aa sequence identity within subgroups while between the subgroups only 31.2-38% aa identity is observed. See for example Collins et al., 1993, Avian Pathology, 22: p. 469-479; Cook et al., 1993, Avian Pathology, 22: 257-273; Bayon-Auboyer et al., J Gen Virol, 81(Pt 11): 2723-33; Seal, 1998, Virus Res, 58(1-2): 45-52; Bayon-Auboyer et al., 1999, Arch Virol, 144(6): 91-109; Juhasz, et al., 1994, J Gen Virol, 75(Pt 11): 2873-80.

A further serotype of APV is provided in WO00/20600, incorporated by reference herein, which describes the Colorado isolate of APV and compared it to known APV or TRT strains with in vitro serum neutralization tests. First, the Colorado isolate was tested against monospecific polyclonal antisera to recognized TRT isolates. The Colorado isolate was not neutralized by monospecific antisera to any of the TRT strains. It was, however, neutralized by a hyperimmune antiserum raised against a subgroup A strain. This antiserum neutralized the homologous virus to a titre of 1:400 and the Colorado isolate to a titer of 1:80. Using the above method, the Colorado isolate was then tested against TRT monoclonal antibodies. In each case, the reciprocal neutralization titer was <10. Monospecific antiserum raised to the Colorado isolate was also tested against TRT strains of both subgroups. None of the TRT strains tested were neutralized by the antiserum to the Colorado isolate.

The Colorado strain of APV does not protect SPF chicks against challenge with either a subgroup A or a subgroup B strain of TRT virus. These results suggest that the Colorado isolate may be the first example of a further serotype of avian pneumovirus (See, Bayon-Auboyer et al., 2000, J. Gen. Vir. 81: 2723-2733).

The avian pneumovirus is a single stranded, non-segmented RNA virus that belongs to the sub-family Pneumovirinae of the family Paramyxoviridae, genus metapneumovirus (Cavanagh and Barrett, 1988, Virus Res. 11: 241-256; Ling et al., 1992, J. Gen. Virol. 73: 1709-1715; Yu et al., 1992, J. Gen. Virol. 73: 1355-1363). The Paramyxoviridae family is divided into two sub-families: the Paramyxovirinae and Pneumovirinae. The subfamily Paramyxovirinae includes, but is not limited to, the genera: Paramyxovirus, Rubulavirus, and Morbillivirus. Recently, the sub-family Pneumovirinae was divided into two genera based on gene order, and sequence homology, i.e. pneumovirus and metapneumovirus (Naylor et al., 1998, J. Gen. Virol., 79: 1393-1398; Pringle, 1998, Arch. Virol. 143: 1449-1159). The pneumovirus genus includes, but is not limited to, human respiratory syncytial virus (hRSV), bovine respiratory syncytial virus (bRSV), ovine respiratory syncytial virus, and mouse pneumovirus. The metapneumovirus genus includes, but is not limited to, European avian pneumovirus (subgroups A and B), which is distinguished from hRSV, the type species for the genus pneumovirus (Naylor et al., 1998, J. Gen. Virol., 79: 1393-1398; Pringle, 1998, Arch. Virol. 143: 1449-1159). The US isolate of APV represents a third subgroup (subgroup C) within metapneumovirus genus because it has been found to be antigenically and genetically different from European isolates (Seal, 1998, Virus Res. 58: 45-52; Senne et al., 1998, In: Proc. 47th WPDC, California, pp. 67-68).

Electron microscopic examination of negatively stained APV reveals pleomorphic, sometimes spherical, virions ranging from 80 to 200 nm in diameter with long filaments ranging from 1000 to 2000 nm in length (Collins and Gough, 1988, J. Gen. Virol. 69: 909-916). The envelope is made of a membrane studded with spikes 13 to 15 nm in length. The nucleocapsid is helical, 14 nm in diameter and has 7 nm pitch. The nucleocapsid diameter is smaller than that of the genera Paramyxovirus and Morbillivirus, which usually have diameters of about 18 nm.

Avian pneumovirus infection is an emerging disease in the USA despite its presence elsewhere in the world in poultry for many years. In May 1996, a highly contagious respiratory disease of turkeys appeared in Colorado, and an APV was subsequently isolated at the National Veterinary Services Laboratory (NVSL) in Ames, Iowa (Senne et al., 1997, Proc. 134th Ann. Mtg., AVMA, pp. 190). Prior to this time, the United States and Canada were considered free of avian pneumovirus (Pearson et al., 1993, In: Newly Emerging and Re-emerging Avian Diseases: Applied Research and Practical Applications for Diagnosis and Control, pp. 78-83; Hecker and Myers, 1993, Vet. Rec. 132:172). Early in 1997, the presence of APV was detected serologically in turkeys in Minnesota. By the time the first confirmed diagnosis was made, APV infections had already spread to many farms. The disease is associated with clinical signs in the upper respiratory tract: foamy eyes, nasal discharge and swelling of the sinuses. It is exacerbated by secondary infections. Morbidity in infected birds can be as high as 100%. The mortality can range from 1 to 90% and is highest in six to twelve week old poults.

Avian pneumovirus is transmitted by contact. Nasal discharge, movement of affected birds, contaminated water, contaminated equipment; contaminated feed trucks and load-out activities can contribute to the transmission of the virus. Recovered turkeys are thought to be carriers. Because the virus is shown to infect the epithelium of the oviduct of laying turkeys and because APV has been detected in young poults, egg transmission is considered a possibility.

A significant portion of human respiratory disease is caused by members of the viral sub-families Paramyxovirinae and Pneumovirinae, there still remains a need for an effective vaccine to confer protection against a variety of viruses that result in respiratory tract infection.

Citation or discussion of a reference herein shall not be construed as an admission that such is prior art to the present invention.

### 3. SUMMARY OF THE INVENTION

The present invention relates to recombinant parainfluenza virus cDNA and RNA that may be engineered to express heterologous or non-native gene products, in particular, to express antigenic polypeptides and peptides. In one embodiment, the present invention relates to recombinant bovine or human parainfluenza viruses which are engineered to express heterologous antigens or immunogenic and/or antigenic fragments of heterologous antigens. In another embodiment of the invention, the recombinant bovine or human parainfluenza viruses are engineered to express sequences that are non-native to the PIV genome, including mutated PIV nucleotide sequences. In particular, the invention relates to recombinant Kansas-strain bovine parainfluenza type 3 virus as well as cDNA and RNA molecules coding for the same. The present invention also relates to recombinant PIV that contain modifications that result in chimeric viruses with phenotypes more suitable for use in vaccine formulations.

The present invention provides for the first time a chimeric PIV formulated as a vaccine that is able to confer protection against various viral infections, in particular, viruses that result in respiratory tract infections. In a specific embodiment, the present invention provides a vaccine that is able to confer protection against parainfluenza, influenza, or respiratory syncytial viral infection. The present invention provides for the first time a vaccine that is able to confer protection against metapneumovirus infection in a mammalian host.

In accordance with the present invention, a recombinant virus is one derived from a bovine parainfluenza virus or a human parainfluenza virus that is encoded by endogenous or native genomic sequences or non-native genomic sequences. In accordance with the invention, a non-native sequence is one that is different from the native or endogenous genomic sequence due to one or more mutations, including, but not limited to, point mutations, rearrangements, insertions, deletions, etc. to the genomic sequence that may or may not result in a phenotypic change.

In accordance with the present invention, a chimeric virus of the invention is a recombinant bPIV or hPIV which further comprises one or more heterologous nucleotide sequences. In accordance with the invention, a chimeric virus may be encoded by a nucleotide sequence in which heterologous nucleotide sequences have been added to the genome or in which nucleotide sequences have been replaced with heterologous nucleotide sequences.

The present invention also relates to engineered recombinant parainfluenza viruses and viral vectors that encode combinations of heterologous sequences which encode gene products, including but not limited to, genes from different strains of PIV, influenza virus, respiratory syncytial virus, mammalian metapneumovirus (e.g., human metapneumovirus), avian pneumovirus, measles, mumps, other viruses, pathogens, cellular genes, tumor antigens, or combinations thereof. Furthermore, the invention relates to engineered recombinant parainfluenza viruses that contain a nucleotide sequence derived from a metapneumovirus in combination with a nucleotide sequence derived from a respiratory syncytial virus, and further in combination with a nucleotide sequence derived from a human parainfluenza virus. The invention also encompasses recombinant parainfluenza vectors and viruses that are engineered to encode genes from different species and strains of the parainfluenza virus, including the F and HN genes of human PIV3.

In one embodiment, the PIV vector of the invention is engineered to express one or more heterologous sequences, wherein the heterologous sequences encode gene products that are preferably antigenic gene products. In a preferred embodiment, the PIV vector of the invention expresses one, two or three heterologous sequences that encode antigenic polypeptides and peptides. In some embodiments, the heterologous sequences are derived from the same virus or from different viruses. In a preferred embodiment, the heterologous sequences encode heterologous gene products that are antigenic polypeptides from another species of PIV, such as a human PIV, a mutant strain of PIV, or from another negative strand RNA virus, including but not limited to, influenza virus, respiratory syncytial virus (RSV), mammalian metapneumovirus (e.g., human metapneumovirus (hMPV)), and avian pneumovirus. In one embodiment, the heterologous sequence encodes an immunogenic and/or antigenic fragment of a heterologous gene product.

In a preferred embodiment, the recombinant PIV is a bovine PIV type 3, or an attenuated human PIV type 3. In one embodiment, the sequences encoding fusion (F) protein, hemagglutinin (HN) glycoprotein, or other non-essential genes of the PIV genome are deleted and are substituted by heterologous antigenic sequences. In yet another embodiment, the PIV genome contains mutations or modifications, in addition to the heterologous nucleotide sequences, that result in a chimeric virus having a phenotype that is more suitable for use in vaccine formulations, e.g., an attenuated phenotype or a phenotype with enhanced antigenicity.

In a specific embodiment, the heterologous nucleotide sequence to be inserted into the PIV genome is derived from the nucleotide sequences encoding a F protein, a G protein or an HN protein. In certain embodiments, the nucleotide sequence to be inserted encodes a chimeric F protein, a chimeric G protein or a chimeric HN protein. In a specific embodiment, the F protein comprises an ectodomain of a F protein of a metapneumovirus, a transmembrane domain of a F protein of a parainfluenza virus, and a luminal domain of a F protein of a parainfluenza virus. In certain embodiments, the nucleotide sequence to be inserted encodes a F protein, wherein the transmembrane domain of the F protein is deleted so that a soluble F protein is expressed.

In another specific embodiment, the invention provides a chimeric virus comprising a PIV genome comprising a heterologous nucleotide sequence derived from a metapneumovirus. In a specific embodiment, the PIV virus is a Kansas-strain bovine parainfluenza type 3 virus. In other embodiments, the PIV virus is a human parainfluenza virus with an attenuated phenotype. In yet other embodiments, the invention provides a chimeric bovine parainfluenza virus type 3/human parainfluenza virus engineered to contain human parainfluenza F and HN genes in a bovine parainfluenza backbone. The chimeric virus may further comprise a heterologous nucleotide sequence derived from a metapneumovirus, and/or further comprise a heterologous nucleotide sequence derived from a respiratory syncytial virus.

In certain embodiments, the virus of the invention comprises heterologous nucleotide sequences derived from at least two different genes of a metapneumovirus. In a specific embodiment, the heterologous sequence is derived from a metapneumovirus, e.g., avian pneumovirus and human metapneumovirus. More specifically, the heterologous sequence is derived from an avian pneumovirus, including avian pneumovirus type A, B, C or D, preferably C.

The present invention also provides vaccine preparations and immunogenic compositions comprising chimeric PIV expressing one or more heterologous antigenic sequences. In a specific embodiment, the present invention provides multivalent vaccines, including bivalent and trivalent vaccines. The multivalent vaccines of the invention may be administered in the form of one PIV vector expressing each heterologous antigenic sequence or two or more PIV vectors each encoding different heterologous antigenic sequences. In one embodiment, the vaccine preparation of the invention comprises chimeric PIV expressing one, two or three heterologous polypeptides, wherein the heterologous polypeptides can be encoded by sequences derived from one strain of the same virus, different strains of the same virus, or different viruses. Preferably, the heterologous antigenic sequences are derived from a negative strand RNA virus, including but not limited to, influenza virus, parainfluenza virus, respiratory syncytial virus (RSV), mammalian metapneumovirus (e.g., human metapneumovirus (hMPV)), and avian pneumovirus (APV). The heterologous antigenic sequences include, but are not limited to, sequences that encode human parainfluenza virus F or HN protein, F protein of RSV, HA protein of influenza virus type A, B, and C, and F protein of human MPV and avian pneumovirus. More preferably, the vaccine preparation of the invention comprises attenuated chimeric viruses that are viable and infectious. In a preferred embodiment, the recombinant PIV is a bovine PIV type 3, or an attenuated strain of human PIV.

In one embodiment, the vaccine preparation comprises the chimeric virus of the present invention, wherein the F, HN, or some other nonessential genes of the PIV genome have been substituted or deleted. In a preferred embodiment, the vaccine preparation of the present invention is prepared by engineering a strain of PIV with an attenuated phenotype in an intended host. In another preferred embodiment, the vaccine preparation of the present invention is prepared by engineering an attenuated strain of PIV.

In another embodiment, the heterologous nucleotide sequence is added to the complete PIV genome. In certain embodiments, the PIV genome is engineered so that the heterologous sequences are inserted at position one, two, three, four, five or six, so that the heterologous sequences are expressed as the first, second, third, fourth, fifth, or sixth gene of the viral genome. In specific embodiments, the heterologous sequence is inserted at position one, two, or three of the viral genome. In certain embodiments, the intergenic region between the end of the coding sequence of an inserted heterologous gene and the start of the coding sequence of the downstream gene is altered to a desirable length, resulting in enhanced expression of the heterologous sequence or enhanced growth of the chimeric virus. Alternatively, the intergenic region is altered to a desirable length, with a potential to alter the expression of the heterologous sequence or growth of the recombinant or chimeric virus, e.g., attenuated phenotype. In some embodiments, both the position of the insertion and the length of the intergenic region flanking a heterologous nucleotide sequence are engineered to select a recombinant or chimeric virus with desirable levels of expression of the heterologous sequence and desirable viral growth characteristics.

In certain embodiments, the invention provides a vaccine formulation comprising the recombinant or chimeric virus of the invention and a pharmaceutically acceptable excipient. In specific embodiments, the vaccine formulation of the invention is used to modulate the immune response of a subject, such as a human, a primate, a horse, a cow, a sheep, a pig, a goat, a dog, a cat, a rodent or a subject of avian species. In a more specific embodiment, the vaccine is used to modulate the immune response of a human infant or a child. In another embodiment, the present invention relates to vaccine formulations for veterinary uses. The vaccine preparation of the invention can be administered alone or in combination with other vaccines or other prophylactic or therapeutic agents.

TABLE 3.1
CONVENTIONS AND ABBREVIATIONS

| | |
|---|---|
| cDNA | complementary DNA |
| CPE | cytopathic effects |
| L | large protein |
| M | matrix protein (lines inside of envelope) |
| F | fusion glycoprotein |
| HN | hemagglutinin-neuraminidase glycoprotein |
| N, NP or | nucleoprotein (associated with RNA and |
| NC | required for polymerase activity) |
| P | phosphoprotein |
| MOI | multiplicity of infection |
| NA | neuraminidase (envelope glycoprotein) |
| PIV | parainfluenza virus |
| bPIV | bovine parainfluenza virus |
| bPIV3 | bovine parainfluenza virus type 3 |
| hPIV | human parainfluenza virus |
| hPIV3 | human parainfluenza virus type 3 |
| bPIV/hPIV | recombinant bPIV with hPIV sequences |
| or b/h PIV | |
| b/h PIV3 or | recombinant bPIV type 3 with hPIV type |
| bPIV3/hPIV3 | 3 sequences |
| nt | nucleotide |
| RNP | ribonucleoprotein |
| rRNP | recombinant RNP |
| vRNA | genomic virus RNA |
| cRNA | antigenomic virus RNA |
| hMPV | human metapneumovirus |
| APV | avian pneumovirus |
| dpi | days post-infection |
| HAI | hemagglutination inhibition |
| hpi | hours post-infection |
| POI | point of infection |
| RSV | respiratory syncytial virus |
| SFM | serum-free medium |
| TCID₅₀ | 50% tissue culture infective dose |
| position | when position is used regarding engineering any |
| | virus, it refers to the position of the gene of |
| | the viral genome to be transcribed. For example, |
| | if a gene is located at position one, it is the |
| | first gene of the viral genome to be transcribe; |
| | if a gene is located at position two, it is the |
| | second gene of the viral genome to be transcribed. |
| position 1 | nucleotide position 104 of the genome, or |
| of bPIV3, | alternatively, the position of the first gene |
| b/h PIV3 and | of the viral genome to be transcribed |
| derivatives | |
| thereof | |
| position 2 | nucleotide position 1774 of the genome, or |
| of bPIV3, | alternatively the position between the first |
| b/h PIV3 and | and the second open reading frame of the native |
| derivatives | parainfluenza virus, or alternatively, the |
| thereof | position of the second gene of the viral genome |
| | to be transcribed |
| position 3 | nucleotide position 3724 of the genome, or |
| of bPIV3, | alternatively the position between the second |
| b/h PIV3 and | and the third open reading frame of the native |
| derivatives | parainfluenza virus, or alternatively, the |
| thereof | position of the third gene of the viral genome |
| | to be transcribed. |
| position 4 | nucleotide position 5042 of the genome, or |
| of bPIV3, | alternatively the position between the third |
| b/h PIV3 and | and the fourth open reading frame of the native |
| derivatives | parainfluenza virus, or alternatively, the |
| thereof | position of the fourth gene of the viral genome |
| | to be transcribed. |
| position 5 | nucleotide position 6790 of the genome, or |
| of bPIV3, | alternatively the position between the fourth |
| b/h PIV3 and | and the fifth open reading frame of the native |
| derivatives | parainfluenza virus, or alternatively, the |
| thereof | position of the fifth gene of the viral genome |
| | to be transcribed. |
| position 6 | nucleotide position 8631 of the genome, or |
| of bPIV3, | alternatively the position between the fifth |
| b/h PIV3 and | and the sixth open reading frame of the native |
| derivatives | parainfluenza virus, or alternatively, the |
| thereof | position of the sixth gene of the viral genome |
| | to be transcribed. |

### 4. DESCRIPTION OF FIGURES

FIG. 1. Pairwise alignments of the amino acid sequence of the F protein of the human metapneumovirus with different F proteins from different avian pneumoviruses. Identical amino acids between the two sequences are indicated by the one-letter-symbol for the amino acid. Conserved amino acid exchanges between the two amino acid sequences are indicated by a “+” sign, and a space indicates a non-conserved amino acid exchange. A) Alignment of the human metapneumoviral F protein with the F protein of an avian pneumovirus isolated from Mallard Duck (85.6% identity in the ectodomain). B) Alignment of the human metapneumoviral F protein with the F protein of an avian pneumovirus isolated from Turkey (subgroup B; 75% identity in the ectodomain).

FIG. 2. PCR fragments from nt 5255 to nt 6255 derived from three different isolates of the b/h PIV3 chimeric virus were amplified. The resulting 1 kb DNA fragments were digested with enzymes specific for the F gene of human PIV3. These enzymes do not cut in the corresponding fragment of bovine PIV3. The 1% agarose gel shows the undigested fragment (lanes 2, 5, and 6) and the Sacl or BgIII digested fragments (lanes 4, 6 and lanes 9, 10, and 11, respectively). The sample in lane 10 is undigested, however, upon a repeat of digestion with BgIII, this sample was cut (data not shown). Lanes 1 and 8 show a DNA size marker.

FIG. 3. PCR fragments from nt 9075 to nt 10469 derived from three different isolates of the b/h PIV3 chimeric virus were amplified. The resulting 1.4 kb DNA fragments were digested with enzymes specific for the L gene of bovine PIV3. These enzymes do not cut in the corresponding fragment of human PIV3. The 1% agarose gel shows the undigested 1.4 kb fragment (lanes 2, 5, and 8). The smaller DNA fragments produced by digestion with BamH1 and PvuII are shown in lanes 3, 4, 6, 7, 9, and 10). Lane 1 shows a DNA size marker.

FIG. 4. Six constructs, including the bPIV3/hPIV3 vector and b/h PIV3 vectored RSV F or G cDNA, are demonstrated. The bovine PIV3 F gene and HN gene are deleted and replaced with human PIV3 F and HN gene respectively. The RSV F or G genes are cloned into either position 1 or position 2. All RSV genes are linked to the bPIV3 N-P intergenic region with the exception of RSV F1*(N-N), which is followed by the shorter bPIV3 N gene stop/N gene start sequences.

FIG. 5. b/h PIV3 vectored RSV F or G gene displayed a positional effect. (A) is a Western blot analysis of chimeric virus-infected cell lysates. F protein was detected using monoclonal antibodies (MAbs) against the RSV F protein, and G protein was detected using polyclonal antibodies (PAbs) against the RSV G protein. A 50 kDa band representing the F₁ fragment was detected in cells infected with all chimeric viruses as well as wild-type RSV. There was a greater accumulation of a 26 kDa F fragment in infected cell lysates of chimeric viruses compared to wild-type RSV. The experiment was done at MOI of 0.1, except that in lane 1, b/h PIV3 vectored RSV F1*N-N infections were repeated at a higher MOI of 1.0. Both the immature and glycosylated forms of RSV G protein that migrated at approximately 50 kDa and 90 kDa were detected. (B) is a Northern blot analysis, which showed that the mRNA transcription correlated with the result of the protein expression demonstrated in FIG. 5A. Equal amounts of total RNA were separated on 1% agarose gels containing 1% formaldehyde and transferred to nylon membranes. The blots were hybridized with digoxigenin (DIG)-UTP-labeled riboprobes synthesized by in vitro transcription using a DIG RNA labeling kit. (C)-D) are growth curves of chimeric viruses comprising b/h PIV3 vectored RSV F or G protein in Vero cells. Vero cells were grown to 90% confluence and infected at an MOI of 0.01 or 0.1. The infected monolayers were incubated at 37° C. Virus titers for each time point harvest were determined by TCID₅₀ assays, which were performed by inspecting visually for CPE following incubation at 37° C. for 6 days.

FIG. 6. The b/h PIV3 vectored enhanced green fluorescence protein (eGFP) constructs. The eGFP gene is introduced into the b/h PIV3 vector sequentially between all genes of PIV3 (only position 1, 2, 3, and 4 are shown here). The eGFP gene was linked to the bPIV3 N-P intergenic region. The b/h GFP 1 construct harbors the eGFP gene cassette in the 3′ most proximal position of the b/h PIV3 genome. The b/h GFP 2 construct contains the eGFP gene cassette between the N and P genes. The b/h GFP 3 construct contains the eGFP gene cassette between the P and M gene, and the b/h GFP4 construct contains the eGFP gene between M and F of b/h PIV3.

FIG. 7. Positional effect of enhanced green fluorescence protein (eGFP) insertions in the b/h PIV3 genome. (A) shows the amount of green cells produced upon infecting Vero cells with b/h PIV3 vectored eGFP 1, 2, and 3 at MOI 0.1 and MOI 0.01 for 20 hours. The green cells were visualized by using a fluorescent microscope. (B) is a Western blot analysis of infected cell lysates. The blots were probed with a GFP MAb as well as a PIV3 PAb. PIV3 antibody was also used to show that the blots had same volume loading. (C) is growth curves of b/h PIV3 vectored GFP constructs (at position 1, 2, and 3) in Vero cells.

FIG. 8. Constructs of b/h PIV3 vectored RSV F gene with different intergenic regions. The three constructs, RSV F1* N-N, RSV F2 N-P, and RSV F1 N-P are the same as the RSV F* (N-N), RSV F2, and RSV F1 in FIG. 4 respectively. The distance between the N gene start sequence and the N gene translation start codon in RSV F1* N-N is only 10 nucleotides (nts) long. In contrast, this distance is 86 nts long in RSV F2 construct. RSV F1* N-N also uses the N gene start sequence rather than the P gene start sequence as is done in RSV F2 construct.

FIG. 9. The length and/or nature of the intergenic region downstream of the inserted RSV gene has an effect on virus replication. (A) Western blot analysis of RSV F protein expression in chimeric viruses. Blots were probed with monoclonal antibodies against the RSV F protein. F1 protein levels expressed by RSV F1 construct and measured at 24 and 48 hours post-infection were close to the levels observed for RSV F2 construct, but much higher than those of RSV F1* N-N construct. (B) is multicycle growth curves comparing the kinetics of virus replication of RSV F1, RSV F1*N-N and RSV F2 constructs in Vero cells at an MOI of 0.1. Virus titers for each time point harvest were determined by plaque assays, which were performed by immunostaining with RSV polyclonal antisera for quantification after 5 days of incubation.

FIG. 10. Constructs of trivalent b/h PIV3 vectored RSV F and hMPV F. Two virus genomes, each comprising a chimeric b/h PIV3 vector and a first heterologous sequences derived from a metapneumovirus F gene and a second heterologous sequence derived from respiratory syncytial virus F gene, are shown here. Virus with either of the constructs has been amplified in Vero cells. The engineered virus as described can be used as a trivalent vaccine against the parainfluenza virus infection, metapneumovirus infection and the respiratory syncytial virus infection.

FIG. 11. A construct harboring two RSV F genes. This construct can be used to determine virus growth kinetics, for RSV F protein production, and replication and immunogenicity in hamsters.

FIG. 12. The chimeric b/h PIV3 vectored hMPV F constructs. The F gene of human metapneumovirus (hMPV) was inserted in position 1 or position 2 of the b/h PIV3 genome. The hMPV F gene cassette harbored the bPIV3 N-P intergenic region.

FIG. 13. Immunoprecipitation and replication assays of b/h PIV3 vectored hMPV F gene (at position 2 or position 1). (A) shows the immunoprecipitation of hMPV F protein using guinea pig or human anti-hMPV antiserum. A specific band migrating at approximately 80 kDa was observed in the lysates of b/h PIV3 vectored hMPV F2 and hMPV F1. This size corresponds to the F precursor protein, F₀. Non-specific bands of different sizes were also observed in the b/h PIV3 and mock control lanes. (B) shows growth curves that were performed to determine the kinetics of virus replication of b/h PIV3/hMPV F2 and compare it to those observed for b/h PIV3 and b/h PIV3/RSV F2 in Vero cells at an MOI of 0.1. (C)-(D) are growth curves that were performed to determine the kinetics of virus replication of b/h PIV3/hMPV F1 and compare it to those observed for b/h PIV3/hMPV F2 and b/h PIV3 in Vero cells at an MOI of 0.01 or 0.1.

FIG. 14. (A) and (B): A diagram of the viral RNA genomes of the b/h PIV3 vectored RSV F vaccine candidates. b/h PIV3/RSV F2 contained the native RSV F gene in PIV3 genome position two, while b/h PIV3/sol RSV F2 expressed a soluble RSV F lacking the trans-membrane and cytosolic domains. The removal of the trans-membrane domain and cytosolic tail of the RSV F protein was accomplished by deleting 50 amino acids at the C-terminus. The PIV3 gene stop and gene start sequences of the sol RSV F gene cassette were not altered such that the RSV F2 and sol RSV F2 gene cassettes were identical, with the exception of the 50 amino acids deletion. It was expected that the sol RSV F protein could not be incorporated into the virion envelope.

FIG. 15. Immunostained b/h PIV3/hMPV F1 and b/h PIV3/hMPV F2. (A) the b/h PIV3/hMPV F1 virus were diluted and used to infect subconfluent Vero cells. Infected cells were overlayed with optiMEM media containing gentamycin and incubated at 35° C. for 5 days. Cells were fixed and immunostained with guinea pig anti-hMPV sera. Expression of hMPV F is visualized by specific color development in the presence of the AEC substrate system. (B) the b/h PIV3/hMPV F2 virus were diluted and used to infect Vero cells. Infected cells were overlayed with 1% methyl cellulose in EMEM/L-15 medium (JRH Biosciences; Lenexa, Kans.). Cells were incubated, fixed and then immunostained with anti-hMPV guinea pig sera. The anti-hMPV guinea pig serum is specific for hMPV 001 protein.

FIG. 16. Virion fractionation of b/h PIV3 vectored RSV genes on sucrose gradients. These series experiments investigate whether the RSV proteins were incorporated into the b/h PIV3 virion. (A) shows control gradient of free RSV F (generated in baculovirus and C-terminally truncated). Majority of free RSV F was present in fractions 3, 4, 5, and 6. (B) shows that the biggest concentration of RSV virions was observed in fractions 10, 11 and 12. The RSV fractions were probed with RSV polyclonal antiserum as well as RSV F MAb. The fractions that contained the greatest amounts of RSV virions also showed the strongest signal for RSV F, suggesting that the RSV F protein co-migrated and associated with RSV virion. The last figure on (B) also shows that the fractions 10, 11 and 12 displayed the highest virus titer by plaque assay. (C) The b/h PIV3 virions may be more pleiomorphic and thus the spread of the peak fractions containing b/h PIV3 virions was more broad. (D) Sucrose gradient fractions of b/h PIV3/RSV F2 were analyzed with both a PIV polyclonal antiserum and an RSV F MAb. The fractions containing most of the virions were fractions 11, 12, 13 and 14, as shown by Western using the PIV3 antiserum. Correspondingly, these were also the fractions that displayed the highest amounts of RSV F protein. Some free RSV F was also present in fractions 5 and 6. Fractions 11, 12, 13 and 14 displayed the peak virus titers. (E) The fractions containing the most virions of b/h PIV3/RSV G2 (9, 10, 11 and 12) also showed the strongest signal for RSV G protein. Again, these were the fractions with the highest virus titers.

FIG. 17. A schematic outline of the AGM primate study design from Day −14 to Day 56. Serum was collected at the indicated time points (arrow). Initial vaccinations on Day 1 and RSV challenge administration on Day 28 are indicated.

FIG. 18. Effects of MOI on infectious virus titers. Cultures were incubated at 37±1° C. and 5±1% CO₂ post-infection.

FIG. 19. Effects of POI and post-infection temperature on infectious virus titers. Vero cultures were infected with the b/h PIV3/RSV F2 virus at MOI 0.01 at either (a) 3 days postseeding (1.1×10⁷ cells/flask), or (b) 5 days post-seeding (3.3×10⁷ cells/flask).

FIG. 20. Effects of pre-infection addition of serum on infectious virus titers. Vero cells were cultured for 3 days pre-infection in one of the following conditions: (a) OPTI PRO SFM supplemented with 4 mM glutamine, (b) OPTI PRO SFM supplemented with 4 mM glutamine and 0.5% (v/v) serum, and (c) OPTI PRO SFM supplemented with 4 mM glutamine and 2% (v/v) serum. Prior to infection, the spent culture medium was removed and the cells were rinsed with DPBS. The cultures were infected with the b/h PIV3/RSV F2 virus at MOI 0.001 and incubated at 33±1° C., 5±1% CO₂ post-infection.

FIG. 21. Expression profile of PIV-3 HN viral protein. Cells were fixed at various times post-infection (36 hpi, 60 hpi, 88 hpi, 112 hpi, 130 hpi, and 155 hpi, respectively) and were incubated with a PIV-3 HN monoclonal antibody of mouse origin followed by a fluorescence-labeled goat anti-mouse antibody.

FIG. 22. Expression profile of PIV-3 F viral protein. Cells were fixed at various times post-infection (36 hpi, 60 hpi, 88 hpi, 112 hpi, 130 hpi, and 155 hpi, respectively) and were incubated with a PIV-3 F monoclonal antibody of mouse origin followed by a fluorescence-labeled goat anti-mouse antibody.

FIG. 23. Expression profile of RSV F viral protein. Cells were fixed at various times post-infection (36 hpi, 60 hpi, 88 hpi, 112 hpi, 130 hpi, and 155 hpi, respectively) and were incubated with an RSV F monoclonal antibody of human origin followed by a fluorescence-labeled goat anti-human antibody.

FIG. 24. Effects of pre-infection addition of serum on infectious virus titers. Vero cells were cultured in duplicate sets of Roller Bottles for 3 days pre-infection in one of the following conditions: (a) OPTI PRO SFM supplemented with 4 mM glutamine, (b) OPTI PRO SFM supplemented with 4 mM glutamine and 0.5% (v/v) serum, and (c) OPTI PRO SFM supplemented with 4 mM glutamine and 2% (v/v) serum.

### 5. DESCRIPTION OF THE INVENTION

The present invention relates to recombinant parainfluenza cDNA and RNA constructs, including but not limited to, recombinant bovine and human PIV cDNA and RNA constructs, that may be used to express heterologous or non-native sequences.

In accordance with the present invention, a recombinant virus is one derived from a bovine parainfluenza virus or a human parainfluenza virus that is encoded by endogenous or native genomic sequences or non-native genomic sequences. In accordance with the invention, a non-native sequence is one that is different from the native or endogenous genomic sequence due to one or more mutations, including, but not limited to, point mutations, rearrangements, insertions, deletions, etc. to the genomic sequence that may or may not result in a phenotypic change.

In accordance with the present invention, a chimeric virus of the invention is a recombinant bPIV or hPIV which further comprises one or more heterologous nucleotide sequences. In accordance with the invention, a chimeric virus may be encoded by a nucleotide sequence in which heterologous nucleotide sequences have been added to the genome or in which nucleotide sequences have been replaced with heterologous nucleotide sequences. These recombinant and chimeric viruses and expression products may be used as vaccines suitable for administration to humans or animals. For example, the chimeric viruses of the invention may be used in vaccine formulations to confer protection against pneumovirus, respiratory syncytial virus, parainfluenza virus, or influenza virus infection.

In one embodiment, the invention relates to PIV cDNA and RNA constructs that are derived from human or bovine PIV variants and are engineered to express one, two, or three heterologous sequences, preferably heterologous genes encoding foreign antigens and other products from a variety of pathogens, cellular genes, tumor antigens, and viruses. In particular, the heterologous sequences are derived from morbillivrus or a negative strand RNA virus, including but not limited to, influenza virus, respiratory syncytial virus (RSV), mammalian metapneumovirus (e.g., human metapneumovirus variants A1, A2, B1, and B2), and avian pneumovirus subgroups A, B, C and D. The mammalian MPVs can be a variant A1, A2, B1 or B2 mammalian MPV. However, the mammalian MPVs of the present invention may encompass additional variants of MPV yet to be identified, and are not limited to variants A1, A2, B1, or B2. In another embodiment of the invention, the heterologous sequences are non-native PIV sequences, including mutated PIV sequences. In some embodiments, the heterologous sequences are derived from the same or from different viruses.

In a specific embodiment, the virus of the invention is a recombinant PIV comprising heterologous nucleotide sequences derived from human metapneumovirus or avian pneumovirus. The heterologous sequences to be inserted into the PIV genome include, but are not limited to, the sequences encoding the F, G and HN genes of human metapneumovirus variants A1, A2, B1 or B2, sequences encoding the F, G and HN genes of avian pneumovirus type A, B, C or D, and immunogenic and/or antigenic fragments thereof.

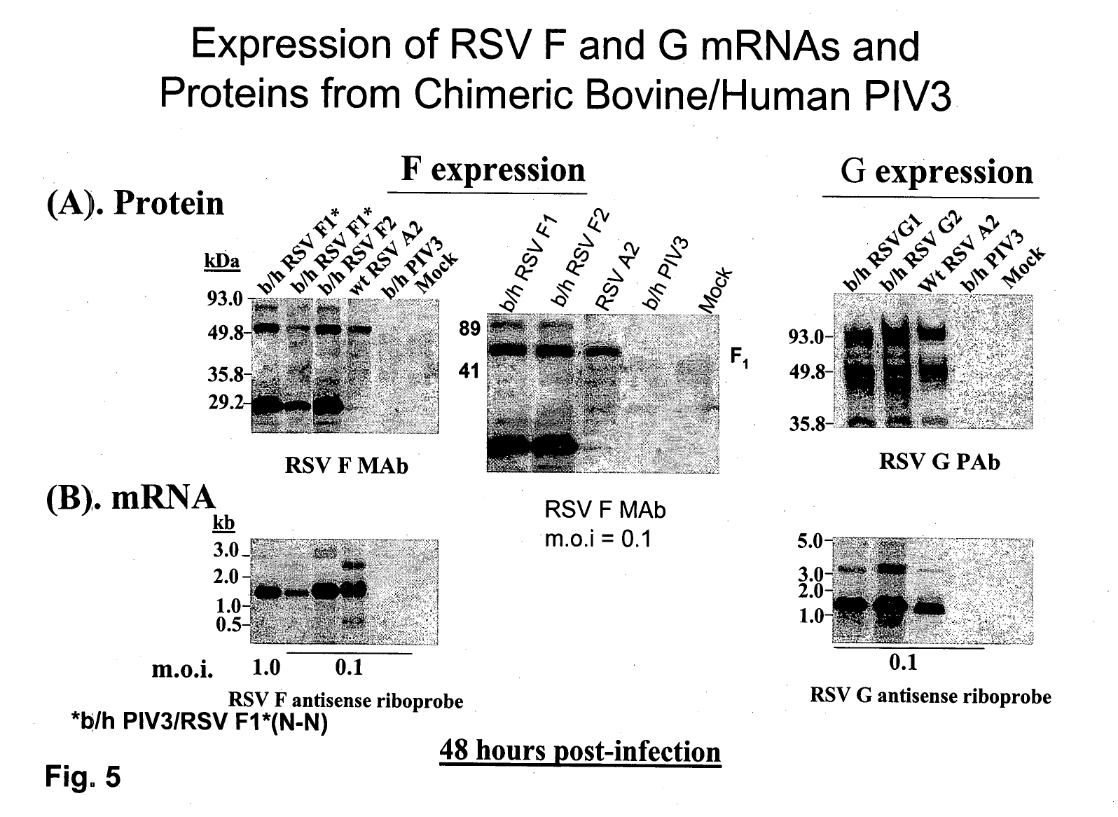

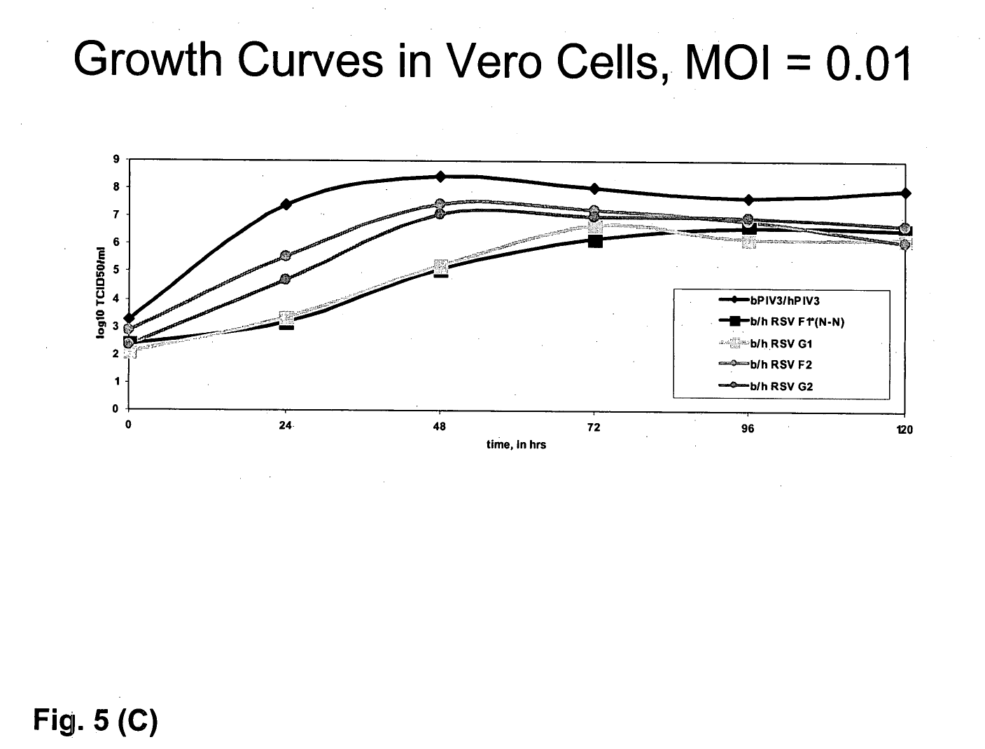

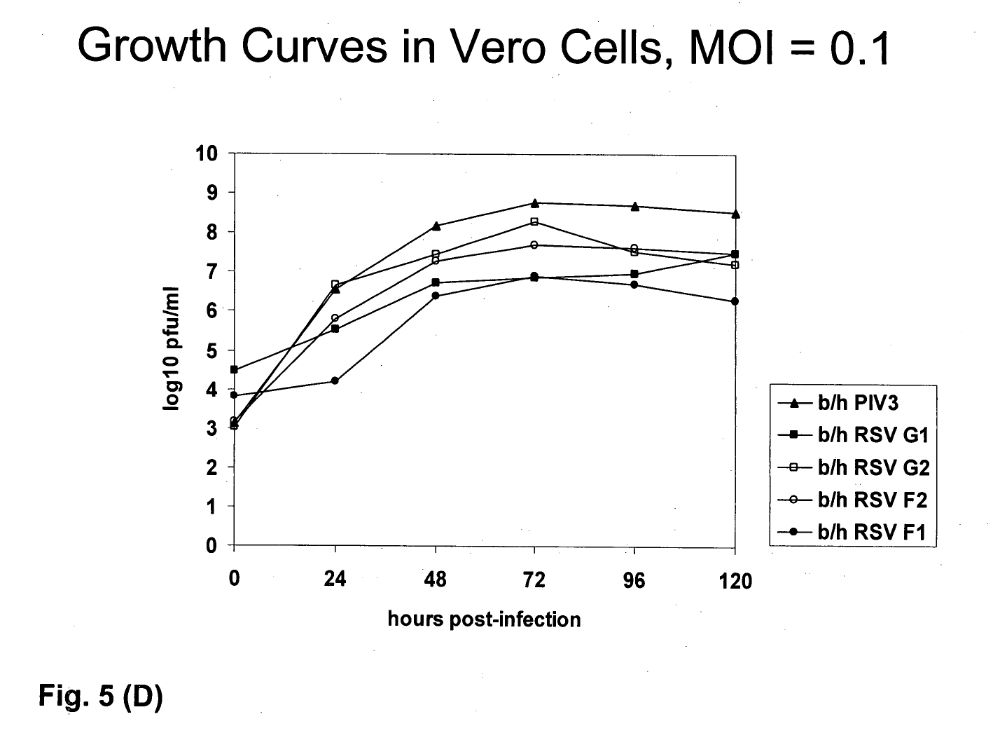

In certain embodiments, the heterologous nucleotide sequence is added to the viral genome. In alternative embodiments, the heterologous nucleotide sequence is exchanged for an endogenous nucleotide sequence. The heterologous nucleotide sequence may be added or inserted at various positions of the PIV genome, e.g., at position 1, 2, 3, 4, 5, or 6. In a preferred embodiment, the heterologous nucleotide sequence is added or inserted at position 1. In another preferred embodiment, the heterologous nucleotide sequence is added or inserted at position 2. In even another preferred embodiment, the heterologous nucleotide sequence is added or inserted at position 3. Inserting or adding heterologous nucleotide sequences at the lower-numbered positions of the virus generally results in stronger expression of the heterologous nucleotide sequence compared to insertion at higher-numbered positions. This is due to a transcriptional gradient that occurs across the genome of the virus. However, virus replication efficiency must also be considered. For example, in the b/h PIV3 chimeric virus of the invention, insertion of a heterologous gene at position 1 delays replication kinetics in vitro and to a lesser degree also in vivo (see section 8, example 3 and FIG. 5 as well as section 26, example 21). Therefore, inserting heterologous nucleotide sequences at lower-numbered positions is the preferred embodiment of the invention if strong expression of the heterologous nucleotide sequence is desired. Most preferably, a heterologous sequence is inserted at position 2 of a b/h PIV3 genome if strong expression of the heterologous sequence is desired. (See section 5.1.2. infra and section 8, example 3).

In some other embodiments, the recombinant or chimeric PIV genome is engineered such that the intergenic region between the end of the coding sequence of the heterologous gene and the start of the coding sequence of the downstream gene is altered. In yet some other embodiments, the virus of the invention comprises a recombinant or chimeric PIV genome engineered such that the heterologous nucleotide sequence is inserted at a position selected from the group consisting of positions 1, 2, 3, 4, 5, and 6, and the intergenic region between the heterologous nucleotide sequence and the next downstream gene is altered. Appropriate assays may be used to determine the best mode of insertion (i.e., which position to insert, and the length of the intergenic region) to achieve appropriate levels of gene expression and viral growth characteristics. For detail, see Section 5.1.2., infra.

In certain embodiments, the chimeric virus of the invention contains two different heterologous nucleotide sequences. The different heterologous nucleotide sequences may be inserted at various positions of the PIV genome. In a preferred embodiment, one heterologous nucleotide sequence is inserted at position 1 and another heterologous nucleotide sequence is added or inserted at position 2 or 3. In other embodiments of the invention, additional heterologous nucleotide sequences are inserted at higher-numbered positions of the PIV genome. In accordance with the present invention, the position of the heterologous sequence refers to the order in which the sequences are transcribed from the viral genome, e.g., a heterologous sequence at position 1 is the first gene sequence to be transcribed from the genome.

In certain embodiments of the invention, the heterologous nucleotide sequence to be inserted into the genome of the virus of the invention is derived from a negative strand RNA virus, including but not limited to, influenza virus, parainfluenza virus, respiratory syncytial virus, mammalian metapneumovirus, and avian pneumovirus. In a specific embodiment of the invention, the heterologous nucleotide sequence is derived from a human metapneumovirus. In another specific embodiment, the heterologous nucleotide sequence is derived from an avian pneumovirus. More specifically, the heterologous nucleotide sequence of the invention encodes a F, G or SH gene or a portion thereof of a human or avian metapneumovirus. In specific embodiments, a heterologous nucleotide sequences can be any one of SEQ ID NO:1 through SEQ ID NO:5, SEQ ID NO:14, and SEQ ID NO:15 (see Table 16). In certain specific embodiments, the nucleotide sequence encodes a protein of any one of SEQ ID NO:6 through SEQ ID NO:13, SEQ ID NO:16, and SEQ ID NO:17 (see Table 16). In certain specific embodiments, the nucleotide sequence encodes a protein of any one of SEQ ID NO: 314 through 389.

In specific embodiments of the invention, a heterologous nucleotide sequence of the invention is derived from a type A avian pneumovirus. In other specific embodiments of the invention, a heterologous nucleotide sequence of the invention is derived from a type B avian pneumovirus. In even other specific embodiments of the invention, a heterologous nucleotide sequence of the invention is derived from a type C avian pneumovirus. Phylogenetic analyses show that type A and type B are more closely related to each other than they are to type C (Seal, 2000, Animal Health Res. Rev. 1(1):67-72). Type A and type B are found in Europe whereas type C was first isolated in the U.S.

In another embodiment of the invention, the heterologous nucleotide sequence encodes a chimeric polypeptide, wherein the ectodomain contains antigenic sequences derived from a virus other than the strain of PIV from which the vector backbone is derived, and the trans membrane and luminal domains are derived from PIV sequences. The resulting chimeric virus would impart antigenicity of the negative strand RNA virus of choice and would have an attenuated phenotype.

In a specific embodiment of the invention, the heterologous nucleotide sequence encodes a chimeric F protein. Particularly, the ectodomain of the chimeric F protein is the ectodomain of a metapneumovirus, so that a human metapneumovirus or avian pneumovirus, and the transmembrane domain as well as the luminal domain are the transmembrane and luminal domains of a parainfluenza virus, such as a human or a bovine parainfluenza virus. While not bound by any theory, insertion of a chimeric F protein may further attenuate the virus in an intended host but retain the antigenicity of the F protein attributed by its ectodomain.

The chimeric viruses of the invention may be used in vaccine formulations to confer protection against various infections, including but not limited to, pneumovirus infection, respiratory syncytial virus infection, parainfluenza virus infection, influenza virus infection, or a combination thereof. The present invention provides vaccine preparations comprising chimeric PIV expressing one or more heterologous antigenic sequences, including bivalent and trivalent vaccines. The bivalent and trivalent vaccines of the invention may be administered in the form of one PIV vector expressing each heterologous antigenic sequences or two or more PIV vectors each encoding different heterologous antigenic sequences. Preferably, the heterologous antigenic sequences are derived from a negative strand RNA virus, including but not limited to, influenza virus, parainfluenza virus, respiratory syncytial virus (RSV), mammalian metapneumovirus (e.g., human metapneumovirus) and avian pneumovirus. Thus, the chimeric virions of the present invention may be engineered to create, e.g., anti-human influenza vaccine, anti-human parainfluenza vaccine, anti-human RSV vaccine, and anti-human metapneumovirus vaccine. Preferably, the vaccine preparation of the invention comprises attenuated chimeric viruses that are viable and infectious. The vaccine preparation of the invention can be administered alone or in combination with other vaccines or other prophylactic or therapeutic agents.

The present invention also relates to the use of viral vectors and chimeric viruses to formulate vaccines against a broad range of viruses and/or antigens including tumor antigens. The viral vectors and chimeric viruses of the present invention may be used to modulate a subject's immune system by stimulating a humoral immune response, a cellular immune response or by stimulating tolerance to an antigen. As used herein, a subject refers to a human, a primate, a horse, a cow, a sheep, a pig, a goat, a dog, a cat, a rodent and a member of avian species. When delivering tumor antigens, the invention may be used to treat subjects having disease amenable to immune response mediated rejection, such as non-solid tumors or solid tumors of small size. It is also contemplated that delivery of tumor antigens by the viral vectors and chimeric viruses described herein will be useful for treatment subsequent to removal of large solid tumors. The invention may also be used to treat subjects who are suspected of having cancer.

The invention may be divided into the following stages solely for the purpose of description and not by way of limitation: (a) construction of recombinant cDNA and RNA templates; (b) expression of heterologous gene products using recombinant cDNA and RNA templates; and (c) rescue of the heterologous genes in recombinant virus particles.

5.1. Construction of the Recombinant cDNA and RNA

The present invention encompasses recombinant or chimeric viruses encoded by viral vectors derived from the genomes of parainfluenza virus, including both bovine parainfluenza virus and mammalian parainfluenza virus. In accordance with the present invention, a recombinant virus is one derived from a bovine parainfluenza virus or a mammalian parainfluenza virus that is encoded by endogenous or native genomic sequences or non-native genomic sequences. In accordance with the invention, a non-native sequence is one that is different from the native or endogenous genomic sequence due to one or more mutations, including, but not limited to, point mutations, rearrangements, insertions, deletions etc. to the genomic sequence that may or may not result a phenotypic change. The recombinant viruses of the invention encompass those viruses encoded by viral vectors derived from the genomes of parainfluenza virus, including both bovine and mammalian parainfluenza virus, and may or may not, include nucleic acids that are non-native to the viral genome. In accordance with the present invention, a viral vector which is derived from the genome of a parainfluenza virus is one that contains a nucleic acid sequence that encodes at least a part of one ORF of a parainfluenza virus.

The present invention also encompasses recombinant viruses comprising a viral vector derived from a bovine and/or mammalian PIV genome which contains sequences which result in a virus having a phenotype more suitable for use in vaccine formulations, e.g., attenuated phenotype or enhanced antigenicity. The mutations and modifications can be in coding regions, in intergenic regions and in the leader and trailer sequences of the virus.

In accordance with the present invention, the viral vectors of the invention are derived from the genome of a mammalian parainfluenza virus, in particular a human parainfluenza virus (hPIV). In particular embodiments of the invention, the viral vector is derived from the genome of a human parainfluenza virus type 3. In accordance with the present invention, these viral vectors may or may not include nucleic acids that are non-native to the viral genome.

In accordance with the present invention, the viral vectors of the inventions are derived from the genome of a bovine parainfluenza virus (bPIV). In particular embodiments of the invention, the viral vector is derived from the genome of bovine parainfluenza virus type 3. In accordance to the present invention, these viral vectors may or may include nucleic acids that are non-native to the viral genome.

In accordance with the invention, a chimeric virus is a recombinant bPIV or hPIV which further comprises a heterologous nucleotide sequence. In accordance with the invention, a chimeric virus may be encoded by a nucleotide sequence in which heterologous nucleotide sequence have been added to the genome or in which endogenous or native nucleotide sequence have been replaced with heterologous nucleotide sequence. In accordance with the invention, the chimeric viruses are encoded by the viral vectors of the invention which further comprise a heterologous nucleotide sequence. In accordance with the present invention, a chimeric virus is encoded by a viral vector that may or may not include nucleic acids that are non-native to the viral genome. In accordance with the invention, a chimeric virus is encoded by a viral vector to which heterologous nucleotide sequences have been added, inserted or substituted for native or non-native sequences.

A chimeric virus may be of particular use for the generation of recombinant vaccines protecting against two or more viruses (Tao et al., J. Virol. 72, 2955-2961; Durbin et al., 2000, J. Virol. 74, 6821-6831; Skiadopoulos et al., 1998, J. Virol. 72, 1762-1768 (1998); Teng et al., 2000, J. Virol. 74, 9317-9321). For example, it can be envisaged that a hPIV or bPIV virus vector expressing one or more proteins of another negative strand RNA virus, e.g., MPV, or a RSV vector expressing one or more proteins of MPV will protect individuals vaccinated with such vector against both virus infections. A similar approach can be envisaged for other paramyxoviruses. Attenuated and replication-defective viruses may be of use for vaccination purposes with live vaccines as has been suggested for other viruses. (See, PCT WO 02/057302, at pp. 6 and 23, incorporated by reference herein).

In accordance with the present invention the heterologous to be incorporated into the viral vectors encoding the recombinant or chimeric viruses of the invention include sequences obtained or derived from different strains of metapneumovirus, strains of avian pneumovirus, and other negative strand RNA viruses, including, but not limited to, RSV, PIV, influenza virus and other viruses, including morbillivirus.

In certain embodiments of the invention, the chimeric or recombinant viruses of the invention are encoded by viral vectors derived from viral genomes wherein one or more sequences, intergenic regions, termini sequences, or portions or entire ORF have been substituted with a heterologous or non-native sequence. In certain embodiments of the invention, the chimeric viruses of the invention are encoded by viral vectors derived from viral genomes wherein one or more heterologous sequences have been added to the vector.

A specific embodiment of the present invention is a chimeric virus comprising a backbone encoded by nucleotide sequences derived from a parainfluenza virus genome. In a preferred embodiment, the PIV genome is derived from bovine PIV, such as the Kansas strain of bPIV3, or from human PIV. In a preferred embodiment, the PIV genome is derived from the Kansas strain of bPIV3, in which bovine parainfluenza virus nucleotide sequences have been substituted with heterologous sequences or in which heterologous sequences have been added to the complete bPIV genome. A further specific embodiment of the present invention is a chimeric virus comprising a backbone encoded by nucleotide sequences derived from human parainfluenza virus type 3 genome, in which human parainfluenza virus nucleotide sequences have been substituted with heterologous sequences or in which heterologous sequences have been added to the complete hPIV genome. An additional specific embodiment of the present invention is a chimeric virus comprising a backbone encoded by nucleotide sequences derived from bovine parainfluenza virus genome, such as the Kansas strain of bPIV3, in which (a) the bovine parainfluenza virus F gene and HN gene have been substituted with the F gene and the HN gene of the human parainfluenza virus (bPIV/hPIV), and in which (b) heterologous sequences have been added to the complete bPIV genome.

The present invention also encompasses chimeric viruses comprising a backbone encoded by nucleotide sequences derived from the bPIV, the hPIV, or the bPIV/hPIV genome containing mutations or modifications, in addition to heterologous sequences, that result in a chimeric virus having a phenotype more suitable for use in vaccine formulations, e.g., attenuated phenotype or enhanced antigenicity. In accordance with this particular embodiment of the invention, a heterologous sequence in the context of a bovine PIV3 backbone may be any sequence heterologous to bPIV3.

Another specific embodiment of the present invention is a chimeric virus comprising a backbone encoded by nucleotide sequences derived from human PIV 1, 2, or 3 in which hPIV nucleotide sequences have been substituted with heterologous sequences or in which heterologous sequences have been added to the complete hPIV genome, with the proviso that the resulting chimeric virus is not a chimeric hPIV3 in which the hemagglutinin-neuraminidase and fusion glycoproteins have been replaced by those of hPIV1. The present invention also encompasses chimeric viruses, comprising a backbone encoded by nucleotide sequences derived from a hPIV genome, containing mutations or modifications, in addition to heterologous sequences, that result in a chimeric virus having a phenotype more suitable for use in vaccine formulations, e.g., attenuated phenotype or enhanced antigenicity.

Heterologous gene coding sequences flanked by the complement of the viral polymerase binding site/promoter, e.g., the complement of 3′-PIV virus terminus of the present invention, or the complements of both the 3′- and 5′-PIV virus termini may be constructed using techniques known in the art. The resulting RNA templates may be of the negative-polarity and can contain appropriate terminal sequences that enable the viral RNA-synthesizing apparatus to recognize the template. Alternatively, positive-polarity RNA templates, that contain appropriate terminal sequences which enable the viral RNA-synthesizing apparatus to recognize the template, may also be used. Recombinant DNA molecules containing these hybrid sequences can be cloned and transcribed by a DNA-directed RNA polymerase, such as bacteriophage T7 polymerase, T3 polymerase, the SP6 polymerase or a eukaryotic polymerase such as polymerase I and the like, for the in vitro or in vivo production of recombinant RNA templates that possess the appropriate viral sequences and that allow for viral polymerase recognition and activity.

In one embodiment, the PIV vector of the invention expresses one, two, or three heterologous sequences, encoding antigenic polypeptides and peptides. In some embodiments, the heterologous sequences are derived from the same virus or from different viruses. In certain embodiments, more than one copy of the same heterologous nucleotide sequences are inserted in the genome of a bovine parainfluenza virus, human parainfluenza virus, or bPIV/hPIV chimeric vector. In a preferred embodiment, two copies of the same heterologous nucleotide sequences are inserted to the genome of the virus of the invention. In some embodiments, the heterologous nucleotide sequence is derived from a metapneumovirus, such as human metapneumovirus or an avian pneumovirus. In specific embodiments, the heterologous nucleotide sequence derived from a metapneumovirus is a F gene of the metapneumovirus. In other specific embodiments, the heterologous nucleotide sequence derived from a metapneumovirus is a G gene of the metapneumovirus. In some other embodiments, the heterologous nucleotide sequence is derived from a respiratory syncytial virus. In specific embodiments, the heterologous nucleotide sequence derived from respiratory syncytial virus is a F gene of the respiratory syncytial virus. In other specific embodiments, the heterologous nucleotide sequence derived from respiratory syncytial virus is a G gene of the respiratory syncytial virus. When one or more heterologous nucleotide sequences are inserted, the position of the insertion and the length of the intergenic region of each inserted copy can be manipulated and determined by different assays according to section 5.1.2. infra.

In certain embodiments, rescue of the chimeric virus or expression products may be achieved by reverse genetics in host cell systems where the host cells are transfected with chimeric cDNA or RNA constructs. The RNA templates of the present invention are prepared by transcription of appropriate DNA sequences with a DNA-directed RNA polymerase. The RNA templates of the present invention may be prepared either in vitro or in vivo by transcription of appropriate DNA sequences using a DNA-directed RNA polymerase such as bacteriophage T7 polymerase, T3 polymerase, the SP6 polymerase, or a eukaryotic polymerase such as polymerase I. In certain embodiments, the RNA templates of the present invention may be prepared either in vitro or in vivo by transcription of appropriate DNA sequences using a plasmid-based expression system as described in Hoffmann et al., 2000, Proc. Natl. Acad. Sci. USA 97: 6108-6113 or the unidirectional RNA polymerase I-polymerase II transcription system as described in Hoffmann and Webster, 2000, J. Gen. Virol. 81: 2843-2847. The resulting RNA templates of negative-polarity would contain appropriate terminal sequences that would enable the viral RNA-synthesizing apparatus to recognize the template. Alternatively, positive-polarity RNA templates that contain appropriate terminal sequences and enable the viral RNA-synthesizing apparatus to recognize the template may also be used. Expression from positive polarity RNA templates may be achieved by transfection of plasmids having promoters that are recognized by the DNA-dependent RNA polymerase. For example, plasmid DNA, encoding positive RNA templates under the control of a T7 promoter, can be used in combination with the vaccinia virus or fowlpox T7 system.

Bicistronic mRNAs can be constructed to permit internal initiation of translation of viral sequences and to allow for the expression of foreign protein coding sequences from the regular terminal initiation site, or vice versa. Alternatively, a foreign protein may be expressed from an internal transcriptional unit in which the transcriptional unit has an initiation site and polyadenylation site. In another embodiment, the foreign gene is inserted into a PIV gene such that the resulting expressed protein is a fusion protein.

In certain embodiments, the invention relates to trivalent vaccines comprising a virus of the invention. In specific embodiments, the virus used for a trivalent vaccine is a chimeric bovine parainfluenza type 3/human parainfluenza type3 virus containing a first heterologous nucleotide sequence derived from respiratory syncytial virus, and a second heterologous nucleotide sequence derived from a metapneumovirus, such as human metapneumovirus or avian pneumovirus. In an exemplary embodiment, such a trivalent vaccine would be specific to (a) the gene products of the F gene and the HN gene of the human parainfluenza virus; (b) the protein encoded by the heterologous nucleotide sequence derived from a respiratory syncytial virus; and (c) the protein encoded by the heterologous nucleotide sequence derived from a metapneumovirus. In a preferred embodiment, the first heterologous nucleotide sequence is the F gene of the respiratory syncytial virus and is inserted in position 1, and the second heterologous nucleotide sequence is the F gene of the human metapneumovirus and is inserted in position 3. Many more combinations are encompassed by the present invention and some are shown by way of example in Table 1. For other combinations the F or G gene of an avian pneumovirus could be used. Further, nucleotide sequences encoding chimeric F proteins could be used (see supra). In some less preferred embodiments, the heterologous nucleotide sequence can be inserted at higher-numbered positions of the viral genome.

TABLE 1
Exemplary arrangements of heterologous nucleotide sequences
in the viruses used for trivalent vaccines.

| Combination | Position 1 | Position 2 | Position 3 |
|---|---|---|---|
| 1 | F-gene of hMPV | F-gene of RSV | — |
| 2 | F-gene of RSV | F-gene of hMPV | — |
| 3 | — | F-gene of hMPV | F-gene of RSV |
| 4 | — | F-gene of RSV | F-gene of hMPV |
| 5 | F-gene of hMPV | — | F-gene of RSV |
| 6 | F-gene of RSV | — | F-gene of hMPV |
| 7 | G-gene of hMPV | G-gene of RSV | — |
| 8 | G-gene of RSV | G-gene of hMPV | — |
| 9 | — | G-gene of hMPV | G-gene of RSV |
| 10 | — | G-gene of RSV | G-gene of hMPV |
| 11 | G-gene of hMPV | — | G-gene of RSV |
| 12 | G-gene of RSV | — | G-gene of hMPV |
| 13 | F-gene of hMPV | G-gene of RSV | — |
| 14 | G-gene of RSV | F-gene of hMPV | — |
| 15 | — | F-gene of hMPV | G-gene of RSV |
| 16 | — | G-gene of RSV | F-gene of hMPV |
| 17 | F-gene of hMPV | — | G-gene of RSV |
| 18 | G-gene of RSV | — | F-gene of hMPV |
| 19 | G-gene of hMPV | F-gene of RSV | — |
| 20 | F-gene of RSV | G-gene of hMPV | — |
| 21 | — | G-gene of hMPV | F-gene of RSV |
| 22 | — | F-gene of RSV | G-gene of hMPV |
| 23 | G-gene of hMPV | — | F-gene of RSV |
| 24 | F-gene of RSV | — | G-gene of hMPV |

In some other embodiments, the intergenic region between a heterologous sequence and the start of the coding sequence of the downstream gene can be altered. For example, each gene listed on Table 1 may have a desirable length of the intergenic region. In an exemplary embodiment, a trivalent vaccine comprises a b/h PIV3 vector with a F gene of respiratory syncytial virus inserted at position 1, an altered intergenic region of 177 nucleotides (originally 75 nucleotides to the downstream N gene start codon AUG), and a F gene of human metapneumovirus inserted at position 3 with its natural intergenic region. Many more combinations are encompassed by the present invention, as each insertion of a heterologous nucleotide sequence may be manipulated according to section 5.1.2., infra.

In a broader embodiment, the expression products and chimeric virions of the present invention may be engineered to create vaccines against a broad range of pathogens, including viral antigens, tumor antigens and auto antigens involved in autoimmune disorders. One way to achieve this goal involves modifying existing PIV genes to contain foreign sequences in their respective external domains. Where the heterologous sequences are epitopes or antigens of pathogens, these chimeric viruses may be used to induce a protective immune response against the disease agent from which these determinants are derived.

One approach for constructing these hybrid molecules is to insert the heterologous nucleotide sequence into a DNA complement of a PIV genome, e.g., a hPIV, a bPIV, or a bPIV/hPIV, so that the heterologous sequence is flanked by the viral sequences required for viral polymerase activity; i.e., the viral polymerase binding site/promoter, hereinafter referred to as the viral polymerase binding site, and a polyadenylation site. In a preferred embodiment, the heterologous coding sequence is flanked by the viral sequences that comprise the replication promoters of the 5′ and 3′ termini, the gene start and gene end sequences, and the packaging signals that are found in the 5′ and/or the 3′ termini. In an alternative approach, oligonucleotides encoding the viral polymerase binding site, e.g., the complement of the 3′-terminus or both termini of the virus genomic segment can be ligated to the heterologous coding sequence to construct the hybrid molecule. The placement of a foreign gene or segment of a foreign gene within a target sequence was formerly dictated by the presence of appropriate restriction enzyme sites within the target sequence. However, recent advances in molecular biology have lessened this problem greatly. Restriction enzyme sites can readily be placed anywhere within a target sequence through the use of site-directed mutagenesis (e.g., see, for example, the techniques described by Kunkel, 1985, Proc. Natl. Acad. Sci. U.S.A. 82; 488). Variations in polymerase chain reaction (PCR) technology, described infra, also allow for the specific insertion of sequences (i.e., restriction enzyme sites) and also allow for the facile construction of hybrid molecules. Alternatively, PCR reactions could be used to prepare recombinant templates without the need of cloning. For example, PCR reactions could be used to prepare double-stranded DNA molecules containing a DNA-directed RNA polymerase promoter (e.g., bacteriophage T3, T7 or SP6) and the hybrid sequence containing the heterologous gene and the PIV polymerase binding site. RNA templates could then be transcribed directly from this recombinant DNA. In yet another embodiment, the recombinant RNA templates may be prepared by ligating RNAs specifying the negative polarity of the heterologous gene and the viral polymerase binding site using an RNA ligase.

In addition, one or more nucleotides can be added at the 3′ end of the HN gene in the untranslated region to adhere to the “Rule of Six” which may be important in successful virus rescue. The “Rule of Six” applies to many paramyxoviruses and requires that the number of nucleotides of an RNA genome be a factor of six to be functional. The addition of nucleotides can be accomplished by techniques known in the art such as using a commercial mutagenesis kits like the QuikChange mutagenesis kit (Stratagene). After addition of the appropriate number of nucleotides, the correct DNA fragment, for example, a DNA fragment of the hPIV3 F and HN gene, can then be isolated upon digestion with the appropriate restriction enzyme and gel purification. Sequence requirements for viral polymerase activity and constructs that may be used in accordance with the invention are described in the subsections below.

Without being bound by theory, several parameters affect the rate of replication of the recombinant virus and the level of expression of the heterologous sequence. In particular, the position of the heterologous sequence in bPIV, hPIV, b/h PIV and the length of the intergenic region that flanks the heterologous sequence determine rate of replication and expression level of the heterologous sequence.

In certain embodiments, the leader and or trailer sequence of the virus are modified relative to the wild type virus. In certain more specific embodiments, the lengths of the leader and/or trailer are altered. In other embodiments, the sequence(s) of the leader and/or trailer are mutated relative to the wild type virus.

The production of a recombinant virus of the invention relies on the replication of a partial or full-length copy of the negative sense viral RNA (vRNA) genome or a complementary copy thereof (cRNA). This vRNA or cRNA can be isolated from infectious virus, produced upon in-vitro transcription, or produced in cells upon transfection of nucleic acids. Second, the production of recombinant negative strand virus relies on a functional polymerase complex. Typically, the polymerase complex of pneumoviruses consists of N, P, L and possibly M2 proteins, but is not necessarily limited thereto.

Polymerase complexes or components thereof can be isolated from virus particles, isolated from cells expressing one or more of the components, or produced upon transfection of specific expression vectors.

Infectious copies of MPV can be obtained when the above mentioned vRNA, cRNA, or vectors expressing these RNAs are replicated by the above mentioned polymerase complex 16 (Schnell et al., 1994, EMBO J. 13: 4195-4203; Collins et al., 1995, PNAS 92: 11563-11567; Hoffmann et al., 2000, PNAS 97: 6108-6113; Bridgen et al., 1996, PNAS 93: 15400-15404; Palese et al., 1996, PNAS 93: 11354-11358; Peeters et al., 1999, J. Virol. 73: 5001-5009; Durbin et al., 1997, Virology 235: 323-332).

The invention provides a host cell comprising a nucleic acid or a vector according to the invention. Plasmid or viral vectors containing the polymerase components of PIV (presumably N, P, L and M2, but not necessarily limited thereto) are generated in prokaryotic cells for the expression of the components in relevant cell types (bacteria, insect cells, eukaryotic cells). Plasmid or viral vectors containing full-length or partial copies of the PIV genome will be generated in prokaryotic cells for the expression of viral nucleic acids in vitro or in vivo. The latter vectors may contain other viral sequences for the generation of chimeric viruses or chimeric virus proteins, may lack parts of the viral genome for the generation of replication defective virus, and may contain mutations, deletions or insertions for the generation of attenuated viruses.

Infectious copies of PIV (being wild type, attenuated, replication-defective or chimeric) can be produced upon co-expression of the polymerase components according to the state-of-the-art technologies described above.

In addition, eukaryotic cells, transiently or stably expressing one or more full-length or partial PIV proteins can be used. Such cells can be made by transfection (proteins or nucleic acid vectors), infection (viral vectors) or transduction (viral vectors) and may be useful for complementation of mentioned wild type, attenuated, replication-defective or chimeric viruses.

5.1.1. Heterologous Gene Sequences to be Inserted

The present invention encompass engineering recombinant bovine or human parainfluenza viruses to express one or more heterologous sequences, wherein the heterologous sequences encode gene products or fragments of gene products that are preferably antigenic and/or immunogenic. As used herein, the term “antigenic” refers to the ability of a molecule to bind antibody or MHC molecules. The term “immunogenic” refers to the ability of a molecule to generate immune response in a host.

In a preferred embodiment, the heterologous nucleotide sequence to be inserted is derived from a negative strand RNA virus, including but not limited to, influenza virus, parainfluenza virus, respiratory syncytial virus, mammalian metapneumovirus (e.g., human metapneumovirus) and avian pneumovirus. In a preferred embodiment, the heterologous sequence to be inserted includes, but is not limited to, a sequence that encodes a F or HN gene of human PIV, a F gene of RSV, a HA gene of influenza virus type A, B, or C, a F gene of human MPV, a F gene of avian pneumovirus, or an immunogenic and/or antigenic fragment thereof.

In some embodiments, the heterologous nucleotide sequence to be inserted is derived from a human metapneumovirus and/or an avian pneumovirus. In certain embodiments, the heterologous nucleotide sequence to be inserted is derived from (a) a human metapneumovirus and a respiratory syncytial virus; and/or (b) an avian pneumovirus and a respiratory syncytial virus.

In certain preferred embodiments of the invention, the heterologous nucleotide sequence to be inserted is derived from a F gene from a human metapneumovirus and/or an avian pneumovirus. In certain embodiments, the F gene is derived from (a) a human metapneumovirus and a respiratory syncytial virus; and/or (b) an avian pneumovirus and a respiratory syncytial virus.

In certain embodiments of the invention, the heterologous nucleotide sequence to be inserted is a G gene derived from a human metapneumovirus and/or an avian pneumovirus. In certain embodiments, the G gene is derived from (a) a human metapneumovirus and a respiratory syncytial virus; and/or (b) an avian pneumovirus and a respiratory syncytial virus.

In certain embodiments, any combination of different F genes and/or different G genes derived from human metapneumovirus, avian pneumovirus, and respiratory syncytial virus can be inserted into the virus of the invention with the proviso that in all embodiments at least one heterologous sequence derived from either human metapneumovirus or avian pneumovirus is present in the recombinant parainfluenza virus of the invention.

In certain embodiments, the nucleotide sequence to be inserted is a nucleotide sequence encoding a F protein derived from a human metapneumovirus. In certain other embodiments, the nucleotide sequence to be inserted is a nucleotide sequence encoding a G protein derived from a human metapneumovirus. In yet other embodiments, the nucleotide sequence to be inserted is a nucleotide sequence encoding a F protein derived from an avian pneumovirus. In yet other embodiments, the nucleotide sequence to be inserted is a nucleotide sequence encoding a G protein derived from an avian pneumovirus. With the proviso that in all embodiments of the invention at least one heterologous nucleotide sequence is derived from a metapneumovirus, the heterologous nucleotide sequence to be inserted encodes a F protein or a G protein of a respiratory syncytial virus.

In certain embodiments, the nucleotide sequence to be inserted encodes a chimeric F protein or a chimeric G protein. A chimeric F protein comprises parts of F proteins from different viruses, such as a human metapneumovirus, avian pneumovirus and/or respiratory syncytial virus. A chimeric G protein comprises parts of G proteins from different viruses, such as a human metapneumovirus, avian pneumovirus and/or respiratory syncytial virus. In a specific embodiment, the F protein comprises an ectodomain of a F protein of a metapneumovirus, a transmembrane domain of a F protein of a parainfluenza virus, and luminal domain of a F protein of a parainfluenza virus. In certain embodiments, the nucleic acid to be inserted encodes a F protein, wherein the transmembrane domain of the F protein is deleted so that a soluble F protein is expressed.

In certain specific embodiments, the heterologous nucleotide sequence of the invention is any one of SEQ ID NO:1 through SEQ ID NO:5, SEQ ID NO:14, and SEQ ID NO:15 (see Table 16). In certain specific embodiments, the nucleotide sequence encodes a protein of any one of SEQ ID NO:6 through SEQ ID NO:13, SEQ ID NO:16, and SEQ ID NO:17 (see Table 16). In certain specific embodiments, the nucleotide sequence encodes a protein of any one of SEQ ID NO. 314 to 389.

For heterologous nucleotide sequences derived from respiratory syncytial virus see, e.g., PCT/US98/20230, which is hereby incorporated by reference in its entirety.

In a preferred embodiment, heterologous gene sequences that can be expressed into the chimeric viruses of the invention include but are not limited to those encoding antigenic epitopes and glycoproteins of viruses, such as influenza glycoproteins, in particular hemagglutinin H5, H7, respiratory syncytial virus epitopes, New Castle Disease virus epitopes, Sendai virus and infectious Laryngotracheitis virus (ILV), that result in respiratory disease. In a most preferred embodiment, the heterologous nucleotide sequences are derived from a metapneumovirus, such as human metapneumovirus and/or avian pneumovirus. In yet another embodiment of the invention, heterologous gene sequences that can be engineered into the chimeric viruses of the invention include, but are not limited to, those encoding viral epitopes and glycoproteins of viruses, such as hepatitis B virus surface antigen, hepatitis A or C virus surface glycoproteins of Epstein Barr virus, glycoproteins of human papilloma virus, simian virus 5 or mumps virus, West Nile virus, Dengue virus, glycoproteins of herpesviruses, VPI of poliovirus, and sequences derived from a human immunodeficiency virus (HIV), preferably type 1 or type 2. In yet another embodiment, heterologous gene sequences that can be engineered into chimeric viruses of the invention include, but are not limited to, those encoding Marek's Disease virus (MDV) epitopes, epitopes of infectious Bursal Disease virus (IBDV), epitopes of Chicken Anemia virus, infectious laryngotracheitis virus (ILV), Avian Influenza virus (AIV), rabies, feline leukemia virus, canine distemper virus, vesicular stomatitis virus, and swinepox virus (see Fields et al. (ed.), 1991, FUNDAMENTAL VIROLOGY, Second Edition, Raven Press, New York, incorporated by reference herein in its entirety).

Other heterologous sequences of the present invention include those encoding antigens that are characteristic of autoimmune diseases. These antigens will typically be derived from the cell surface, cytoplasm, nucleus, mitochondria and the like of mammalian tissues, including antigens characteristic of diabetes mellitus, multiple sclerosis, systemic lupus erythematosus, rheumatoid arthritis, pernicious anemia, Addison's disease, scleroderma, autoimmune atrophic gastritis, juvenile diabetes, and discold lupus erythromatosus.

Antigens that are allergens generally include proteins or glycoproteins, including antigens derived from pollens, dust, molds, spores, dander, insects and foods. In addition, antigens that are characteristic of tumor antigens typically will be derived from the cell surface, cytoplasm, nucleus, organelles and the like of cells of tumor tissue. Examples include antigens characteristic of tumor proteins, including proteins encoded by mutated oncogenes; viral proteins associated with tumors; and glycoproteins. Tumors include, but are not limited to, those derived from the types of cancer: lip, nasopharynx, pharynx and oral cavity, esophagus, stomach, colon, rectum, liver, gall bladder, pancreas, larynx, lung and bronchus, melanoma of skin, breast, cervix, uterine, ovary, bladder, kidney, uterus, brain and other parts of the nervous system, thyroid, prostate, testes, Hodgkin's disease, non-Hodgkin's lymphoma, multiple myeloma and leukemia.

In one specific embodiment of the invention, the heterologous sequences are derived from the genome of human immunodeficiency virus (HIV), preferably human immunodeficiency virus-1 or human immunodeficiency virus-2. In another embodiment of the invention, the heterologous coding sequences may be inserted within a PIV gene coding sequence such that a chimeric gene product, that contains the heterologous peptide sequence within the PIV viral protein, is expressed. In such an embodiment of the invention, the heterologous sequences may also be derived from the genome of a human immunodeficiency virus, preferably of human immunodeficiency virus-1 or human immunodeficiency virus-2.

In instances whereby the heterologous sequences are HIV-derived, such sequences may include, but are not limited to sequences derived from the env gene (i.e., sequences encoding all or part of gp160, gp120, and/or gp41), the pol gene (i.e., sequences encoding all or part of reverse transcriptase, endonuclease, protease, and/or integrase), the gag gene (i.e., sequences encoding all or part of p7, p6, p55, p17/18, p24/25) tat, rev, nef, vif, vpu, vpr, and/or vpx.

In another embodiment, heterologous gene sequences that can be engineered into the chimeric viruses include those that encode proteins with immunopotentiating activities. Examples of immunopotentiating proteins include, but are not limited to, cytokines, interferon type 1, gamma interferon, colony stimulating factors, and interleukin-1, -2, -4, -5, -6, -12.

In addition, other heterologous gene sequences that may be engineered into the chimeric viruses include those encoding antigens derived from bacteria such as bacterial surface glycoproteins, antigens derived from fungi, and antigens derived from a variety of other pathogens and parasites. Examples of heterologous gene sequences derived from bacterial pathogens include, but are not limited to, those encoding antigens derived from species of the following genera: Salmonella, Shigella, Chlamydia, Helicobacter, Yersinia, Bordatella, Pseudomonas, Neisseria, Vibrio, Haemophilus, Mycoplasma, Streptomyces, Treponema, Coxiella, Ehrlichia, Brucella, Streptobacillus, Fusospirocheta, Spirillum, Ureaplasma, Spirochaeta, Mycoplasma, Actinomycetes, Borrelia, Bacteroides, Trichomoras, Branhamella, Pasteurella, Clostridium, Corynebacterium, Listeria, Bacillus, Erysipelothrix, Rhodococcus, Escherichia, Klebsiella, Pseudomanas, Enterobacter, Serratia, Staphylococcus, Streptococcus, Legionella, Mycobacterium, Proteus, Campylobacter, Enterococcus, Acinetobacter, Morganella, Moraxella, Citrobacter, Rickettsia, Rochlimeae, as well as bacterial species such as: P. aeruginosa; E. coli, P. cepacia, S. epidermis, E. faecalis, S. pneumonias, S. aureus, N. meningitidis, S. pyogenes, Pasteurella multocida, Treponema pallidum, and P. mirabilis.

Examples of heterologous gene sequences derived from pathogenic fungi, include, but are not limited to, those encoding antigens derived from fungi such as Cryptococcus neoformans; Blastomyces dermatitidis; Aiellomyces dermatitidis; Histoplasma capsulatum; Coccidioides immitis; Candida species, including C. albicans, C. tropicalis, C. parapsilosis, C. guilliermondii and C. krusei, Aspergillus species, including A. fumigatus, A. flavus and A. niger, Rhizopus species; Rhizomucor species; Cunninghammella species; Apophysomyces species, including A. saksenaea, A. mucor and A. absidia; Sporothrix schenckii, Paracoccidioides brasiliensis; Pseudallescheria boydii, Torulopsis glabrata; Trichophyton species, Microsporum species and Dermatophyres species, as well as any other yeast or fungus now known or later identified to be pathogenic.

Finally, examples of heterologous gene sequences derived from parasites include, but are not limited to, those encoding antigens derived from members of the Apicomplexa phylum such as, for example, Babesia, Toxoplasma, Plasmodium, Eimeria, Isospora, Atoxoplasma, Cystoisospora, Hammondia, Besniotia, Sarcocystis, Frenkelia, Haemoproteus, Leucocytozoon, Theileria, Perkinsus and Gregarina spp.; Pneumocystis carinii; members of the Microspora phylum such as, for example, Nosema, Enterocytozoon, Encephalitozoon, Septata, Mrazekia, Amblyospora, Ameson, Glugea, Pleistophora and Microsporidium spp.; and members of the Ascetospora phylum such as, for example, Haplosporidium spp., as well as species including Plasmodium falciparum, P. vivax, P. ovale, P. malaria; Toxoplasma gondii; Leishmania mexicana, L. tropica, L. major, L. aethiopica, L. donovani, Trypanosoma cruzi, T brucei, Schistosoma mansoni, S. haematobium, S. japonium; Trichinella spiralis; Wuchereria bancrofti; Brugia malayli; Entamoeba histolytica; Enterobius vermiculoarus; Taenia solium, T. saginata, Trichomonas vaginatis, T hominis, T tenax; Giardia lamblia; Cryptosporidium parvum; Pneumocytis carinii, Babesia bovis, B. divergens, B. microti, Isospora belli, L hominis; Dientamoeba fragilis; Onchocerca volvulus; Ascaris lumbricoides; Necator americanis; Ancylostoma duodenale; Strongyloides stercoralis; Capillaria philippinensis; Angiostrongylus cantonensis; Hymenolepis nana; Diphyllobothrium latum; Echinococcus granulosus, E. multilocularis; Paragonimus westermani, P. caliensis; Chlonorchis sinensis; Opisthorchis felineas, G. Viverini, Fasciola hepatica, Sarcoptes scabiei, Pediculus humanus; Phthirlus pubis; and Dermatobia hominis, as well as any other parasite now known or later identified to be pathogenic.

5.1.2. Metapneumoviral Sequences to be Inserted

The invention relates to nucleic acid sequences of a mammalian MPV, proteins of a mammalian MPV, and antibodies against proteins of a mammalian MPV. The invention further relates to homologs of nucleic acid sequences of a mammalian MPV and homologs of proteins of a mammalian MPV. The invention further relates to nucleic acid sequences encoding fusion proteins, wherein the fusion protein contains a protein of a mammalian MPV or a fragment thereof and one or more peptides or proteins that are not derived from mammalian MPV. In a specific embodiment, a fusion protein of the invention contains a protein of a mammalian MPV or a fragment thereof and a peptide tag, such as, but not limited to a polyhistidine tag. The invention further relates to fusion proteins, wherein the fusion protein contains a protein of a mammalian MPV or a fragment thereof and one or more peptides or proteins that are not derived from mammalian MPV. The invention also relates to derivatives of nucleic acids encoding a protein of a mammlian MPV. The invention also relates to derivatives of proteins of a mammalian MPV. A derivative can be, but is not limited to, mutant forms of the protein, such as, but not limited to, additions, deletions, truncations, substitutions, and inversions. A derivative can further be a chimeric form of the protein of the mammalian MPV, wherein at least one domain of the protein is derived from a different protein. A derivative can also be a form of a protein of a mammalian MPV that is covalently or non-covalently linked to another molecule, such as, e.g., a drug.

The viral isolate termed NL/1/00 (also 00-1) is a mammalian MPV of variant A1 and its genomic sequence is shown in SEQ ID NO:95. The viral isolate termed NL/17/00 is a mammalian MPV of variant A2 and its genomic sequence is shown in SEQ ID NO:96. The viral isolate termed NL/1/99 (also 99-1) is a mammalian MPV of variant B1 and its genomic sequence is shown in SEQ ID NO:94. The viral isolate termed NL/1/94 is a mammalian MPV of variant B2 and its genomic sequence is shown in SEQ ID NO:97. A list of sequences disclosed in the present application and the corresponding SEQ ID Nos is set forth in Table 16.

The protein of a mammalian MPV can be a an N protein, a P protein, a M protein, a F protein, a M2-1 protein or a M2-2 protein or a fragment thereof. A fragment of a protein of a mammlian MPV can be can be at least 25 amino acids, at least 50 amino acids, at least 75 amino acids, at least 100 amino acids, at least 125 amino acids, at least 150 amino acids, at least 175 amino acids, at least 200 amino acids, at least 225 amino acids, at least 250 amino acids, at least 275 amino acids, at least 300 amino acids, at least 325 amino acids, at least 350 amino acids, at least 375 amino acids, at least 400 amino acids, at least 425 amino acids, at least 450 amino acids, at least 475 amino acids, at least 500 amino acids, at least 750 amino acids, at least 1000 amino acids, at least 1250 amino acids, at least 1500 amino acids, at least 1750 amino acids, at least 2000 amino acids or at least 2250 amino acids in length. A fragment of a protein of a mammlian MPV can be can be at most 25 amino acids, at most 50 amino acids, at most 75 amino acids, at most 100 amino acids, at most 125 amino acids, at most 150 amino acids, at most 175 amino acids, at most 200 amino acids, at most 225 amino acids, at most 250 amino acids, at most 275 amino acids, at most 300 amino acids, at most 325 amino acids, at most 350 amino acids, at most 375 amino acids, at most 400 amino acids, at most 425 amino acids, at most 450 amino acids, at most 475 amino acids, at most 500 amino acids, at most 750 amino acids, at most 1000 amino acids, at most 1250 amino acids, at most 1500 amino acids, at most 1750 amino acids, at most 2000 amino acids or at most 2250 amino acids in length.

In certain embodiments of the invention, the protein of a mammalian MPV is a N protein, wherein the N protein is phylogenetically closer related to a N protein of a mammalian MPV, such as the N protein encoded by, e.g., the viral genome of SEQ ID NO:94, SEQ ID NO:95, SEQ ID NO:96, or SEQ ID NO:97, (see also Table 16 for a description of the SEQ ID Nos) than it is related to the N protein of APV type C. In certain embodiments of the invention, the protein of a mammalian MPV is a P protein, wherein the P protein is phylogenetically closer related to a P protein of a mammalian MPV, such as the P protein encoded by, e.g., the viral genome of SEQ ID NO:NO:94, SEQ ID NO:95, SEQ ID NO:96, or SEQ ID NO:97, than it is related to the N protein of APV type C. In certain embodiments of the invention, the protein of a mammalian MPV is a M protein, wherein the M protein is closer related to a M protein of a mammalian MPV, such as the M protein encoded by, e.g., the viral genome of SEQ ID NO:94, SEQ ID NO:95, SEQ ID NO:96, or SEQ ID NO:97, than it is related to the M protein of APV type C. In certain embodiments of the invention, the protein of a mammalian MPV is a F protein, wherein the F protein is phylogenetically closer related to a F protein of a mammalian MPV, such as the F protein encoded by, e.g., the viral genome of SEQ ID NO:94, SEQ ID NO:95, SEQ ID NO:96, or SEQ ID NO:97, than it is related to the F protein of APV type C. In certain embodiments of the invention, the protein of a mammalian MPV is a M2-1 protein, wherein the M2-1 protein is phylogenetically closer related to a M2-1 protein of a mammalian MPV, such as the M2-1 protein encoded by, e.g., the viral genome of SEQ ID NO:94, SEQ ID NO:95, SEQ ID NO:96, or SEQ ID NO:97, than it is related to the M2-1 protein of APV type C. In certain embodiments of the invention, the protein of a mammalian MPV is a M2-2 protein, wherein the M2-2 protein is phylogenetically closer related to a M2-2 protein of a mammalian MPV, such as the M2-2 protein encoded by, e.g., the viral genome of SEQ ID NO:94, SEQ ID NO:95, SEQ ID NO:96, or SEQ ID NO:97, than it is related to the M2-2 protein of APV type C. In certain embodiments of the invention, the protein of a mammalian MPV is a G protein, wherein the G protein is phylogenetically closer related to a G protein of a mammalian MPV, such as the G protein encoded by, e.g., the viral genome of SEQ ID NO:94, SEQ ID NO:95, SEQ ID NO:96, or SEQ ID NO:97, than it is related to any protein of APV type C. In certain embodiments of the invention, the protein of a mammalian MPV is a SH protein, wherein the SH protein is phylogenetically closer related to a SH protein of a mammalian MPV, such as the SH protein encoded by, e.g., the viral genome of SEQ ID NO:94, SEQ ID NO:95, SEQ ID NO:96, or SEQ ID NO:97, than it is related to any protein of APV type C. In certain embodiments of the invention, the protein of a mammalian MPV is a L protein, wherein the L protein is phylogenetically closer related to a L protein of a mammalian MPV, such as the SH protein encoded by, e.g., the viral genome of SEQ ID NO:94, SEQ ID NO:95, SEQ ID NO:96, or SEQ ID NO:97, than it is related to any protein of APV type C.

In certain embodiments of the invention, the protein of a mammalian MPV is a N protein, wherein the N protein is at least 60%, at least 65%, at least 70%, at least 75%, at least 80%, at least 85%, at least 90%, at least 95%, at least 98%, at least 99%, or at least 99.5% identical to the amino acid sequence of a N protein encoded by the viral genome of SEQ ID NO:94, SEQ ID NO:95, SEQ ID NO:96, or SEQ ID NO:97 (the amino acid sequences of the respective N proteins are disclosed in SEQ ID NO:366-369; see also Table 16). In certain embodiments of the invention, the protein of a mammalian MPV is a N protein, wherein the P protein is at least 60%, at least 65%, at least 70%, at least 75%, at least 80%, at least 85%, at least 90%, at least 95%, at least 98%, at least 99%, or at least 99.5% identical to the amino acid sequence of a P protein encoded by the viral genome of SEQ ID NO:94, SEQ ID NO:95, SEQ ID NO:96, or SEQ ID NO:97 (the amino acid sequences of the respective P proteins are disclosed in SEQ ID NO:78-85; see also Table 16). In certain embodiments of the invention, the protein of a mammalian MPV is a M protein, wherein the M protein is at least 60%, at least 65%, at least 70%, at least 75%, at least 80%, at least 85%, at least 90%, at least 95%, at least 98%, at least 99%, or at least 99.5% identical to the amino acid sequence of a M protein encoded by the viral genome of SEQ ID NO:94, SEQ ID NO:95, SEQ ID NO:96, or SEQ ID NO:97 (the amino acid sequences of the respective M proteins are disclosed in SEQ ID NO:358-361; see also Table 16). In certain embodiments of the invention, the protein of a mammalian MPV is a F protein, wherein the F protein is at least 60%, at least 65%, at least 70%, at least 75%, at least 80%, at least 85%, at least 90%, at least 95%, at least 98%, at least 99%, or at least 99.5% identical to the amino acid sequence of a F protein encoded by the viral genome of SEQ ID NO:94, SEQ ID NO:95, SEQ ID NO:96, or SEQ ID NO:97 (the amino acid sequences of the respective F proteins are disclosed in SEQ ID NO:18-25; see also Table 16). In certain embodiments of the invention, the protein of a mammalian MPV is a M2-1 protein, wherein the M2-1 protein is at least 60%, at least 65%, at least 70%, at least 75%, at least 80%, at least 85%, at least 90%, at least 95%, at least 98%, at least 99%, or at least 99.5% identical to the amino acid sequence of a M2-1 protein encoded by the viral genome of SEQ ID NO:94, SEQ ID NO:95, SEQ ID NO:96, or SEQ ID NO:97 (the amino acid sequences of the respective M2-1 proteins are disclosed in SEQ ID NO:42-49; see also Table 16). In certain embodiments of the invention, the protein of a mammalian MPV is a M2-2 protein, wherein the M2-2 protein is at least 60%, at least 65%, at least 70%, at least 75%, at least 80%, at least 85%, at least 90%, at least 95%, at least 98%, at least 99%, or at least 99.5% identical to the amino acid sequence of a M2-2 protein encoded by the viral genome of SEQ ID NO:94, SEQ ID NO:95, SEQ ID NO:96, or SEQ ID NO:97 (the amino acid sequences of the respective M2-2 proteins are disclosed in SEQ ID NO:50-57; see also Table 16). In certain embodiments of the invention, the protein of a mammalian MPV is a G protein, wherein the G protein is at least 60%, at least 65%, at least 70%, at least 75%, at least 80%, at least 85%, at least 90%, at least 95%, at least 98%, at least 99%, or at least 99.5% identical to the amino acid sequence of a G protein encoded by the viral genome of SEQ ID NO:94, SEQ ID NO:95, SEQ ID NO:96, or SEQ ID NO:97 (the amino acid sequences of the respective G proteins are disclosed in SEQ ID NO:26-33; see also Table 16). In certain embodiments of the invention, the protein of a mammalian MPV is a SH protein, wherein the SH protein is at least 60%, at least 65%, at least 70%, at least 75%, at least 80%, at least 85%, at least 90%, at least 95%, at least 98%, at least 99%, or at least 99.5% identical to the amino acid sequence of a SH protein encoded by the viral genome of SEQ ID NO:94, SEQ ID NO:95, SEQ ID NO:96, or SEQ ID NO:97 (the amino acid sequences of the respective SH proteins are disclosed in SEQ ID NO:86-93; see also Table 16). In certain embodiments of the invention, the protein of a mammalian MPV is a L protein, wherein the L protein is at least 60%, at least 65%, at least 70%, at least 75%, at least 80%, at least 85%, at least 90%, at least 95%, at least 98%, at least 99%, or at least 99.5% identical to the amino acid sequence of a L protein encoded by the viral genome of SEQ ID NO:94, SEQ ID NO:95, SEQ ID NO:96, or SEQ ID NO:97 (the amino acid sequences of the respective L proteins are disclosed in SEQ ID NO:34-41; see also Table 16).

A fragment of a protein of mammalian MPV is at least 60%, at least 65%, at least 70%, at least 75%, at least 80%, at least 85%, at least 90%, at least 95%, at least 98%, at least 99%, or at least 99.5% identical to the homologous protein encoded by the virus of SEQ ID NO:94, SEQ ID NO:95, SEQ ID NO:96, or SEQ ID NO:97 over the portion of the protein that is homologous to the fragment. In a specific, illustrative embodiment, the invention provides a fragment of the F protein of a mammalian MPV that contains the ectodomain of the F protein and homologs thereof. The homolog of the fragment of the F protein that contains the ectodomain is at least 60%, at least 65%, at least 70%, at least 75%, at least 80%, at least 85%, at least 90%, at least 95%, at least 98%, at least 99%, or at least 99.5% identical to the corresponding fragment containing the ectodomain of the F protein encoded by a virus of SEQ ID NO:94, SEQ ID NO:95, SEQ ID NO:96, or SEQ ID NO:97 (the amino acid sequences of the respective F proteins are disclosed in SEQ ID NO:18-25; see also Table 16).

In certain embodiments, the invention provides a protein of a mammalian MPV of subgroup A and fragments thereof. The invention provides a N protein of a mammalian MPV of subgroup A, wherein the N protein is phylogenetically closer related to the N protein encoded by a virus of SEQ ID NO:95 or SEQ ID NO:96 than it is related to the N protein encoded by a virus encoded by SEQ ID NO:94 or SEQ ID NO:97. The invention provides a G protein of a mammalian MPV of subgroup A, wherein the G protein is phylogenetically closer related to the G protein encoded by a virus of SEQ ID NO:95 or SEQ ID NO:96 than it is related to the G protein encoded by a virus encoded by SEQ ID NO:94 or SEQ ID NO:97. The invention provides a P protein of a mammalian MPV of subgroup A, wherein the P protein is phylogenetically closer related to the P protein encoded by a virus of SEQ ID NO:95 or SEQ ID NO:96 than it is related to the P protein encoded by a virus encoded by SEQ ID NO:94 or SEQ ID NO:97. The invention provides a M protein of a mammalian MPV of subgroup A, wherein the M protein is phylogenetically closer related to the M protein encoded by a virus of SEQ ID NO:95 or SEQ ID NO:96 than it is related to the M protein encoded by a virus encoded by SEQ ID NO:94 or SEQ ID NO:97. The invention provides a N protein of a mammalian MPV of subgroup A, wherein the F protein is phylogenetically closer related to the F protein encoded by a virus of SEQ ID NO:95 or SEQ ID NO:96 than it is related to the F protein encoded by a virus encoded by SEQ ID NO:94 or SEQ ID NO:97. The invention provides a M2-1 protein of a mammalian MPV of subgroup A, wherein the M2-1 protein is phylogenetically closer related to the M2-1 protein encoded by a virus of SEQ ID NO:95 or SEQ ID NO:96 than it is related to the M2-1 protein encoded by a virus encoded by SEQ ID NO:94 or SEQ ID NO:97. The invention provides a M2-2 protein of a mammalian MPV of subgroup A, wherein the M2-2 protein is phylogenetically closer related to the M2-2 protein encoded by a virus of SEQ ID NO:95 or SEQ ID NO:96 than it is related to the M2-2 protein encoded by a virus encoded by SEQ ID NO:94 or SEQ ID NO:97. The invention provides a SH protein of a mammalian MPV of subgroup A, wherein the SH protein is phylogenetically closer related to the SH protein encoded by a virus of SEQ ID NO:95 or SEQ ID NO:96 than it is related to the SH protein encoded by a virus encoded by SEQ ID NO:94 or SEQ ID NO:97. The invention provides a L protein of a mammalian MPV of subgroup A, wherein the L protein is phylogenetically closer related to the L protein encoded by a virus of SEQ ID NO:95 or SEQ ID NO:96 than it is related to the L protein encoded by a virus encoded by SEQ ID NO:94 or SEQ ID NO:97.

In other embodiments, the invention provides a protein of a mammalian MPV of subgroup B or fragments thereof. The invention provides a N protein of a mammalian MPV of subgroup B, wherein the N protein is phylogenetically closer related to the N protein encoded by a virus of SEQ ID NO:94 or SEQ ID NO:97 than it is related to the N protein encoded by a virus encoded by SEQ ID NO:95 or SEQ ID NO:96. The invention provides a G protein of a mammalian MPV of subgroup A, wherein the G protein is phylogenetically closer related to the G protein encoded by a virus of SEQ ID NO:94 or SEQ ID NO:97 than it is related to the G protein encoded by a virus encoded by SEQ ID NO:95 or SEQ ID NO:96. The invention provides a P protein of a mammalian MPV of subgroup A, wherein the P protein is phylogenetically closer related to the P protein encoded by a virus of SEQ ID NO:94 or SEQ ID NO:97 than it is related to the P protein encoded by a virus encoded by SEQ ID NO:95 or SEQ ID NO:96. The invention provides a M protein of a mammalian MPV of subgroup A, wherein the M protein is phylogenetically closer related to the M protein encoded by a virus of SEQ ID NO:94 or SEQ ID NO:97 than it is related to the M protein encoded by a virus encoded by SEQ ID NO:95 or SEQ ID NO:96. The invention provides a N protein of a mammalian MPV of subgroup A, wherein the F protein is phylogenetically closer related to the F protein encoded by a virus of SEQ ID NO:94 or SEQ ID NO:97 than it is related to the F protein encoded by a virus encoded by SEQ ID NO:95 or SEQ ID NO:96. The invention provides a M2-1 protein of a mammalian MPV of subgroup A, wherein the M2-1 protein is phylogenetically closer related to the M2-1 protein encoded by a virus of SEQ ID NO:94 or SEQ ID NO:97 than it is related to the M2-1 protein encoded by a virus encoded by SEQ ID NO:95 or SEQ ID NO:96. The invention provides a M2-2 protein of a mammalian MPV of subgroup A, wherein the M2-2 protein is phylogenetically closer related to the M2-2 protein encoded by a virus of SEQ ID NO:94 or SEQ ID NO:97 than it is related to the M2-2 protein encoded by a virus encoded by SEQ ID NO:95 or SEQ ID NO:96. The invention provides a SH protein of a mammalian MPV of subgroup A, wherein the SH protein is phylogenetically closer related to the SH protein encoded by a virus of SEQ ID NO:94 or SEQ ID NO:97 than it is related to the SH protein encoded by a virus encoded by SEQ ID NO:95 or SEQ ID NO:96. The invention provides a L protein of a mammalian MPV of subgroup A, wherein the L protein is phylogenetically closer related to the L protein encoded by a virus of SEQ ID NO:94 or SEQ ID NO:97 than it is related to the L protein encoded by a virus encoded by SEQ ID NO:95 or SEQ ID NO:96.

The invention provides a G protein of a mammalian MPV variant B1, wherein the G protein of a mammalian MPV variant B1 is phylogenetically closer related to the G protein of the prototype of variant B1, isolate NL/1/99, than it is related to the G protein of the prototype of variant A1, isolate NL/1/00, the G protein of the prototype of A2, isolate NL/17/00, or the G protein of the prototype of B2, isolate NL/1/94. The invention provides a G protein of a mammalian MPV variant B1, wherein the amino acid sequence of the G protein is at least 66%, at least 70%, at least 75%, at least 80%, at least 85%, at least 90%, at least 95%, at least 98%, or at least 99% or at least 99.5% identical to the G protein of a mammalian MPV variant B1 as represented by the prototype NL/1/99 (SEQ ID NO:28). In a specific embodiment, the G protein of a mammalian MPV has the amino acid sequence of SEQ ID NO:119-153. The invention provides a N protein of a mammalian MPV variant B1, wherein the N protein of a mammalian MPV variant B1 is phylogenetically closer related to the N protein of the prototype of variant B1, isolate NL/1/99, than it is related to the N protein of the prototype of variant A1, isolate NL/1/00, the N protein of the prototype of A2, isolate NL/17/00, or the N protein of the prototype of B2, isolate NL/1/94. The invention provides a N protein of a mammalian MPV variant B1, wherein the amino acid sequence of the N proteint is at least 98.5% or at least 99% or at least 99.5% identical to the N protein of a mammalian MPV variant B1 as represented by the prototype NL/1/99 (SEQ ID NO:72). The invention provides a P protein of a mammalian MPV variant B1, wherein the P protein of a mammalian MPV variant B1 is phylogenetically closer related to the P protein of the prototype of variant B1, isolate NL/1/99, than it is related to the P protein of the prototype of variant A1, isolate NL/1/00, the P protein of the prototype of A2, isolate NL/17/00, or the P protein of the prototype of B2, isolate NL/1/94. The invention provides a P protein of a mammalian MPV variant B1, wherein the amino acid sequence of the P protein is at least 96%, at least 98%, or at least 99% or at least 99.5% identical the P protein of a mammalian MPV variant B1 as represented by the prototype NL/1/99 (SEQ ID NO:80). The invention provides a M protein of a mammalian MPV variant B1, wherein the M protein of a mammalian MPV variant B1 is phylogenetically closer related to the M protein of the prototype of variant B1, isolate NL/1/99, than it is related to the M protein of the prototype of variant A1, isolate NL/1/00, the M protein of the prototype of A2, isolate NL/17/00, or the M protein of the prototype of B2, isolate NL/1/94. The invention provides a M protein of a mammalian MPV variant B1, wherein the amino acid sequence of the M protein is identical to the M protein of a mammalian MPV variant B1 as represented by the prototype NL/1/99 (SEQ ID NO:64). The invention provides a F protein of a mammalian MPV variant B1, wherein the F protein of a mammalian MPV variant B1 is phylogenetically closer related to the F protein of variant B1, isolate NL/1/99, than it is related to the F protein of variant A1, isolate NL/1/00, the F protein of prototype A2, isolate NL/17/00, or the F protein of the prototype of B2, isolate NL/1/94. The invention provides a F protein of mammalian MPV variant B1, wherein the amino acid sequence of the F protein is identical at least 99% identical, to the F protein of a mammalian MPV variant B1 as represented by the prototype NL/1/99 (SEQ ID NO:20). In a specific embodiment, the F protein of a mammalian MPV has the amino acid sequence of SEQ ID NO: 248-327. The invention provides a M2-1 protein of a mammalian MPV variant B1, wherein the M2-1 protein of a mammalian MPV variant B1 is phylogenetically closer related to the M2-1 protein of the prototype of variant B1, isolate NL/1/99, than it is related to the M2-1 protein of the prototype of variant A1, isolate NL/1/00, the M2-1 protein of the prototype of A2, isolate NL/17/00, or the M2-1 protein of the prototype of B2, isolate NL/1/94. The invention provides a M2-1 protein of a mammalian MPV variant B1, wherein the amino acid sequence of the M2-1 protein is at least 98% or at least 99% or at least 99.5% identical the M2-1 protein of a mammalian MPV variant B1 as represented by the prototype NL/1/99 (SEQ ID NO:44). The invention provides a M2-2 protein of a mammalian MPV variant B1, wherein the M2-2 protein of a mammalian MPV variant B1 is phylogenetically closer related to the M2-2 protein of the prototype of variant B1, isolate NL/1/99, than it is related to the M2-2 protein of the prototype of variant A1, isolate NL/1/00, the M2-2 protein of the prototype of A2, isolate NL/17/00, or the M2-2 protein of the prototype of B2, isolate NL/1/94. The invention provides a M2-2 protein of a mammalian MPV variant B1, wherein the amino acid sequence of the M2-2 protein is at least 99% or at least 99.5% identical the M2-2 protein of a mammalian MPV variant B1 as represented by the prototype NL/1/99 (SEQ ID NO:52). The invention provides a SH protein of a mammalian MPV variant B1, wherein the SH protein of a mammalian MPV variant B1 is phylogenetically closer related to the SH protein of the prototype of variant B1, isolate NL/1/99, than it is related to the SH protein of the prototype of variant A1, isolate NL/1/00, the SH protein of the prototype of A2, isolate NL/17/00, or the SH protein of the prototype of B2, isolate NL/1/94. The invention provides a SH protein of a mammalian MPV variant B1, wherein the amino acid sequence of the SH protein is at least 83%, at least 85%, at least 90%, at least 95%, at least 98%, or at least 99% or at least 99.5% identical the SH protein of a mammalian MPV variant B1 as represented by the prototype NL/1/99 (SEQ ID NO:88). The invention provides a L protein of a mammalian MPV variant B1, wherein the L protein of a mammalian MPV variant B1 is phylogenetically closer related to the L protein of the prototype of variant B1, isolate NL/1/99, than it is related to the L protein of the prototype of variant A1, isolate NL/1/00, the L protein of the prototype of A2, isolate NL/17/00, or the L protein of the prototype of B2, isolate NL/1/94. The invention provides a L protein of a mammalian MPV variant B1, wherein the amino acid sequence of the L protein is at least 99% or at least 99.5% identical the L protein a mammalian MPV variant B1 as represented by the prototype NL/1/99 (SEQ ID NO:36).

The invention provides a G protein of a mammalian MPV variant A1, wherein the G protein of a mammalian MPV variant A1 is phylogenetically closer related to the G protein of the prototype of variant A1, isolate NL/1/00, than it is related to the G protein of the prototype of variant B1, isolate NL/1/99, the G protein of the prototype of A2, isolate NL/17/00, or the G protein of the prototype of B2, isolate NL/1/94. The invention provides a G protein of a mammalian MPV variant A1, wherein the amino acid sequence of the G protein is at least 66%, at least 70%, at least 75%, at least 80%, at least 85%, at least 90%, at least 95%, at least 98%, or at least 99% or at least 99.5% identical to the G protein of a mammalian MPV variant A1 as represented by the prototype NL/1/00 (SEQ ID NO:26). The invention provides a N protein of a mammalian MPV variant A1, wherein the N protein of a mammalian MPV variant A1 is phylogenetically closer related to the N protein of the prototype of variant A1, isolate NL/1/00, than it is related to the N protein of the prototype of variant B1, isolate NL/1/99, the N protein of the prototype of A2, isolate NL/17/00, or the N protein of the prototype of B2, isolate NL/1/94. The invention provides a N protein of a mammalian MPV variant A1, wherein the amino acid sequence of the N protein is at least 99.5% identical to the N protein of a mammalian MPV variant A1 as represented by the prototype NL/1/00 (SEQ ID NO:70). The invention provides a P protein of a mammalian MPV variant A1, wherein the P protein of a mammalian MPV variant A1 is phylogenetically closer related to the P protein of the prototype of variant A1, isolate NL/1/00, than it is related to the P protein of the prototype of variant B1, isolate NL/1/99, the P protein of the prototype of A2, isolate NL/17/00, or the P protein of the prototype of B2, isolate NL/1/94. The invention provides a P protein of a mammalian MPV variant A1, wherein the amino acid sequence of the P protein is at least 96%, at least 98%, or at least 99% or at least 99.5% identical to the P protein of a mammalian MPV variant A1 as represented by the prototype NL/1/00 (SEQ ID NO:78). The invention provides a M protein of a mammalian MPV variant A1, wherein the M protein of a mammalian MPV variant A1 is phylogenetically closer related to the M protein of the prototype of variant A1, isolate NL/1/00, than it is related to the M protein of the prototype of variant B1, isolate NL/1/99, the M protein of the prototype of A2, isolate NL/17/00, or the M protein of the prototype of B2, isolate NL/1/94. The invention provides a M protein of a mammalian MPV variant A1, wherein the amino acid sequence of the M protein is at least 99% or at least 99.5% identical to the M protein of a mammalian MPV variant A1 as represented by the prototype NL/1/00 (SEQ ID NO:62). The invention provides a F protein of a mammalian MPV variant A1, wherein the F protein of a mammalian MPV variant A1 is phylogenetically closer related to the F protein of the prototype of variant A1, isolate NL/1/00, than it is related to the F protein of the prototype of variant B1, isolate NL/1/99, the F protein of the prototype of A2, isolate NL/17/00, or the F protein of the prototype of B2, isolate NL/1/94. The invention provides a F protein of a mammalian MPV variant A1, wherein the amino acid sequence of the F protein is at least 98% or at least 99% or at least 99.5% identical to the F protein of a mammalian MPV variant A1 as represented by the prototype NL/1/00 (SEQ ID NO:18). The invention provides a M2-1 protein of a mammalian MPV variant A1, wherein the M2-1 protein of a mammalian MPV variant A1 is phylogenetically closer related to the M2-1 protein of the prototype of variant A1, isolate NL/1/00, than it is related to the M2-1 protein of the prototype of variant B1, isolate NL/1/99, the M2-1 protein of the prototype of A2, isolate NL/17/00, or the M2-1 protein of the prototype of B2, isolate NL/1/94. The invention provides a M2-1 protein of a mammalian MPV variant A1, wherein the amino acid sequence of the M2-1 protein is at least 99% or at least 99.5% identical to the M2-1 protein of a mammalian MPV variant A1 as represented by the prototype NL/1/00 (SEQ ID NO:42). The invention provides a M2-2 protein of a mammalian MPV variant A1, wherein the M2-2 protein of a mammalian MPV variant A1 is phylogenetically closer related to the M2-2 protein of the prototype of variant A1, isolate NL/1/00, than it is related to the M2-2 protein of the prototype of variant B1, isolate NL/1/99, the M2-2 protein of the prototype of A2, isolate NL/17/00, or the M2-2 protein of the prototype of B2, isolate NL/1/94. The invention provides a M2-2 protein of a mammalian MPV variant A1, wherein the amino acid sequence of the M2-2 protein is at least 96% or at least 99% or at least 99.5% identical to the M2-2 protein of a mammalian MPV variant A1 as represented by the prototype NL/1/00 (SEQ ID NO:50). The invention provides a SH protein of a mammalian MPV variant A1, wherein the SH protein of a mammalian MPV variant A1 is phylogenetically closer related to the SH protein of the prototype of variant A1, isolate NL/1/00, than it is related to the SH protein of the prototype of variant B1, isolate NL/1/99, the SH protein of the prototype of A2, isolate NL/17/00, or the SH protein of the prototype of B2, isolate NL/1/94. The invention provides a SH protein of a mammalian MPV variant A1, wherein the amino acid sequence of the SH protein is at least 84%, at least 90%, at least 95%, at least 98%, or at least 99% or at least 99.5% identical to the SH protein of a mammalian MPV variant A1 as represented by the prototype NL/1/00 (SEQ ID NO:86). The invention provides a L protein of a mammalian MPV variant A1, wherein the L protein of a mammalian MPV variant A1 is phylogenetically closer related to the L protein of the prototype of variant A1, isolate NL/1/00, than it is related to the L protein of the prototype of variant B1, isolate NL/1/99, the L protein of the prototype of A2, isolate NL/17/00, or the L protein of the prototype of B2, isolate NL/1/94. The invention provides a L protein of a mammalian MPV variant A1, wherein the amino acid sequence of the L protein is at least 99% or at least 99.5% identical to the L protein of a virus of a mammalian MPV variant A1 as represented by the prototype NL/1/00 (SEQ ID NO:34).

The invention provides a G protein of a mammalian MPV variant A2, wherein the G protein of a mammalian MPV variant A2 is phylogenetically closer related to the G protein of the prototype of variant A2, isolate NL/17/00, than it is related to the G protein of the prototype of variant B1, isolate NL/1/99, the G protein of the prototype of A1, isolate NL/1/00, or the G protein of the prototype of B2, isolate NL/1/94. The invention provides a G protein of a mammalian MPV variant A2, wherein the amino acid sequence of the G protein is at least 66%, at least 70%, at least 75%, at least 80%, at least 85%, at least 90%, at least 95%, at least 98%, at least 99% or at least 99.5% identical to the G protein of a mammalian MPV variant A2 as represented by the prototype NL/17/00 (SEQ ID NO:27). The invention provides a N protein of a mammalian MPV variant A2, wherein the N protein of a mammalian MPV variant A2 is phylogenetically closer related to the N protein of the prototype of variant A2, isolate NL/17/00, than it is related to the N protein of the prototype of variant B1, isolate NL/1/99, the N protein of the prototype of A1, isolate NL/1/00, or the N protein of the prototype of B2, isolate NL/1/94. The invention provides a N protein of a mammalian MPV variant A2, wherein the amino acid sequence of the N protein at least 99.5% identical to the N protein of a mammalian MPV variant A2 as represented by the prototype NL/17/00(SEQ ID NO:71). The invention provides a P protein of a mammalian MPV variant A2, wherein the P protein of a mammalian MPV variant A2 is phylogenetically closer related to the P protein of the prototype of variant A2, isolate NL/17/00, than it is related to the P protein of the prototype of variant B1, isolate NL/1/99, the P protein of the prototype of A1, isolate NL/1/00, or the P protein of the prototype of B2, isolate NL/1/94. The invention provides a P protein of a mammalian MPV variant A2, wherein the amino acid sequence of the P protein is at least 96%, at least 98%, at least 99% or at least 99.5% identical to the P protein of a mammalian MPV variant A2 as represented by the prototype NL/17/00 (SEQ ID NO:79). The invention provides a M protein of a mammalian MPV variant A2, wherein the M protein of a mammalian MPV variant A2 is phylogenetically closer related to the M protein of the prototype of variant A2, isolate NL/17/00, than it is related to the M protein of the prototype of variant B1, isolate NL/1/99, the M protein of the prototype of A1, isolate NL/1/00, or the M protein of the prototype of B2, isolate NL/1/94. The invention provides a M protein of a mammalian MPV variant A2, wherein the the amino acid sequence of the M protein is at least 99%, or at least 99.5% identical to the M protein of a mammalian MPV variant A2 as represented by the prototype NL/17/00 (SEQ ID NO:63). The invention provides a F protein of a mammalian MPV variant A2, wherein the F protein of a mammalian MPV variant A2 is phylogenetically closer related to the F protein of the prototype of variant A2, isolate NL/17/00, than it is related to the F protein of the prototype of variant B1, isolate NL/1/99, the F protein of the prototype of A1, isolate NL/1/00, or the F protein of the prototype of B2, isolate NL/1/94. The invention provides a F protein of a mammalian MPV variant A2, wherein the amino acid sequence of the F protein is at least 98%, at least 99% or at least 99.5% identical to the F protein of a mammalian MPV variant A2 as represented by the prototype NL/17/00(SEQ ID NO:19). The invention provides a M2-1 protein of a mammalian MPV variant A2, wherein the M2-1 protein of a mammalian MPV variant A2 is phylogenetically closer related to the M2-1 protein of the prototype of variant A2, isolate NL/17/00, than it is related to the M2-1 protein of the prototype of variant B1, isolate NL/1/99, the M2-1 protein of the prototype of A1, isolate NL/1/00, or the M2-1 protein of the prototype of B2, isolate NL/1/94. The invention provides a M2-1 protein of a mammalian MPV variant A2, wherein the amino acid sequence of the M2-1 protein is at least 99%, or at least 99.5% identical to the M2-1 protein of a mammalian MPV variant A2 as represented by the prototype NL/17/00 (SEQ ID NO: 43). The invention provides a M2-2 protein of a mammalian MPV variant A2, wherein the M2-2 protein of a mammalian MPV variant A2 is phylogenetically closer related to the M2-2 protein of the prototype of variant A2, isolate NL/17/00, than it is related to the M2-2 protein of the prototype of variant B1, isolate NL/1/99, the M2-2 protein of the prototype of A1, isolate NL/1/00, or the M2-2 protein of the prototype of B2, isolate NL/1/94. The invention provides a M2-2 protein of a mammalian MPV variant A2, wherein the amino acid sequence of the M2-2 protein is at least 96%, at least 98%, at least 99% or at least 99.5% identical to the M2-2 protein of a mammalian MPV variant A2 as represented by the prototype NL/17/00 (SEQ ID NO:51). The invention provides a SH protein of a mammalian MPV variant A2, wherein the SH protein of a mammalian MPV variant A2 is phylogenetically closer related to the SH protein of the prototype of variant A2, isolate NL/17/00, than it is related to the SH protein of the prototype of variant B1, isolate NL/1/99, the SH protein of the prototype of A1, isolate NL/1/00, or the SH protein of the prototype of B2, isolate NL/1/94. The invention provides a SH protein of a mammalian MPV variant A2, wherein the amino acid sequence of the SH protein is at least 84%, at least 85%, at least 90%, at least 95%, at least 98%, at least 99% or at least 99.5% identical to the SH protein of a mammalian MPV variant A2 as represented by the prototype NL/17/00(SEQ ID NO:87). The invention provides a L protein of a mammalian MPV variant A2, wherein the L protein of a mammalian MPV variant A2 is phylogenetically closer related to the L protein of the prototype of variant A2, isolate NL/17/00, than it is related to the L protein of the prototype of variant B1, isolate NL/1/99, the L protein of the prototype of A1, isolate NL/1/00, or the L protein of the prototype of B2, isolate NL/1/94. The invention provides a L protein of a mammalian MPV variant A2, wherein the amino acid sequence of the L protein is at least 99% or at least 99.5% identical to the L protein of a mammalian MPV variant A2 as represented by the prototype NL/17/00 (SEQ ID NO:35).

The invention provides a G protein of a mammalian MPV variant B2, wherein the G protein of a mammalian MPV variant B2 is phylogenetically closer related to the G protein of the prototype of variant B2, isolate NL/1/94, than it is related to the G protein of the prototype of variant B1, isolate NL/1/99, the G protein of the prototype of A1, isolate NL/1/00, or the G protein of the prototype of A2, isolate NL/17/00. The invention provides a G protein of a mammalian MPV variant B2, wherein the amino acid sequence of the G protein is at least 66%, at least 70%, at least 75%, at least 80%, at least 85%, at least 90%, at least 95%, at least 98%, or at least 99% or at least 99.5% identical to the G protein of a mammalian MPV variant B2 as represented by the prototype NL/1/94 (SEQ ID NO:29). The invention provides a N protein of a mammalian MPV variant B2, wherein the N protein of a mammalian MPV variant B2 is phylogenetically closer related to the N protein of the prototype of variant B2, isolate NL/1/94, than it is related to the N protein of the prototype of variant B1, isolate NL/1/99, the N protein of the prototype of A1, isolate NL/1/00, or the N protein of the prototype of A2, isolate NL/17/00. The invention provides a N protein of a mammalian MPV variant B2, wherein the amino acid sequence of the N protein is at least 99% or at least 99.5% identical to the N protein of a mammalian MPV variant B2 as represented by the prototype NL/1/94 (SEQ ID NO:73). The invention provides a P protein of a mammalian MPV variant B2, wherein the P protein of a mammalian MPV variant B2 is phylogenetically closer related to the P protein of the prototype of variant B2, isolate NL/1/94, than it is related to the P protein of the prototype of variant B1, isolate NL/1/99, the P protein of the prototype of A1, isolate NL/1/00, or the P protein of the prototype of A2, isolate NL/17/00. The invention provides a P protein of a mammalian MPV variant B2, wherein the amino acid sequence of the P protein is at least 96%, at least 98%, or at least 99% or at least 99.5% identical to the P protein of a mammalian MPV variant B2 as represented by the prototype NL/1/94(SEQ ID NO:81). The invention provides a M protein of a mammalian MPV variant B2, wherein the M protein of a mammalian MPV variant B2 is phylogenetically closer related to the M protein of the prototype of variant B2, isolate NL/1/94, than it is related to the M protein of the prototype of variant B1, isolate NL/1/99, the M protein of the prototype of A1, isolate NL/1/00, or the M protein of the prototype of A2, isolate NL/17/00. The invention provides a M protein of a mammalian MPV variant B2, wherein the amino acid sequence of its M protein is identical to the M protein of a mammalian MPV variant B2 as represented by the prototype NL/1/94 (SEQ ID NO:65). The invention provides a F protein of a mammalian MPV variant B2, wherein the F protein of a mammalian MPV variant B2 is phylogenetically closer related to the F protein of the prototype of variant B2, isolate NL/1/94, than it is related to the F protein of the prototype of variant B1, isolate NL/1/99, the F protein of the prototype of A1, isolate NL/1/00, or the F protein of the prototype of A2, isolate NL/17/00. The invention provides a F protein of a mammalian MPV variant B2, wherein the amino acid sequence of the F protein is at least 99% or at least 99.5% identical to the F protein of a mammalian MPV variant B2 as represented by the prototype NL/1/94 (SEQ ID NO:21). The invention provides a M2-1 protein of a mammalian MPV variant B2, wherein the M2-1 protein of a mammalian MPV variant B2 is phylogenetically closer related to the M2-1 protein of the prototype of variant B2, isolate NL/1/94, than it is related to the M2-1 protein of the prototype of variant B1, isolate NL/1/99, the M2-1 protein of the prototype of A1, isolate NL/1/00, or the M2-1 protein of the prototype of A2, isolate NL/17/00. The invention provides a M2-1 protein of a mammalian MPV variant B2, wherein the amino acid sequence of the M2-1 protein is at least 98% or at least 99% or at least 99.5% identical to the M2-1 protein of a mammalian MPV variant B2 as represented by the prototype NL/1/94 (SEQ ID NO:45). The invention provides a M2-2 protein of a mammalian MPV variant B2, wherein the M2-2 protein of a mammalian MPV variant B2 is phylogenetically closer related to the M2-2 protein of the prototype of variant B2, isolate NL/1/94, than it is related to the M2-2 protein of the prototype of variant B1, isolate NL/1/99, the M2-2 protein of the prototype of A1, isolate NL/1/00, or the M2-2 protein of the prototype of A2, isolate NL/17/00. The invention provides a M2-2 protein of a mammalian MPV variant B2, wherein the amino acid sequence is at least 99% or at least 99.5% identical to the M2-2 protein of a mammalian MPV variant B2 as represented by the prototype NL/1/94 (SEQ ID NO:53). The invention provides a SH protein of a mammalian MPV variant B2, wherein the SH protein of a mammalian MPV variant B2 is phylogenetically closer related to the SH protein of the prototype of variant B2, isolate NL/1/94, than it is related to the SH protein of the prototype of variant B1, isolate NL/1/99, the SH protein of the prototype of A1, isolate NL/1/00, or the SH protein of the prototype of A2, isolate NL/17/00. The invention provides a SH protein of a mammalian MPV variant B2, wherein the amino acid sequence of the SH protein is at least 84%, at least 85%, at least 90%, at least 95%, at least 98%, or at least 99% or at least 99.5% identical to the SH protein of a mammalian MPV variant B2 as represented by the prototype NL/1/94 (SEQ ID NO:89). The invention provides a L protein of a mammalian MPV variant B2, wherein the L protein of a mammalian MPV variant B2 is phylogenetically closer related to the L protein of the prototype of variant B2, isolate NL/1/94, than it is related to the L protein of the prototype of variant B1, isolate NL/1/99, the L protein of the prototype of A1, isolate NL/1/00, or the L protein of the prototype of A2, isolate NL/17/00. The invention provides a L protein of a mammalian MPV variant B2, wherein the and/or if the amino acid sequence of the L protein is at least 99% or at least 99.5% identical to the L protein of a mammalian MPV variant B2 as represented by the prototype NL/1/94 (SEQ ID NO:37).

In certain embodiments, the percentage of sequence identity is based on an alignment of the full length proteins. In other embodiments, the percentage of sequence identity is based on an alignment of contiguous amino acid sequences of the proteins, wherein the amino acid sequences can be 25 amino acids, 50 amino acids, 75 amino acids, 100 amino acids, 125 amino acids, 150 amino acids, 175 amino acids, 200 amino acids, 225 amino acids, 250 amino acids, 275 amino acids, 300 amino acids, 325 amino acids, 350 amino acids, 375 amino acids, 400 amino acids, 425 amino acids, 450 amino acids, 475 amino acids, 500 amino acids, 750 amino acids, 1000 amino acids, 1250 amino acids, 1500 amino acids, 1750 amino acids, 2000 amino acids or 2250 amino acids in length.

The invention further provides nucleic acid sequences derived from a mammalian MPV. The invention also provides derivatives of nucleic acid sequences derived from a mammalian MPV. In certain specific embodiments the nucleic acids are modified.

In certain embodiments, a nucleic acid of the invention encodes a G protein, a N protein, a P protein, a M protein, a F protein, a M2-1 protein, a M2-2 protein, a SH protein, or a L protein of a mammalian MPV as defined above. In certain embodiments, a nucleic acid of the invention encodes a G protein, a N protein, a P protein, a M protein, a F protein, a M2-1 protein, a M2-2 protein, a SH protein, or a L protein of subgroup A of a mammalian MPV as defined above. In a specific embodiment, the G gene of a mammalian MPV has the nucleotide sequence of SEQ ID NO:98-132. In a specific embodiment, the F gene of a mammalian MPV has the nucleotide sequence of SEQ ID NO:168-247. In certain embodiments, a nucleic acid of the invention encodes a G protein, a N protein, a P protein, a M protein, a F protein, a M2-1 protein, a M2-2 protein, a SH protein, or a L protein of subgroup B of a mammalian MPV as defined above. In certain embodiments, a nucleic acid of the invention encodes a G protein, a N protein, a P protein, a M protein, a F protein, a M2-1 protein, a M2-2 protein, a SH protein, or a L protein of variant A1 of a mammalian MPV as defined above. In certain embodiments, a nucleic acid of the invention encodes a G protein, a N protein, a P protein, a M protein, a F protein, a M2-1 protein, a M2-2 protein, a SH protein, or a L protein of variant A2 of a mammalian MPV as defined above. In certain embodiments, a nucleic acid of the invention encodes a G protein, a N protein, a P protein, a M protein, a F protein, a M2-1 protein, a M2-2 protein, a SH protein, or a L protein of variant B1 of a mammalian MPV as defined above. In certain embodiments, a nucleic acid of the invention encodes a G protein, a N protein, a P protein, a M protein, a F protein, a M2-1 protein, a M2-2 protein, a SH protein, or a L protein of variant B2 of a mammalian MPV as defined above.

In certain embodiments, the invention provides a nucleotide sequence that is at least 50%, at least 55%, at least 60%, at least 65%, at least 70%, at least 75%, at least 75%, at least 80%, at least 85%, at least 90%, at least 95%, at least 98%, at least 99%, or at least 99.5% identical to the nucleotide sequence of SEQ ID NO:94, SEQ ID NO:95, SEQ ID NO:96, or SEQ ID NO:97. In certain embodiments, the nucleic acid sequence of the invention, is at least 50%, at least 55%, at least 60%, at least 65%, at least 70%, at least 75%, at least 75%, at least 80%, at least 85%, at least 90%, at least 95%, at least 98%, at least 99%, or at least 99.5% identical to a fragment of the nucleotide sequence of SEQ ID NO:94, SEQ ID NO:95, SEQ ID NO:96, or SEQ ID NO:97, wherein the fragment is at least 25 nucleotides, at least 50 nucleotides, at least 75 nucleotides, at least 100 nucleotides, at least 150 nucleotides, at least 200 nucleotides, at least 250 nucleotides, at least 300 nucleotides, at least 400 nucleotides, at least 500 nucleotides, at least 750 nucleotides, at least 1,000 nucleotides, at least 1,250 nucleotides, at least 1,500 nucleotides, at least 1,750 nucleotides, at least 2,000 nucleotides, at least 2,00 nucleotides, at least 3,000 nucleotides, at least 4,000 nucleotides, at least 5,000 nucleotides, at least 7,500 nucleotides, at least 10,000 nucleotides, at least 12,500 nucleotides, or at least 15,000 nucleotides in length. In a specific embodiment, the nucleic acid sequence of the invention is at least 50%, at least 55%, at least 60%, at least 65%, at least 70%, at least 75%, at least 75%, at least 80%, at least 85%, at least 90%, at least 95%, at least 98%, at least 99%, or at least 99.5% or 100% identical to one of the nucleotide sequences of SEQ ID NO:98-132; SEQ ID NO:168-247; SEQ ID NO:22-25; SEQ ID NO:30-33; SEQ ID NO:38-41; SEQ ID NO:46-49; SEQ ID NO:54-57; SEQ ID NO:58-61; SEQ ID NO:66-69; SEQ ID NO:74-77; SEQ ID NO:82-85; or SEQ ID NO:90-93.

In specific embodiments of the invention, a nucleic acid sequence of the invention is capable of hybridizing under low stringency, medium stringency or high stringency conditions to one of the nucleic acid sequences of SEQ ID NO:94, SEQ ID NO:95, SEQ ID NO:96, or SEQ ID NO:97. In specific embodiments of the invention, a nucleic acid sequence of the invention is capable of hybridizing under low stringency, medium stringency or high stringency conditions to one of the nucleic acid sequences of SEQ ID NO:98-132; SEQ ID NO:168-247; SEQ ID NO:22-25; SEQ ID NO:30-33; SEQ ID NO:38-41; SEQ ID NO:46-49; SEQ ID NO:54-57; SEQ ID NO:58-61; SEQ ID NO:66-69; SEQ ID NO:74-77; SEQ ID NO:82-85; or SEQ ID NO:90-93. In certain embodiments, a nucleic acid hybridizes over a length of at least 25 nucleotides, at least 50 nucleotides, at least 75 nucleotides, at least 100 nucleotides, at least 150 nucleotides, at least 200 nucleotides, at least 250 nucleotides, at least 300 nucleotides, at least 400 nucleotides, at least 500 nucleotides, at least 750 nucleotides, at least 1,000 nucleotides, at least 1,250 nucleotides, at least 1,500 nucleotides, at least 1,750 nucleotides, at least 2,000 nucleotides, at least 2,00 nucleotides, at least 3,000 nucleotides, at least 4,000 nucleotides, at least 5,000 nucleotides, at least 7,500 nucleotides, at least 10,000 nucleotides, at least 12,500 nucleotides, or at least 15,000 nucleotides with the nucleotide sequence of SEQ ID NO:94, SEQ ID NO:95, SEQ ID NO:96, or SEQ ID NO:97.

The invention further provides antibodies and antigen-binding fragments that bind specifically to a protein of a mammalian MPV. An antibody of the invention binds specifically to a G protein, a N protein, a P protein, a M protein, a F protein, a M2-1 protein, a M2-2 protein, a SH protein, or a L protein of a mammalian MPV. In specific embodiments, the antibody is a human antibody or a humanized antibody. In certain embodiments, an antibody of the invention binds specifically to a G protein, a N protein, a P protein, a M protein, a F protein, a M2-1 protein, a M2-2 protein, a SH protein, or a L protein of a virus of subgroup A of a mammalian MPV. In certain other embodiments, an antibody of the invention binds specifically to a G protein, a N protein, a P protein, a M protein, a F protein, a M2-1 protein, a M2-2 protein, a SH protein, or a L protein of a virus of subgroup B of a mammalian MPV. In certain, more specific, embodiments, an antibody of the invention binds specifically to a G protein, a N protein, a P protein, a M protein, a F protein, a M2-1 protein, a M2-2 protein, a SH protein, or a L protein of a virus of variant A1 of a mammalian MPV. In other embodiments, the antibody of the invention binds specifically to a G protein, a N protein, a P protein, a M protein, a F protein, a M2-1 protein, a M2-2 protein, a SH protein, or a L protein of a virus of subgroup A2 of a mammalian MPV. In certain embodiments, an antibody of the invention binds specifically to a G protein, a N protein, a P protein, a M protein, a F protein, a M2-1 protein, a M2-2 protein, a SH protein, or a L protein of a virus of subgroup B 1 of a mammalian MPV. In certain other embodiments, an antibody of the invention binds specifically to a G protein, a N protein, a P protein, a M protein, a F protein, a M2-1 protein, a M2-2 protein, a SH protein, or a L protein of a virus of subgroup B2 of a mammalian MPV.

5.1.3. Insertion of the Heterologous Gene Sequence

Insertion of a foreign gene sequence into a viral vector of the invention can be accomplished by either a complete replacement of a viral coding region with a heterologous sequence, or by a partial replacement of the same, or by adding the heterologous nucleotide sequence to the viral genome. Complete replacement would probably best be accomplished through the use of PCR-directed mutagenesis. Briefly, PCR-primer A would contain, from the 5′ to 3′ end: a unique restriction enzyme site, such as a class IIS restriction enzyme site (i.e., a “shifter” enzyme; that recognizes a specific sequence but cleaves the DNA either upstream or downstream of that sequence); a stretch of nucleotides complementary to a region of the PIV gene; and a stretch of nucleotides complementary to the carboxy-terminus coding portion of the heterologous sequence. PCR-primer B would contain from the 5′ to 3′ end: a unique restriction enzyme site; a stretch of nucleotides complementary to a PIV gene; and a stretch of nucleotides corresponding to the 5′ coding portion of the foreign gene. After a PCR reaction using these primers with a cloned copy of the foreign gene, the product may be excised and cloned using the unique restriction sites. Digestion with the class IIS enzyme and transcription with the purified phage polymerase would generate an RNA molecule containing the exact untranslated ends of the PIV gene with a foreign gene insertion. In an alternate embodiment, PCR-primed reactions could be used to prepare double-stranded DNA containing the bacteriophage promoter sequence, and the hybrid gene sequence so that RNA templates can be transcribed directly without cloning.

A heterologous nucleotide sequence can be added or inserted at various positions of the virus of the invention. In one embodiment, the heterologous nucleotide sequence is added or inserted at position 1. In another embodiment, the heterologous nucleotide sequence is added or inserted at position 2. In another embodiment, the heterologous nucleotide sequence is added or inserted at position 3. In another embodiment, the heterologous nucleotide sequence is added or inserted at position 4. In another embodiment, the heterologous nucleotide sequence is added or inserted at position 5. In yet another embodiment, the heterologous nucleotide sequence is added or inserted at position 6. As used herein, the term “position” refers to the position of the heterologous nucleotide sequence on the viral genome to be transcribed, e.g., position 1 means that it is the first gene to be transcribed, and position 2 means that it is the second gene to be transcribed. Inserting heterologous nucleotide sequences at the lower-numbered positions of the virus generally results in stronger expression of the heterologous nucleotide sequence compared to insertion at higher-numbered positions due to a transcriptional gradient that occurs across the genome of the virus. However, the transcriptional gradient also yields specific ratios of viral mRNAs. Insertion of foreign genes will perturb these ratios and result in the synthesis of different amounts of viral proteins that may influence virus replication. Thus, both the transcriptional gradient and the replication kinetics must be considered when choosing an insertion site. For example, insertion of heterologous nucleotide sequence at position 2 of the b/h PIV3 vector results in the best replication rate and expression level of the heterologous gene. Inserting heterologous nucleotide sequences at lower-numbered positions is the preferred embodiment of the invention if strong expression of the heterologous nucleotide sequence is desired. In a preferred embodiment, the heterologous sequence is added or inserted at position 1, 2 or 3.

When inserting a heterologous nucleotide sequence into the virus of the invention, the intergenic region between the end of the coding sequence of the heterologous gene and the start of the coding sequence of the downstream gene can be altered to achieve a desired effect. As used herein, the term “intergenic region” refers to nucleotide sequence between the stop signal of one gene and the start codon (e.g., AUG) of the coding sequence of the next downstream open reading frame. An intergenic region may comprise a non-coding region of a gene, i.e., between the transcription start site and the start of the coding sequence (AUG) of the gene. This non-coding region occurs naturally in bPIV3 mRNAs and other viral genes, which is illustrated as non-limiting examples in Table 2:

TABLE 2
Lengths of Non-coding Regions for bPIV3 mRNAs

| CTT [Gene Start] | AUG | |
|---|---|---|
| N | 45 | nucleotides |
| P | 68 | nucleotides |
| M | 21 | nucleotides |
| F | 201 | nucleotides |
| HN | 62 | nucleotides |
| L | 12 | nucleotides |
| b/h RSV F1 | 10 | nucleotides |
| b/h RSV F2 | 86 | nucleotides |
| b/h RSV F1 NP-P | 83 | nucleotides |

In various embodiments, the intergenic region between the heterologous nucleotide sequence and the downstream gene can be engineered, independently from each other, to be at least 10 nt in length, at least 20 nt in length, at least 30 nt in length, at least 50 nt in length, at least 75 nt in length, at least 100 nt in length, at least 125 nt in length, at least 150 nt in length, at least 175 nt in length or at least 200 nt in length. In certain embodiments, the intergenic region between the heterologous nucleotide sequence and the downstream gene can be engineered, independently from each other, to be at most 10 nt in length, at most 20 nt in length, at most 30 nt in length, at most 50 nt in length, at most 75 nt in length, at most 100 nt in length, at most 125 nt in length, at most 150 nt in length, at most 175 nt in length or at most 200 nt in length. In various embodiments, the non-coding region of a desired gene in a virus genome can also be engineered, independently from each other, to be at least 10 nt in length, at least 20 nt in length, at least 30 nt in length, at least 50 nt in length, at least 75 nt in length, at least 100 nt in length, at least 125 nt in length, at least 150 nt in length, at least 175 nt in length or at least 200 nt in length. In certain embodiments, the non-coding region of a desired gene in a virus genome can also be engineered, independently from each other, to be at most 10 nt in length, at most 20 nt in length, at most 30 nt in length, at most 50 nt in length, at most 75 nt in length, at most 100 nt in length, at most 125 nt in length, at most 150 nt in length, at most 175 nt in length or at most 200 nt in length.

When inserting a heterologous nucleotide sequence, the positional effect and the intergenic region manipulation can be used in combination to achieve a desirable effect. For example, the heterologous nucleotide sequence can be added or inserted at a position selected from the group consisting of position 1, 2, 3, 4, 5, and 6, and the intergenic region between the heterologous nucleotide sequence and the next downstream gene can be altered (see Table 3). In an examplary embodiment, hRSV F gene is inserted at position 1 of a b/h PIV3 vector, and the intergenic region between F gene and N gene (i.e., the next downstream gene of F) is altered to 177 nucleotides. Many more combinations are encompassed by the present invention and some are shown by way of example in Table 3.

TABLE 3
Examples of mode of insertion of heterologous nucleotide sequences

| | Position 1 | Position 2 | Position 3 | Position 4 | Position 5 | Position 6 |
|---|---|---|---|---|---|---|
| IGR^{a} | 10-20 | 10-20 | 10-20 | 10-20 | 10-20 | 10-20 |
| IGR | 21-40 | 21-40 | 21-40 | 21-40 | 21-40 | 21-40 |
| IGR | 41-60 | 41-60 | 41-60 | 41-60 | 41-60 | 41-60 |
| IGR | 61-80 | 61-80 | 61-80 | 61-80 | 61-80 | 61-80 |
| IGR | 81-100 | 81-100 | 81-100 | 81-100 | 81-100 | 81-100 |
| IGR | 101-120 | 101-120 | 101-120 | 101-120 | 101-120 | 101-120 |
| IGR | 121-140 | 121-140 | 121-140 | 121-140 | 121-140 | 121-140 |
| | | | | | 121- | |
| IGR | 141-160 | 141-160 | 141-160 | 141-160 | 141-160 | 141-160 |
| IGR | 161-180 | 161-180 | 161-180 | 161-180 | 161-180 | 161-180 |
| IGR | 181-200 | 181-200 | 181-200 | 181-200 | 181-200 | 181-200 |
| IGR | 201-220 | 201-220 | 201-220 | 201-220 | 201-220 | 201-220 |
| IGR | 221-240 | 221-240 | 221-240 | 221-240 | 221-240 | 221-240 |
| IGR | 241-260 | 241-260 | 241-260 | 241-260 | 241-260 | 241-260 |
| IGR | 261-280 | 261-280 | 261-280 | 261-280 | 261-280 | 261-280 |
| IGR | 281-300 | 281-300 | 281-300 | 281-300 | 281-300 | 281-300 |

^{a}Intergenic Region, measured in nucleotide.

Depending on the purpose (e.g., to have strong immunogenicity) of the inserted heterologous nucleotide sequence, the position of the insertion and the length of the intergenic region of the inserted heterologous nucleotide sequence can be determined by various indexes including, but not limited to, replication kinetics and protein or mRNA expression levels, measured by following non-limiting examples of assays: plaque assay, fluorescent-focus assay, infectious center assay, transformation assay, endpoint dilution assay, efficiency of plating, electron microscopy, hemagglutination, measurement of viral enzyme activity, viral neutralization, hemagglutination inhibition, complement fixation, immunostaining, immunoprecipitation and immunoblotting, enzyme-linked immunosorbent assay, nucleic acid detection (e.g., Southern blot analysis, Northern blot analysis, Western blot analysis), growth curve, employment of a reporter gene (e.g., using a reporter gene, such as Green Fluorescence Protein (GFP) or enhanced Green Fluorescence Protein (eGFP), integrated to the viral genome the same fashion as the interested heterologous gene to observe the protein expression), or a combination thereof. Procedures of performing these assays are well known in the art (see, e.g., Flint et al., PRINCIPLES OF VIROLOGY, MOLECULAR BIOLOGY, PATHOGENESIS, AND CONTROL, 2000, ASM Press pp 25-56, the entire text is incorporated herein by reference), and non-limiting examples are given in the Example sections, infra.

For example, expression levels can be determined by infecting cells in culture with a virus of the invention and subsequently measuring the level of protein expression by, e.g., Western blot analysis or ELISA using antibodies specific to the gene product of the heterologous sequence, or measuring the level of RNA expression by, e.g., Northern blot analysis using probes specific to the heterologous sequence. Similarly, expression levels of the heterologous sequence can be determined by infecting an animal model and measuring the level of protein expressed from the heterologous sequence of the recombinant virus of the invention in the animal model. The protein level can be measured by obtaining a tissue sample from the infected animal and then subjecting the tissue sample to Western blot analysis or ELISA, using antibodies specific to the gene product of the heterologous sequence. Further, if an animal model is used, the titer of antibodies produced by the animal against the gene product of the heterologous sequence can be determined by any technique known to the skilled artisan, including but not limited to, ELISA.

As the heterologous sequences can be homologous to a nucleotide sequence in the genome of the virus, care should be taken that the probes and the antibodies are indeed specific to the heterologous sequence or its gene product.

In certain specific embodiments, expression levels of F-protein of RSV or hMPV from chimeric b/h PIV3 RSV or b/h PIV3 hMPV or b/h PIV3 RSV F and hMPV F can be determined by any technique known to the skilled artisan. Expression levels of the F-protein can be determined by infecting cells in a culture with the chimeric virus of the invention and measuring the level of protein expression by, e.g., Western blot analysis or ELISA using antibodies specific to the F-protein and/or the G-protein of hMPV, or measuring the level of RNA expression by, e.g., Northern blot analysis using probes specific to the F-gene and/or the G-gene of human metapneumovirus. Similarly, expression levels of the heterologous sequence can be determined using an animal model by infecting an animal and measuring the level of F-protein and/or G-protein in the animal model. The protein level can be measured by obtaining a tissue sample from the infected animal and then subjecting the tissue sample to Western blot analysis or ELISA using antibodies specific to F-protein and/or G-protein of the heterologous sequence. Further, if an animal model is used, the titer of antibodies produced by the animal against F-protein and/or G-protein can be determined by any technique known to the skilled artisan, including but not limited to, ELISA.

The rate of replication of a recombinant virus of the invention can be determined by any technique known to the skilled artisan.

In certain embodiments, to facilitate the identification of the optimal position of the heterologous sequence in the viral genome and the optimal length of the intergenic region, the heterologous sequence encodes a reporter gene. Once the optimal parameters are determined, the reporter gene is replaced by a heterologous nucleotide sequence encoding an antigen of choice. Any reporter gene known to the skilled artisan can be used with the methods of the invention. For more detail, see section 5.5.

The rate of replication of the recombinant virus can be determined by any standard technique known to the skilled artisan. The rate of replication is represented by the growth rate of the virus and can be determined by plotting the viral titer over the time post infection. The viral titer can be measured by any technique known to the skilled artisan. In certain embodiments, a suspension containing the virus is incubated with cells that are susceptible to infection by the virus. Cell types that can be used with the methods of the invention include, but are not limited to, Vero cells, LLC-MK-2 cells, Hep-2 cells, LF1043 (HEL) cells, MRC-5 cells, WI-38 cells, 293 T cells, QT 6 cells, QT 35 cells, chicken embryo fibroblast (CEF), or tMK cells. Subsequent to the incubation of the virus with the cells, the number of infected cells is determined. In certain specific embodiments, the virus comprises a reporter gene. Thus, the number of cells expressing the reporter gene is representative of the number of infected cells. In a specific embodiment, the virus comprises a heterologous nucleotide sequence encoding for eGFP, and the number of cells expressing eGFP, i.e., the number of cells infected with the virus, is determined using FACS.

In certain embodiments, the replication rate of the recombinant virus of the invention is at most 20% of the replication rate of the wild type virus from which the recombinant virus is derived under the same conditions. The same conditions refer to the same initial titer of virus, the same strain of cells, the same incubation temperature, growth medium, number of cells and other test conditions that may affect the replication rate. For example, the replication rate of b/h PIV3 with RSV's F gene in position 1 is at most 20% of the replication rate of bPIV3.

In certain embodiments, the replication rate of the recombinant virus of the invention is at most 5%, at most 10%, at most 20%, at most 30%, at most 40%, at most 50%, at most 75%, at most 80%, at most 90% of the replication rate of the wild type virus from which the recombinant virus is derived under the same conditions. In certain embodiments, the replication rate of the recombinant virus of the invention is at least 5%, at least 10%, at least 20%, at least 30%, at least 40%, at least 50%, at least 75%, at least 80%, at least 90% of the replication rate of the wild type virus from which the recombinant virus is derived under the same conditions. In certain embodiments, the replication rate of the recombinant virus of the invention is between 5% and 20%, between 10% and 40%, between 25% and 50%, between 40% and 75%, between 50% and 80%, or between 75% and 90% of the replication rate of the wild type virus from which the recombinant virus is derived under the same conditions.

In certain embodiments, the expression level of the heterologous sequence in the recombinant virus of the invention is at most 20% of the expression level of the F-protein of the wild type virus from which the recombinant virus is derived under the same conditions. The same conditions refer to the same initial titer of virus, the same strain of cells, the same incubation temperature, growth medium, number of cells and other test conditions that may affect the replication rate. For example, the expression level of the heterologous sequence of the F-protein of MPV in position 1 of bPIV3 is at most 20% of the expression level of the bovine F-protein of bPIV3.

In certain embodiments, the expression level of the heterologous sequence in the recombinant virus of the invention is at most 5%, at most 10%, at most 20%, at most 30%, at most 40%, at most 50%, at most 75%, at most 80%, at most 90% of the expression level of the F-protein of the wild type virus from which the recombinant virus is derived under the same conditions. In certain embodiments, the expression level of the heterologous sequence in the recombinant virus of the invention is at least 5%, at least 10%, at least 20%, at least 30%, at least 40%, at least 50%, at least 75%, at least 80%, at least 90% of the expression level of the F-protein of the wild type virus from which the recombinant virus is derived under the same conditions. In certain embodiments, the expression level of the heterologous sequence in the recombinant virus of the invention is between 5% and 20%, between 10% and 40%, between 25% and 50%, between 40% and 75%, between 50% and 80%, or between 75% and 90% of the expression level of the F-protein of the wild type virus from which the recombinant virus is derived under the same conditions.

5.1.4. Insertion of the heterologous Gene Sequence Into the HN Gene

The protein responsible for the hemagglutinin and neuraminidase activities of PIV are coded for by a single gene, HN. The HN protein is a major surface glycoprotein of the virus. For a variety of viruses, such as parainfluenza, the hemagglutinin and neuraminidase proteins have been shown to contain a number of antigenic sites. Consequently, this protein is a potential target for the humoral immune response after infection. Therefore, substitution of antigenic sites of HN with a portion of a foreign protein may provide for a vigorous humoral response against this foreign peptide. If a sequence is inserted within the HN molecule, and it is expressed on the outside surface of the HN, it will be immunogenic. For example, a peptide derived from gp160 of HIV could replace an antigenic site of the HN protein, resulting in a humoral immune response to both gp160 and the HN protein. In a different approach, the foreign peptide sequence may be inserted within the antigenic site without deleting any viral sequences. Expression products of such constructs may be useful in vaccines against the foreign antigen, and may indeed circumvent a problem discussed earlier, that of propagation of the recombinant virus in the vaccinated host. An intact HN molecule with a substitution only in antigenic sites may allow for HN function and thus allow for the construction of a viable virus. Therefore, this virus can be grown without the need for additional helper functions. The virus may also be attenuated in other ways to avoid any danger of accidental escape.

Other hybrid constructions may be made to express proteins on the cell surface or enable them to be released from the cell. As a surface glycoprotein, HN has an amino-terminal cleavable signal sequence necessary for transport to the cell surface, and a carboxy-terminal sequence necessary for membrane anchoring. In order to express an intact foreign protein on the cell surface, it may be necessary to use these HN signals to create a hybrid protein. In this case, the fusion protein may be expressed as a separate fusion protein from an additional internal promoter. Alternatively, if only the transport signals are present and the membrane anchoring domain is absent, the protein may be secreted out of the cell.

5.1.5. Construction of Bicistronic RNA

Bicistronic mRNA could be constructed to permit internal initiation of translation of viral sequences and allow for the expression of foreign protein coding sequences from the regular terminal initiation site. Alternatively, a bicistronic mRNA sequence may be constructed wherein the viral sequence is translated from the regular terminal open reading frame, while the foreign sequence is initiated from an internal site. Certain internal ribosome entry site (IRES) sequences may be utilized. The IRES sequences that are chosen should be short enough to avoid interference with parainfluenza packaging limitations. Thus, it is preferable that the IRES chosen for such a bicistronic approach be no more than 500 nucleotides in length, with less than 250 nucleotides being of ideal length. In a specific embodiment, the IRES is derived from a picornavirus and does not include any additional picornaviral sequences. Preferred IRES elements include, but are not limited to, the mammalian BiP IRES and the hepatitis C virus IRES.

Alternatively, a foreign protein may be expressed from a new internal transcriptional unit in which the transcriptional unit has an initiation site and polyadenylation site. In another embodiment, the foreign gene is inserted into a PIV gene such that the resulting expressed protein is a fusion protein.

5.2. Expression of Heterologous Gene Products Using Recombinant cDNA and RNA Templates

The recombinant templates prepared as described above can be used in a variety of ways to express the heterologous gene products in appropriate host cells or to create chimeric viruses that express the heterologous gene products. In one embodiment, the recombinant cDNA can be used to transfect appropriate host cells and the resulting RNA may direct the expression of the heterologous gene product at high levels. Host cell systems which provide for high levels of expression include continuous cell lines that supply viral functions such as cell lines superinfected with PIV, cell lines engineered to complement PIV functions, etc.

In an alternate embodiment of the invention, the recombinant templates may be used to transfect cell lines that express a viral polymerase protein in order to achieve expression of the heterologous gene product. To this end, transformed cell lines that express a polymerase protein such as the L protein may be utilized as appropriate host cells. Host cells may be similarly engineered to provide other viral functions or additional functions such as HN, NP or N.

In another embodiment, a helper virus may provide the RNA polymerase protein utilized by the cells in order to achieve expression of the heterologous gene product. In yet another embodiment, cells may be transfected with vectors encoding viral proteins such as the N or NP, P, M2-1 and L proteins.

Different technique may be used to detect the expression of heterologous gene products (see, e.g., Flint et al., PRINCIPLES OF VIROLOGY, MOLECULAR BIOLOGY, PATHOGENESIS, AND CONTROL, 2000, ASM Press pp 25-56, the entire text is incorporated herein by reference). In an examplary assay, cells infected with the virus are permeabilized with methanol or acetone and incubated with an antibody raised against the heterologous gene products. A second antibody that recognizes the first antibody is then added. This second antibody is usually conjugated to an indicator so that the expression of heterologous gene products may be visualized or detected.

5.3. Rescue of Recombinant Virus Particles

In order to prepare chimeric virus, modified cDNAs, virus RNAs, or RNA coding for the PIV genome and/or foreign proteins in the plus or minus sense may be used to transfect cells that provide viral proteins and functions required for replication and rescue. Alternatively, cells may be transfected with helper virus before, during, or after transfection by the DNA or RNA molecule coding for the PIV genome and/or foreign proteins. The synthetic recombinant plasmid PIV DNAs and RNAs can be replicated and rescued into infectious virus particles by any number of techniques known in the art, as described in U.S. Pat. No. 5,166,057 issued Nov. 24, 1992; in U.S. Pat. No. 5,854,037 issued Dec. 29, 1998; in European Patent Publication EP 0702085A1, published Feb. 20, 1996; in U.S. patent application Ser. No. 09/152,845; in International Patent Publications PCT WO97/12032 published Apr. 3, 1997; WO96/34625 published Nov. 7, 1996; in European Patent Publication EP-A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 47SA1 published Jun. 25, 1997, each of which is incorporated by reference herein in its entirety.

In one embodiment of the present invention, synthetic recombinant viral RNAs that contain the non-coding regions of the negative strand virus RNA essential for the recognition by viral polymerases and for packaging signals necessary to generate a mature virion, may be prepared. There are a number of different approaches that may be used to apply the reverse genetics approach to rescue negative strand RNA viruses. First, the recombinant RNAs are synthesized from a recombinant DNA template and reconstituted in vitro with purified viral polymerase complex to form recombinant ribonucleoproteins (RNPs) that can be used to transfect cells. In another approach, a more efficient transfection is achieved if the viral polymerase proteins are present during transcription of the synthetic RNAs either in vitro or in vivo. With this approach the synthetic RNAs may be transcribed from cDNA plasmids that are either co-transcribed in vitro with cDNA plasmids encoding the polymerase proteins, or transcribed in vivo in the presence of polymerase proteins, i.e., in cells which transiently or constitutively express the polymerase proteins.

In additional approaches described herein, the production of infectious chimeric virus may be replicated in host cell systems that express a PIV viral polymerase protein (e.g., in virus/host cell expression systems; transformed cell lines engineered to express a polymerase protein, etc.), so that infectious chimeric viruses are rescued. In this instance, helper virus need not be utilized since this function is provided by the viral polymerase proteins expressed.

In accordance with the present invention, any technique known to those of skill in the art may be used to achieve replication and rescue of recombinant and chimeric viruses. One approach involves supplying viral proteins and functions required for replication in vitro prior to transfecting host cells. In such an embodiment, viral proteins may be supplied in the form of wild type virus, helper virus, purified viral proteins or recombinantly expressed viral proteins. The viral proteins may be supplied prior to, during or post transcription of the synthetic cDNAs or RNAs encoding the chimeric virus. The entire mixture may be used to transfect host cells. In another approach, viral proteins and functions required for replication may be supplied prior to or during transcription of the synthetic cDNAs or RNAs encoding the chimeric virus. In such an embodiment, viral proteins and functions required for replication are supplied in the form of wild type virus, helper virus, viral extracts, synthetic cDNAs or RNAs that express the viral proteins are introduced into the host cell via infection or transfection. This infection/transfection takes place prior to or simultaneous to the introduction of the synthetic cDNAs or RNAs encoding the chimeric virus.

In a particularly desirable approach, cells engineered to express all viral genes of the recombinant or chimeric virus of the invention may result in the production of infectious chimeric virus that contain the desired genotype; thus eliminating the need for a selection system. Theoretically, one can replace any one of the six genes or part of any one of the six genes encoding structural proteins of PIV with a foreign sequence. However, a necessary part of this equation is the ability to propagate the defective virus (defective because a normal viral gene product is missing or altered). A number of possible approaches are available to circumvent this problem. In one approach, a virus having a mutant protein can be grown in cell lines that are constructed to constitutively express the wild type version of the same protein. By this way, the cell line complements the mutation in the virus. Similar techniques may be used to construct transformed cell lines that constitutively express any of the PIV genes. These cell lines which are made to express the viral protein may be used to complement the defect in the recombinant virus and thereby propagate it. Alternatively, certain natural host range systems may be available to propagate recombinant virus.

In yet another embodiment, viral proteins and functions required for replication may be supplied as genetic material in the form of synthetic cDNAs or RNAs so that they are co-transcribed with the synthetic cDNAs or RNAs encoding the chimeric virus. In a particularly desirable approach, plasmids that express the chimeric virus and the viral polymerase and/or other viral functions are co-transfected into host cells. For example, plasmids encoding the genomic or antigenomic PIV RNA, either wild type or modified, may be co-transfected into host cells with plasmids encoding the PIV viral polymerase proteins NP or N, P, M2-1 or L. Alternatively, rescue of chimeric b/h PIV3 virus may be accomplished by the use of Modified Vaccinia Virus Ankara (MVA) encoding T7 RNA polymerase, or a combination of MVA and plasmids encoding the polymerase proteins (N, P, and L). For example, MVA-T7 or Fowl Pox-T7 can be infected into Vero cells, LLC-MK-2 cells, Hep-2 cells, LF1043 (HEL) cells, tMK cells, LLC-MK2, HUT 292, FRHL-2 (rhesus), FCL-1 (green monkey), WI-38 (human), MRC-5 (human) cells, 293 T cells, QT 6 cells, QT 35 cells and CEF cells. After infection with MVA-T7 or Fow Pox-T7, a full length antigenomic b/h PIV3 cDNA may be transfected into the HeLa or Vero cells together with the NP, P, M2-1 and L encoding expression plasmids. Alternatively, the polymerase may be provided by plasmid transfection. The cells and cell supernatant can subsequently be harvested and subjected to a single freeze-thaw cycle. The resulting cell lysate may then be used to infect a fresh HeLa or Vero cell monolayer in the presence of 1-beta-D-arabinofuranosylcytosine (ara C), a replication inhibitor of vaccinia virus, to generate a virus stock. The supernatant and cells from these plates can then be harvested, freeze-thawed once, and the presence of bPIV3 virus particles detected by immunostaining of virus plaques using PIV3-specific antiserum.

Another approach to propagating the recombinant virus involves co-cultivation with wild-type virus. This could be done by simply taking recombinant virus and co-infecting cells with this and another wild-type virus (preferably a vaccine strain). The wild-type virus should complement for the defective virus gene product and allow growth of both the wild-type and recombinant virus. Alternatively, a helper virus may be used to support propagation of the recombinant virus.

In another approach, synthetic templates may be replicated in cells co-infected with recombinant viruses that express the PIV virus polymerase protein. In fact, this method may be used to rescue recombinant infectious virus in accordance with the invention. To this end, the PIV polymerase protein may be expressed in any expression vector/host cell system, including but not limited to viral expression vectors (e.g., vaccinia virus, adenovirus, baculovirus, etc.) or cell lines that express a polymerase protein (e.g., see Krystal et al., 1986, Proc. Natl. Acad. Sci. USA 83: 2709-2713). Moreover, infection of host cells expressing all six PIV proteins may result in the production of infectious chimeric virus particles. It should be noted that it may be possible to construct a recombinant virus without altering virus viability. These altered viruses would then be growth competent and would not need helper functions to replicate.

In certain embodiments, conditions for the propagation of virus are optimized in order to produce a robust and high-yielding cell culture (which would be beneficial, e.g., for manufacture the virus vaccine candidates of the invention). Critical parameters can be identified, and the production process can be first optimized in small-scale experiments to determine the scalability, robustness, and reproducibility (an example of this optimization process is provided in Section 36) and subsequently adapted to large scale production of virus. In certain embodiments, the virus that is propagated using the methods of the invention is PIV. In certain embodiments, the virus that is propagated using the methods of the invention is a recombinant or a chimeric PIV. In certain embodiments, the virus that is propagated using the methods of the invention is a virus of one of the following viral families Adenoviridae, Arenaviridae, Astroviridae, Baculoviridae, Bunyaviridae, Caliciviridae, Caulimovirus, Coronaviridae, Cystoviridae, Filoviridae, Flaviviridae, Hepadnaviridae, Herpesviridae, Hypoviridae, Idaeovirus, Inoviridae, Iridoviridae, Leviviridae, Lipothrixviridae, Luteovirus, Machlomovirus, Marafivirus, Microviridae, Myoviridae, Necrovirus, Nodaviridae, Orthomyxoviridae, Papovaviridae, Paramyxoviridae, Partitiviridae, Parvoviridae, Phycodnaviridae, Picornaviridae, Plasmaviridae, Podoviridae, Polydnaviridae, Potyviridae, Poxyiridae, Reoviridae, Retroviridae, Rhabdoviridae, Sequiviridae, Siphoviridae, Sobemovirus, Tectiviridae, Tenuivirus, Tetraviridae, Tobamovirus, Tobravirus, Togaviridae, Tombusviridae, Totiviridae, Trichovirus, Mononegavirales. In certain embodiments, the virus that is propagated with the methods of the invention is an RNA virus. In certain embodiments, the virus is not a virus of the family Herpesviridae. In certain embodiments, the virus is not HSV.

In certain embodiments, a cell culture infected with a virus or a viral construct of interest is incubated at a lower post-infection incubation temperature as compared to the standard incubation temperature for the cells in culture. In a specific embodiment, a cell culture infected with a viral construct of interest is incubated at 33° C. or about 33° C. (e.g., 33±1° C.). In certain embodiments, the post-infection incubation temperature is about 25° C., 26° C., 27° C., 28° C., 29° C., 30° C., 31° C., 32° C., 33° C., 34° C., 35° C., 36° C. or 37° C.

In certain embodiments, virus is propagated by incubating a cells before infection with the virus at a temperature optimized for the growth of the cells and subsequent to infection of the cells with the virus, i.e., post-infection, the temperature is shifted to a lower temperature. In certain embodiments the shift is at least 1° C., 2° C., 3° C., 4° C., 5° C., 6° C., 7° C., 8° C., 9° C., 10° C., 11° C., or at least 12° C. In certain embodiments the shift is at most 1° C., 2° C., 3° C., 4° C., 5° C., 6° C., 7° C., 8° C., 9° C., 10° C., 1°° C., or at most 12° C. In a specific embodiment, the shift is 4° C.

In certain embodiments, the cells are cultured in a medium containing serum before infection with a virus or a viral construct of interest and the cells are cultured in a medium without serum after infection with the virus or viral construct. For a more detailed description of growing infected cells without serum, see the section entitled “Plasmid-Only Recovery of Virus in Serum Free Media.” In a specific embodiment, the serum is fetal bovine serum and is present a concentration of 5% of culture volume, 2% of culture volume, or 0.5% of culture volume.

In certain embodiments, virus is propagated by incubating cells that are infected with the virus in the absence of serum. In certain embodiments, virus is propagated by incubating cells that are infected with the virus in a culture medium containing less than 5% of serum, less than 2.5% of serum, less than 1% of serum, less than 0.1% of serum, less than 0.01% of serum, or less than 0.001% of serum.

In certain embodiments, the cells are incubated before infection with the virus in medium containing serum. In certain embodiments, subsequent to infection of the cells with the virus, the cells are incubated in the absence of serum. In other embodiments, the cells are first incubated in medium containing serum; the cells are then transferred into medium without serum; and subsequently, the cells are infected with the virus and further incubated in the absence of virus.

In certain embodiments, the cells are transferred from medium containing serum into medium in the absence of serum, by removing the serum-containing medium from the cells and adding the medium without serum. In other embodiments, the cells are centrifuged and the medium containing serum is removed and medium without serum is added. In certain embodiments, the cells are washed with medium without serum to ensure that cells once infected with the virus are incubated in the absence of serum. In certain, more specific embodiments, the cells are washed with medium without serum at least one time, two times, three times, four times, five times, or at least ten times.

In yet other embodiments, cells are cultured in a medium containing serum and at a temperature that is optimal for the growth of the cells before infection with a virus or a viral construct, and the cell culture is incubated at a lower temperature (relative to the standard incubation temperature for the corresponding virus or viral vector) after infection with the viral construct of interest. In a specific embodiment, cells are cultured in a medium containing serum before infection with a viral construct of interest at 37° C., and the cell culture is incubated at 33° C. or about 33° C. (e.g., 33±1° C.) after infection with the viral construct of interest.

In even other embodiments, cells are cultured in a medium containing serum and at a temperature that is optimal for the growth of the cells before infection with a virus or a viral construct, and the cell culture is incubated without serum at a lower temperature (relative to the standard incubation temperature for the corresponding virus or viral vector) after infection with the viral construct of interest. In a specific embodiment, cells are cultured in a medium containing serum before infection with a viral construct of interest at 37° C., and the cell culture is incubated without serum at 33° C. or about 33° C. (e.g., 33±1° C.) after infection with the viral construct of interest.

The viral constructs and methods of the present invention can be used for commercial production of viruses, e.g., for vaccine production. For commercial production of a vaccine, it is preferred that the vaccine contains only inactivated viruses or viral proteins that are completely free of infectious virus or contaminating viral nucleic acid, or alternatively, contains live attenuated vaccines that do not revert to virulence. Contamination of vaccines with adventitious agents introduced during production should also be avoided. Methods known in the art for large scale production of viruses or viral proteins can be used for commercial production of a vaccine of the invention. In one embodiment, for commercial production of a vaccine of the invention, cells are cultured in a bioreactor or fermenter. Bioreactors are available in volumes from under 1 liter to in excess of 100 liters, e.g., Cyto3 Bioreactor (Osmonics, Minnetonka, Minn.); NBS bioreactors (New Brunswick Scientific, Edison, N.J.); and laboratory and commercial scale bioreactors from B. Braun Biotech International (B. Braun Biotech, Melsungen, Germany). In another embodiment, small-scale process optimization studies (e.g., see Example 31 (Section 36)) are performed before the commercial production of the virus, and the optimized conditions are selected and used for the commercial production of the virus.

In certain embodiments of the invention, virus can be recovered without helper virus. More specifically, virus can be recovered by introducing into a cell a plasmid encoding the viral genome and plasmids encoding viral proteins required for replication and rescue. In certain embodiments, the cell is grown and maintained in serum-free medium. In certain embodiments, the plasmids are introduced into the cell by electroporation. In a specific embodiment, a plasmid encoding the antigenomic cDNA of the virus under the control of the T7 promoter, a plasmid encoding the T7 RNA polymerase, and plasmids encoding the N protein, P protein, and L protein, respectively, under control of the T7 promoter are introduced into SF Vero cells by electroporation. Vero cells were obtained from ATCC and adapted to grow in serum-free media according to the following steps (developed by Mike Berry's laboratory).
1. Thaw ATCC CCL-81 Vial in DMEM+5% v/v FBS in T-25 flask P121;
2. Expand 5 passages in DMEM+5% v/v FBS P126;
3. Directly transfer FBS grown cells to OptiPRO (Invitrogen Corporation) in T-225 flasks;
4. Expand 7 passages in OptiPRO;
5. Freeze down Pre-Master Cell Bank Stock at Passage 133-7;
6. Expand 4 passages in OptiPRO;
7. Freeze down Master Cell Bank Stock at Passage 137;
8. Expand 4 passages in OptiPRO;
9. Freeze down Working Cell Bank Stock at Passage 141; and
10. Thaw and expand for electroporation and virus amplification.

Methods for the rescue of viral particles are described above in this Section.

In certain embodiments, the cells used for viral rescue are cells that can be grown and/or maintained without the addition of components derived from animals or humans. In certain embodiments, the cells used for viral rescue are cells that are adapted to growth without serum. In a specific embodiment, SF Vero cells are used for the rescue of virus. In certain embodiments, the cells are grown and/or maintained in OptiPRO SFM (Invitrogen Corporation) supplemented with 4 mM L-glutamine. In certain embodiments, the cells are grown in medium that is supplemented with serum but for rescue of viral particles the cells are transferred into serum-free medium. In a specific embodiment, the cells are washed in serum-free medium to ensure that the viral rescue takes place in a serum-free environment.

The plasmids are introduced into the cells by any method known to the skilled artisan that can be used with the cells, e.g., by calcium phosphate transfection, DEAE-Dextran transfection, electroporation or liposome mediated transfection (see Chapter 9 of Short Protocols in Molecular Biology, Ausubel et al. (editors), John Wiley & Sons, Inc., 1999). In specific embodiments, electroporation is used to introduce the plasmid DNA into the cells. SF Vero cells are resistant to lipofection. To select cells that have been transfected with the required plasmids, the plasmids can also carry certain markers. Such markers include, but are not limited to, resistancy to certain antibiotics (e.g., kanamycin, blasticidin, ampicillin, Hygromycin B, Puromycin and Zeocin™), makers that confer certain autotrophic properties on a cell that lacks this property without the marker, or a marker can also be a gene that is required for the growth of a cell but that is mutated in the cells into which the plasmid is introduced.

The transcription of the viral genome and/or the viral genes are under transcriptional control of a promoter. Thus, the sequences encoding the viral genome or the viral proteins are operatively linked to the promoter sequence. Any promoter/RNA polymerase system known to the skilled artisan can be used with the methods of the present invention. In certain embodiments, the promoter can be a promoter that allows transcription by an RNA polymerase endogenous to the cell, e.g., a promoter sequences that are recognized by a cellular DNA dependent RNA polymerases, such as RNA polymerase I (Pol I) or RNA polymerase II (Pol II). In certain embodiments, the promoter can be an inducible promoter. In certain embodiments, the promoter can be a promoter that allows transcription by an RNA polymerase that is not endogenous to the cell. In certain, more specific embodiments, the promoter is a T3 promoter, T7 promoter, SP6 promoter, or CMV promoter. Depending on the type of promoter used, a plasmid encoding the RNA polymerase that recognizes the promoter is also introduced into the cell to provide the appropriate RNA polymerase. In specific embodiments, the RNA polymerase is T3 RNA polymerase, T7 RNA polymerase, SP6 RNA polymerase, or CMV RNA polymerase. In a specific embodiment, the viral genes and the viral genome are transcribed under the control of a T7 promoter and a plasmid encoding the T7 RNA polymerase is introduced to provide the T7 RNA polymerase. The transcription of the polymerase can be under the control of any promoter system that would function in the cell type used. In a specific embodiment, the CMV promoter is used.

The viral genome can be in the plus or minus orientation. Thus, the viral genome can be transcribed from the genetic material to generate either a positive sense copy of the viral genome (antigenome copy) or a negative sense copy of the viral genome (genomic copy). In certain embodiments, the viral genome is a recombinant, chimeric and/or attenuated virus of the invention. In certain embodiments, the efficiency of viral replication and rescue may be enhanced if the viral genome is of hexamer length. In order to ensure that the viral genome is of the appropriate length, the 5′ or 3′ end may be defined using ribozyme sequences, including, Hepatitis Delta Virus (HDV) ribozyme sequence, Hammerhead ribozyme sequences, or fragments thereof, which retain the ribozyme catalytic activity.

In certain embodiments, the viral proteins required for replication and rescue include the N, P, and L gene. In certain, more specific, embodiments, the viral proteins required for replication and rescue include the N, P, M2-1 and L gene.

5.4. Attenuation of Recombinant Viruses

The recombinant viruses of the invention can be further genetically engineered to exhibit an attenuated phenotype. In particular, the recombinant viruses of the invention exhibit an attenuated phenotype in a subject to which the virus is administered as a vaccine. Attenuation can be achieved by any method known to a skilled artisan. Without being bound by theory, the attenuated phenotype of the recombinant virus can be caused, e.g., by using a virus that naturally does not replicate well in an intended host (e.g., using a bovine PIV3 vector in human), by reduced replication of the viral genome, by reduced ability of the virus to infect a host cell, or by reduced ability of the viral proteins to assemble to an infectious viral particle relative to the wild type strain of the virus. The viability of certain sequences of the virus, such as the leader and the trailer sequence can be tested using a minigenome assay (see section 5.5.1).

The attenuated phenotypes of a recombinant virus of the invention can be tested by any method known to the artisan (see, e.g., section 5.5). A candidate virus can, for example, be tested for its ability to infect a host or for the rate of replication in a cell culture system. In certain embodiments, a mini-genome system is used to test the attenuated virus when the gene that is altered is N, P, L, M2 or a combination thereof. In certain embodiments, growth curves at different temperatures are used to test the attenuated phenotype of the virus. For example, an attenuated virus is able to grow at 35° C., but not at 39° C. or 40° C. In certain embodiments, different cell lines can be used to evaluate the attenuated phenotype of the virus. For example, an attenuated virus may only be able to grow in monkey cell lines but not the human cell lines, or the achievable virus titers in different cell lines are different for the attenuated virus. In certain embodiments, viral replication in the respiratory tract of a small animal model, including but not limited to, hamsters, cotton rats, mice and guinea pigs, is used to evaluate the attenuated phenotypes of the virus. In other embodiments, the immune response induced by the virus, including but not limited to, the antibody titers (e.g., assayed by plaque reduction neutralization assay or ELISA) is used to evaluate the attenuated phenotypes of the virus. In a specific embodiment, the plaque reduction neutralization assay or ELISA is carried out at a low dose. In certain embodiments, the ability of the recombinant virus to elicit pathological symptoms in an animal model can be tested. A reduced ability of the virus to elicit pathological symptoms in an animal model system is indicative of its attenuated phenotype. In a specific embodiment, the candidate viruses are tested in a monkey model for nasal infection, indicated by mucous production.

The viruses of the invention can be attenuated such that one or more of the functional characteristics of the virus are impaired. In certain embodiments, attenuation is measured in comparison to the wild type strain of the virus from which the attenuated virus is derived. In other embodiments, attenuation is determined by comparing the growth of an attenuated virus in different host systems. Thus, for a non-limiting example, a bovine PIV3 is said to be attenuated when grown in a human host if the growth of the bovine PIV3 in the human host is reduced compared to the growth of the bovine PIV3 in a bovine host.

In certain embodiments, the attenuated virus of the invention is capable of infecting a host, is capable of replicating in a host such that infectious viral particles are produced. In comparison to the wild type strain, however, the attenuated strain grows to lower titers or grows more slowly. Any technique known to the skilled artisan can be used to determine the growth curve of the attenuated virus and compare it to the growth curve of the wild type virus. For exemplary methods see Example section, infra. In a specific embodiment, the attenuated virus grows to a titer of less than 10⁵ pfu/ml, of less than 10⁴ pfu/ml, of less than 10³ pfu/ml, or of less than 10² pfu/ml in Vero cells under conditions as described.

In certain embodiments, the attenuated virus of the invention (e.g., a chimeric PIV3) cannot replicate in human cells as well as the wild type virus (e.g., wild type PIV3) does. However, the attenuated virus can replicate well in a cell line that lack interferon functions, such as Vero cells.

In other embodiments, the attenuated virus of the invention is capable of infecting a host, of replicating in the host, and of causing proteins of the virus of the invention to be inserted into the cytoplasmic membrane, but the attenuated virus does not cause the host to produce new infectious viral particles. In certain embodiments, the attenuated virus infects the host, replicates in the host, and causes viral proteins to be inserted in the cytoplasmic membrane of the host with the same efficiency as the wild type mammalian virus. In other embodiments, the ability of the attenuated virus to cause viral proteins to be inserted into the cytoplasmic membrane into the host cell is reduced compared to the wild type virus. In certain embodiments, the ability of the attenuated mammalian virus to replicate in the host is reduced compared to the wild type virus. Any technique known to the skilled artisan can be used to determine whether a virus is capable of infecting a mammalian cell, of replicating within the host, and of causing viral proteins to be inserted into the cytoplasmic membrane of the host. For illustrative methods see section 5.5.

In certain embodiments, the attenuated virus of the invention is capable of infecting a host. In contrast to a wild type PIV, however, the attenuated PIV cannot be replicated in the host. In a specific embodiment, the attenuated virus can infect a host and can cause the host to insert viral proteins in its cytoplasmic membranes, but the attenuated virus is incapable of being replicated in the host. Any method known to the skilled artisan can be used to test whether the attenuated virus has infected the host and has caused the host to insert viral proteins in its cytoplasmic membranes.

In certain embodiments, the ability of the attenuated mammalian virus to infect a host is reduced compared to the ability of the wild type virus to infect the same host. Any technique known to the skilled artisan can be used to determine whether a virus is capable of infecting a host. For illustrative methods see section 5.5.

In certain embodiments, mutations (e.g., missense mutations) are introduced into the genome of the virus to generated a virus with an attenuated phenotype. Mutations (e.g., missense mutations) can be introduced into the N-gene, the P-gene, the F-gene, the M2-gene, the M2-1-gene, the M2-2-gene, the SH-gene, the G-gene or the L-gene of the recombinant virus. Mutations can be additions, substitutions, deletions, or combinations thereof. In specific embodiments, a single amino acid deletion mutation for the N, P, L or M2 proteins are introduced, which can be screened for functionality in the mini-genome assay system and be evaluated for predicted functionality in the virus. In more specific embodiments, the missense mutation is a cold-sensitive mutation. In other embodiments, the missense mutation is a heat-sensitive mutation. In one embodiment, major phosphorylation sites of P protein of the virus is removed. In another embodiment, a mutation or mutations are introduced into the L gene of the virus to generate a temperature sensitive strain. In yet another embodiment, the cleavage site of the F gene is mutated in such a way that cleavage does not occur or occurs at very low efficiency.

In other embodiments, deletions are introduced into the genome of the recombinant virus. In more specific embodiments, a deletion can be introduced into the N-gene, the P-gene, the F-gene, the M2-gene, the M2-1-gene, the M2-2-gene, the SH-gene, the G-gene or the L-gene of the recombinant virus. In specific embodiments, the deletion is in the M2-gene of the recombinant virus of the present invention. In other specific embodiments, the deletion is in the SH-gene of the recombinant virus of the present invention. In yet another specific embodiment, both the M2-gene and the SH-gene are deleted.

In certain embodiments, the intergenic region of the recombinant virus is altered. In one embodiment, the length of the intergenic region is altered. See Section 5.1.2. for illustrative examples. In another embodiment, the intergenic regions are shuffled from 5′ to 3′ end of the viral genome.

In other embodiments, the genome position of a gene or genes of the recombinant virus is changed. In one embodiment, the F or G gene is moved to the 3′ end of the genome. In another embodiment, the N gene is moved to the 5′ end of the genome.

In certain embodiments, attenuation of the virus is achieved by replacing a gene of the wild type virus with a gene of a virus of a different species. In illustrative embodiments, the N-gene, the P-gene, the F-gene, the M2-gene, the M2-1-gene, the M2-2-gene, the SH-gene, the HN-gene or the L-gene of bPIV3 is replaced with the N-gene, the P-gene, the F-gene, the M2-gene, the M2-1-gene, the M2-2-gene, the SH-gene, the HN-gene or the L-gene, respectively, of hPIV3. In other illustrative embodiments, the N-gene, the P-gene, the F-gene, the M2-gene, the M2-1-gene, the M2-2-gene, the SH-gene, the HN-gene or the L-gene of hPIV3 is replaced with the N-gene, the P-gene, the F-gene, the M2-gene, the M2-1-gene, the M2-2-gene, the SH-gene, the HN-gene or the L-gene, respectively, of bPIV3. In a preferred embodiment, attenuation of the virus is achieved by replacing one or more polymerase associated genes (e.g., N, P, L or M2) with genes of a virus of a different species.

In certain embodiments, attenuation of the virus is achieved by replacing or deleting one or more specific domains of a protein of the wild type virus with domains derived from the corresponding protein of a virus of a different species. In an illustrative embodiment, the ectodomain of a F protein of bPIV3 is replaced with an ectodomain of a F protein of a metapneumovirus. In a preferred embodiment, one or more specific domains of L, N, or P protein are replaced with domains derived from corresponding proteins of a virus of a different species. In another illustrative embodiment, the transmembrane domain of the F protein is deleted so that a soluble F protein is expressed.

In certain embodiments of the invention, the leader and/or trailer sequence of the recombinant virus of the invention can be modified to achieve an attenuated phenotype. In certain, more specific embodiments, the leader and/or trailer sequence is reduced in length relative to the wild type virus by at least 1 nucleotide, at least 2 nucleotides, at least 3 nucleotides, at least 4 nucleotides, at least 5 nucleotides or at least 6 nucleotides. In certain other, more specific embodiments, the sequence of the leader and/or trailer of the recombinant virus is mutated. In a specific embodiment, the leader and the trailer sequence are 100% complementary to each other. In other embodiments, 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides are not complementary to each other where the remaining nucleotides of the leader and the trailer sequences are complementary to each other. In certain embodiments, the non-complementary nucleotides are identical to each other. In certain other embodiments, the non-complementary nucleotides are different from each other. In other embodiments, if the non-complementary nucleotide in the trailer is purine, the corresponding nucleotide in the leader sequence is also a purine. In other embodiments, if the non-complementary nucleotide in the trailer is pyrimidine, the corresponding nucleotide in the leader sequence is also a purine.

When a live attenuated vaccine is used, its safety must also be considered. The vaccine must not cause disease. Any techniques known in the art that can make a vaccine safe may be used in the present invention. In addition to attenuation techniques, other techniques may be used. One non-limiting example is to use a soluble heterologous gene that cannot be incorporated into the virion membrane. For example, a single copy of the soluble RSV F gene, a version of the RSV gene lacking the transmembrane and cytosolic domains, can be used. Since it cannot be incorporated into the virion membrane, the virus tropism is not expected to change.

Various assays can be used to test the safety of a vaccine. See section 5.5., infra. Particularly, sucrose gradients and neutralization assays can be used. A sucrose gradient assay can be used to determine whether a heterologous protein is inserted in a virion. If the heterologous protein is inserted in the virion, the virion should be tested for its ability to cause symptoms even if the parental strain does not cause symptoms. Without bound by theory, if the heterologous protein is incorporated in the virion, the virus may have acquired new, possibly pathological, properties.

5.5. Measurement of Viral Titer, Expression of Antigenic Sequences, Immunogenicity and Other Characteristics of Chimeric Viruses

A number of assays may be employed in accordance with the present invention in order to determine the rate of growth of a chimeric or recombinant virus in a cell culture system, an animal model system or in a subject. A number of assays may also be employed in accordance with the present invention in order to determine the requirements of the chimeric and recombinant viruses to achieve infection, replication and packaging of virions.

The assays described herein may be used to assay viral titre over time to determine the growth characteristics of the virus. In a specific embodiment, the viral titre is determined by obtaining a sample from the infected cells or the infected subject, preparing a serial dilution of the sample and infecting a monolayer of cells that are susceptible to infection with the virus at a dilution of the virus that allows for the emergence of single plaques. The plaques can then be counted and the viral titre express as plaque forming units per milliliter of sample. In a specific embodiment of the invention, the growth rate of a virus of the invention in a subject is estimated by the titer of antibodies against the virus in the subject. Without being bound by theory, the antibody titer in the subject reflects not only the viral titer in the subject but also the antigenicity. If the antigenicity of the virus is constant, the increase of the antibody titer in the subject can be used to determine the growth curve of the virus in the subject. In a preferred embodiment, the growth rate of the virus in animals or humans is best tested by sampling biological fluids of a host at multiple time points post-infection and measuring viral titer.

The expression of heterologous gene sequence in a cell culture system or in a subject can be determined by any technique known to the skilled artisan. In certain embodiments, the expression of the heterologous gene is measured by quantifying the level of the transcript. The level of the transcript can be measured by Northern blot analysis or by RT-PCR using probes or primers, respectively, that are specific for the transcript. The transcript can be distinguished from the genome of the virus because the virus is in the antisense orientation whereas the transcript is in the sense orientation. In certain embodiments, the expression of the heterologous gene is measured by quantifying the level of the protein product of the heterologous gene. The level of the protein can be measured by Western blot analysis using antibodies that are specific to the protein.

In a specific embodiment, the heterologous gene is tagged with a peptide tag. The peptide tag can be detected using antibodies against the peptide tag. The level of peptide tag detected is representative for the level of protein expressed from the heterologous gene. Alternatively, the protein expressed from the heterologous gene can be isolated by virtue of the peptide tag. The amount of the purified protein correlates with the expression level of the heterologous gene. Such peptide tags and methods for the isolation of proteins fused to such a peptide tag are well known in the art. A variety of peptide tags known in the art may be used in the modification of the heterologous gene, such as, but not limited to, the immunoglobulin constant regions, polyhistidine sequence (Petty, 1996, Metal-chelate affinity chromatography, in Current Protocols in Molecular Biology, volume 1-3 (1994-1998). Ed. by Ausubel, F. M., Brent, R., Kunston, R. E., Moore, D. D., Seidman, J. G., Smith, J. A. and Struhl, K. Published by John Wiley and sons, Inc., USA, Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67: 21-30), various cellulose binding domains (U.S. Pat. Nos. 5,496,934; 5,202,247; 5,137,819; Tomme et al., 1994, Protein Eng. 7: 117-123), and the FLAG epitope (Short Protocols in Molecular Biology, 1999, Ed. Ausubel et al., John Wiley & Sons, Inc., Unit 10.11) etc. Other peptide tags are recognized by specific binding partners and thus facilitate isolation by affinity binding to the binding partner, which is preferably immobilized and/or on a solid support. As will be appreciated by those skilled in the art, many methods can be used to obtain the coding region of the above-mentioned peptide tags, including but not limited to, DNA cloning, DNA amplification, and synthetic methods. Some of the peptide tags and reagents for their detection and isolation are available commercially.

Samples from a subject can be obtained by any method known to the skilled artisan. In certain embodiments, the sample consists of nasal aspirate, throat swab, sputum or broncho-alveolar lavage.

5.5.1. Minigenome Constructs

Minireplicon constructs can be generated to contain an antisense reporter gene. Any reporter gene known to the skilled artisan can be used with the invention. In a specific embodiment, the reporter gene is CAT. In certain embodiments, the reporter gene can be flanked by the negative-sense bPIV or hPIV leader linked to the hepatitis delta ribozyme (Hep-d Ribo) and T7 polymerase termination (T-T7) signals, and the bPIV or hPIV trailer sequence preceded by the T7 RNA polymerase promoter.

In certain embodiments, the plasmid encoding the minireplicon is transfected into a host cell. The host cell expresses T7 RNA polymerase, the N gene, the P gene, the L gene, and the M2.1 gene. In certain embodiments, the host cell is transfected with plasmids encoding T7 RNA polymerase, the N gene, the P gene, the L gene, and the M2.1 gene. In other embodiments, the plasmid encoding the minireplicon is transfected into a host cell and the host cell is infected with a helper virus.

The expression level of the reporter gene and/or its activity can be assayed by any method known to the skilled artisan, such as, but not limited to, the methods described in section 5.5.6.

In certain, more specific, embodiments, the minireplicon comprises the following elements, in the order listed: T7 RNA Polymerase or RNA polymerase I, leader sequence, gene start, GFP, trailer sequence, Hepatitis delta ribozyme sequence or RNA polymerase I termination sequence. If T7 is used as RNA polymerase, Hepatitis delta ribozyme sequence should be used as termination sequence. If RNA polymerase I is used, RNA polymerase I termination sequence may be used as a termination signal. Dependent on the rescue system, the sequence of the minireplicon can be in the sense or antisense orientation. In certain embodiments, the leader sequence can be modified relative to the wild type leader sequence of the virus of the invention. The leader sequence can optionally be preceded by an AC. The T7 promoter sequence can be with or without a G-doublet or triplet, where the G-doublet or triplet provides for increased transcription.

In a specific embodiment, a cell is infected with a virus of the invention at T0. 24 hours later, at T24, the cell is transfected with a minireplicon construct. 48 hours after T0 and 72 hours after T0, the cells are tested for the expression of the reporter gene. If a fluorescent reporter gene product is used (e.g., GFP), the expression of the reporter gene can be tested using FACS.

In another embodiment, a cell is transfected with six plasmids at T=0 hours. Cells are then harvested at T=40 hours and T=60 hours and analyzed for CAT or GFP expression.

In another specific embodiment, a cell is infected with MVA-T7 at T0. 1 hour later, at T1, the cell is transfected with a minireplicon construct. 24 hours after T0, the cell is infected with a virus of the invention. 72 hours after T0, the cells are tested for the expression of the reporter gene. If a fluorescent reporter gene product is used (e.g., GFP), the expression of the reporter gene can be tested using FACS.

5.5.2. Measurement of Incidence of Infection Rate

The incidence of infection can be determined by any method well-known in the art, including but not limited to, the testing of clinical samples (e.g., nasal swabs) for the presence of an infection, e.g., hMPV, RSV, hPIV, or bPIV/hPIV components can be detected by immunofluorescence assay (IFA) using an anti-hMPV-antigen antibody, an anti-RSV-antigen antibody, an anti-hPIV-antigen antibody, and/or an antibody that is specific to the gene product of the heterologous nucleotide sequence, respectively.

In certain embodiments, samples containing intact cells can be directly processed, whereas isolates without intact cells should first be cultured on a permissive cell line (e.g. HEp-2 cells). In an illustrative embodiment, cultured cell suspensions are cleared by centrifugation at, e.g., 300×g for 5 minutes at room temperature, followed by a PBS, pH 7.4 (Ca++ and Mg++ free) wash under the same conditions. Cell pellets are resuspended in a small volume of PBS for analysis. Primary clinical isolates containing intact cells are mixed with PBS and centrifuged at 300×g for 5 minutes at room temperature. Mucus is removed from the interface with a sterile pipette tip and cell pellets are washed once more with PBS under the same conditions. Pellets are then resuspended in a small volume of PBS for analysis. Five to ten microliters of each cell suspension are spotted per 5 mm well on acetone washed 12-well HTC supercured glass slides and allowed to air dry. Slides are fixed in cold (−20° C.) acetone for 10 minutes. Reactions are blocked by adding PBS-1% BSA to each well followed by a 10 minute incubation at room temperature. Slides are washed three times in PBS-0.1% Tween-20 and air dried. Ten microliters of each primary antibody reagent diluted to 250 ng/ml in blocking buffer is spotted per well and reactions are incubated in a humidified 37° C. environment for 30 minutes. Slides are then washed extensively in three changes of PBS-0.1% Tween-20 and air dried. Ten microliters of appropriate secondary conjugated antibody reagent diluted to 250 ng/ml in blocking buffer are spotted per respective well and reactions are incubated in a humidified 37° C. environment for an additional 30 minutes. Slides are then washed in three changes of PBS-0.1% Tween-20. Five microliters of PBS-50% glycerol-10 mM Tris pH 8.0-1 mM EDTA are spotted per reaction well and slides are mounted with cover slips. Each reaction well is subsequently analyzed by fluorescence microscopy at 200× power using a B-2A filter (EX 450-490 nm). Positive reactions are scored against an autofluorescent background obtained from unstained cells or cells stained with secondary reagent alone. RSV positive reactions are characterized by bright fluorescence punctuated with small inclusions in the cytoplasm of infected cells.

5.5.3. Measurement of Serum Titer

Antibody serum titer can be determined by any method well-known in the art, for example, but not limited to, the amount of antibody or antibody fragment in serum samples can be quantitated by a sandwich ELISA. Briefly, the ELISA consists of coating microtiter plates overnight at 4° C. with an antibody that recognizes the antibody or antibody fragment in the serum. The plates are then blocked for approximately 30 minutes at room temperature with PBS-Tween-0.5% BSA. Standard curves are constructed using purified antibody or antibody fragment diluted in PBS-BSA-BSA, and samples are diluted in PBS-BSA. The samples and standards are added to duplicate wells of the assay plate and are incubated for approximately 1 hour at room temperature. Next, the non-bound antibody is washed away with PBS-TWEEN and the bound antibody is treated with a labeled secondary antibody (e.g., horseradish peroxidase conjugated goat-anti-human IgG) for approximately 1 hour at room temperature. Binding of the labeled antibody is detected by adding a chromogenic substrate specific for the label and measuring the rate of substrate turnover, e.g., by a spectrophotometer. The concentration of antibody or antibody fragment levels in the serum is determined by comparison of the rate of substrate turnover for the samples to the rate of substrate turnover for the standard curve.

5.5.4. Challenge Studies

This assay is used to determine the ability of the recombinant viruses of the invention and of the vaccines of the invention to prevent lower respiratory tract viral infection in an animal model system, including but not limited to, cotton rats, Syrian Golden hamsters, and Balb/c mice. The recombinant virus and/or the vaccine can be administered by intravenous (IV) route, by intramuscular (IM) route or by intranasal route (IN). The recombinant virus and/or the vaccine can be administered by any technique well-known to the skilled artisan. This assay is also used to correlate the serum concentration of antibodies with a reduction in lung titer of the virus to which the antibodies bind.

On day 0, groups of animals, including but not limited to, cotton rats (Sigmodon hispidis, average weight 100 g), cynomolgous macacques (average weight 2.0 kg) and hamsters (e.g., Syrian Golden hamsters) are inoculated with the recombinant virus or the vaccine of interest or BSA by intramuscular injection, by intravenous injection, or by intranasal route. Prior to, concurrently with, or subsequent to administration of the recombinant virus or the vaccine of the invention, the animals are infected with wild type virus wherein the wild type virus is the virus against which the vaccine was generated. In certain embodiments, the animals are infected with the wild type virus at least 1 day, at least 2 days, at least 3 days, at least 4 days, at least 5 days, at least 6 days, at least 1 week, at least 2 weeks, at least 3 weeks or at least 4 weeks subsequent to the administration of the recombinant virus and/or the vaccine of the invention. In a preferred embodiment, the animals are infected with the wild type virus 21 days subsequent to the administration of the recombinant virus and/or the vaccine of the invention. In another preferred embodiment, the animals are infected with the wild type virus 28 days subsequent to the administration of the recombinant virus and/or the vaccine of the invention.

After the infection, the animals are sacrificed, and their nasal turbinate tissue and/or lung tissue are harvested and virus titers are determined by appropriate assays, e.g., plaque assay and TCID₅₀ assay. Bovine serum albumin (BSA) 10 mg/kg can be used as a negative control. Antibody concentrations in the serum at the time of challenge can be determined using a sandwich ELISA.

5.5.5. Clinical Trials

Vaccines of the invention or fragments thereof that have been tested in in vitro assays and animal models may be further evaluated for safety, tolerance, immunogenicity, infectivity and pharmacokinetics in groups of normal healthy human volunteers, including all age groups. In a preferred embodiment, the healthy human volunteers are infants at about 6 weeks of age or older, children and adults. The volunteers are administered intranasally, intramuscularly, intravenously or by a pulmonary delivery system in a single dose of a recombinant virus of the invention and/or a vaccine of the invention. Multiple doses of virus and/or vaccine of the invention may be required in seronegative children 6 to 60 months of age. Multiple doses of virus and/or vaccine of the invention may also be required in the first six months of life to stimulate local and systemic immunity and to overcome neutralization by maternal antibody. In a preferred embodiment, a primary dosing regimen at 2, 4, and 6 months of age and a booster dose at the beginning of the second year of life are used. A recombinant virus of the invention and/or a vaccine of the invention can be administered alone or concurrently with pediatric vaccines recommended at the corresponding ages.

In a preferred embodiment, double-blind randomized, placebo-controlled clinical trials are used. In a specific embodiment, a computer generated randomization schedule is used. For example, each subject in the study will be enrolled as a single unit and assigned a unique case number. Multiple subjects within a single family will be treated as individuals for the purpose of enrollment. Parent/guardian, subjects, and investigators will remain blinded to which treatment group subjects have been assigned for the duration of the study. Serologic and virologic studies will be performed by laboratory personnel blinded to treatment group assignment. However, it is expected that isolation of the vaccine virus from nasal wash fluid obtained after vaccination will identify likely vaccinees to the virology laboratory staff. The serologic and virologic staff are separate and the serology group will be prevented from acquiring any knowledge of the culture results.

Each volunteer is preferably monitored for at least 12 hours prior to receiving the recombinant virus of the invention and/or a vaccine of the invention, and each volunteer will be monitored for at least fifteen minutes after receiving the dose at a clinical site. Then volunteers are monitored as outpatients on days 1-14, 21, 28, 35, 42, 49, and 56 postdose. In a preferred embodiment, the volunteers are monitored for the first month after each vaccination as outpatients. All vaccine related serious adverse events will be reported for the entire duration of the trial. A serious adverse event is defined as an event that 1) results in death, 2) is immediately life threatening, 3) results in permanent or substantial disability, 4) results in or prolongs an existing in-patient hospitalization, 5) results in a congenital anomaly, 6) is a cancer, or 7) is the result of an overdose of the study vaccine. Serious adverse events that are not vaccine related will be reported beginning on the day of the first vaccination (Day 0) and continue for 30 days following the last vaccination. Non-vaccine related serious adverse events will not be reported for 5 to 8 months after the 30 day reporting period following the last vaccination. A dose of vaccine/placebo will not be given if a child has a vaccine-related serous adverse event following the previous dose. Any adverse event that is not considered vaccine related, but which is of concern, will be discussed by the clinical study monitor and the medical monitor before the decision to give another dose is made.

Blood samples are collected via an indwelling catheter or direct venipuncture (e.g., by using 10 ml red-top Vacutainer tubes) at the following intervals: (1) prior to administering the dose of the recombinant virus of the invention and/or a vaccine of the invention; (2) during the administration of the dose of the recombinant virus of the invention and/or a vaccine of the invention; (3) 5 minutes, 10 minutes, 15 minutes, 20 minutes, 30 minutes, 1 hour, 2 hours, 4 hours, 8 hours, 12 hours, 24 hours, and 48 hours after administering the dose of the recombinant virus of the invention and/or a vaccine of the invention; and (4) 3 days, 7 days 14 days, 21 days, 28 days, 35 days, 42 days, 49 days, and 56 days after administering the dose of the recombinant virus of the invention and/or a vaccine of the invention. In a specific embodiment, a total of 5 blood draws (3-5 ml each) are obtained, each just prior to the first, third and booster doses and approximately one month following the third dose and booster dose of administration of the vaccine or placebo. Samples are allowed to clot at room temperature and the serum is collected after centrifugation.

Sera are tested for strain-specific serum hemaglutination inhibition (HAI) antibody levels against the virus of the invention. Other indicators of immunogenicity such as IgG, IgA, or neutralizing antibodies are also tested. Serum antibody responses to one or more of the other vaccines given concurrently may be measured. The amount of antibodies generated against the recombinant virus of the invention and/or a vaccine of the invention in the samples from the patients can be quantitated by ELISA. T-cell immunity (cytotoxic and helper responses) in PBMC and lung and nasal ravages can also be monitored.

The concentration of antibody levels in the serum of volunteers are corrected by subtracting the predose serum level (background level) from the serum levels at each collection interval after administration of the dose of recombinant virus of the invention and/or a vaccine of the invention. For each volunteer the pharmacokinetic parameters are computed according to the model-independent approach (Gibaldi et al., eds., 1982, Pharmacokinetics, 2^{nd} edition, Marcel Dekker, New York) from the corrected serum antibody or antibody fragment concentrations.

Nasal washes obtained approximately 2, 3, 4, 5, 6, 7 or 8 days after each doses of vaccine/placebo will be cultured to detect shedding of the vaccine virus of the invention. In a preferred embodiment, nasal washes obtained 7 days after each doses of vaccine/placebo are cultured. A nasopharyngeal swab, a throat swab, or a nasal wash is also used to determine the presence of other viruses in volunteers with medically attended febrile illness (rectal temperature greater than or equal to 102° F.) and/or croup, bronchiolitis, or pneumonia at any time during the study. Samples are shipped on dry ice to designated site for study. Assays for isolation and quantitation of the vaccine virus of the invention and immunostaining assays using MAb to identify the vaccine virus of the invention are used (examples of such assays are given in the Example sections, infra). Nasal wash specimens may be tested for other viruses and immune responses including IgG, IgA, and neutralizing antibody.

5.5.6. Reporter Genes

In certain embodiments, assays for measurement of reporter gene expression in tissue culture or in animal models can be used with the methods of the invention. The nucleotide sequence of the reporter gene is cloned into the virus, such as bPIV, hPIV, or b/hPIV3, wherein (i) the position of the reporter gene is changed and (ii) the length of the intergenic regions flanking the reporter gene are varied. Different combinations are tested to determine the optimal rate of expression of the reporter gene and the optimal replication rate of the virus comprising the reporter gene.

In certain embodiments, minigenome constructs are generated to include a reporter gene. The construction of minigenome constructs is described in section 5.5.1.

The abundance of the reporter gene product can be determined by any technique known to the skilled artisan. Such techniques include, but are not limited to, Northern blot analysis or Western blot analysis using probes or antibodies, respectively, that are specific to the reporter gene.

In certain embodiments, the reporter gene emits a fluorescent signal that can be detected in a FACS. FACS can be used to detect cells in which the reporter gene is expressed.

Techniques for practicing the specific aspect of this invention will employ, unless otherwise indicated, conventional techniques of molecular biology, microbiology, and recombinant DNA manipulation and production, which are routinely practiced by one of skill in the art. See, e.g., Sambrook, 1989, Molecular Cloning, A Laboratory Manual, Second Edition; DNA Cloning, Volumes I and II (Glover, Ed. 1985); and Transcription and Translation (Hames & Higgins, Eds. 1984).

The biochemical activity of the reporter gene product represents the expression level of the reporter gene. The total level of reporter gene activity depends also on the replication rate of the recombinant virus of the invention. Thus, to determine the true expression level of the reporter gene from the recombinant virus, the total expression level should be divided by the titer of the recombinant virus in the cell culture or the animal model.

Reporter genes that can be used with the methods of invention include, but are not limited to, the genes listed in the Table 4 below:

TABLE 4
Reporter genes and the biochemical properties
of the respective reporter gene products

| Reporter Gene | Protein Activity & Measurement |
|---|---|
| CAT (chloramphenicol | Transfers radioactive acetyl groups to |
| acetyltransferase) | chloramphenicol or detection by thin layer |
| | chromatography and autoradiography |
| GAL (b-galactosidase) | Hydrolyzes colorless galactosides to yield |
| | colored products. |
| GUS (b-glucuronidase) | Hydrolyzes colorless glucuronides to yield |
| | colored products. |
| LUC (luciferase) | Oxidizes luciferin, emitting photons |
| GFP (green fluorescent | fluorescent protein without substrate |
| protein) | |
| SEAP (secreted alkaline | luminescence reaction with suitable substrates |
| phosphatase) | or with substrates that generate chromophores |
| HRP (horseradish | in the presence of hydrogen oxide, oxidation of |
| peroxidase) | 3,3′,5,5′-tetramethylbenzidine to form a colored |
| | complex |
| AP (alkaline | luminescence reaction with suitable substrates |
| phosphatase) | or with substrates that generate chromophores |

The abundance of the reporter gene can be measured by, inter alia, Western blot analysis or Northern blot analysis or any other technique used for the quantification of transcription of a nucleotide sequence, the abundance of its mRNA its protein (see Short Protocols in Molecular Biology, Ausubel et al. (editors), John Wiley & Sons, Inc., 4^{th} edition, 1999). In certain embodiments, the activity of the reporter gene product is measured as a readout of reporter gene expression from the recombinant virus. For the quantification of the activity of the reporter gene product, biochemical characteristics of the reporter gene product can be investigated (see Table 1). The methods for measuring the biochemical activity of the reporter gene products are well-known to the skilled artisan. A more detailed description of illustrative reporter genes that can be used with the methods of the invention is set forth below.

Luciferase

Luciferases are enzymes that emit light in the presence of oxygen and a substrate (luciferin) and which have been used for real-time, low-light imaging of gene expression in cell cultures, individual cells, whole organisms, and transgenic organisms (reviewed by Greer & Szalay, 2002, Luminescence 17(1): 43-74).

As used herein, the term “luciferase” as used in relation to the invention is intended to embrace all luciferases, or recombinant enzymes derived from luciferases that have luciferase activity. The luciferase genes from fireflies have been well characterized, for example, from the Photinus and Luciola species (see, e.g., International Patent Publication No. WO 95/25798 for Photinus pyralis, European Patent Application No. EP 0 524 448 for Luciola cruciata and Luciola lateralis, and Devine et al., 1993, Biochim. Biophys. Acta 1173(2): 121-132 for Luciola mingrelica. Other eucaryotic luciferase genes include, but are not limited to, the sea panzy (Renilla reniformis, see, e.g., Lorenz et al., 1991, Proc Natl Acad Sci USA 88(10): 4438-4442), and the glow worm (Lampyris noctiluca, see e.g., Sula-Newby et al., 1996, Biochem J. 313: 761-767). Bacterial luciferin-luciferase systems include, but are not limited to, the bacterial lux genes of terrestrial Photorhabdus luminescens (see, e.g., Manukhov et al., 2000, Genetika 36(3): 322-30) and marine bacteria Vibrio fischeri and Vibrio harveyi (see, e.g., Miyamoto et al., 1988, J Biol. Chem. 263(26): 13393-9, and Cohn et al., 1983, Proc Natl Acad Sci USA., 80(1): 120-3, respectively). The luciferases encompassed by the present invention also includes the mutant luciferases described in U.S. Pat. No. 6,265,177 to Squirrell et al., which is hereby incorporated by reference in its entirety.

Green Fluorescent Protein

Green fluorescent protein (“GFP”) is a 238 amino acid protein with amino acids 65 to 67 involved in the formation of the chromophore that does not require additional substrates or cofactors to fluoresce (see, e.g., Prasher et al., 1992, Gene 111: 229-233; Yang et al., 1996, Nature Biotechnol. 14: 1252-1256; and Cody et al., 1993, Biochemistry 32: 1212-1218).

As used herein, the term “green fluorescent protein” or “GFP” as used in relation to the invention is intended to embrace all GFPs (including the various forms of GFPs that exhibit colors other than green), or recombinant enzymes derived from GFPs that have GFP activity. The native gene for GFP was cloned from the bioluminescent jellyfish Aequorea victoria (see, e.g., Morin et al., 1972, J. Cell Physiol. 77: 313-318). Wild type GFP has a major excitation peak at 395 nm and a minor excitation peak at 470 nm. The absorption peak at 470 nm allows the monitoring of GFP levels using standard fluorescein isothiocyanate (FITC) filter sets. Mutants of the GFP gene have been found useful to enhance expression and to modify excitation and fluorescence. For example, mutant GFPs with alanine, glycine, isoleucine, or threonine substituted for serine at position 65 result in mutant GFPs with shifts in excitation maxima and greater fluorescence than wild type protein when excited at 488 nm (see, e.g., Heim et al., 1995, Nature 373: 663-664); U.S. Pat. No. 5,625,048; Delagrave et al., 1995, Biotechnology 13: 151-154; Cormack et al., 1996, Gene 173: 33-38; and Cramer et al., 1996, Nature Biotechnol. 14: 315-319). The ability to excite GFP at 488 nm permits the use of GFP with standard fluorescence activated cell sorting (“FACS”) equipment. In another embodiment, GFPs are isolated from organisms other than the jellyfish, such as, but not limited to, the sea pansy, Renilla reriformis.

EGFP is a red-shifted variant of wild-type GFP (3-5) which has been optimized for brighter fluorescence and higher expression in mammalian cells. (Excitation maximum=488 nm; emission maximum=507 nm) EGFP encodes the GFPmut1 variant which contains the double-amino-acid substitution of Phe-64 to Leu and Ser-65 to Thr. The coding sequence of the EGFP gene contains more than 190 silent base changes which correspond to human codon-usage preferences.

Beta Galactosidase

Beta galactosidase (“β-gal”) is an enzyme that catalyzes the hydrolysis of b-galactosides, including lactose, and the galactoside analogs o-nitrophenyl-β-D-galactopyranoside (“ONPG”) and chlorophenol red-b-D-galactopyranoside (“CPRG”) (see, e.g., Nielsen et al., 1983 Proc Natl Acad Sci USA 80(17): 5198-5202; Eustice et al., 1991, Biotechniques 11: 739-742; and Henderson et al., 1986, Clin. Chem. 32: 1637-1641). The β-gal gene functions well as a reporter gene because the protein product is extremely stable, resistant to proteolytic degradation in cellular lysates, and easily assayed. When ONPG is used as the substrate, β-gal activity can be quantitated with a spectrophotometer or a microplate reader.

As used herein, the term “beta galactosidase” or “β-gal” as used in relation to the invention is intended to embrace all b-gals, including lacZ gene products, or recombinant enzymes derived from b-gals which have b-gal activity. The b-gal gene functions well as a reporter gene because the protein product is extremely stable, resistant to proteolytic degradation in cellular lysates, and easily assayed. In an embodiment where ONPG is the substrate, b-gal activity can be quantitated with a spectrophotometer or microplate reader to determine the amount of ONPG converted at 420 nm. In an embodiment when CPRG is the substrate, b-gal activity can be quantitated with a spectrophotometer or microplate reader to determine the amount of CPRG converted at 570 to 595 nm.

Chloramphenicol Acetyltransferase

Chloramphenicol acetyl transferase (“CAT”) is commonly used as a reporter gene in mammalian cell systems because mammalian cells do not have detectable levels of CAT activity. The assay for CAT involves incubating cellular extracts with radiolabeled chloramphenicol and appropriate co-factors, separating the starting materials from the product by, for example, thin layer chromatography (“TLC”), followed by scintillation counting (see, e.g., U.S. Pat. No. 5,726,041, which is hereby incorporated by reference in its entirety).

As used herein, the term “chloramphenicol acetyltransferase” or “CAT” as used in relation to the invention is intended to embrace all CATs, or recombinant enzymes derived from CAT which have CAT activity. While it is preferable that a reporter system which does not require cell processing, radioisotopes, and chromatographic separations would be more amenable to high through-put screening, CAT as a reporter gene may be preferable in situations when stability of the reporter gene is important. For example, the CAT reporter protein has an in vivo half life of about 50 hours, which is advantageous when an accumulative versus a dynamic change type of result is desired.

Secreted Alkaline Phosphatase

The secreted alkaline phosphatase (“SEAP”) enzyme is a truncated form of alkaline phosphatase, in which the cleavage of the transmembrane domain of the protein allows it to be secreted from the cells into the surrounding media.

As used herein, the term “secreted alkaline phosphatase” or “SEAP” as used in relation to the invention is intended to embrace all SEAP or recombinant enzymes derived from SEAP which have alkaline phosphatase activity. SEAP activity can be detected by a variety of methods including, but not limited to, measurement of catalysis of a fluorescent substrate, immunoprecipitation, HPLC, and radiometric detection. The luminescent method is preferred due to its increased sensitivity over calorimetric detection methods. The advantages of using SEAP is that a cell lysis step is not required since the SEAP protein is secreted out of the cell, which facilitates the automation of sampling and assay procedures. A cell-based assay using SEAP for use in cell-based assessment of inhibitors of the Hepatitis C virus protease is described in U.S. Pat. No. 6,280,940 to Potts et al. which is hereby incorporated by reference in its entirety.

5.5.7. Cell Culture Systems, Embryonated Eggs, and Animal Models

Cell culture systems known in the art can be used to propagate or test activities of the viruses of the present invention. (See e.g., Flint et al., PRINCIPLES OF VIROLOGY, MOLECULAR BIOLOGY, PATHOGENESIS, AND CONTROL, 2000, ASM Press pp 25-29, the entire text is incorporated herein by reference). Examples of such cell culture systems include, but are not limited to, primary cell culture that are prepared from animal tissues (e.g., cell cultures derived from monkey kidney, human embryonic amnion, kidney, and foreskin, and chicken or mouse embryos); diploid cell strains that consist of a homogeneous population of a single type and can divide up to 100 times before dying (e.g., cell culture derived from human embryos, such as the WI-38 strain derived from human embryonic lung); and continuous cell lines consist of a single cell type that can be propagated indefinitely in culture (e.g., HEp-2 cells, Hela cells, Vero cells, L and 3T3 cells, and BHK-21 cells).

Viruses of the invention can also be propagated in embryonated chicken eggs. At 5 to 14 days after fertilization, a hole is drilled in the shell and virus is injected into the site appropriate for its replication.

Any animal models known in the art can be used in the present invention to accomplish various purposes, such as to determine the effectiveness and safeness of vaccines of the invention. Examples of such animal models include, but are not limited to, cotton rats (Sigmodon hispidis), hamsters, mice, monkeys, and chimpanzees. In a preferred embodiment, Syrian Golden hamsters are used.

5.5.8. Neutralization Assay

Neutralization assays can be carried out to address the important safety issue of whether the heterologous surface glycoproteins are incorporated into the virion which may result in an altered virus tropism phenotype. As used herein, the term “tropism” refers to the affinity of a virus for a particular cell type. Tropism is usually determined by the presence of cell receptors on specific cells which allow a virus to enter that and only that particular cell type. A neutralization assay is performed by using either MAbs of the heterologous surface glycoprotein (non-limiting example is the F protein of a negative strand RNA virus) or polyclonal antiserum comprising antibodies against the heterologous surface glycoprotein. Different dilution of the antibodies are tested to see whether the chimeric virus of the invention can be neutralized. The heterologous surface glycoprotein should not be present on the virion surface in an amount sufficient to result in antibody binding and neutralization.

5.5.9. Sucrose Gradient Assay

The question of whether the heterologous proteins are incorporated into the virion can be further investigated by use of a biochemical assay. Infected cell lysates can be fractionated in 20-60% sucrose gradients, various fractions are collected and analyzed for the presence and distribution of heterologous proteins and the vector proteins by Western blot. The fractions and the virus proteins can also be assayed for peak virus titers by plaque assay. Examples of sucrose gradient assay are given in section 23, infra. When the heterologous proteins are associated with the virion, they will co-migrate with the virion.

5.6. Vaccine Formulations using the Chimeric Viruses

The invention encompasses vaccine formulations comprising the engineered negative strand RNA virus of the present invention. The recombinant PIV viruses of the present invention may be used as a vehicle to express foreign epitopes that induce a protective response to any of a variety of pathogens. In a specific embodiment, the invention encompasses the use of recombinant bPIV viruses or attenuated hPIV that have been modified in vaccine formulations to confer protection against hPIV infection.

The vaccine preparations of the invention encompass multivalent vaccines, including bivalent and trivalent vaccine preparations. The bivalent and trivalent vaccines of the invention may be administered in the form of one PIV vector expressing each heterologous antigenic sequence or two or more PIV vectors each encoding different heterologous antigenic sequences. For example, a first chimeric PIV expressing one or more heterologous antigenic sequences can be administered in combination with a second chimeric PIV expressing one or more heterologous antigenic sequences, wherein the heterologous antigenic sequences in the second chimeric PIV are different from the heterologous antigenic sequences in the first chimeric PIV. The heterologous antigenic sequences in the first and the second chimeric PIV can be derived from the same virus but encode different proteins, or derived from different viruses. In a preferred embodiment, the heterologous antigenic sequences in the first chimeric PIV are derived from respiratory syncytial virus, and the heterologous antigenic sequences in the second chimeric PIV are derived from human metapneumovirus. In another preferred embodiment, the heterologous antigenic sequences in the first chimeric PIV are derived from respiratory syncytial virus, and the heterologous antigenic sequences in the second chimeric PIV are derived from avian pneumovirus.

In certain preferred embodiments, the vaccine formulation of the invention is used to protect against infections caused by a negative strand RNA virus, including but not limited to, influenza virus, parainfluenza virus, respiratory syncytial virus, and mammalian metapneumovirus (e.g., human metapneumovirus). More specifically, the vaccine formulation of the invention is used to protect against infections by a human metapneumovirus and/or an avian pneumovirus. In certain embodiments, the vaccine formulation of the invention is used to protect against infections by (a) a human metapneumovirus and a respiratory syncytial virus; and/or (b) an avian pneumovirus and a respiratory syncytial virus.

In a preferred embodiment, the invention provides a proteinaceous molecule or metapneumovirus-specific viral protein or functional fragment thereof encoded by a nucleic acid according to the invention. Useful proteinaceous molecules are for example derived from any of the genes or genomic fragments derivable from a virus according to the invention. Particularly useful are the F, SH and/or G protein or antigenic fragments thereof for inclusion as antigen or subunit immunogen, but inactivated whole virus can also be used. Particularly useful are also those proteinaceous substances that are encoded by recombinant nucleic acid fragments that are identified for phylogenetic analyses, of course preferred are those that are within the preferred bounds and metes of ORFs useful in phylogenetic analyses, in particular for eliciting MPV specific antibody or T cell responses, whether in vivo (e.g. for protective purposes or for providing diagnostic antibodies) or in vitro (e.g. by phage display technology or another technique useful for generating synthetic antibodies).

A pharmaceutical composition comprising a virus, a nucleic acid, a proteinaceous molecule or fragment thereof, an antigen and/or an antibody according to the invention can for example be used in a method for the treatment or prevention of a MPV infection and/or a respiratory illness comprising providing an individual with a pharmaceutical composition according to the invention. This is most useful when said individual is a human, specifically when said human is below 5 years of age, since such infants and young children are most likely to be infected by a human MPV as provided herein. Generally, in the acute phase patients will suffer from upper respiratory symptoms predisposing for other respiratory and other diseases. Also lower respiratory illnesses may occur, predisposing for more and other serious conditions. The compositions of the invention can be used for the treatment of immuno-compromised individuals including cancer patients, transplant recipients and the elderly.

The invention also provides methods to obtain an antiviral agent useful in the treatment of respiratory tract illness comprising establishing a cell culture or experimental animal comprising a virus according to the invention, treating said culture or animal with an candidate antiviral agent, and determining the effect of said agent on said virus or its infection of said culture or animal. The invention also provides use of an antiviral agent according to the invention for the preparation of a pharmaceutical composition, in particular for the preparation of a pharmaceutical composition for the treatment of respiratory tract illness, specifically when caused by an MPV infection or related disease, and provides a pharmaceutical composition comprising an antiviral agent according to the invention, useful in a method for the treatment or prevention of an MPV infection or respiratory illness, said method comprising providing an individual with such a pharmaceutical composition.

In certain embodiments of the invention, the vaccine of the invention comprises mammalian metapneumovirus. In certain, more specific embodiments, the mammalian metapneumovirus is a human metapneumovirus. In a preferred embodiment, the mammalian metapneumovirus to be used in a vaccine formulation has an attenuated phenotype. For methods to achieve an attenuated phenotype, see section 5.4.

The invention provides vaccine formulations for the prevention and treatment of infections with PIV, RSV, APV, and/or hMPV. In certain embodiments, the vaccine of the invention comprises recombinant and chimeric viruses of the invention. In certain embodiments, the virus is attenuated.

In a specific embodiment, the vaccine comprises APV and the vaccine is used for the prevention and treatment for hMPV infections in humans. Without being bound by theory, because of the high degree of homology of the F protein of APV with the F protein of hMPV, infection with APV will result in the production of antibodies in the host that will cross-react with hMPV and protect the host from infection with hMPV and related diseases.

In another specific embodiment, the vaccine comprises hMPV and the vaccine is used for the prevention and treatment for APV infection in birds, such as, but not limited to, in turkeys. Without being bound by theory, because of the high degree of homology of the F protein of APV with the F protein of hMPV, infection with hMPV will result in the production of antibodies in the host that will cross-react with APV and protect the host from infection with APV and related diseases.

In certain embodiments, the vaccine formulation of the invention is used to protect against infections by (a) a human metapneumovirus and a human parainfluenza virus; and/or (b) an avian pneumovirus and a human parainfluenza virus and related diseases.

In certain embodiments, the vaccine formulation of the invention is used to protect against infections by (a) a human metapneumovirus, a respiratory syncytial virus, and a human parainfluenza virus; and/or (b) an avian pneumovirus, a respiratory syncytial virus, and a human parainfluenza virus and related diseases.

In certain embodiments, the vaccine formulation of the invention is used to protect against infections by a human metapneumovirus, a respiratory syncytial virus, and a human parainfluenza virus. In certain other embodiments, the vaccine formulation of the invention is used to protect against infections by an avian pneumovirus, a respiratory syncytial virus, and a human parainfluenza virus, and related diseases.

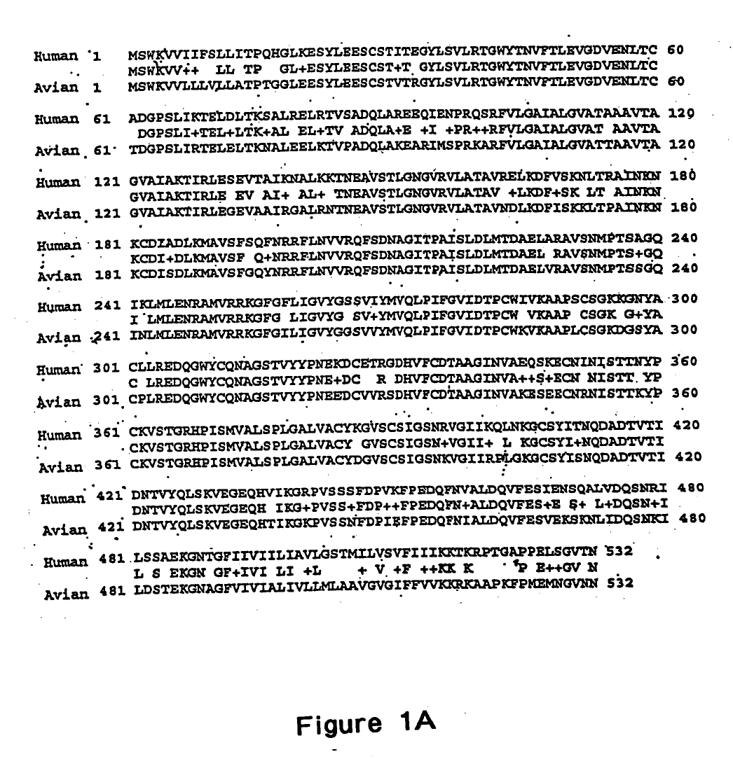

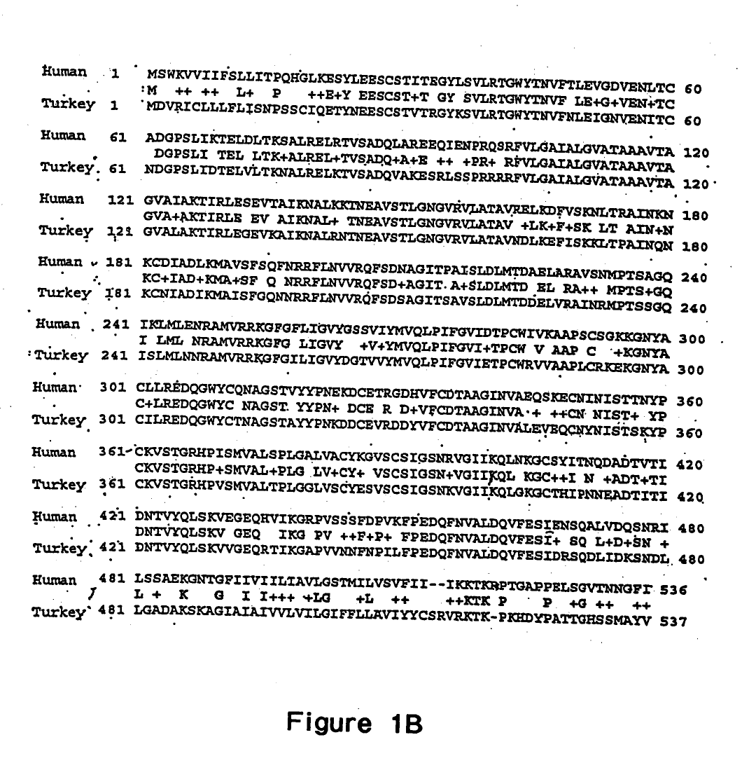

Due to the high degree of homology among the F proteins of different viral species, for exemplary amino acid sequence comparisons see FIG. 1, the vaccine formulations of the invention can be used for protection from viruses different from the one from which the heterologous nucleotide sequence encoding the F protein was derived. In a specific exemplary embodiment, a vaccine formulation contains a virus comprising a heterologous nucleotide sequence derived from an avian pneumovirus type A, and the vaccine formulation is used to protect from infection by avian pneumovirus type A and avian pneumovirus type B. In another specific exemplary embodiment, a vaccine formulation contains a virus comprising a heterologous nucleotide sequence derived from an avian pneumovirus subgroup C, and the vaccine formulation is used to protect from infection by avian pneumovirus subgroup C and avian pneumovirus subgroup D.

The invention encompasses vaccine formulations to be administered to humans and animals that are useful to protect against PIV, hMPV, APV (including APV C and APV D), influenza, RSV, Sendai virus, mumps, laryngotracheitis virus, simianvirus 5, human papillomavirus, as well as other viruses, pathogens and related diseases. The invention further encompasses vaccine formulations to be administered to humans and animals that are useful to protect against human metapneumovirus infections, avian pneumovirus infections, and related diseases.

In one embodiment, the invention encompasses vaccine formulations that are useful against domestic animal disease causing agents including rabies virus, feline leukemia virus (FLV) and canine distemper virus. In yet another embodiment, the invention encompasses vaccine formulations that are useful to protect livestock against vesicular stomatitis virus, rabies virus, rinderpest virus, swinepox virus, and further, to protect wild animals against rabies virus.

Attenuated viruses generated by the reverse genetics approach can be used in the vaccine and pharmaceutical formulations described herein. Reverse genetics techniques can also be used to engineer additional mutations to other viral genes important for vaccine production. For example, mutations in the 5′ non-coding region may affect mRNA translation, mutations in capsid proteins are believed to influence viral assembly, and temperature-sensitive and cold-adapted mutants are often less pathogenic than the parental virus. (see, e.g., Flint et al., PRINCIPLES OF VIROLOGY, MOLECULAR BIOLOGY, PATHOGENESIS, AND CONTROL, 2000, ASM Press pp 670-683, the entire text is incorporated herein by reference). The epitopes of useful vaccine strain variants can be engineered into the attenuated virus. Alternatively, completely foreign epitopes, including antigens derived from other viral or non-viral pathogens can be engineered into the attenuated strain. For example, antigens of non-related viruses such as HIV (gp160, gp120, gp41), parasite antigens (e.g., malaria), bacterial or fungal antigens, or tumor antigens can be engineered into the attenuated strain. Alternatively, epitopes which alter the tropism of the virus in vivo can be engineered into the chimeric attenuated viruses of the invention.

Virtually any heterologous gene sequence may be constructed into the chimeric viruses of the invention for use in vaccines. Preferably, moieties and peptides that act as biological response modifiers are constructed into the chimeric viruses of the invention for use in vaccines. Preferably, epitopes that induce a protective immune response to any of a variety of pathogens, or antigens that bind neutralizing antibodies may be expressed by or as part of the chimeric viruses. For example, heterologous gene sequences that can be constructed into the chimeric viruses of the invention include, but are not limited to influenza and parainfluenza hemagglutinin neuraminidase and fusion glycoproteins such as the HN and F genes of human PIV3. In yet another embodiment, heterologous gene sequences that can be engineered into the chimeric viruses include those that encode proteins with immunomodulating activities. Examples of immunomodulating proteins include, but are not limited to, cytokines, interferon type 1, gamma interferon, colony stimulating factors, interleukin-1, -2, -4, -5, -6, -12, and antagonists of these agents.

In addition, heterologous gene sequences that can be constructed into the chimeric viruses of the invention for use in vaccines include but are not limited to sequences derived from a human immunodeficiency virus (HIV), preferably type 1 or type 2. In a preferred embodiment, an immunogenic HIV-derived peptide that may be the source of an antigen may be constructed into a chimeric PIV that may then be used to elicit a vertebrate immune response. Such HIV-derived peptides may include, but are not limited to, sequences derived from the env gene (i.e., sequences encoding all or part of gp160, gp120, and/or gp41), the pol gene (i.e., sequences encoding all or part of reverse transcriptase, endonuclease, protease, and/or integrase), the gag gene (i.e., sequences encoding all or part of p7, p6, p55, p17/18, p24/25), tat, rev, nef, vif, vpu, vpr, and/or vpx.

Other heterologous sequences may be derived from hepatitis B virus surface antigen (HBsAg); hepatitis A or C virus surface antigens, the glycoproteins of Epstein Barr virus; the glycoproteins of human papillomavirus; the glycoproteins of respiratory syncytial virus, parainfluenza virus, Sendai virus, simianvirus 5 or mumps virus; the glycoproteins of influenza virus; the glycoproteins of herpesviruses; VP1 of poliovirus; antigenic determinants of non-viral pathogens such as bacteria and parasites, to name but a few. In another embodiment, all or portions of immunoglobulin genes may be expressed. For example, variable regions of anti-idiotypic immunoglobulins that mimic such epitopes may be constructed into the chimeric viruses of the invention.

Other heterologous sequences may be derived from tumor antigens, and the resulting chimeric viruses can be used to generate an immune response against the tumor cells leading to tumor regression in vivo. These vaccines may be used in combination with other therapeutic regimens, including but not limited to, chemotherapy, radiation therapy, surgery, bone marrow transplantation, etc. for the treatment of tumors. In accordance with the present invention, recombinant viruses may be engineered to express tumor-associated antigens (TAAs), including but not limited to, human tumor antigens recognized by T cells (Robbins and Kawakami, 1996, Curr. Opin. Immunol. 8: 628-636, incorporated herein by reference in its entirety), melanocyte lineage proteins, including gp100, MART-1/MelanA, TRP-1 (gp75), tyrosinase; Tumor-specific widely shared antigens, MAGE-1, MAGE-3, BAGE, GAGE-1, GAGE-1, N-acetylglucosaminyltransferase-V, p15; Tumor-specific mutated antigens, β-catenin, MUM-1, CDK4; Nonmelanoma antigens for breast, ovarian, cervical and pancreatic carcinoma, HER-2/neu, human papillomavirus-E6, -E7, MUC-1.

In even other embodiments, a heterologous nucleotide sequence is derived from a metapneumovirus, such as human metapneumovirus and/or avian pneumovirus. In even other embodiments, the virus of the invention contains two different heterologous nucleotide sequences wherein one is derived from a metapneumovirus, such as human metapneumovirus and/or avian pneumovirus, and the other one is derived from a respiratory syncytial virus. The heterologous nucleotide sequence encodes a F protein or a G protein of the respective virus. In a specific embodiment, a heterologous nucleotide sequences encodes a chimeric F protein, wherein the chimeric F protein contains the ectodomain of a F protein of a metapneumovirus and the transmembrane domain as well as the luminal domain of a F protein of a parainfluenza virus.

Either a live recombinant viral vaccine or an inactivated recombinant viral vaccine can be formulated. A live vaccine may be preferred because multiplication in the host leads to a prolonged stimulus of similar kind and magnitude to that occurring in natural infections, and therefore, confers substantial, long-lasting immunity. Production of such live recombinant virus vaccine formulations may be accomplished using conventional methods involving propagation of the virus in cell culture or in the allantois of the chick embryo followed by purification. Additionally, as bPIV has been demonstrated to be non-pathogenic in humans, this virus is highly suited for use as a live vaccine.

In this regard, the use of genetically engineered PIV (vectors) for vaccine purposes may desire the presence of attenuation characteristics in these strains. The introduction of appropriate mutations (e.g., deletions) into the templates used for transfection may provide the novel viruses with attenuation characteristics. For example, specific missense mutations that are associated with temperature sensitivity or cold adaption can be made into deletion mutations. These mutations should be more stable than the point mutations associated with cold or temperature sensitive mutants and reversion frequencies should be extremely low.

Alternatively, chimeric viruses with “suicide” characteristics may be constructed. Such viruses would go through only one or a few rounds of replication within the host. When used as a vaccine, the recombinant virus would go through limited replication cycle(s) and induce a sufficient level of immune response but it would not go further in the human host and cause disease. Recombinant viruses lacking one or more of the PIV genes or possessing mutated PIV genes would not be able to undergo successive rounds of replication. Defective viruses can be produced in cell lines which permanently express such a gene(s). Viruses lacking an essential gene(s) would be replicated in these cell lines, however, when administered to the human host, they would not be able to complete a round of replication. Such preparations may transcribe and translate—in this abortive cycle—a sufficient number of genes to induce an immune response. Alternatively, larger quantities of the strains could be administered, so that these preparations serve as inactivated (killed) virus vaccines. For inactivated vaccines, it is preferred that the heterologous gene product be expressed as a viral component, so that the gene product is associated with the virion. The advantage of such preparations is that they contain native proteins and do not undergo inactivation by treatment with formalin or other agents used in the manufacturing of killed virus vaccines. Alternatively, mutated PIV made from cDNA may be highly attenuated so that it replicates for only a few rounds.

In certain embodiments, the vaccine of the invention comprises an attenuated virus. Without being bound by theory, the attenuated virus can be effective as a vaccine even if the attenuated virus is incapable of causing a cell to generate new infectious viral particles because the viral proteins are inserted in the cytoplasmic membrane of the host thus stimulating an immune response.

In another embodiment of this aspect of the invention, inactivated vaccine formulations may be prepared using conventional techniques to “kill” the chimeric viruses. Inactivated vaccines are “dead” in the sense that their infectivity has been destroyed. Ideally, the infectivity of the virus is destroyed without affecting its immunogenicity. In order to prepare inactivated vaccines, the chimeric virus may be grown in cell culture or in the allantois of the chick embryo, purified by zonal ultracentrifugation, inactivated by formaldehyde or β-propiolactone, and pooled. The resulting vaccine is usually inoculated intramuscularly.

Inactivated viruses may be formulated with a suitable adjuvant in order to enhance the immunological response. Such adjuvants may include but are not limited to mineral gels, e.g., aluminum hydroxide; surface active substances such as lysolecithin, pluronic polyols, polyanions; peptides; oil emulsions; and potentially useful human adjuvants such as BCG, Corynebacterium parvum, ISCOMS, and virosomes.

Many methods may be used to introduce the vaccine formulations described above, these include but are not limited to oral, intradermal, intramuscular, intraperitoneal, intravenous, subcutaneous, percutaneous, and intranasal and inhalation routes. It may be preferable to introduce the chimeric virus vaccine formulation via the natural route of infection of the pathogen for which the vaccine is designed.

In certain embodiments, the invention relates to immunogenic compositions. The immunogenic compositions comprise a chimeric PIV. In certain embodiments, the immunogenic composition comprises an attenuated chimeric PIV. In certain embodiments, the immunogenic composition further comprises a pharmaceutically acceptable carrier.

Various techniques may be used to evaluate the effectiveness and safeness of a vaccine according to the present invention. An effective vaccine is a vaccine that protects vaccinated individuals from illness due to pathogens, by invoking proper innate, cellular, and humoral responses with minimal side effect. The vaccine must not cause disease. Any techniques that are able to measure the replication of the virus and the immune response of the vaccinated subject may be used to evaluate the vaccine. For example, challenge studies and clinical trials can be used. See Section 5.5.4. and Section 5.5.5. Non-limiting examples are also given in the Example sections, infra.

5.6.1. Dosage Regimens and Administration of the Vaccines or Immunogenic Preparations of the Invention

The present invention provides vaccines and immunogenic preparations comprising chimeric PIV expressing one or more heterologous or non-native antigenic sequences. The vaccines or immunogenic preparations of the invention encompass single or multivalent vaccines, including bivalent and trivalent vaccines. The vaccines or immunogenic formulations of the invention are useful in providing protections against various viral infections. Particularly, the vaccines or immunogenic formulations of the invention provide protection against respiratory tract infections in a host.

A recombinant virus and/or a vaccine or immunogenic formulation of the invention can be administered alone or in combination with other vaccines. Preferably, a vaccine or immunogenic formulation of the invention is administered in combination with other vaccines or immunogenic formulations that provide protection against respiratory tract diseases, such as but not limited to, respiratory syncytial virus vaccines, influenza vaccines, measles vaccines, mumps vaccines, rubella vaccines, pneumococcal vaccines, rickettsia vaccines, staphylococcus vaccines, whooping cough vaccines or vaccines against respiratory tract cancers. In a preferred embodiment, the virus and/or vaccine of the invention is administered concurrently with pediatric vaccines recommended at the corresponding ages. For example, at two, four or six months of age, the virus and/or vaccine of the invention can be administered concurrently with DtaP (IM), Hib (IM), Polio (IPV or OPV) and Hepatitis B (IM). At twelve or fifteen months of age, the virus and/or vaccine of the invention can be administered concurrently with Hib (IM), Polio (IPV or OPV), MMRII® (SubQ); Varivax® (SubQ), and hepatitis B (IM). The vaccines that can be used with the methods of invention are reviewed in various publications, e.g., The Jordan Report 2000, Division of Microbiology and Infectious Diseases, National Institute of Allergy and Infectious Diseases, National Institutes of Health, United States, the content of which is incorporated herein by reference in its entirety.

A vaccine or immunogenic formulation of the invention may be administered to a subject per se or in the form of a pharmaceutical or therapeutic composition. Pharmaceutical compositions comprising an adjuvant and an immunogenic antigen of the invention (e.g., a virus, a chimeric virus, a mutated virus) may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the immunogenic antigen of the invention into preparations which can be used pharmaceutically. Proper formulation is, or amongst others, dependent upon the route of administration chosen.

When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum hydroxide, aluminum phosphate gel, Freund's Complete Adjuvant, Freund's Incomplete Adjuvant, squalene or squalane oil-in-water adjuvant formulations, biodegradable and biocompatible polyesters, polymerized liposomes, triterpenoid glycosides or saponins (e.g., QuilA and QS-21, also sold under the trademark STIMULON, ISCOPREP), N-acetyl-muramyl-L-threonyl-D-isoglutamine (Threonyl-MDP, sold under the trademark TERMURTIDE), LPS, monophosphoryl Lipid A (3D-MLAsold under the trademark MPL).

The subject to which the vaccine or an immunogenic composition of the invention is administered is preferably a mammal, most preferably a human, but can also be a non-human animal, including but not limited to, primates, cows, horses, sheep, pigs, fowl (e.g., chickens, turkeys), goats, cats, dogs, hamsters, mice and rodents.

Many methods may be used to introduce the vaccine or the immunogenic composition of the invention, including but not limited to, oral, intradermal, intramuscular, intraperitoneal, intravenous, subcutaneous, percutaneous, intranasal and inhalation routes, and via scarification (scratching through the top layers of skin, e.g., using a bifurcated needle).

For topical administration, the vaccine or immunogenic preparations of the invention may be formulated as solutions, gels, ointments, creams, suspensions, etc. as are well-known in the art.

For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents. Alternatively, the proteins may be in powder form for constitution with a suitable vehicle, e.g., sterile pyrogen-free water, before use.

Determination of an effective amount of the vaccine or immunogenic formulation for administration is well within the capabilities of those skilled in the art, especially in light of the detailed disclosure provided herein.

An effective dose can be estimated initially from in vitro assays. For example, a dose can be formulated in animal models to achieve an induction of an immunity response using techniques that are well known in the art. One having ordinary skill in the art could readily optimize administration to all animal species based on results described herein. Dosage amount and interval may be adjusted individually. For example, when used as an immunogenic composition, a suitable dose is an amount of the composition that when administered as described above, is capable of eliciting an antibody response. When used as a vaccine, the vaccine or immunogenic formulations of the invention may be administered in about 1 to 3 doses for a 1-36 week period. Preferably, 1 or 2 doses are administered, at intervals of about 2 weeks to about 4 months, and booster vaccinations may be given periodically thereafter. Alternate protocols may be appropriate for individual animals. A suitable dose is an amount of the vaccine formulation that, when administered as described above, is capable of raising an immunity response in an immunized animal sufficient to protect the animal from an infection for at least 4 to 12 months. In general, the amount of the antigen present in a dose ranges from about 1 pg to about 100 mg per kg of host, typically from about 10 pg to about 1 mg, and preferably from about 100 pg to about 1 μg. Suitable dose range will vary with the route of injection and the size of the patient, but will typically range from about 0.1 mL to about 5 mL.

In a specific embodiment, the viruses and/or vaccines of the invention are administered at a starting single dose of at least 10³ TCID₅₀, at least 10⁴ TCID₅₀, at least 10⁵ TCID₅₀, at least 10⁶ TCID₅₀. In another specific embodiment, the virus and/or vaccines of the invention are administered at multiple doses. In a preferred embodiment, a primary dosing regimen at 2, 4, and 6 months of age and a booster dose at the beginning of the second year of life are used. More preferably, each dose of at least 10⁵ TCID₅₀, or at least 10⁶ TCID₅₀ is given in a multiple dosing regimen. The replication rate of a virus can be used as an index to adjust the dosage of a vaccine in a clinical trial. For example, assays to test the replication rate of a virus (e.g., a growth curve, see Section 5.5. for available assays) can be used to compare the replication rate of the viruses and/or vaccines of the invention to that of the bPIV3, which was demonstrated in previous studies (see Clements et al., J. Clin. Microbiol. 29: 1175-82 (1991); Karron et al., J. Infect. Dis. 171: 1107-14 (1995); Karron et al., Ped. Inf. Dis. J. 5: 650-654 (1996). These studies showed that a bovine PIV3 vaccine is generally safe and well tolerated by healthy human volunteers, including adults, children 6-60 months of age, and infants 2-6 months of age. In these studies, subjects have received at least a single dose of bPIV3 vaccine from 10³ TCID₅₀ to 10⁶ TCID₅₀. Twelve children received two doses of 10⁵ TCID₅₀ PIV3 vaccine instead of one dose without untoward effects.). A comparable replication rate as to bPIV3 suggests that a comparable dosage may be used in a clinical trial. A lower replication rate compared to that of bPIV3 suggests that a higher dosage can be used.

5.6.2. Target Populations

In certain embodiments of the invention, the target population for the therapeutic and diagnostic methods of the invention is defined by age. In certain embodiments, the target population for the therapeutic and/or diagnostic methods of the invention is characterized by a disease or disorder in addition to a respiratory tract infection.

In a specific embodiment, the target population encompasses young children, below 2 years of age. In a more specific embodiment, the children below the age of 2 years do not suffer from illnesses other than respiratory tract infection.

In other embodiments, the target population encompasses patients above 5 years of age. In a more specific embodiment, the patients above the age of 5 years suffer from an additional disease or disorder including cystic fibrosis, leukaemia, and non-Hodgkin lymphoma, or recently received bone marrow or kidney transplantation.

In a specific embodiment of the invention, the target population encompasses subjects in which the hMPV infection is associated with immunosuppression of the hosts. In a specific embodiment, the subject is an immunocompromised individual.

In certain embodiments, the target population for the methods of the invention encompasses the elderly.

In a specific embodiment, the subject to be treated with the methods of the invention was infected with hMPV in the winter months.

The following examples are illustrative, but not limiting, of the present invention. Cells and Viruses used in the examples are maintained as follows: the RSV A2 strain, the bovine parainfluenza type 3/human parainfluenza type 3 (b/h PIV3) virus, the human metapneumovirus NL/1/00 strain (hMPV), the bovine parainfluenza type 3/human parainfluenza type 3 vectored RSV viruses (b/h PIV3/RSV viruses), and the bovine parainfluenza type 3/human parainfluenza type vectored human metapneumovirus (b/h PIV3/hMPV) were grown in Vero cells in Opti-MEM (Gibco/BRL) in the presence of gentamicin. The modified vaccinia virus Ankara (MVA-T7) or fowl-pox-T7 (FP-T7) which expressed the phage T7 RNA polymerase were grown in chicken embryonic kidney cells (SPAFAS). Vero, HeLa and Hep-2 cells were maintained in MEM (JRH Biosciences) supplemented with 10% fetal bovine serum (FBS), 2 mM L-glutamine, non-essential amino acids, and antibiotics.

### 6. EXAMPLE 1

### Construction and Cloning of Chimeric Bovine Parainfluenza 3/Human Parainfluenza 3 cDNA

In order to substitute the F and HN genes of bPIV3 with those of hPIV3, additional restriction enzyme sites were introduced into the infectious bPIV3 cDNA. Using site-directed mutagenesis, a unique Nhe I site was introduced at nucleotide position 5041 and a Sal I site was introduced at nt 8529 of the bPIV3 cDNA. The modified full-length bPIV3 cDNA was treated with Nhe I and Sal I restriction enzymes and a ˜14 kb DNA fragment encompassing all of the viral bPIV3 sequences except the F and HN genes, was isolated by gel purification.

To obtain the hPIV3 F and HN gene sequences, a 10 cm dish of confluent Vero cells was infected with a strain of hPIV3 (hPIV3/Tex/12084/1983). After 3 days of incubation at 37° C., the cells were harvested and total RNA was isolated using RNA STAT-LS 50 (Tel-Test Inc.). Viral cDNA was generated by reverse transcription using a hPIV3 specific oligo annealing at position 4828 of the hPIV3 genome. The hPIV3 F and HN genes were amplified by PCR (polymerase chain reaction) using Taq polymerase. The PCR product was cloned into the pT/A TOPO cloning vector (Invitrogen) and from two clones (#11 and #14) the hPIV3 F and HN genes were sequenced. Sequence analysis revealed that for clone #11, the F gene was correct, but the HN gene contained aberrant sequences; for clone #14, the HN gene was correct, but the F gene contained aberrant stop codons. Thus, a plasmid, containing functional hPIV3 F and HN genes, was constructed by combining the correct F gene of #11 with the correct HN gene of #14 in the following manner. Both hPIV3 plasmids (#11 and #14) were digested with Nhe1 and EcoR1. A 1.6 kb fragment harboring the correct F gene was isolated from clone #11 and a 8.5 kb fragment containing the correct HN gene and plasmid sequences, was isolated from clone #14. The two fragments were ligated to produce the intact hPIV3 F and HN genes-containing plasmid. The correct sequence was confirmed by DNA sequence analysis. Finally, a single nucleotide was added at the 3′ end of the HN gene in the untranslated region to satisfy the “Rule of Six.” The addition of the single nucleotide was accomplished by using the QuikChange mutagenesis kit (Stratagene) and was confirmed by DNA sequencing. The correct hPIV3 F and HN gene DNA fragment was then isolated by digestion with Nhe 1 and Sal 1 and a 3.5 kb DNA fragment was gel purified.

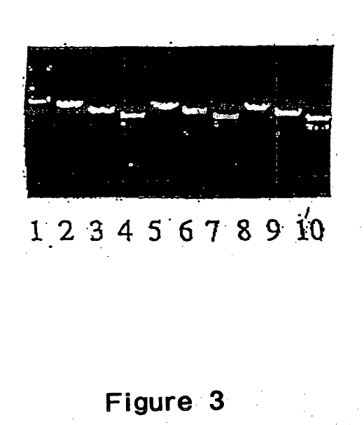

The full-length b/h PIV3 chimeric cDNA was constructed by ligating the 14.5 kb DNA fragment harboring bPIV3 sequences described above and the 3.5 kb DNA fragment containing the hPIV3 F and HN genes (see FIG. 3). The full-length chimeric plasmid DNA was confirmed by extensive restriction enzyme mapping. In addition, the M/F and HN/L gene junctions of the chimeric construct were confirmed by DNA sequencing to both contain bPIV3 and hPIV3 sequences as well as a Nhe 1 and a Sal 1 restriction enzyme site, respectively.

### 7. EXAMPLE 2

### Construction and Cloning of Chimeric Bovine Parainfluenza 3/Human Parainfluenza 3 Vectored Respiratory Syncytial Virus F or G cDNAs

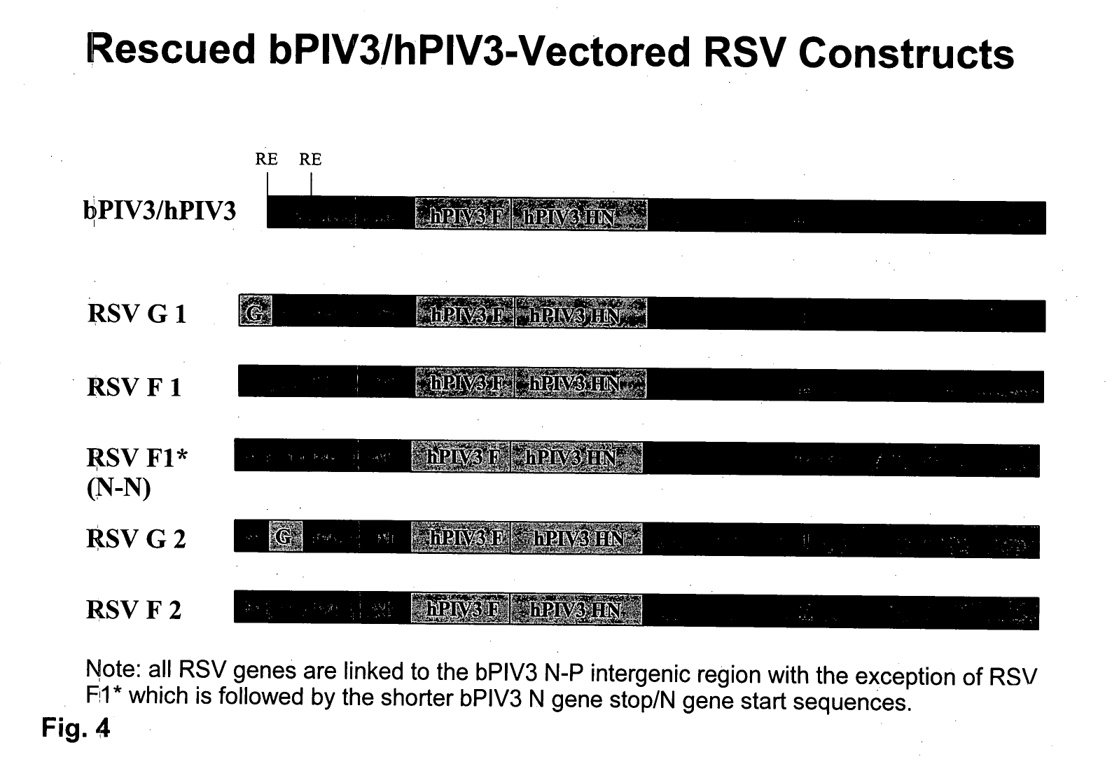

In order to determine the effects of RSV antigen insertions in position 1 or 2 of the b/h PIV3 genome on virus replication, respiratory syncytial virus (RSV) F and G genes were cloned into different positions of the chimeric bovine parainfluenza 3/human parainfluenza 3 vector (b/h PIV3 vector). See FIG. 4.

In order to insert foreign genes into the bovine/human (b/h) PIV3 cDNA, AvrII restriction enzyme sites were introduced in the b/h PIV3 cDNA plasmid (Haller et al., 2000; 2001, this is the same construct as in Example 6) by site-directed mutagenesis using the QuickChange kit (Stratagene). One AvrII site was introduced at nucleotide (nt) 104 in the b/h PIV3 genome altering four nucleotides using the following oligo 5′GAA ATC CTA AGA CCC TAG GCA TGT TGA GTC3′ and its complement. This restriction enzyme site was used to insert the RSV genes in the first (most 3′) position of the viral genome. Another AvrII site was introduced in the N-P intergenic region at nt 1774 changing two nucleotides using the following oligo 5′CCACAACTCAATCAACCTAGGATTCATGGAAGACAATG 3′ and its complement. This restriction site was used to insert the RSV genes in the second position between the N and P genes of b/h PIV3 (FIG. 4). Full-length b/h PIV3 cDNAs harboring the AvrII sites at nts 104 and 1774 were tested for functionality by recovering viruses by reverse genetics.

Construction of RSV G cassette (N-P gene stop/start): A DNA fragment was generated that contained the bPIV3 N-P intergenic region as well as the 3′ end sequences of the RSV G gene, using the b/h PIV3 cDNA as PCR template. This fragment was generated by PCR using the following oligos: 5′CCCAACACACCACGCCAGTAGTCACAA AGAGATGACCACTATCAC3′ and 5′CCCAAGCTTCCTAGGTGAATCTTTG GTTGATTGAGTTGTGG3′. This fragment was then used to carry out overlapping PCR to add the bPIV3 N-P intergenic region to the RSV G gene. For the second PCR reaction, a plasmid containing the RSV G and F gene was used as a DNA template, the oligo 5′CAGCGGATCCTAGGGGAGAAAAGTGTCGAAGAAAAATGTCC3′ and an oligo generated from the short PCR fragment above were used as primers. The resulting PCR fragment containing the RSV G gene linked to the bPIV3 N-P intergenic region and flanking AvrII restriction enzyme sites, was cloned into pGEM3. The RSV G gene was sequenced to confirm the presence of an intact open reading frame and the predicted amino acid sequences. The DNA fragments harboring the RSV G gene were inserted into the first or second position using the AvrII restriction enzyme sites into a subclone harboring only the first 5200 nucleotides of the bPIV3 (1-5 bPIV3) genome that was linearized with AvrII. As used herein and other Examples, 1-5 bPIV3 refers to the nucleotide 1 to 5196 (or 5200) of bovine PIV3 genome. There is a BstB1 site at this location.

Construction of RSV F cassette (N-P gene start/stop): The RSV F gene fragment was isolated by PCR from a full-length bPIV3/RSV F+G cDNA plasmid using oligos that added AvrII sites at the 5′ and 3′ end of the RSV F gene, and introduced into the 1-5 bPIV3 plasmid containing the first 5200 nucleotides of the bPIV3 genome and the AvrII site at nt 1774, which was linearized with AvrII. The bPIV3 N-P intergenic region was isolated by PCR using 1-5 bPIV3/RSV G2 as a template. The oligo 5′GACGCGTCGACCACAAAGAGATGACCACTATCACC 3′ and an oligo annealing in the bPIV3 F open reading frame were used to generate a PCR fragment containing the bPIV3 N-P intergenic region, AvrII site, and bPIV3 sequences up to nt 5200. The PCR fragment was digested with SalI and NheI, and added to the 1-5 bPIV3 plasmid harboring the RSV F gene in position 2, which was digested with SalI and NheI. To introduce the RSV F gene containing the N-P intergenic region into position 1, the 1.8 kb RSV F cassette was excised using AvrII, and ligated into 1-5 bPIV3 containing the AvrII site at nt 104, which was linearized with AvrII.

Construction of the RSV F cassette with a short intergenic region (N stop/N start):

The generation of the RSV F gene with the short N-N intergenic region was accomplished by performing a PCR reaction using 1-5 bPIV3/RSV F2 as a template, the oligo 5′GCGCGTCGACCAAGTAAGAAAAACTTAGGATTAAAGAACCCTAGGACTGTA3′, and an oligo annealing upstream of the 5′ end of the RSV F gene encompassing the AvrII restriction enzyme site. The PCR product containing the RSV F gene and the short N-N intergenic region, was digested with AvrII and introduced into 1-5 bPIV3 nt 104 which was linearized with AvrII.

The RSV G and RSV F gene cassettes were sequenced to confirm the presence of an intact open reading frame, the predicted amino acid sequences, and to verify the rule of six. The RSV G and RSV F transcriptional units were inserted into the first or second position using the AvrII restriction enzyme sites into a subclone1-5 bPIV3 that was linearized with AvrII. After confirming proper orientation by restriction enzyme mapping, the plasmids harboring the RSV genes in the first position were digested with SphI and BssHII and 4 kb (1-5 bPIV3/RSV G1) or 4.8 kb (1-5 bPIV3/RSV F1) DNA fragments were isolated. In a second cloning step, the remainder of the b/h PIV3 genome was added as a SphI-BssHII 15.1 kb DNA fragment, yielding full-length cDNAs. The bPIV3 subclones, harboring the RSV genes in the second position, were cut with SphI and NheI, and 5.8 kb (bPIV3/RSV G2) and a 6.5 kb (bPIV3/RSV F2) DNA fragments were isolated. In a second cloning step, the rest of the b/h PIV3 genome was ligated as an NheI-SphI DNA fragment of 14 kb in size. The full-length chimeric b/h PIV3/RSV plasmids were propagated in STBL-2 cells (Gibco/BRL) that provided high yields of full-length virus cDNA plasmids.

### 8. EXAMPLE 3

### Bovine Parainfluenza 3/Human Parainfluenza 3 Vectored Respiratory Syncytial Virus F or G Displayed a Positional Effect with Regards to mRNA Production and Protein Expression as Well as Virus Replication In Vitro

Three experiments were performed to confirm the effective expression of the RSV F or G gene in the constructs of Example 2, and to determine positional effects of gene insertions in the PIV3 genome.

First, in order to demonstrate RSV protein expression by the chimeric viruses, a Western blot of chimeric virus-infected cell lysates was carried out and probed with RSV-specific antisera. See FIG. 5A. Western blots were performed as follows: Chimeric viruses were used to infect (70-80%) subconfluent Vero cells at a MOI of 0.1 or 1.0. Forty-eight hours post infection the media overlay was removed and infected monolayers were washed once with 1 ml of PBS. The cells were subsequently lysed in 400 μl of Laemmli buffer (Bio-Rad) containing 0.05% β-Mercaptoethanol (Sigma). 15 μl of each sample was separated on 12% Tris-HCl Ready Gel (Bio-Rad) and transferred to nylon membranes using a semi-dry transfer cell (Bio-Rad). Nylon membranes were rinsed in PBS [pH 7.6] containing 0.5% (v/v) Tween-20 (Sigma) (PBST) and blocked with PBST containing 5% (w/v) dry milk (PBST-M) for 20-30 minutes at room temperature. Membranes were incubated with either a mixture of RSV F monoclonal antibodies (WHO 1269, 1200, 1153, 1112, 1243, 1107, see Beeler and Coelingh, J. Virol. (1989) 63(7):2941-50, which is incorporated herein by reference) at a 1:1000 dilution in PBST-M or RSV G 10181 polyclonal antibody (Orbigen) at a 1:2000 dilution in PBST-M for 1 hour at room temperature. Following four washes with PBST, the membranes were incubated with a secondary horseradish peroxidase-conjugated goat anti-mouse antibody (Dako) at a 1:2000 dilution in PBST-M for 1 hour at room temperature. Membranes were washed 4 times with PBST and developed using a chemiluminescence substrate (Amersham Pharmacia) and exposed to Biomax Light Film (Kodak) for visualization of protein bands.

Consistent with the reduced replication efficiency of b/h/RSV F1*N-N in Vero cells (FIG. 5C, see below), the amount of RSV F1 detected at 48 hours post infection was about 10 times less than that present in b/h PIV3/RSV F2 or wild-type RSV A2 infected cells (compare lanes 2, 3, and 4, FIG. 5A). A ˜50 kDa band representing the RSV F1 fragment was detected in cells infected with b/h PIV3/RSV F1 and b/h PIV3/RSV F2 as well as wild-type RSV. b/h PIV3/RSV F1 expressed RSV F1 protein levels at 48 hrs post-infection (hpi) similar to those observed for b/h PIV3/RSV F2. Only low levels of F0 were detected in cells infected with b/h PIV3/RSV F1 and b/h PIV3/RSV F2 indicating that the F0 precursors were efficiently processed during infections as was also observed in wild-type RSV infections. As expected, b/h PIV3 and mock-infected cell lysates did not yield a signal for RSV F protein. A smaller band of ˜26 kDa was observed in the b/h PIV3/RSV F1 and F2 lysates that was not present in wild type RSV lysates. This band represents a proteolytic fragment of RSV F protein not produced in wild type RSV-infected cells. The absence of the proteolytic fragment in RSV-infected cells may be due to the presence of the complete set of RSV proteins. When b/h PIV3/RSV F1*N-N infections were repeated at a higher MOI of 1.0 (FIG. 5A, lane 1), the F1 fragment in b/h PIV3/RSV F1 infected cells accumulated to wild-type RSV levels at 48 hours post-infection. The relative amount of the 50 kDa and 26 kDa F1 fragments in b/h PIV3/RSV F1 or b/h PIV3/RSV F2 infected cells was approximately 1:5.

The relative expression of RSV G in b/h PIV3/RSV G1, b/h PIV3/RSV G2 and wild-type RSV infected cells at a MOI of 0.1 at 48 hour post infection is shown in FIG. 5A. Both the immature and glycosylated forms of RSV G that migrated at approximately 50 kDa and 90 kDa, respectively, were detected. b/h PIV3/RSV G1 infected cells showed levels of RSV G expression similar to that seen in wild-type RSV infected cells (lanes 1 and 3, FIG. 5A). However, in b/h PIV3/RSV G2 infected cells, the accumulation of RSV G was about 2-3 times more than that present in wild-type RSV infected cells (lanes 2 and 3, FIG. 5A). The higher levels of RSV G expression may be due to the more 3′ proximal position of the RSV G gene in the PIV3 genome compared to its position in the RSV genome. Higher levels of expression were not observed for RSV G in position 1 which may be due an attenuated virus replication phenotype. RSV G-specific bands were not observed in cell lysates derived from b/h PIV3 or mock-infected cells. Collectively, these data showed that the chimeric b/h PIV3/RSV efficiently expressed the RSV proteins in either position 1 or 2. Equivalent expression levels of RSV proteins by b/h PIV3 were observed independent of whether position 1 or 2 was used, although position 2 appeared to express slightly higher levels of RSV G protein. Antigen expression levels at position 1 or position 2 of the PIV3 genome were similar such that either position can be used for gene insertion.

Next, Northern blot analysis showed that the mRNA transcription correlated with the result of the protein expression demonstrated by the Western blot, see FIG. 5B. Northern blot was performed as follows: total cellular RNA was prepared from virus-infected cells using Trizol LS (Life Technologies). The RNA was further purified by one phenol-chloroform extraction and precipitated with ethanol. RNA pellets were resuspended in diethyl pyrocarbonate-treated water and stored at −80° C. Equal amounts of total RNA were separated on 1% agarose gels containing 1% formaldehyde and transferred to nylon membranes (Amersham Pharmacia Biotech) using a Turboblotter apparatus (Schleicher & Schuell). The blots were hybridized with digoxigenin (DIG)-UTP-labeled riboprobes synthesized by in vitro transcription using a DIG RNA labeling kit (Roche Molecular Biochemicals). Hybridization was carried out at 68° C. for 12 h in Express Hyb solution (Clontech). The blots were washed at 68° C. twice with 2×SSC (1×SSC contained 0.015 M NaCl with 0.015 M sodium citrate)-0.1% sodium dodecyl sulfate (SDS) followed by one wash with 0.5×SSC-0.1% SDS and a final wash with 0.1×SSC-0.1% SDS. Signals from the hybridized probes were detected by using a DIG-Luminescent detection kit (Roche Molecular Biochemicals) and visualized by exposure to BioMax ML film (Kodak).

Northern analysis of b/h PIV3/RSV F1*N-N, b/h PIV3/RSV F2, b/h PIV3/RSV G1 and b/h PIV3/RSV G2 showed that the viral mRNA levels for RSV F or RSV G correlated well with the RSV protein levels observed (FIG. 5B). The lowest levels of RSV F mRNAs were observed for b/h PIV3/RSV F1*N-N which also displayed the least amount of RSV F protein produced. b/h PIV3/RSV G1 produced less RSV G mRNAs resulting in lower RSV G protein levels than was observed for b/h PIV3/RSV G2.

Finally, growth of different virus (with RSV F or G gene at either position 1 or position 2) correlates with the results of the protein expression and the RNA transcription. The growth curve showed in FIG. 5C was obtained as follows: Vero cells were grown to 90% confluence and infected at an MOI of 0.01 or 0.1 with b/h PIV3, b/h PIV3 RSV F1, b/h PIV3 RSV G1, b/h PIV3 RSV F2, and b/h PIV3 RSV G2. The infected monolayers were incubated at 37° C. At 0, 24, 48, 72, 96 and 120 hours post-infection, cells and media were harvested together and stored at −70° C. Virus titers for each time point harvest were determined by TCID₅₀ or plaque assays in Vero cells. TCID₅₀ assays were inspected visually for CPE following incubation at 37° C. for 6 days, while plaque assays were immunostained with RSV polyclonal antisera for quantification after 5 days of incubation.

At an MOI of 0.01 in Vero cells, the chimeric viruses harboring the RSV G or F genes in the first position (b/h PIV3 RSV G1 and b/h PIV3 RSV F1*N-N) replicated at a slower rate, yielded lower peak titers, and exhibited a greater lag phase than the viruses that contained the RSV genes in the second position. Peak titers of b/h PIV3/RSV F1*N-N and b/h PIV3/RSV G1 at 96 hours post-infection were 10^{6.7} and 10^{5.5} TCID₅₀/ml, respectively (FIG. 5C). In contrast, peak titers of b/h PIV3/RSV F2 and b/h PIV3/RSV G2 were 10^{8.0} and 10^{7.4} at 72 and 96 hours post-infection, respectively (FIG. 5C). The b/h PIV3 control virus displayed peak titers of 10^{8.0} TCID50/ml, respectively (FIG. 5C). The b/h PIV3/RSV F2 yielded 1.3 log₁₀ higher titers than b/h PIV3/RSV F1*N-N. The b/h PIV3/RSV G2 replicated to 1.9 log₁₀ higher titers than b/h PIV3/RSV G1. Collectively, the data showed that b/h PIV3 expressing RSV proteins in genome positions 1 or 2 replicated to peak titers of 10⁶-10⁸ PFU/ml in Vero cells. Viruses harboring the antigen insertion in position 2 replicated more efficiently in tissue culture than those containing foreign genes in position 1.

To determine whether higher titers of b/h PIV3/RSV F1*N-N and b/h PIV3/RSV G1 could be achieved at all, the growth curves were repeated at a higher MOI of 0.1. At an MOI of 0.1, the peak titers of b/h PIV3/RSV F1*N-N and b/h PIV3/RSV G1 increased by 0.5 to 1.3 log₁₀ (data not shown). The lag phases of these viruses were reduced and peak titers were achieved earlier during the growth cycle.

### 9. EXAMPLE 4

### Positional Effect of eGFP Insertions in the Bovine Parainfluenza 3/Human Parainfluenza 3 Genome on Virus Replication

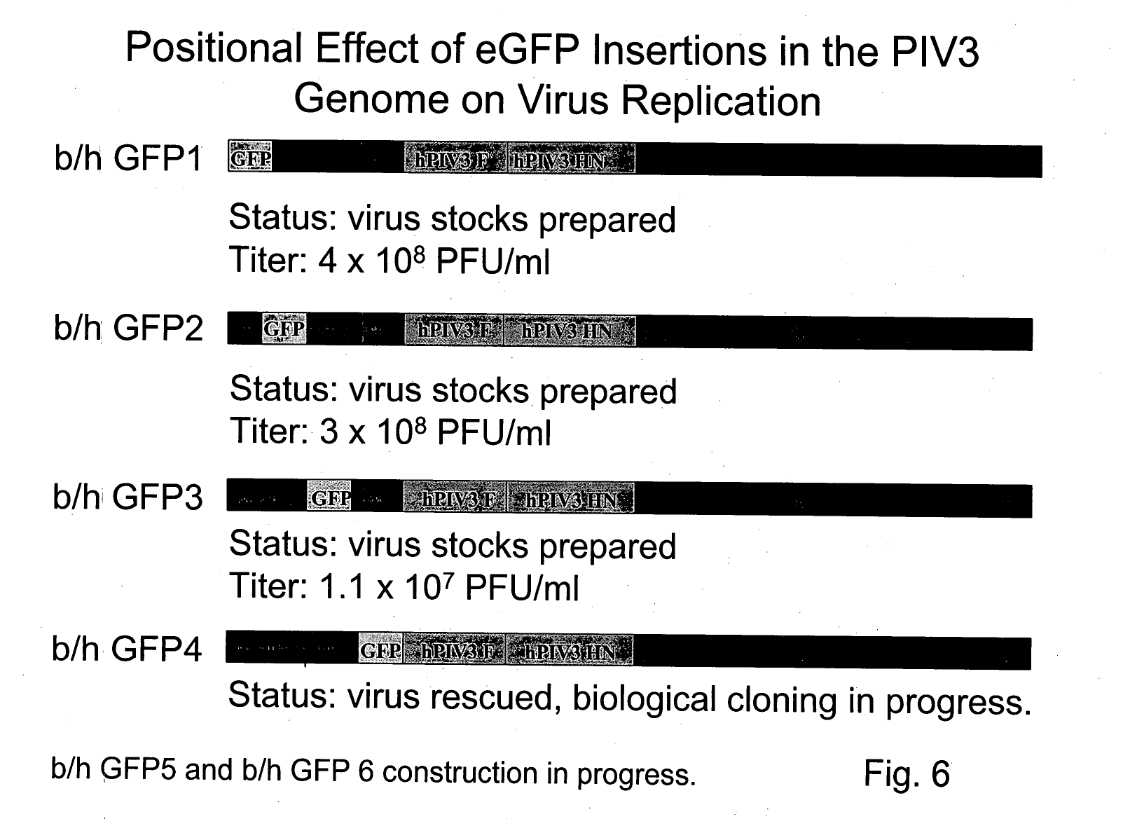

The effect of gene insertions into the bovine/human PIV3 vector backbone was assessed systematically by introducing the eGFP gene sequentially between all genes of PIV3 and observing the effect on virus replication and eGFP expression (FIG. 6). This type of assay investigates the importance of the transcriptional gradient observed for paramyxoviruses that yields specific ratios of viral mRNAs. Insertion of foreign genes will perturb these ratios and result in the synthesis of different amounts of viral proteins which may influence virus replication. The eGFP gene was chosen for this assay since it will not be incorporated into the virion membrane, and therefore should not interfere with viral processes such as packaging, budding, entry, etc. The eGFP gene was inserted into four positions of the b/h PIV3 genome, three of which were characterized for eGFP expression and virus replication. The eGFP gene cassette was linked to the bPIV3 N-P intergenic region. b/h GFP1 harbored the eGFP gene cassette in the 3′ most proximal position of the b/h PIV3 genome. b/h PIV3/GFP2 contained the eGFP gene cassette between the N and P genes of the b/h PIV3 genome. b/h PIV3/GFP3 was located between P and M, and b/h PIV3/GFP4 had the eGFP gene between M and F of b/h PIV3 (FIG. 6).

Construction of the eGFP gene cassette: the template of the eGFP gene is commercially available, e.g., it can be purchased from BD Biosciences (pIRES2-EGFP) or Clontech (pEGFP-N1). See Hoffmann et al., Virology 267: 310-317 (2000). The eGFP gene was isolated by PCR and the bPIV3 N-P intergenic region was added by employing the overlapping PCR method, using the following oligos: 5′ATTCCTAGGATGGTGAGCAAG GGCG3′, 5′GGACGAGCTGTACAAGTAAAAAAATAGCACCTAATCATG3′, and 5′CTACCTAGGTGAATCTTTGGTTG3′. The eGFP cassette was inserted into pCR2.1, sequenced, and adherence to the rule-of-six was confirmed. Then the eGFP cassette was digested with AvrII, gel purified, and inserted into positions 1, 2, 3, and 4 of b/h PIV3 as described below.

Generation of full-length cDNAs harboring the eGFP gene in positions 1 and 2: the eGFP gene cassette was inserted into the 1-5 bPIV3 plasmids which contained bPIV3 sequences from nts 1-5200 and an AvrII restriction enzyme site either at nt 104 (position 1) or nt 1774 (position 2). After confirming proper orientation by restriction enzyme mapping, the plasmid harboring the eGFP gene in the first position was digested with SphI and BssHII and 4 kb (1-5 eGFP1) DNA fragments were isolated. Next, the rest of the b/h PIV3 genome was added as a SphI-BssHII 15.1 kb DNA fragment, yielding full-length cDNAs. For generation of full-length cDNA comprising the eGFP in position 2, the bPIV3 subclones harboring the eGFP genes in the second position were cut with SphI and NheI, and 5.8 kb (1-5 eGFP2) DNA fragments were isolated. Next, the rest of the b/h PIV3 genome was added as an NheI-SphI DNA fragment of 14 kb in size. The full-length chimeric b/h PIV3/eGFP plasmids were propagated in STBL-2 cells (Gibco/BRL) that provided high yields of full-length virus cDNA plasmids.

Generation of full-length cDNAs harboring the eGFP gene in positions 3 and 4: in order to insert the eGFP cassette into position 3 of the b/h PIV3 genome, an AvrII restriction enzyme site was introduced at nt 3730 in the P-M intergenic region of a subclone containing nts 1-5200 of bPIV3, altering two nucleotides. The following oligo and its complement were used in a QuickChange PCR reaction to introduce the AvrII site: 5′GGACTAATCAATCCTAGGAAACAATGAGCATCACC3′. The eGFP cassette was digested with AvrII and ligated into the AvrII linearized 1-5 bPIV3 subclone harboring the AvrII site at nt 3730. A 5.5 kb DNA fragment from SphI to NheI was isolated from the GFP containing subclone and introduced into the b/h PIV3 cDNA digested with SphI and NheI to produce a full-length plasmid. In order to add the eGFP gene cassette into position 4 of the b/h PIV3 genome, a subclone containing b/h PIV3 sequences from nts 1-8500 was generated. This subclone was linearized with NheI (nt 5042), and the eGFP cassette containing compatible AvrII ends was inserted. Then the subclone harboring the eGFP cassette was digested with SphI and XhoI and a 7.1 kb DNA fragment was isolated. The b/h PIV3 plasmid was treated with SphI and XhoI and a 11 kb fragment was produced. These two DNA fragments were ligated to generate b/h PIV3/GFP4.

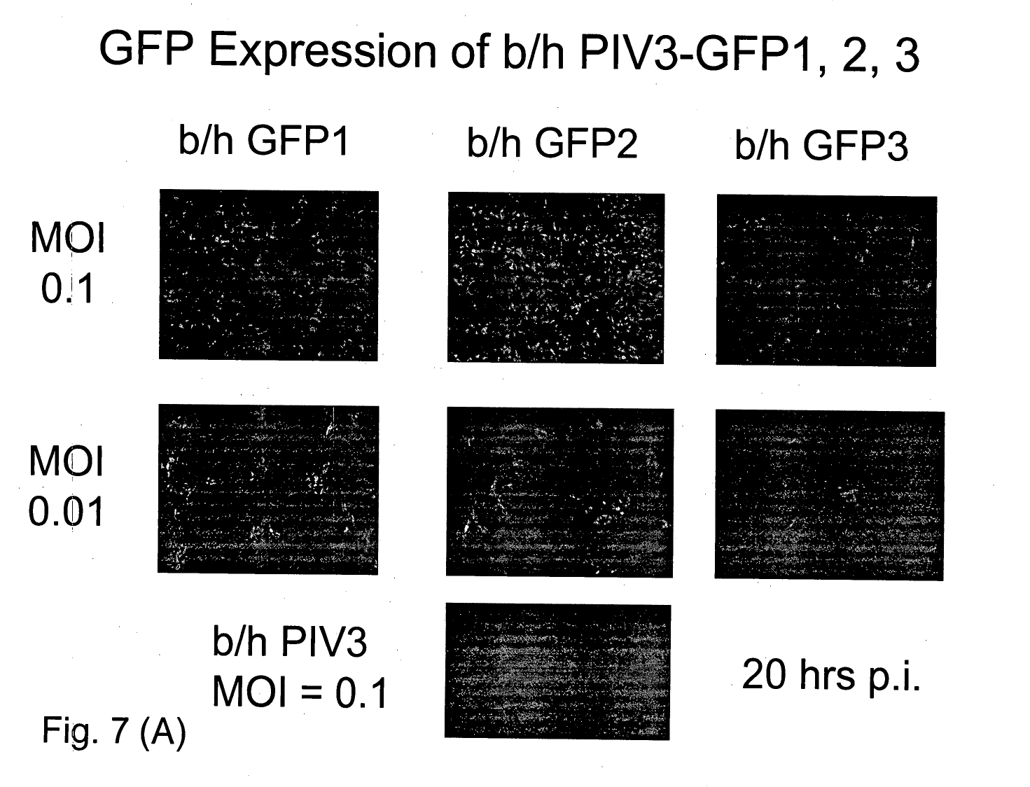

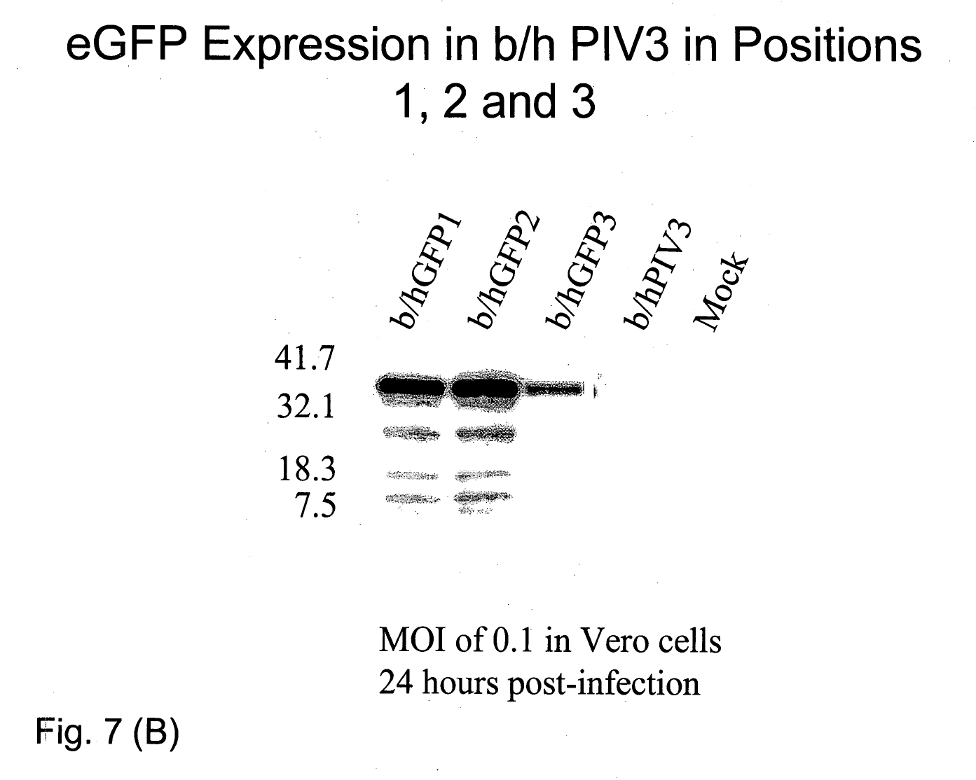

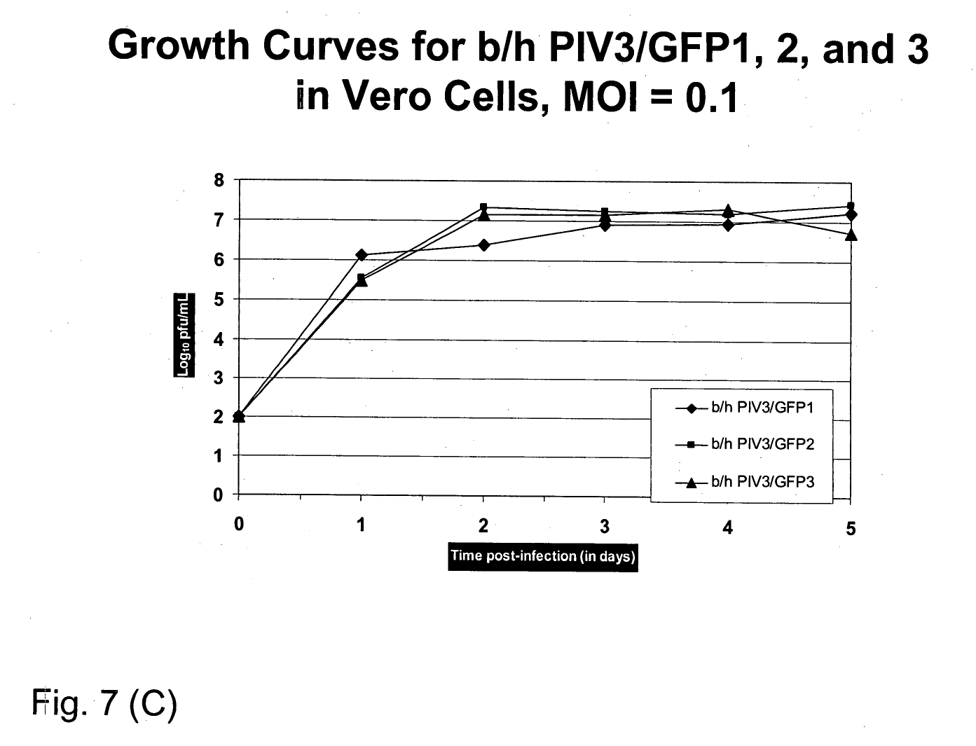

The amount of eGFP produced by b/h PIV3/GFP 1, 2, and 3 was assessed in two ways. First, the amount of green cells produced upon infecting Vero cells with b/h PIV3 GFP1, 2, and 3 at MOIs of 0.1 and 0.01 for 20 hours, was determined using a fluorescent microscope (FIG. 7A). b/h PIV3/GFP3 produced strikingly fewer green cells than b/h PIV3/GFP1 or 2.

Secondly, western analysis was performed on infected cells and the blots were probed with a GFP MAb as well as a PIV3 PAb. The initial observation that b/h PIV3/GFP3 produced dramatically less eGFP protein, was confirmed (FIG. 7B). b/h PIV3 GFP1 and GFP2 produced similar amounts of eGFP protein. The western blots methods controlled for same volume loading by probing with a PIV3 antibody (FIG. 7B). Interestingly, all three viruses showed similar amounts of PIV3 proteins (the HN protein is the most prominent band) produced. These results suggested that b/h PIV3/GFP3 transcribed less GFP mRNAs in position 3 as compared to positions 1 and 2. This data confirmed the presence of a transcriptional gradient of viral mRNAs in paramyxoviruses. The level of production of the PIV3 HN protein was not affected by the eGFP gene insertions (FIG. 7B).

In order to determine whether the GFP gene insertions had an effect on the kinetics of virus replication of b/h PIV3/GFP1,2, and 3, multicycle growth curves in Vero cells were carried out (FIG. 7C). The growth curves showed that b/h PIV3/GFP1 had a delayed onset of virus replication at 24 and 48 hours post-infection than b/h PIV3/GFP2 or GFP3. However, the final peak titers obtained were similar for all three viruses. The kinetics of replication for b/h PIV3/GFP2 and GFP3 were nearly identical (FIG. 7C). Interestingly, the altered ratios of viral mRNAs did not appear to effect virus replication significantly.

### 10. EXAMPLE 5

### Construction and Cloning of Chimeric Bovine Parainfluenza 3/Human Parainfluenza 3 Vectored Respiratory Syncytial Virus F with Different Intergenic Regions

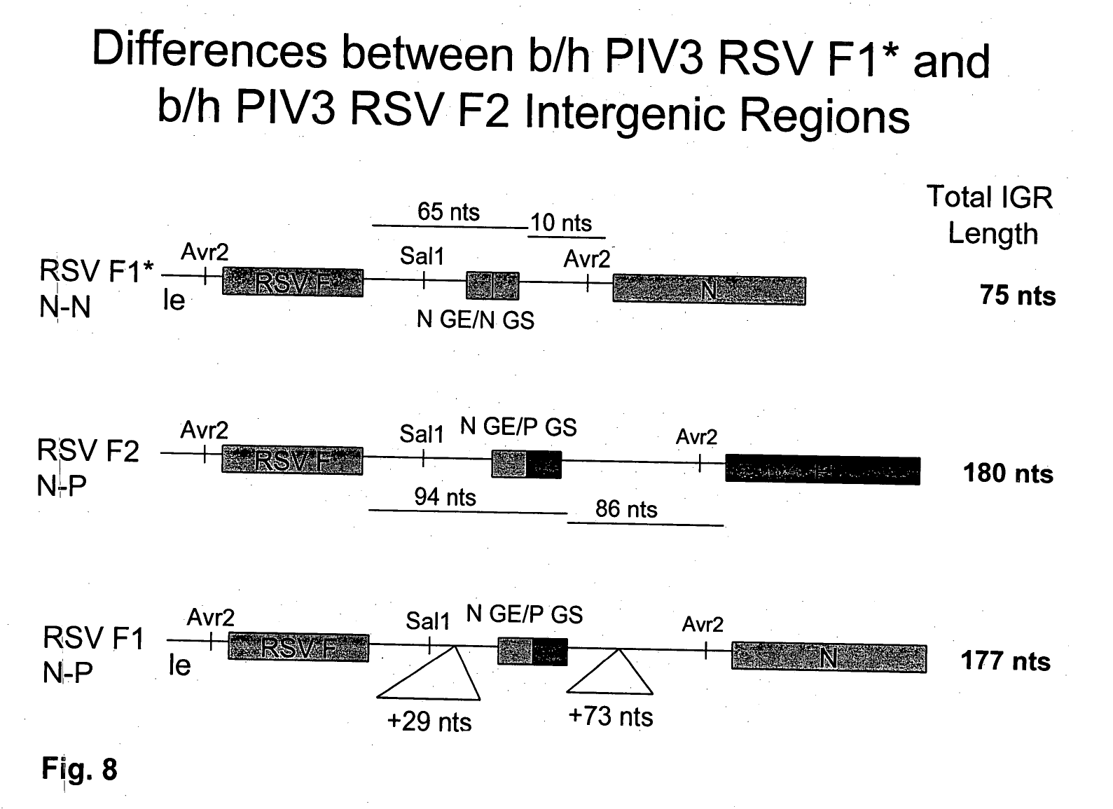

Three different constructs were used to determine the effect of intergenic region (nucleotides between each mRNA, e.g., nucleotides between the F gene and the N gene) on protein expression and viral replication. See FIG. 8. The first construct was b/h PUV3 vectored RSV F1* N-N in position 1, which had a shorter bPIV N gene stop/N gene start sequence (RSV F1* N-N in FIG. 4); the second construct was b/h PIV3 vectored RSV F at position 1 (RSV F2 in FIG. 4); and the last one was b/h PIV3 vectored RSV at position 1 (RSV F1 in FIG. 4). All three constructs were generated according to the cloning strategies described in section 7, Example 2.

The most dramatic difference between the two cassettes is the distance between the N gene start sequence and the N translation start codon in b/h PIV3/RSV F1*N-N which was only 10 nts long. In contrast, this distance is 86 nts long in b/h PIV3/RSV F2. The other difference is the use of the N gene start sequence in b/h PIV3/RSV F1*N-N rather than the P gene start sequence as was done in b/h PIV3/RSV F2. In order to determine whether the distance between the transcription gene start and the translation start of a viral transcription unit has an effect on virus replication, the b/h PIV3/RSV F1 construct was generated that contained the identical RSV F gene cassette as was used for b/h PIV3/RSV F2.

### 11. EXAMPLE 6

### The Length and/or Nature of the Intergenic Region Downstream of the Respiratory Syncytial Virus Gene has an Effect on Virus Replication

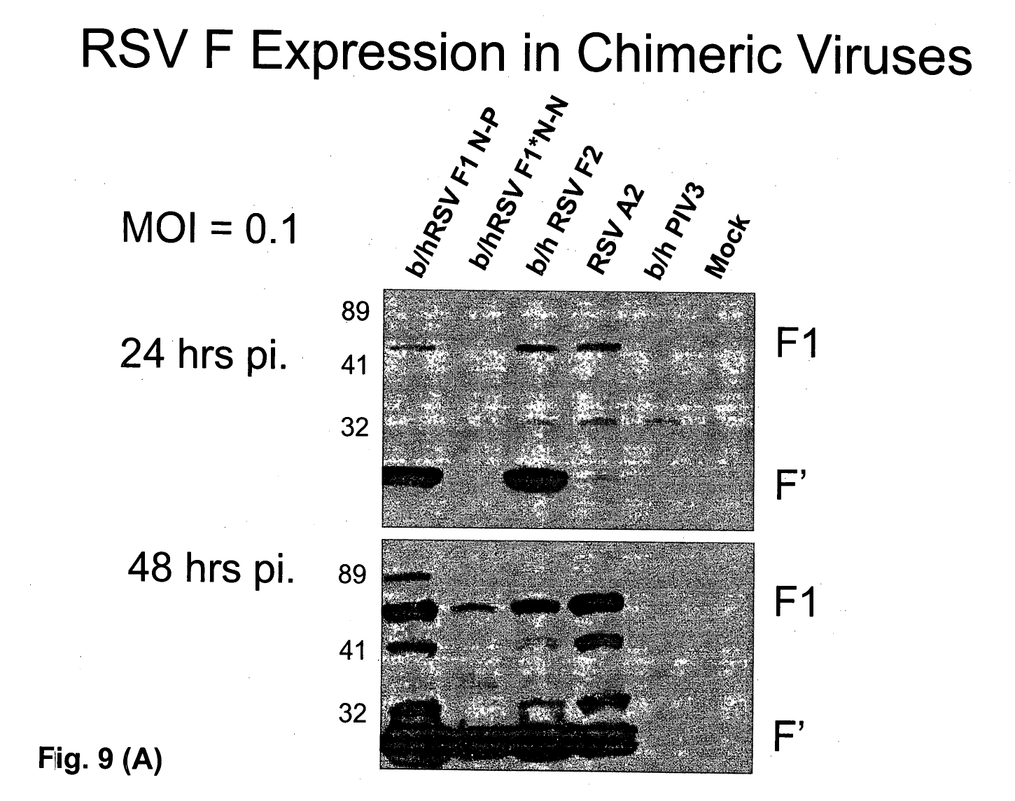

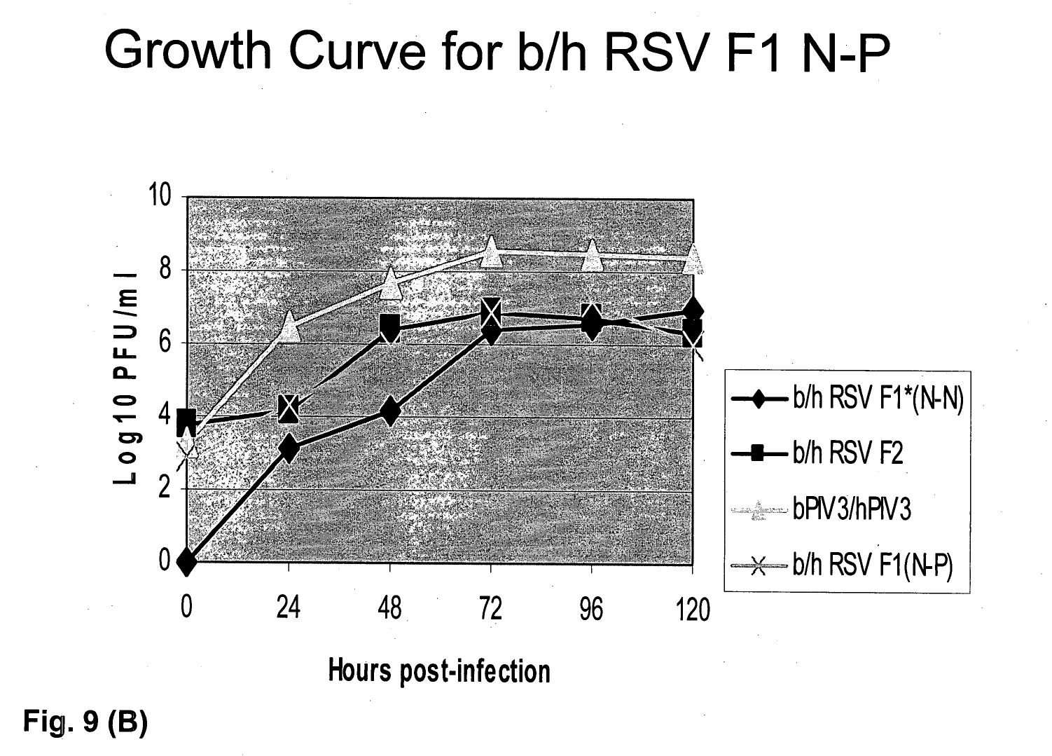

The three constructs in Example 5 were used in the following experiments to determine the effects of the intergenic region on viral protein expression and viral replication. See FIG. 9.

First, RSV F protein expression for b/h PIV3/RSV F1, b/h PIV3/RSV F1*N-N, and b/h PIV3/RSV F2 was compared at 24 and 48 hrs post-infection at an MOI of 0.1 in Vero cells using Western blots. Western blots were performed as follows: Chimeric viruses were used to infect (70-80%) subconfluent Vero cells at a MOI of 0.1. Twenty-four hours and forty-eight hours post infection the media overlay was removed and infected monolayers were washed once with 1 ml of PBS. The cells were subsequently lysed in 400 ml of Laemmli buffer (Bio-Rad) containing 0.05% b-Mercaptoethanol (Sigma). 15 ml of each sample was separated on 12% Tris-HCl Ready Gel (Bio-Rad) and transferred to nylon membranes using a semi-dry transfer cell (Bio-Rad). Nylon membranes were rinsed in PBS (pH 7.6) containing 0.5% (v/v) Tween-20 (Sigma) (PBST) and blocked with PBST containing 5% (w/v) dry milk (PBST-M) for 20-30 minutes at room temperature. Membranes were incubated with either a mixture of RSV F monoclonal antibodies (WHO 1269, 1200, 1153, 1112, 1243, 1107) at a 1:1000 dilution in PBST-M in PBST-M for 1 hour at room temperature. Following 4 washes with PBST, the membranes were incubated with a secondary horseradish peroxidase-conjugated goat anti-mouse antibody (Dako) at a 1:2000 dilution in PBST-M for 1 hour at room temperature. Membranes were washed 4 times with PBST and developed using a chemiluminescence substrate (Amersham Pharmacia) and exposed to Biomax Light Film (Kodak) for visualization of protein bands.

b/h PIV3/RSV F1 expressed RSV F₁ protein levels at 24 and 48 hrs post-infection close to the levels observed for b/h PIV3/RSV F2 but much higher than those of b/h PIV3/RSV F1*N-N. Therefore, the spacing between the gene start element and the translation start codon may be critical for virus replication. The N gene start sequences were changed to P gene start sequences, however this change only incurred the alteration of a single nucleotide. Either of these factors may be responsible for rescuing the RSV F protein expression phenotype.

Next, multicycle growth curves were carried out to compare the kinetics of virus replication of b/h PIV3/RSV F1, b/h PIV3/RSV F1*N-N, and b/h PIV3/RSV F2 in Vero cells at an MOI of 0.1 (see FIG. 9B), which was performed as follows: Vero cells were grown to 90% confluence and infected at an MOI of 0.1 with b/h PIV3, b/h PIV3/RSV F1*N-N, b/h PIV3/RSV F1, and b/h PIV3/RSV F2. The infected monolayers were incubated at 37° C. At 0, 24, 48, 72, and 96 hours post-infection, cells and media were harvested together and stored at −70° C. Virus titers for each time point harvest were determined by plaque assays in Vero cells. The plaque assays were immunostained with RSV polyclonal antisera for quantification after 5 days of incubation.

As was shown on FIG. 9B, the onset of replication of b/h PIV3/RSV F1*N-N was delayed and peak titers were lower than those of b/h PIV3/RSV F2. In contrast, b/h PIV3/RSV F1 displayed a growth curve that was nearly identical to that observed for b/h PIV3/RSV F2.

### 12. EXAMPLE 7

### Cloning of Trivalent Bovine Parainfluenza 3/Human Parainfluenza 3 Vectored Constructs

The following examples relate to the generation of trivalent vaccines that harbor the surface glycoproteins (F and HN) of hPIV3, RSV F, and hMPV F to protect children from disease caused by RSV, hMPV and hPIV3 using a single live attenuated virus vaccine. These trivalent viruses were recovered by reverse genetics.

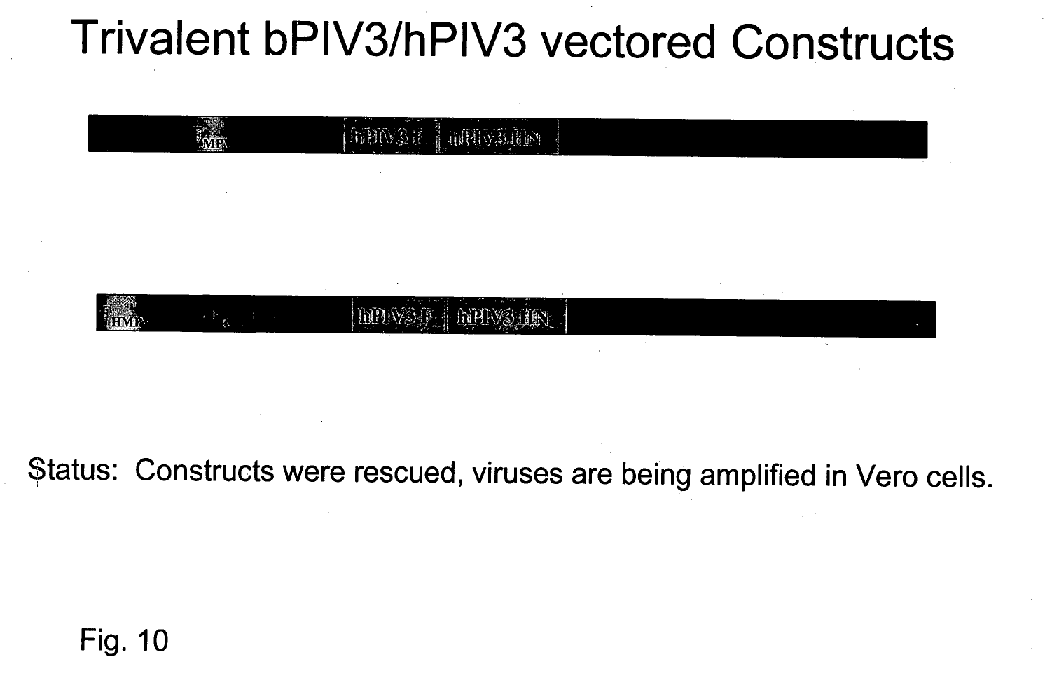

The construction of two virus genomes, each comprising a chimeric b/h PIV3 backbone with two additional heterologous sequence insertions, wherein one heterologous nucleotide sequence is derived from a metapneumovirus F gene and another heterologous nucleotide sequence is derived from a respiratory syncytial virus F gene, were done as follows (see FIG. 10): plasmids b/h PIV3/RSV F2 or b/h PIV3/hMPV F2 was digested with SphI and NheI, and a 6.5 kb fragment was isolated. The full-length cDNA for b/h PIV3 RSV F1 or b/h PIV3/hMPV F1 was digested with SphI and NheI and a 14.8 kb DNA fragment was isolated and ligated with the 6.5 kb DNA fragment derived from plasmid b/h PIV3/RSV F2 or b/h PIV3/hMPV F2 to generate full-length viral cDNAs.

Virus generated from the above described constructs (i.e., with F_{RSV} at position 1 and F_{hMPV} at position 3 and with F_{hMPV} at postion 1 and F_{RSV} at position 3) have been replicated and packaged in Vero cells. The rescued viruses, preferably the virus comprising the first construct as described herein, can be used as a trivalent vaccine against parainfluenza virus infection, metapneumovirus infection, and respiratory syncytial virus infection.

### 13. EXAMPLE 8

### Cloning of Two Respiratory Syncytial Virus F to the Bovine Parainfluenza 3/Human Parainfluenza 3 Vector

Chimeric viruses that carry two copies of the RSV F gene were designed in order to determine whether more RSV protein produced by the chimeric virus will result in an improved immunogenicity. This virus was rescued by reverse genetics, biologically cloned and amplified in Vero cells to yield a virus stock with a titer of 1×10⁶ pfu/ml. This virus, b/h PIV3/RSV F1F2, can be used to assess for virus growth kinetics, for RSV F protein production, and for replication and immunogenicity in hamsters.

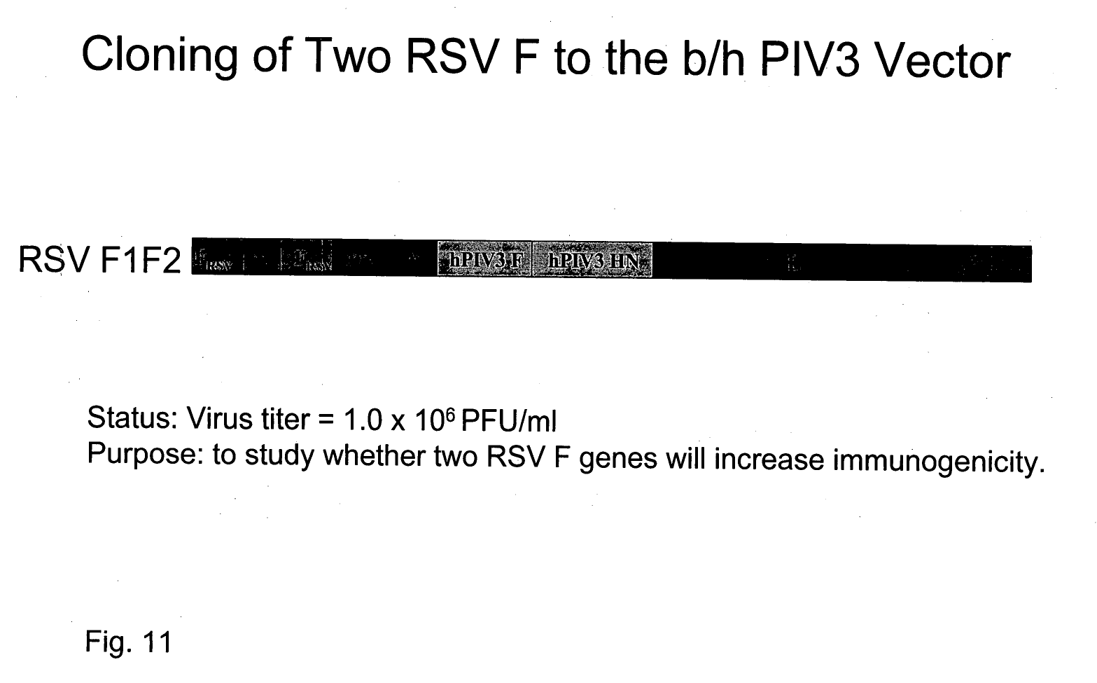

The constructs were generated in the following manner (see FIG. 11): the 1-5 RSV F2 plasmid was digested with SphI and NheI, and a 6.5 kb fragment was isolated. The full-length cDNA for b/h PIV3 RSV F1 was digested with SphI and NheI and a 14.8 kb DNA fragment was isolated and ligated with the 6.5 kb DNA fragment derived from 1-5 bPIV3/RSV F2 to generate full-length viral cDNAs.

### 14. EXAMPLE 9

### Construction and Cloning of Bovine Parainfluenza 3/Human Parainfluenza 3 Vectored Human Metapneumovirus F cDNA

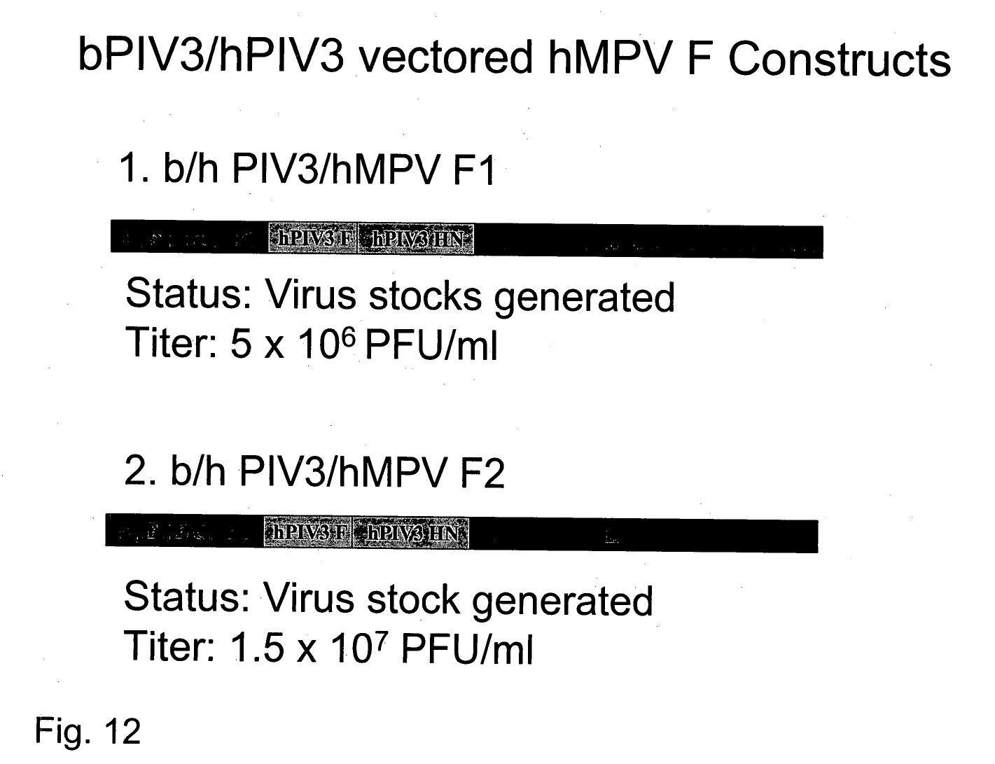

The F gene of human metapneumovirus (hMPV) was inserted in positions 1 and 2 of the b/h PIV3 genome (FIG. 12). The hMPV F gene cassette harbored the bPIV3 N-P intergenic region. A plasmid (pRF515) carrying the hMPV F gene (NL/1/00) was used, and a single nucleotide mutation in the hMPV F gene was corrected (i.e., nucleotide 3352 was corrected from C to T (wild type)), generating pRF515-M4. The bPIV3 N-P intergenic region was added at the 3′ end of the hMPV F gene using overlapping PCR. For hMPV F, the overlapping PCR oligo was 5′GGCTTCATACCACATAATTAGAAAAATAGCA CCTAATCATGTTCTTACAATGGTCGACC 3′. During this cloning step, oligos were used at the 5′ end (5′GCAGCCTAGGCCGCAATAACAATGTCTTGGAAAGTGGTG ATC 3′) and at the 3′ end of the hMPV F gene cassette (5′CTACCTAGGTGAATCTT TGGT TG 3′) in the PCR reaction that contained AvrII restriction enzyme sites. The hMPV F gene cassette was adjusted to conform to the rule of six using QuickChange mutagenesis kit and the following oligos (5′CCTAGGCCGCAATAGACAATGT CTTGG 3′, 5′CCAAGACATT GTCTATTGCGGCCTAGG 3′). Full-length b/h PIV3/hMPV F1 (position 1) and F2 (position 2) cDNA plasmids were generated in the same fashion as described in section 9, Example 4, supra, for b/h PIV3/eGFP1 and eGFP2.

The hMPV F gene cassette was sequenced to confirm the presence of an intact open reading frame, the predicted amino acid sequences, and to verify the rule of six. The hMPV F transcriptional unit was inserted into the first or second position using the AvrII restriction enzyme sites into a subclone 1-5 bPIV3 that was linearized with AvrII. After confirming proper orientation by restriction enzyme mapping, the plasmid harboring the hMPV gene in the first position was digested with SphI and BssHII and 4.8 kb (1-5 hMPV F1) DNA fragment was isolated. The rest of the b/h PIV3 genome was ligated as a SphI-BssHII 15.1 kb DNA fragment, yielding full-length cDNAs. A bPIV3 subclone harboring the hMPV gene in the second position was cut with SphI and NheI, and a 6.5 kb (bPIV3/hMPV F2) DNA fragment was isolated. The rest of the b/h PIV3 genome was ligated as an NheI-SphI DNA fragment of 14 kb in size to generate full-length cDNA plasmids.

### 15. EXAMPLE 10

### Immunoprecipitation and Replication Assays of Bovine Parainfluenza 3/Human Parainfluenza 3 Vectored Human Metapneumovirus F

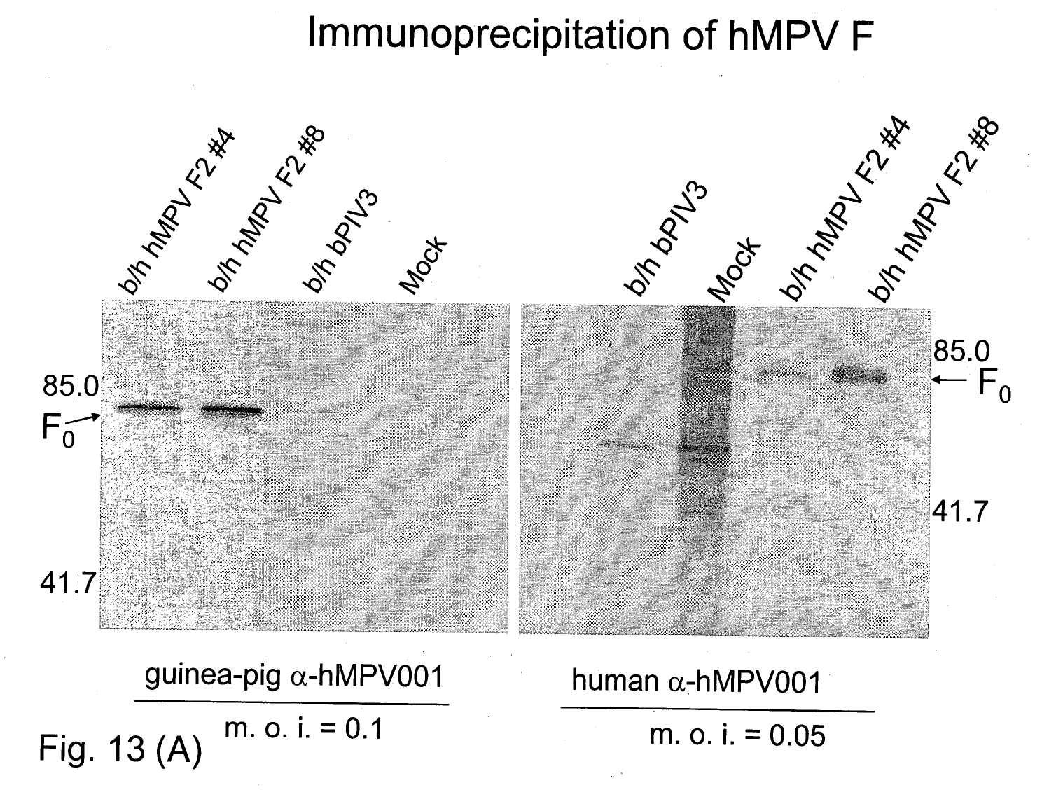

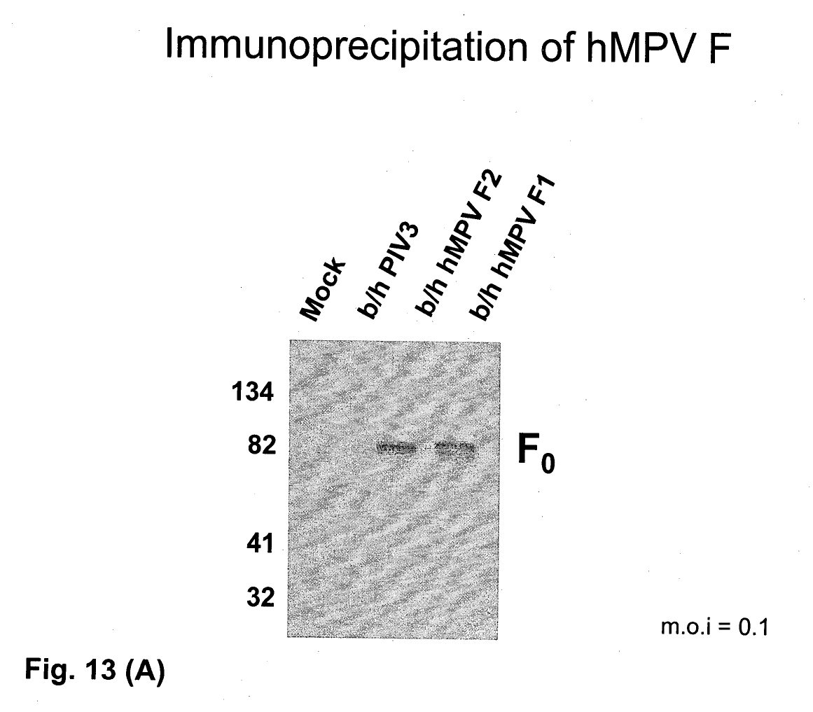

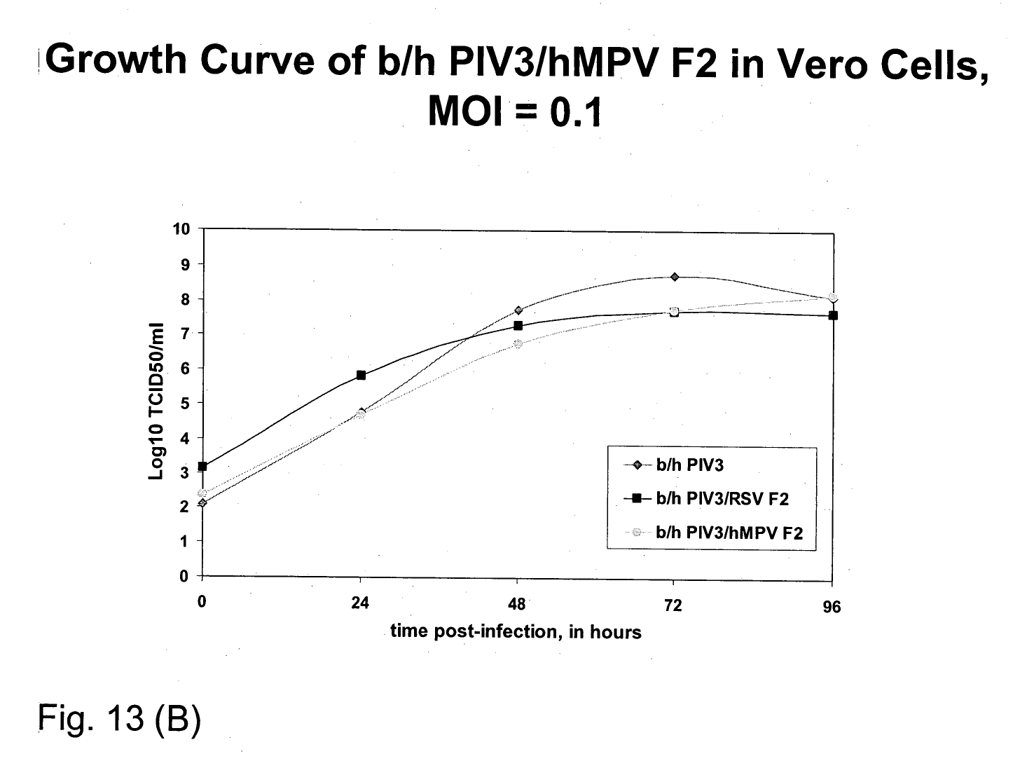

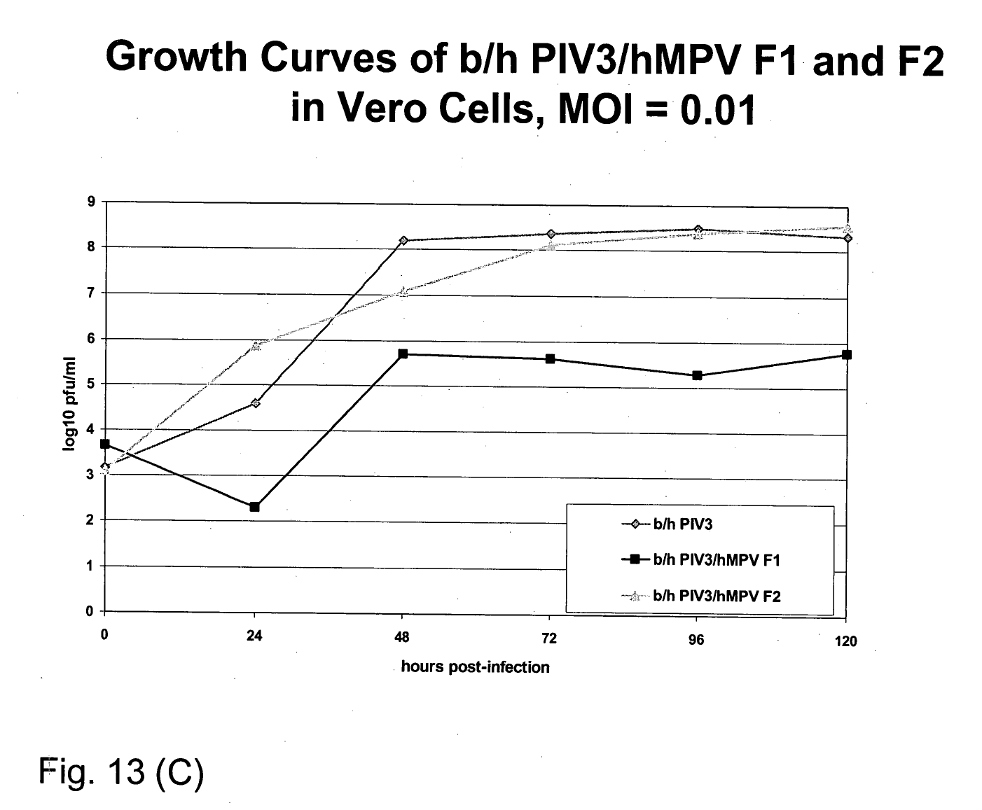

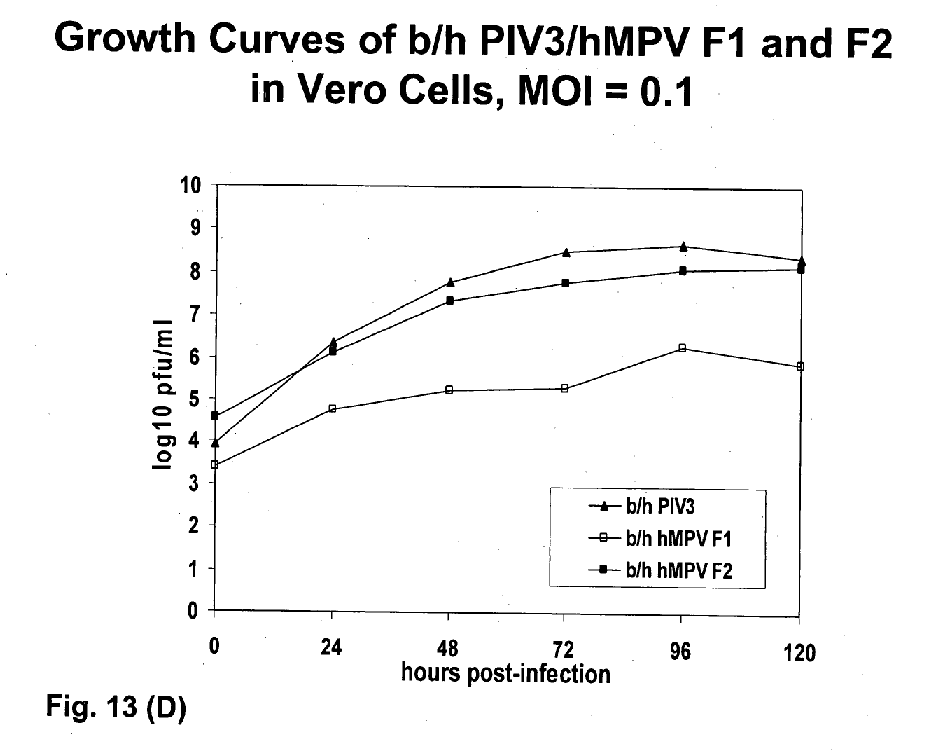

To confirm that the F protein was expressed in the b/h PIV3 vectored human metapneumovirus F at position 2 (hMPV F2), guinea pig or human antiserum were used to immunoprecipitate the hMPV F protein (see FIG. 13A). For immunoprecipitation of the hMPV F protein expressed by b/h PIV3/hMPV, Vero cells were infected with b/h PIV3 or b/h PIV3/hMPV F1 or F2 at an MOI of 0.1 or 0.05. Twenty-four hours post-infection, the cells were washed once with DME without cysteine and minus methionine (ICN) and incubated in the same media for 30 min. The media was removed and 0.5 ml DME lacking cysteine and methionine containing 100 μCi of [³⁵S]-Pro-Mix (Amersham) was added to the cells. The infected cells were incubated in the presence of ³⁵S-isotopes for 5 hours at 37° C. Media was removed and the infected cells were lysed in 0.3 M RIPA buffer containing protease inhibitors. The cell lysate was incubated with guinea pig or human polyclonal antisera against hMPV and bound to IgG-agarose (Sigma). After washing three times with 0.5 M RIPA buffer, the samples were fractionated on a 10% protein gel. The gel was dried and exposed to X-ray film.

The expression of hMPV F protein by b/h PIV3/hMPV F1 and F2 was shown by immunoprecipitation using the guinea pig hMPV antisera (FIG. 13A). Interestingly, a specific band migrating at approximately 80 kDa was observed in the lysates of b/h PIV3/hMPV F1 and F2. This size corresponded to the F precursor protein, F0. Non-specific bands of different sizes were also observed in the b/h PIV3 and mock control lanes (FIG. 13). This data suggested that the b/h PIV3/hMPV F1 and F2 expressed the hMPV F protein. The F1 cleavage product of the F0 precursor were not observed. Analysis of the F protein cleavage site revealed that the hMPV F protein cleavage site consisted of uncharged amino acid residues (RQSRFVL) while related viruses like RSV or APV A have charged amino acids at the F protein processing site, RKRRFLG and RRRRFVL, respectively. It is known from influenza viruses that F proteins with charged amino acids at the cleavage site can process the F protein efficiently and display a virulent phenotype (Hatta et al., Science (2001) 293(5536): 1840-22001). The “weak” cleavage site of the hMPV F protein may be responsible for detecting only the F0 protein since the F1 and F2 fragments would be present only at low levels that may not be detectable with the methods applied. Inefficient F protein cleavage may be one process directing the slow growth of hMPV replication in tissue culture and explain the trypsin requirement of some hMPV strains (van den Hoogen, 2001). However, the hMPV antibody reagents available are limited and these antisera interact only with the precursor of the hMPV F protein. It could also be possible that the cleaved F1 is unstable and thus not easily visualized using this method.

Growth curves were performed to determine the kinetics of virus replication of b/h PIV3/hMPV F2 and compare them to those observed for b/h PIV3 and b/h PIV3/RSV F2 in Vero cells at an MOI of 0.1 (FIG. 13B). The data showed that b/h PIV3/hMPV F2 displayed a delayed onset of replication at 24 hours post-infection compared to b/h PIV3/RSV F2. However, at 48 hours post-infection and beyond, a difference in replication was no longer observed.

Growth curves were also performed to determine the kinetics of viral replication of b/h PIV3/hMPV F1 and compare them to those observed for b/h PIV3/hMPV F2 and b/h PIV3 in Vero cells at an MOI of 0.01 (FIG. 13C). The growth curve was obtained using the same procedure as described in Section 8 for b/h PIV3/RSV chimeric viruses. The data showed that b/h PIV/hMPV F1 had a delayed onset of replication and yields lower peak titers compared to b/h PIV3/hMPV F2 or b/h PIV3. The plaque size of b/h hMPV F1 is also smaller compared to b/h hMPV F2.

The chimeric virus harboring the hMPV F gene in position 2 of the b/h PIV3 genome replicated to levels observed for b/h PIV3. Peak titers observed for b/h PIV3/hMPV F2 at 96 hours post-infection were 8.1 log10 PFU/ml. In contrast, the PIV3 expressing hMPV F protein from position 1 displayed a delayed onset of virus replication, and the peak titers were decreased by 1.8 log10 compared to b/h PIV3/hMPV F2 at 96 hrs post-infection. Only titers of 6.3 log10 PFU/ml were obtained from b/h PIV3/hMPV F1 infected Vero cells. The virus replication defect displayed by b/h PIV3/hMPV F1 was more severe than that of b/h PIV3/RSV G1 or b/h PIV3/RSV F1 suggesting that the nature of the insert may have an effect on virus replication.

Collectively, the data showed that b/h PIV3 expressing an hMPV protein in genome positions 1 or 2 replicated to peak titers of 10⁶-10⁸ PFU/ml in Vero cells. Viruses harboring the antigen insertion in position 2 replicated more efficiently in tissue culture than those containing foreign genes in position 1.

The chimeric viruses, b/h PIV3/hMPV F1 and F2 were also assessed for their ability to infect and replicate in Syrian Golden hamsters (Table 5). The chimeric viruses, b/h PIV3/hMPV F1 and F2, were therefore used to infect Syrian Golden hamsters intranasally and their ability to replicate in the respiratory tract was analyzed (Table 15). Five week old Syrian Golden hamsters (six animals per group) were infected intranasally with 1×10⁶ pfu or 1×10⁴ PFU of b/h PIV3, b/h PIV3/hMPV F1 or F2, or hMPV/NL/1/00 in a 100 μl volume. The different groups were maintained separately in micro-isolator cages. Four days post-infection, the nasal turbinates and lungs of the animals were harvested, homogenized and stored at −70° C. The titers of virus present in the tissues were determined by TCID₅₀ assays in Vero cells. For the challenge studies, the animals were inoculated on day 28 intranasally with 1×10⁶ pfu/ml of hPIV3 or hMPV/NL/1/00. Four days post-challenge, the nasal turbinates and lungs of the animals were isolated and assayed for challenge virus replication by plaque assays on Vero cells that were immunostained for quantification.

TABLE 5
Replication of b/h PIV3 Expressing the hMPV
F Protein in Positions 1 or 2 in Hamsters

Mean virus titer on day 4 post-infection
(log₁₀ TCID₅₀/g tissue ± S.E.) ^{b}

| Virus ^{a} | Nasal turbinates | Lungs |
|---|---|---|
| b/h PIV3 | 4.8 ± 0.2 | 5.6 ± 0.6 |
| b/h hMPV F1 | 5.3 ± 0.5 | 5.7 ± 0.4 |
| b/h hMPV F2 | 5.7 ± 0.5 | 4.6 ± 0.3 |
| hMPV | 5.3 ± 0.1 | 3.6 ± 0.3 |

^{a} Groups of six hamster were inoculated intranasally with 1 × 10⁶ pfu of indicated virus.
^{b} Standard error
Note:
TCID₅₀ assays were read for CPE on Day 10.

The results showed that b/h PIV3/hMPV F1 and F2 replicated in the nasal turbinates of hamsters to high levels of 5.3 and 5.7 log₁₀ TCID₅₀/g tissue, respectively. These titers were similar to those observed for b/h PIV3 (4.8 log₁₀ TCID₅O/g tissue). In comparison, wild type hMPV displayed titers of 5.3 log₁₀ TCID₅₀/g tissue in the upper respiratory tracts of hamsters (Table 5). b/h PIV3/hMPV F1 and F2 replicated to titers of 5.7 and 4.6 log₁₀ TCID₅₀/g tissue in the lungs of hamsters (Table 5). These titers were similar to those observed for b/h PIV3 (5.6 log₁₀ TCID₅O/g tissue). Wild-type hMPV displayed reduced titers of 3.6 log₁₀ TCID₅₀/g tissue in the lower respiratory tract of hamsters (Table 5). These data demonstrated that b/h PIV3/hMPV F1 and F2 could efficiently infect and replicate in the upper and lower respiratory tract of Syrian Golden hamsters. These results suggested that hamsters are a suitable small animal model to study immunogenicity of hMPV as well as hMPV vaccine candidates.

### 16. EXAMPLE 11

### Cloning of the Soluble Respiratory Syncytial Virus F Gene Construct

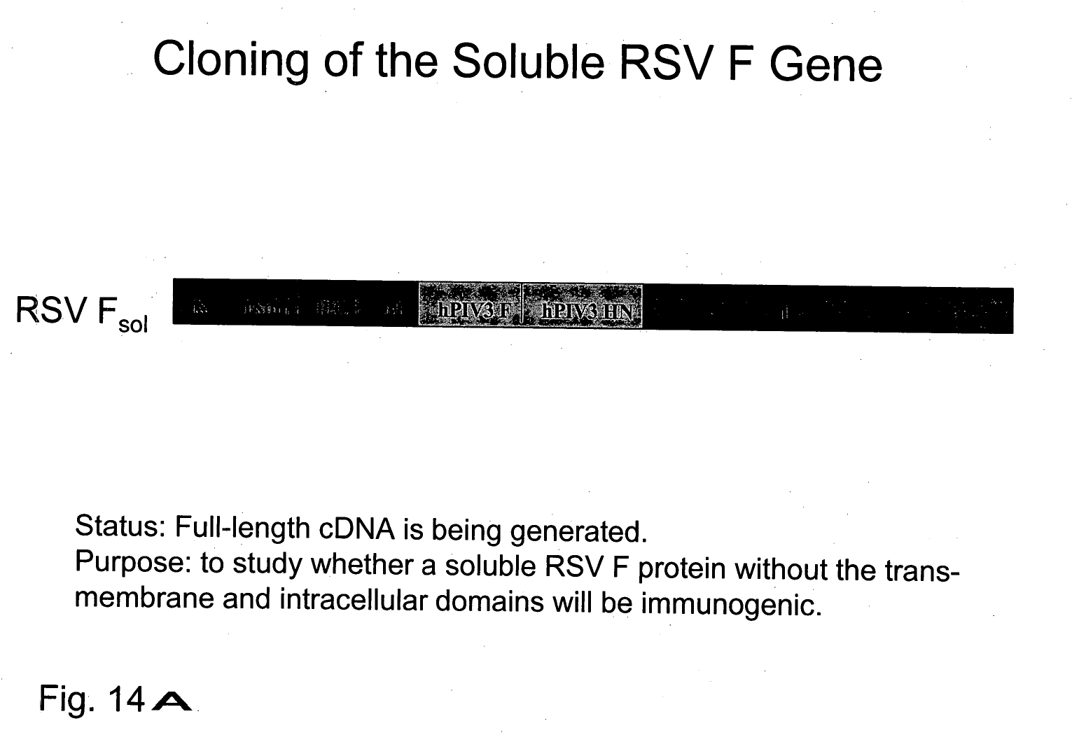

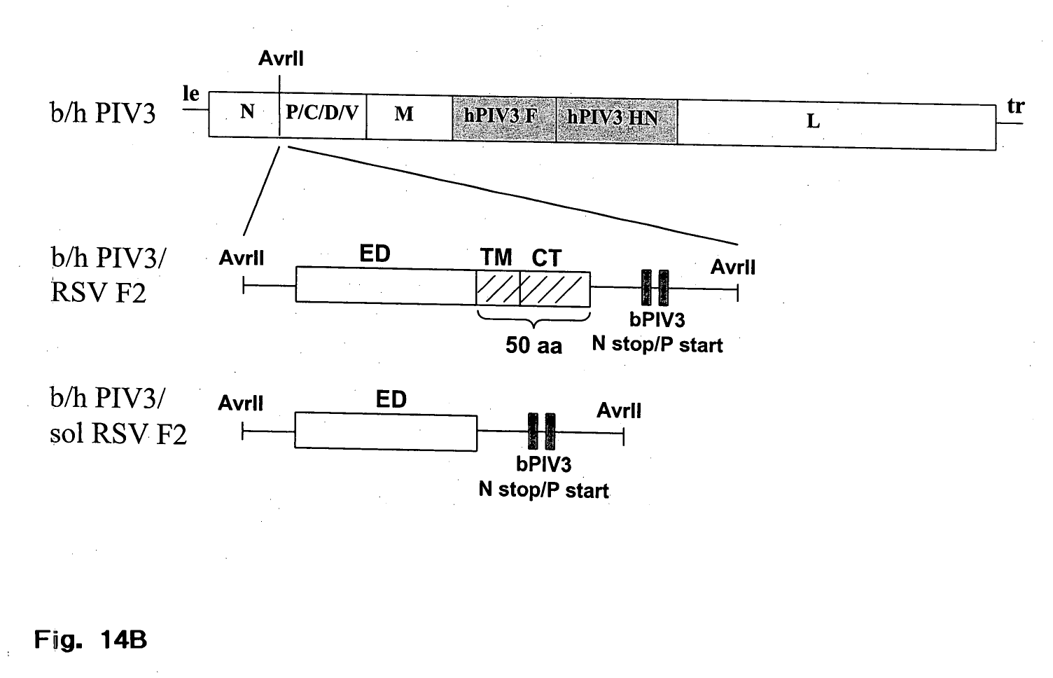

A construct (i.e., b/h PIV3/sol RSV F2) containing a single copy of the soluble RSV F gene, a version of the RSV F gene lacking the transmembrane and cytosolic domains, was also generated (FIG. 14). This construct can be used to test for immunogenicity (soluble RSV F is still expected to elicit an RSV specific immune response). Its advantage would be the inability of the soluble RSV F to be incorporated into the virion membrane. Therefore this virus may be viewed as a safer chimeric virus since its virus tropism is not expected to change. The cDNA plasmid for b/h PIV3/sol RSV F can be rescued by reverse genetics. The b/h PIV3/sol RSV F2 was constructed as follows.

The bovine/human (b/h) PIV3/sol RSV F2 cDNA harbored the fusion (F) and hemagglutinin-neuraminidase (HN) genes derived from human PIV3 while the rest of the viral genome originated from bPIV3. The previously described plasmid 1-5 bPIV3/RSV F2 was used as a DNA template for PCR. This plasmid contained bPIV3 sequences from nucleotides (nt) 1-5200 and the RSV F gene inserted at nt 1774. An oligo which anneals at nt 5946 (in the F gene) of the RSV A2 genome and the oligo 5′CGTGGTCGACCATTG TAAGAACATGATTAGGTGCTATTTTTATTTAATTTGTGGTGGATTTACCGGC3′ were employed to remove the trans-membrane and cytoplasmic domains of RSV F, deleting 150 nucleotides. The resulting PCR fragment was digested with HpaI and SalI and introduced into 1-5 bPIV3/RSV F2 treated with HpaI and SalI to yield the plasmid 1-5 bPIV3/sol RSV F2. The bPIV3 subclone harboring the sol RSV F gene in the second position was cut with SphI and NheI and a 6.3 kb DNA fragment was isolated. The rest of the bovine/human PIV3 genome was ligated as an NheI-SphI DNA fragment of 14 kb in size to generate the full-length b/h PIV3/sol RSV F2 cDNA plasmid. The recombinant virus was recovered by reverse genetics. High titer virus stocks were generated and quantified by plaque assays on Vero cells that were immunoperoxidase stained using RSV goat polyclonal antisera. The virus stocks were stored at −70° C.

### 17. EXAMPLE 12

### Expression of Human Metapneumovirus F in Cells Infected with Bovine Parainfluenza 3/Human Parainfluenza 3 Vectored Human Metapneumovirus F

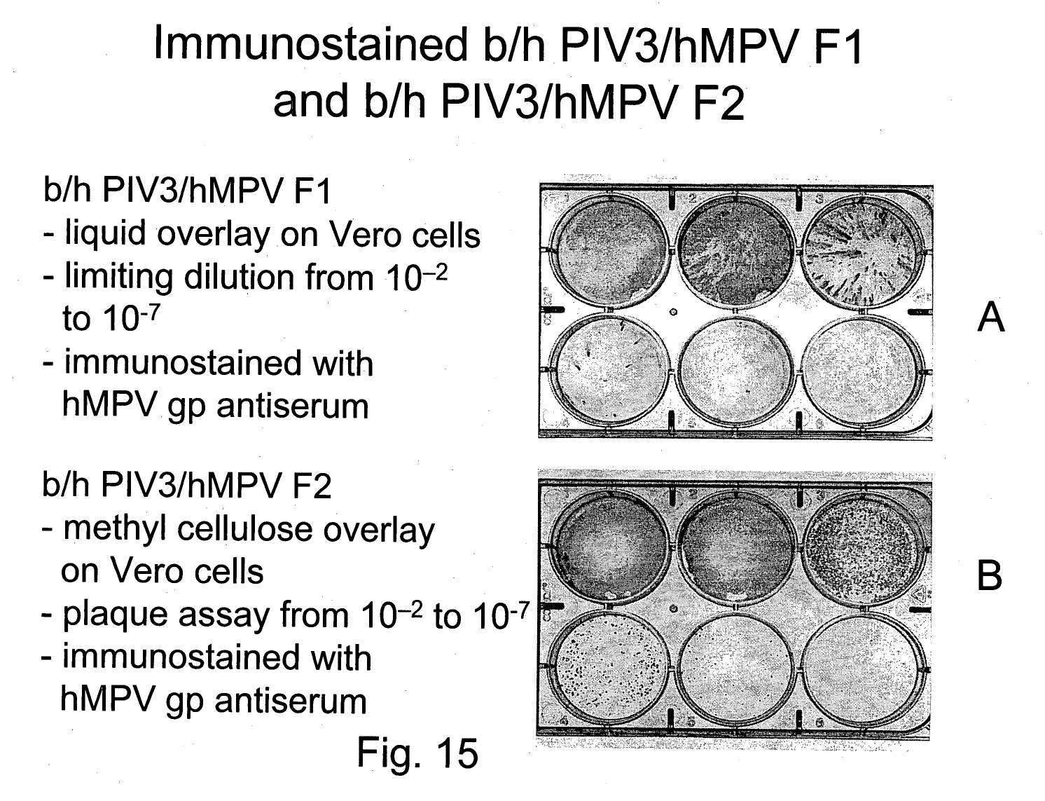

The b/h 104 hMPV F virus stocks were serially diluted 10 fold and used to infect subconfluent Vero cells. Infected cells were overlayed with optiMEM media containing gentamycin and incubated at 35° C. for 5 days. Cells were fixed with 100% methanol and immunostained with 1:1000 dilution of anti-hMPV001 guinea pig sera followed by 1:1000 dilution of anti-guinea pig HRP conjugated antibodies. Expression of hMPV F is visualized by specific color development in the presence of the AEC substrate system (DAKO corporation). See FIG. 15A.

The b/h NP-P hMPV F virus stocks were serially diluted 10 fold and used to infect subconfluent Vero cells. Infected cells were overlayed with 1% methyl cellulose in EMEM/L-15 medium (JRH Biosciences; Lenexa, Kans.) supplemented with 1×L15/MEM media containing penicillin/streptomycin, L-glutamine and fetal bovine serum. Infected cells were incubated at 35° C. for 5 days, fixed with 100% methanol and immunostained with 1:1000 dilution of anti-hMPV001 guinea pig sera followed by 1:1000 dilution of anti-guinea pig HRP conjugated antibodies. (See FIG. 15B). The anti hMPV001 guinea pig serum is specific for hMPV001 proteins and do not bind to b/h PIV3 proteins.

### 18. EXAMPLE 13

### Rescue of Chimeric Bovine Parainfluenza Type 3/Human Parainfluenza Type 3 Virus in Hela Cells and Vero Cells

Rescue of the chimeric b/h PIV3 virus was done using a similar procedure as for bPIV3 rescue. Rescue of b/h PIV3 chimeric virus by reverse genetics was carried out in HeLa cells using LipofecTACE (Gibco/BRL). The 80% confluent HeLa cells, Hep-2 cells, or Vero cells were infected with MVA at an MOI of 4. One hour post-infection, the full-length anti-genomic b/h PIV3 cDNA (4 μg) was transfected into the infected HeLa or Vero cells together with the NP (0.4 μg), P (0.4 μg), and L/pCITE (0.2 μg) expression plasmids. Forty hours post-transfection, the cells and the cell supernatant were harvested (P0) and subjected to a single freeze-thaw cycle. The resulting cell lysate was then used to infect a fresh Vero cell monolayer in the presence of 1-beta-D-arabinofuranosylcytosine (ara C), a replication inhibitor of vaccinia virus, to generate a P1 virus stock. The supernatant and cells from these plates were harvested, freeze-thawed once and the presence of bPIV3 virus particles was assayed for by immunostaining of virus plaques using PIV3-specific antiserum. The cell lysates of the P1 harvest resulted in complete CPE of the Vero cell monolayers and immunostaining indicated the presence of an extensive virus infection.

### 19. EXAMPLE 14

### Rescue of Bovine Parainfluenza Type 3/Human Parainfluenza Type 3 Vectored Human Metapneumovirus F Viruses

The b/h PIV3 viruses expressing hMPV F at position one (b/h 104 hMPV F) or position two (b/h NP-P hMPV F) were obtained as follows. HEp-2 or Vero cells at 80-90% confluency in 6 well dishes were infected with Fowlpox-T7 at a multiplicity of infection (m.o.i) of 0.1 to 0.3. Following infection with Fowlpox-T7, cells were washed once with PBS and transfected with the following amounts of plasmid DNA: full length b/h 104 hMPV F or b/h NP-P hMPV F cDNA 2.0 μg, pCite N 0.4 μg, pCite P 0.4 μg, pCite L 0.2 μg. (The pCite plasmids have a T7 promoter followed by the IRES element derived from the encephalomyocarditis virus (EMCV)). Transfection was performed in the presence of Lipofectamine 2000 (Invitrogen) according to manufacturer's instruction. The transfection reaction was incubated at 33° C. for 5 to 12 hours following which the media containing lipofectamine 2000 was replaced with 2 ml of fresh OptiMEM containing gentamicin. The transfected cells were further incubated at 33° C. for two days. Cells were stabilized with SPG and lyzed by one freeze-thaw cycle at −80° C. The crude cell lysate was used to infect a new Vero monolayer in order to amplify rescued viruses. The chimeric viruses were purified by limiting dilutions in Vero cells and high titer virus stocks of 10⁶-10⁸ PFU/ml were generated. Expression of the hMPV F protein was confirmed by immunostaining with polyclonal hMPV guinea pig antiserum.

### 20. EXAMPLE 15

### Rescue of Bovine Parainfluenza Type 3/Human Parainfluenza Type 3 Vectored Respiratory Syncytial Virus Genes by Reverse Genetics

Infectious virus was recovered by reverse genetics in HeLa or HEp-2 cells using transfection methods described previously (see Example 13). Briefly, HEp-2 or Vero cells at 80-90% confluency in 6 well tissue culture dishes were infected with FP-T7 or MVA-T7 at a multiplicity of infection (m.o.i.) of 0.1-0.3 or 1-5, respectively. Following infection with FP-T7 or MVA-T7, cells were washed once with PBS and transfected with the following amounts of plasmid DNA (2.0 μg full-length b/h PIV3 RSV F or G cDNA, 0.4 μg pCITE/N, 0.4 μg pCITE/P, 0.2 μg pCITE/L). Transfections were performed in the presence of Lipofectamine 2000 (Invitrogen) according to manufacturer's instruction. The transfection reactions were incubated at 33° C. for 5 to 12 hours following which the media containing Lipofectamine 2000 was replaced with 2 ml of fresh OptiMEM containing gentamicin. The transfected cells were incubated further at 33° C. for two days. Cells were stabilized with SPG and lysed with one freeze-thaw cycle at −80° C. The crude cell lysate was used to infect a new Vero cell monolayer in order to amplify rescued viruses. The chimeric viruses were purified by limiting dilutions in Vero cells and high titer virus stocks of 10⁶-10⁸ PFU/ml were generated. The RSV genes of the chimeric viruses were isolated by RT-PCR and the sequences were confirmed. Expression of the RSV proteins was confirmed by immunostaining of infected Vero cell monolayers with RSV goat polyclonal antiserum (Biogenesis).

### 21. EXAMPLE 16

### Confirmation of Chimeric Bovine Parainfluenza Type 3/Human Parainfluenza Type 3 Virus Rescue by RT-PCR

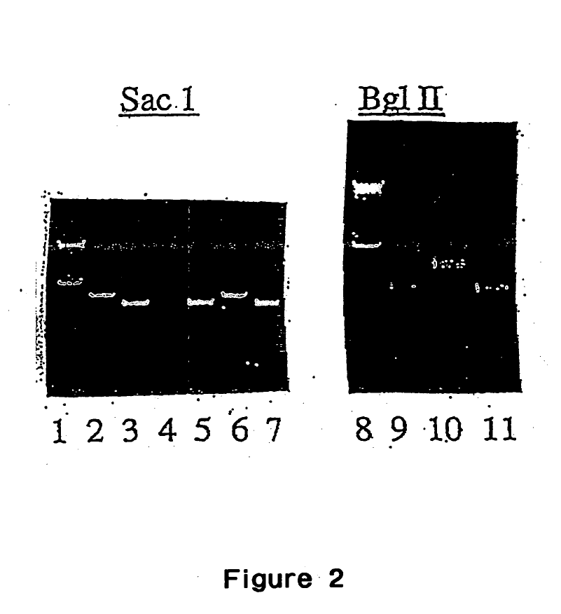

To ascertain that the rescued virus is chimeric in nature, i.e. the virus contains hPIV3 F and HN gene sequences in a bPIV3 backbone, the viral RNA genome was analyzed further by RT-PCR. Vero cells, infected with the P1 virus stock of three independently derived isolates of b/h PIV3 were harvested and total RNA was isolated. The viral RNA was amplified using an oligo that anneals at position 4757 of bPIV3. A viral region from nt 5255 to 6255 was amplified by PCR. The resulting 1 kb PCR fragment should contain hPIV3 sequences. This was confirmed by digestion with enzymes (Sacl and Bgl II) specific for hPIV3 and that do not cut in the complementary region of bPIV3 (see FIG. 2). As expected, Sacl and Bgl II cut the PCR fragment into smaller fragments confirming that the isolated sequences are derived from hPIV3 (see lanes 3, 5, 7). In addition, a region in the polymerase L gene from nt 9075 to nt 10469 was amplified by PCR. This region should contain bPIV3 sequences. Again the resulting 1.4 kb PCR fragment was digested using enzyme specific for bPIV3 (Pvull and BamH1) that do not cut in the equivalent region of hPIV3 (FIG. 3). The 1.4 kb fragment was indeed digested by both Pvull and BamH1 confirming that the polymerase gene is bPIV3 in origin (see lanes 3, 4, 6, 7, 9 and 10 of FIG. 3). In summary, the RT-PCR analysis shows that the rescued b/h PIV3 virus is chimeric in nature. It contains hPIV3 F and HN genes in a bPIV3 genetic backbone.

### 22. EXAMPLE 17

### Genetic Stability of Bovine Parainfluenza Type 3/Human Parainfluenza Type 3 Vectored Respiratory Syncytial Virus and Human Metapneumovirus Genes

In order to demonstrate that the b/h PIV3/RSV and the b/h PIV3/hMPV chimeric viruses are genetically stable and maintain the introduced RSV or hMPV gene cassettes, infected cell lysates were serially blind passaged ten times in Vero cells. Sub-confluent Vero cells in T25 flasks were infected with b/h PIV3/RSV or b/h PIV3/hMPV at an MOI of 0.1 and incubated for 4 days at 33° C. or until CPE was visible. At the end of the incubation period the infected cells and media were harvested, frozen and thawed two times, and the resulting cell lysate was used to infect a new T25 flask of Vero cells. This cycle was repeated ten times. All cell lysates from P1 to P10 were analyzed by plaque assay and immunostaining for expression of RSV or hMPV proteins and virus titers. At passage 10, the RSV F, RSV G, or hMPV F gene cassettes were isolated by RT-PCR from P10 lysates, and were verified by DNA sequence analysis (to identify possible nucleotide alterations). All of the isolates maintained the RSV or hMPV gene cassettes and RSV or hMPV protein expression for the 10 passages analyzed. An increased insert stability of PIV3 expressing RSV or hMPV genes depending on location of gene insertion in the PIV3 genome, position 1 or 2, was not observed.

### 23. EXAMPLE 18

### Virion Fractionation of Bovine Parainfluenza Type 3/Human Parainfluenza Type 3 Vectored Respiratory Syncytial Virus Genes on Sucrose Gradients

The question of whether the RSV proteins were incorporated into the b/h PIV3 virion was investigated further by use of a biochemical assay. Vero cells were inoculated with each of the chimeric b/h PIV3/RSV viruses at an MOI of 0.1. When maximum CPE was visible, the infected monolayers were frozen, thawed, and spun for 10 minutes at 2000 rpm. The clarified supernatants were spun through a 20% sucrose cushion at 100,000×g for 90 minutes. The pellet was then resuspended in PBS and layered gently on top of a 20-66% sucrose gradient. The gradients were spun at 100,000×g for 20 hours to achieve equilibrium. Eighteen 2 ml fractions were harvested starting from the top of the gradient. 0.4 ml of each fraction was removed for virus titer determination. Each fraction was resuspended in 2 volumes of 20% PBS and concentrated by spinning at 100,000×g for 1 hour. The pellet was then resuspended in 0.05 ml Laemmli buffer (Biorad) and analyzed for RSV and PIV3 proteins by Western blot, using an RSV F MAb (NuMax L1FR-S28R), RSV (Biogenesis) and bPIV3 (VMRD) polyclonal antisera. C-terminally truncated RSV F protein expressed in baculovirus that was purified to homogeneity, was also analyzed on a sucrose gradients.

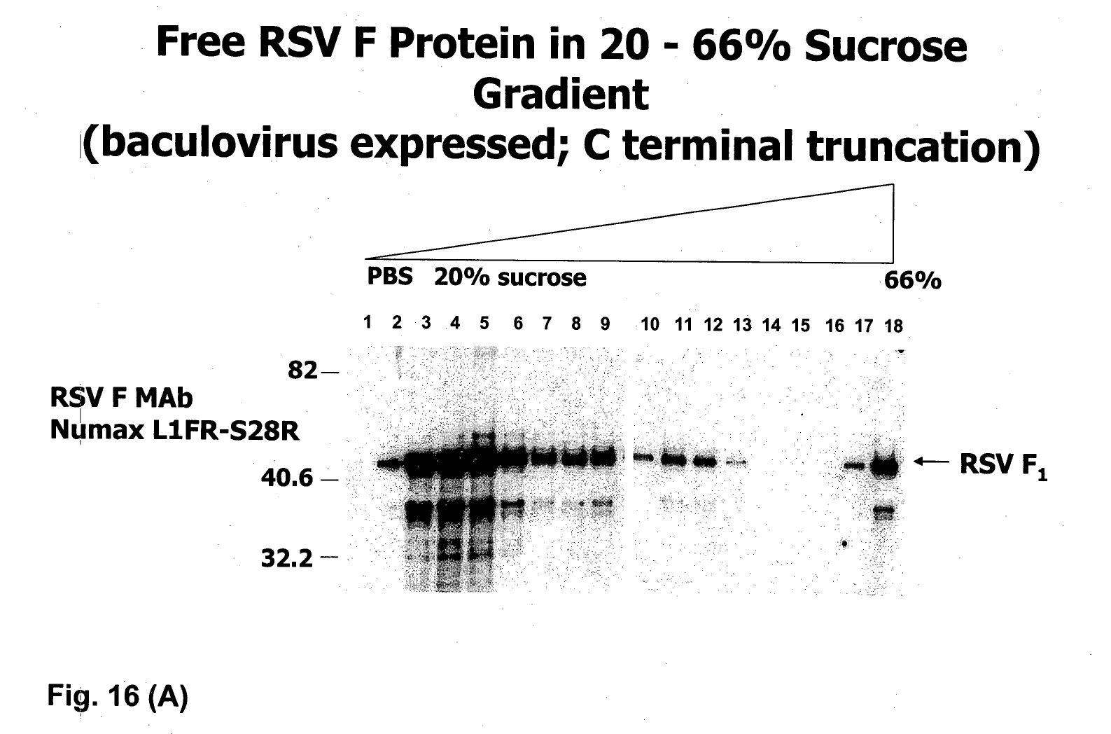

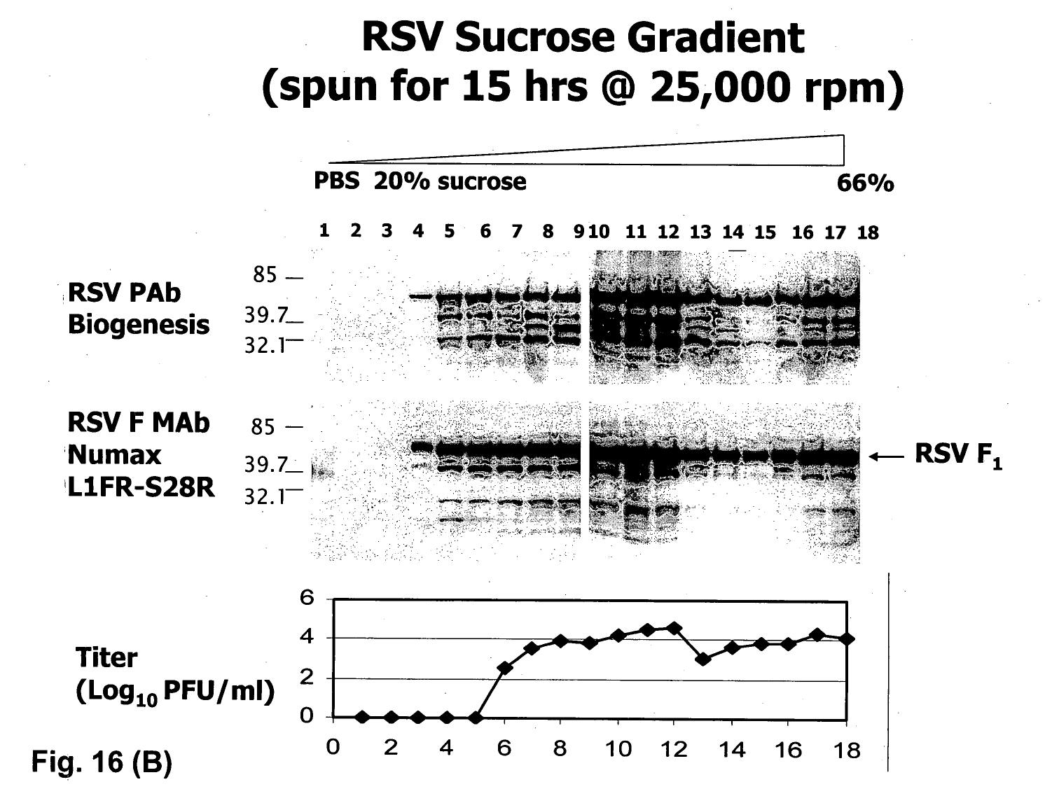

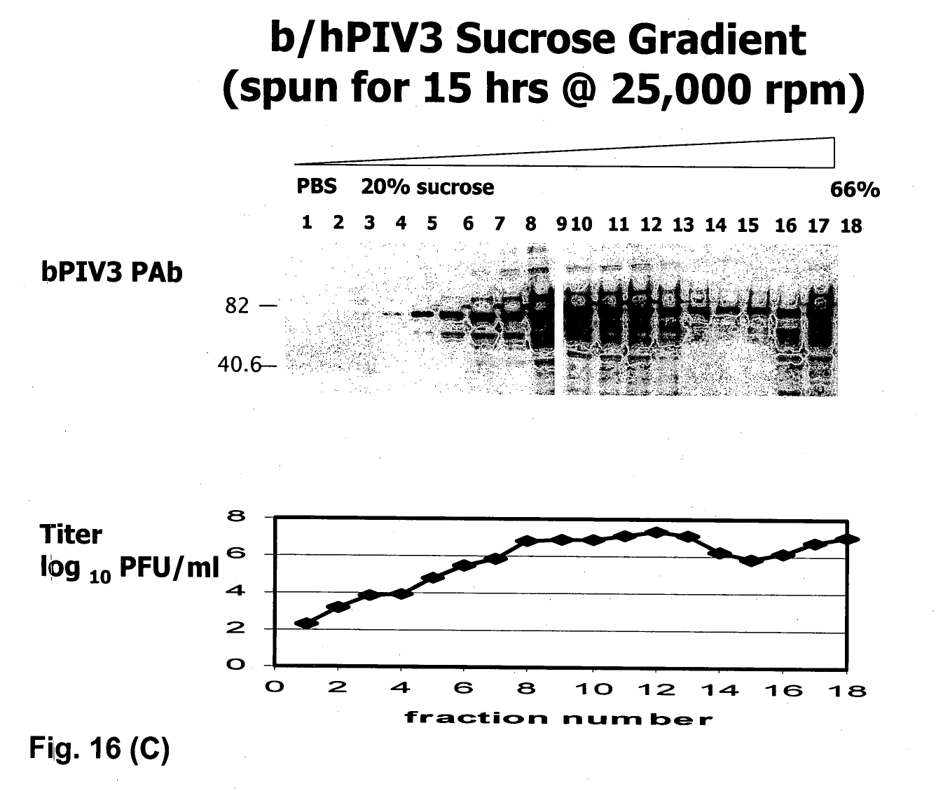

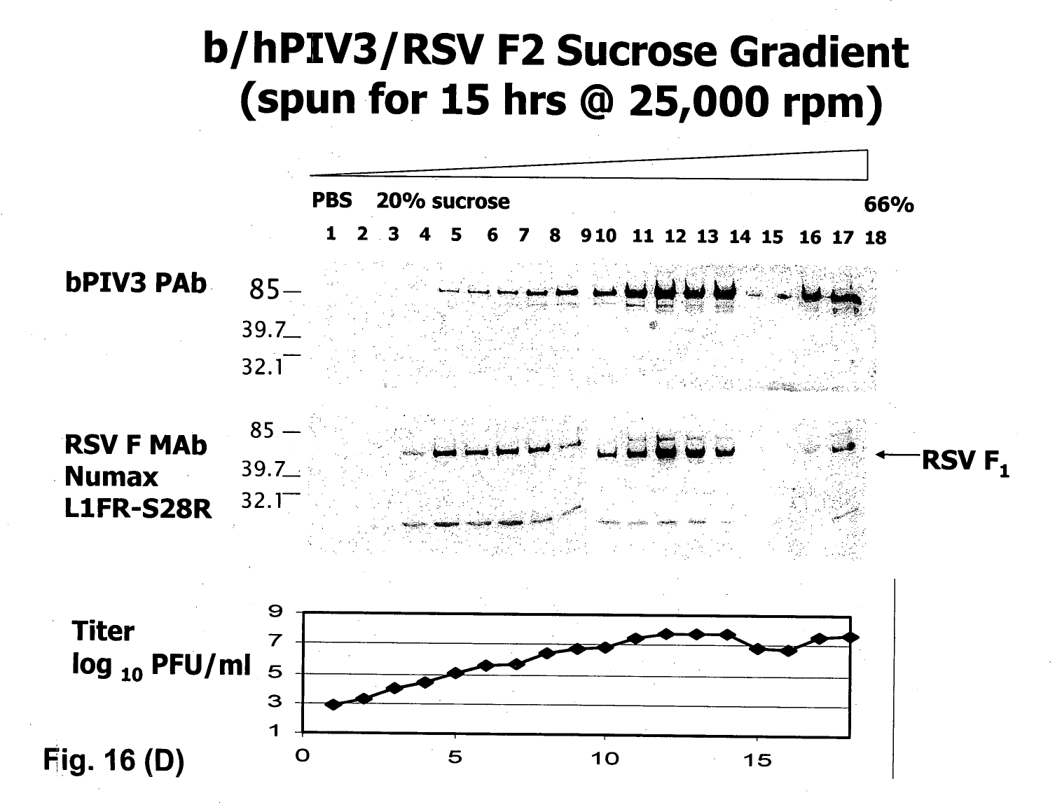

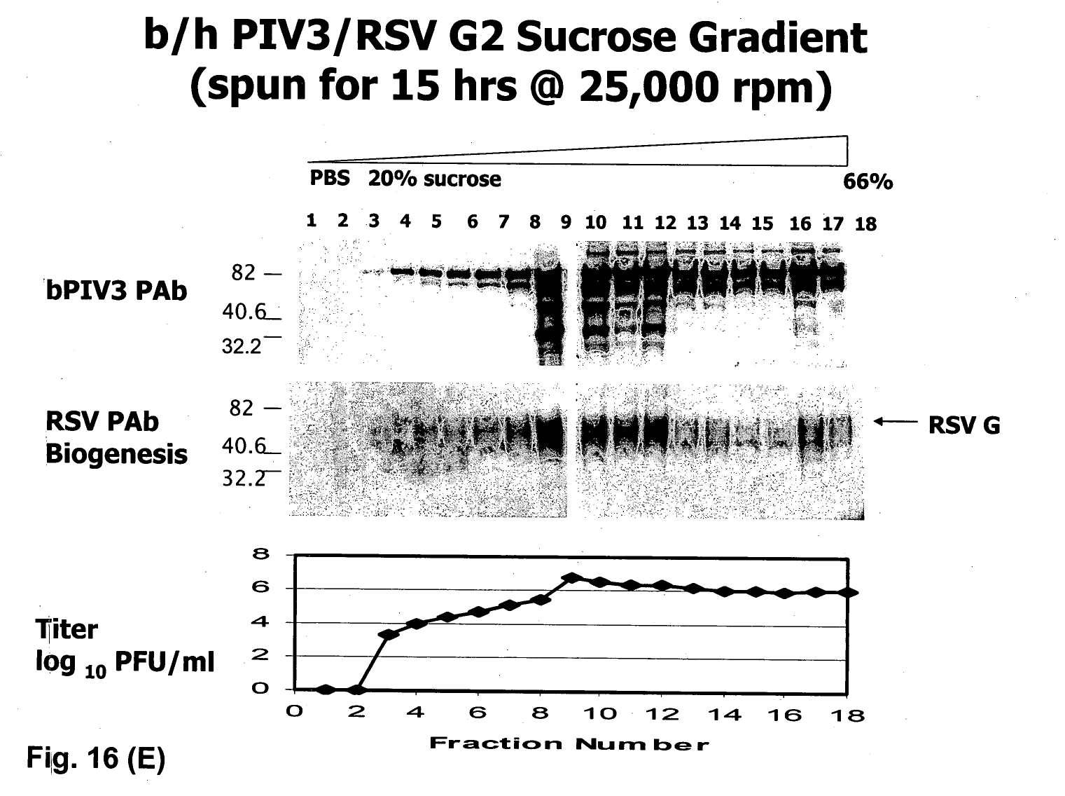

The fractions were also analyzed for peak virus titers by plaque assay. Control gradients of free RSV F (generated in baculovirus and C-terminally truncated), RSV A2, and b/h PIV3 were carried out initially. The majority of free RSV F was present in fractions 3, 4, 5, and 6 in the top portion of the gradient (FIG. 16A). The biggest concentration of RSV virions was observed in fractions 10, 11 and 12 (FIG. 16B). The RSV fractions were probed with RSV polyclonal antiserum as well as with RSV F MAb. The fractions that contained the greatest amounts of RSV virions also showed the strongest signal for RSV F, suggesting that the RSV F protein co-migrated and associated with RSV virions (FIG. 16B). These fractions also displayed the highest virus titers (FIG. 16B). The b/h PIV3 virions may be more pleiomorphic and thus the spread of the peak fractions containing b/h PIV3 virions was more broad. b/h PIV3 virions were present in fractions 9, 10, 11, 12, and 13 (FIG. 16C). Again the fractions harboring the most amounts of virions, also displayed the highest virus titers by plaque assay (FIG. 16C). Sucrose gradient fractions of b/h PIV3/RSV F2 were analyzed with both a PIV3 polyclonal antiserum and an RSV F MAb (FIG. 16D). The fractions containing most of the virions were fractions 11, 12, 13, and 14 as was shown by western using the PIV3 antiserum. Correspondingly, these were also the fractions that displayed the highest amounts of RSV F protein. However, some free RSV F was also present in fractions 5 and 6. Fractions 11, 12, 13 and 14 displayed the peak virus titers (FIG. 16D). Similarly, the fractions containing the most virions of b/h PIV3/RSV G2 (fractions 9, 10, 11, and 12) also showed the strongest signal for RSV G protein (FIG. 16E). Again these were the fractions with the highest virus titers (FIG. 16E). Collectively these data suggested that the majority of the RSV F and G proteins co-migrated and associated with the b/h PUV3 virions. However, some free RSV proteins were also present in the top fractions of the gradients.

### 24. EXAMPLE 19

### The Chimeric Bovine Parainfluenza Type 3/Human Parainfluenza Type 3 vectored Respiratory Syncytial Virus (RSV) could not be Neutralized with RSV Antisera

In order to address the important safety question of whether the RSV surface glycoproteins incorporated into the b/h PIV3 virion resulted in an altered virus tropism phenotype, neutralization assays were carried out (Tables 6 and 7). Neutralization assays were performed for b/h PIV3, b/h PIV3/RSV chimeric viruses or RSV using Vero cells. Serial two-fold dilutions of RSV polyclonal antiserum (Biogenesis; Poole, England), an RSV F MAb (1200 MAb) obtained from Dr. Judy Beeler and the WHO reagent bank (Beeler and Coelingh, J. Virol. (1989) 63(7): 2941-50), and hPIV3 F (C191/9) and HN (68/2) MAbs (van Wyke Coelingh and Tierny, J. Virol. 1989 63(9): 3755-60; van Wyke Coelingh et al., 1985), were incubated with approximately 100 PFU of either b/h PIV3, b/h PIV3/RSV chimeric viruses or RSV in 0.5 ml OptiMEM at RT for 60 min. Following the incubation, virus-serum mixtures were transferred to Vero cell monolayers, incubated at 35C for 1 hour, overlaid with 1% methyl cellulose in EMEM/L-15 medium (JRH Biosciences; Lenexa, Kans.) and incubated at 35 C. Six days post-inoculation, the infected cell monolayers were immunostained. Neutralization titers were expressed as the reciprocal of the highest serum dilution that inhibited 50% of viral plaques. RSV F MAbs (WHO 1200 MAb) neutralized 50% of wildtype RSV A2 at a 1:2000 dilution (Table 6). In contrast, even a dilution of 1:25 did not neutralize any of the chimeric b/h PIV3/RSV. Similarly, a dilution of 1:400 of the polyclonal RSV antiserum (Biogenesis) neutralized 50% of RSV A2, but even a dilution of 1:15.6 did not neutralize b/hPIV3 RSV (Table 6).

TABLE 6
The b/h PIV3 RSV Chimeric Viruses are
not Neutralized by RSV Antibodies

| | |
|---|---|
| | Reciprocal 50% neutralizing |
| Virus used in | antibody dilution |

| neutralization assay | RSV F MAb | RSV Ab |
|---|---|---|
| RSV | 2000 | 400.0 |
| b/h PIV3 | <25 | <15.6 |
| b/h RSV F1*N-N | <25 | <15.6 |
| b/h RSV F2 | <25 | <15.6 |
| b/h RSV G1 | ND | <15.6 |
| b/h RSV G2 | ND | <15.6 |

hPIV3 F MAb C191/9 neutralized 50% of b/h PIV3 as well as the b/h PIV3/RSV at a dilution of 1:500 (Table 7). An hPIV3 HN MAb 68/2 neutralized b/h PIV3 at a dilution of 1:16,000, and the b/h PIV3/RSV at a dilution of 1:32,000 (Table 7).

TABLE 7
The b/h PIV3 RSV Chimeric Viruses
are Neutralized by hPIV3 Mabs

| | |
|---|---|
| | Reciprocal 50% neutralizing |
| Virus used in | antibody dilution |

| neutralization assay | hPIV3 F MAb | hPIV3 HN MAb |
|---|---|---|
| RSV | 62.5 | <500 |
| b/h PIV3 | 500 | 16000 |
| b/h RSV F1*N-N | 500 | 32000 |
| b/h RSV F2 | 500 | 32000 |
| b/h RSV G1 | ND^{d} | 32000 |
| b/h RSV G2 | ND | 32000 |

^{d}not determined.

These assays were also performed using the same conditions but in the presence of guinea pig complement and neutralization of the b/h PIV3/RSV was still not observed. The results obtained using RSV polyclonal as well as RSV F monoclonal antibodies suggested that the RSV F protein expressed by b/h PIV3 was not incorporated into the virion envelope. Albeit the assays used may not have been sufficiently sensitive to detect small amounts of RSV F protein on the virion surface. However, if low levels of RSV F were present on the b/h PIV3/RSV F2 virion surface, the RSV F protein was not able to functionally substitute for the PIV3 F protein. To further study this issue, a b/h PIV3 was generated that expressed a soluble form of the RSV F protein lacking the transmembrane and cytosolic domains, rendering the RSV F protein incapable of being inserted into the virion membrane (FIG. 14). The removal of the transmembrane and cytosolic domains was accomplished by deleting 50 amino acids at the C terminus of the RSV F protein. The bPIV3 gene end and gene start sequences of the sol RSV F gene cassette remained identical to that of the full-length RSV F gene cassette (FIG. 14). Both chimeric b/h PIV3 expressed the native and soluble RSV F proteins efficiently and replicated to high titers of 10⁷-10⁸ PFU/ml in tissue culture. These data further showed that the RSV proteins were not functional, i.e. the RSV F protein could not functionally substitute for the hPIV3 F protein that was blocked by the hPIV3 F antibody. Therefore, a change in virus tropism of the b/h PIV3 expressing foreign antigens derived from RSV and hMPV, is unlikely.

### 25. EXAMPLE 20

### The Chimeric Bovine PIV Demonstrate Attenuated Phenotypes and Elicit Strong Protective Responses when Administered In Vivo

Five week old Syrian Golden hamsters were infected with 5×10⁵ pfu of wildtype bPIV3, recombinant bPIV3, hPIV3, human/bovine PIV3, and placebo. The five different animal groups were kept separate in micro-isolator cages. Four days post-infection, the animals were sacrificed. The nasal turbinates and lungs of the animals were homogenized and stored at −80° C. Virus present in the tissues was determined by TCID₅₀ assays in MDBK cells at 37° C. Virus infection was confirmed by hemabsorption with guinea pig red blood cells. Table 8 shows the replication titers of the different PIV3 strains in hamsters in the lungs and nasal turbinates. Note that recombinant bPIV3 and the b/h PIV3 chimeric viruses are attenuated in the lungs of the hamsters:

TABLE 8
Replication of PIV3 Viruses in Syrian Golden
Hamsters in the Nasal Turbinates and Lungs.
Replication of bPIV3, r-bPIV3, r-bPIV3(1), hPIV3 and Bovine/Human
PIV3(1) in the Upper and Lower Respiratory Tract of Hamsters

| | Mean virus titer on day 4 postinfection | |
|---|---|---|
| | (log₁₀ TCID₅₀/g tissue = S.E.) ^{b} | |
| Virus ^{a} | Nasal turbinates | Lungs |
| bPIV3 | 5.3 ± 0.3 | 5.3 ± 0.2 |
| r-bPIV3 | 5.0 ± 0.3 | 3.5 ± 0.2 |
| r-bPIV3(1) | 5.5 ± 0.2 | 5.4 ± 0.2 |
| hPIV3 | 4.9 ± 0.2 | 5.4 ± 0.2 |
| Bovine/human PIV3(1) | 4.9 ± 0.2 | 4.5 ± 0.2 |

^{a} Groups of four hamsters were inoculated intranasally with 5 × 10⁵ PFU of indicated virus.
^{b} Standard error.

Furthermore, serum samples collected from the hamsters prior to infection and at day 21 post-infection were analyzed in a hemagglutination inhibition assay. The serum samples were treated with receptor destroying enzyme (RDE, DENKA Seiken Co.) and non-specific agglutinins were removed by incubation with guinea pig red blood cells for 1 hour on ice. Wildtype bPIV3 and hPIV3 were added to two-fold serially diluted hamster serum samples. Finally, guinea pig red blood cells (0.5%) were added, and hemagglutination was allowed to occur at room temperature. Table 9 shows the antibody response generated in the hamsters upon being infected with the different PIV3 strains. Note that the b/h PIV3 chimeric virus generates as strong an antibody response against hPIV3 as does wild type hPIV3, far exceeding the response generated by the recombinant or wildtype bPIV3:

TABLE 9
Hemaglutination Inhibition Assay Using Serum from
Hamsters Infected with Different PIV3 Viruses.

| | |
|---|---|
| Virus Used for Inoculation of the | Hamster Serum Titers for |

| Hamsters | wt bPIV3 | HPIV3 |
|---|---|---|
| Recombinant bPIV3 | 1:16 | 1:16 |
| Wt bPIV3 | 1:16 | 1:8 |
| Wt hPIV3 | 1:4 | 1:128 |
| b/h PIV3 chimeric virus | 1:8 | 1:128 |
| Placebo | <1:4 | <1.4 |

These results demonstrate the properties of b/h PIV3 chimeric viruses of the present invention which make these recombinants suitable for use in vaccine formulations. Not only do the b/h PIV3 chimeric viruses demonstrate an attenuated phenotype when administered in vivo, but they also generate as strong an antibody response as the wildtype hPIV3. Thus, because the chimeric viruses of the present invention have a unique combination of having an attenuated phenotype and eliciting as strong an immune response as a wildtype hPIV, these chimeric viruses have the characteristics necessary for successful use in humans to inhibit and/or protect against infection with PIV.

### 26. EXAMPLE 21

### Replication of Bovine Parainfluenza 3/Human Parainfluenza 3 Vectored Respiratory Syncytial Virus G or F Protein in the Upper and Lower Respiratory Tract of Hamsters

Five week old Syrian Golden hamsters (six animals per group) were infected intranasally with 1×10⁶ PFU or 1×10⁴ PFU of b/h PIV3, b/h PIV3/RSV, RSV A2, or placebo medium in a 100 μl volume. The different groups were maintained separately in micro-isolator cages. Four days post-infection, the nasal turbinates and lungs of the animals were harvested, homogenized and stored at −70° C. The titers of virus present in the tissues were determined by TCID₅₀ assays in Vero cells. For the challenge assays, the animals were inoculated on day 28 intranasally with 1×10⁶ pfu/ml of hPIV3 or RSV A2. Four days post-challenge, the nasal turbinates and lungs of the animals were isolated and assayed for challenge virus replication by plaque assays on Vero cells that were immunostained for quantification. Table 10 shows the replication titers of the different strains in hamsters in the lungs and nasal turbinates.

TABLE 10
Replication of bovine/human PIV3 Expressing the RSV G or F proteins
in the Upper and Lower Respiratory Tract of Hamsters.

Mean virus titer on day 4 postinfection
(log₁₀ TCID₅₀/g tissue = S.E.)^{b}

| Virus^{a} | Nasal turbinates | Lungs |
|---|---|---|
| b/h PIV3 | 4.8 ± 0.4 | 4.4 ± 0.3 |
| RSV A2 | 3.4 ± 0.5 | 3.3 ± 0.5 |
| b/h RSV G1 | 4.2 ± 0.7 | 2.9 ± 0.7 |
| b/h RSV F1 | 3.9 ± 0.4 | 2.7 ± 0.2 |
| b/h RSV F1 N-P | 4.6 ± 0.4 | 3.5 ± 0.2 |
| b/h RSV G2 | 4.2 ± 0.9 | 4.3 ± 0.2 |
| b/h RSV F2 | 4.6 ± 0.6 | 4.4 ± 0.5 |

^{a}Groups of four hamsters were inoculated intranaselly with 5 × 10⁶ PFU of indicated virus.
^{b}Standard error.

Syrian Golden hamsters represent a suitable small animal model to evaluate replication and immunogenicity of recombinant bPIV3 and hPIV3 genetically engineered viruses. It was expected that the introduction of the RSV antigens would not alter the ability of the chimeric b/h PIV3 to infect and replicate in hamsters since the foreign antigens were not incorporated into the virion (Table 6 and Table 7). When animals were immunized intranasally, the results showed that all of the chimeric b/h PIV3/RSV replicated to 4.2 to 4.6 log10 TCID₅₀/g tissue in the nasal turbinates of hamsters (Table 10). These levels of replication were similar to those observed for b/h PIV3 which displayed 4.8 log10 TCID₅₀/g tissue (Table 10). Syrian Golden hamsters are only semi-permissive for infection with RSV. The titers of RSV observed in the upper respiratory tract of hamsters were decreased by 1.4 log10 TCID₅₀/g tissue compared to those of b/h PIV3 (Table 10). The b/h PIV3/RSV harboring the RSV gene in position 1, displayed 0.9-1.5 log10 reduced titers in the lungs of hamsters compared to b/h PIV3 (Table 10). In contrast, the b/h PIV3/RSV that contained a gene insertion in position 2, replicated to within 0.1 log10 of the titers observed for b/h PIV3 in the lower respiratory tract of hamsters (Table 10). Chimeric PIV3 harboring foreign genes in position 1 or 2 retained the ability to replicate efficiently in the lower and upper respiratory tract of hamsters to b/h PIV3-like levels. The introduction of an additional gene into the b/h PIV3 genome in positions 1 or 2 did not attenuate the virus significantly for in vivo virus replication.

### 27. EXAMPLE 22

### Bovine Parainfluenza 3/Human Parainfluenza 3 Vectored Respiratory Syncytial Virus Immunized Hamsters were Protected Upon Challenge with Human Parainfluenza 3 and Respiratory Syncytial Virus A2

In order to evaluate whether the levels of replication observed for b/h PIV3/RSV were sufficient to elicit a protective immune response in hamsters, the animals were challenged intranasally with 10⁶ PFU RSV or hPIV3 per animal on Day 28 post-vaccination. Animals immunized with b/h PIV3/RSV were protected completely from hPIV3 and RSV (Table 11). RSV challenge virus was detected at very low levels and hPIV3 challenge virus was not observed at all in the upper and lower respiratory tract of hamsters. Only the animals that had received placebo medium displayed 4.4 and 4.1 log10 TCID₅₀/g tissue of hPIV3, and 3.6 and 3.1 log10 pfu/g tissue of RSV in the upper and lower respiratory tracts (Table 11). This study also showed that animals immunized with RSV were not protected from challenge with hPIV3. Similarly, animals vaccinated with hPIV3 displayed high titers of RSV challenge virus (Table 11).

TABLE 11
b/h PIV3/RSV Immunized Hamsters were Protected
Upon Challenge with hPIV3 and RSV A2

| | Challenge Virus: | | | |
|---|---|---|---|---|
| | hPIV3 | | RSV A2 | |
| | Mean Virus Titer on | | Mean Virus Titer on | |
| | Day 4 Post-challenge | | Day 4 Post-challenge | |
| | (log₁₀ TCID₅₀/g tissue | | (log₁₀ pfu/g tissue | |
| | ± S.E.)^{b,c} | | ± S.E.)^{b} | |
| Immunizing | Nasal | | Nasal | |
| Virus^{a} | turbinates | Lungs | Turbinates | Lungs |
| b/h PIV3 | <1.2 ± 0.0 | <1.0 ± 0.1 | ND | ND |
| b/h RSV G1 | <1.2 ± 0.1 | <1.1 ± 0.1 | <1.0 ± 0.3 | <0.7 ± 0.1 |
| b/h RSV F1 | <1.2 ± 0.2 | <1.0 ± 0.0 | <1.1 ± 0.5 | <0.6 ± 0.0 |
| b/h RSV F1 NP-P | <1.0 ± 0.0 | <1.0 ± 0.0 | <0.8 ± 0.1 | <0.5 ± 0.0 |
| b/h RSV G2 | <1.2 ± 0.2 | <1.1 ± 0.2 | <0.8 ± 0.1 | <0.8 ± 0.3 |
| b/h RSV F2 | <1.2 ± 0.1 | <1.0 ± 0.1 | <1.3 ± 0.6 | <1.6 ± 1.0 |
| RSV A2 | 4.5 ± 0.6 | 4.8 ± 0.6 | <0.6 ± 0.2 | <0.6 ± 0.1 |
| Placebo | 4.4 ± 0.1 | 4.1 ± 0.1 | 3.6 ± 0.8 | 3.1 ± 0.7 |

^{a}Virus used to immunize groups of six hamsters on day 0.
^{b}On day 28, the hamsters were challenged with 10⁶ pfu of hPIV3 or RSV A2. Four days post-challenge, the lungs and nasal turbinates of the animals were harvested.
^{c}Standard error.

### 28. EXAMPLE 23

### Vaccination of Hamsters with Bovine Parainfluenza 3/Human Parainfluenza 3 Vectored Respiratory Syncytial Virus Induces Serum HAI and Neutralizing Antibodies

Prior to administering the challenge dose, serum samples were obtained on Day 28 from the b/h PIV3/RSV immunized animals. The hamster sera were analyzed for the presence or RSV neutralizing antibodies using a 50% plaque reduction assay, and for PIV3 HAI serum antibodies by carrying out hemaggluination inhibition (HAI) assays (Table 12). 50% plaque reduction assay (neutralization assay) was carried out as follows: the hamster sera were two-fold serially diluted, and incubated with 100 PFU of RSV A2 for one hour. Then the virus-serum mixtures were transferred to Vero cell monolayers and overlaid with methylcellulose. After 5 days of incubation at 35° C., the monolayers were immunostained using RSV polyclonal antiserum for quantification. Hemagglutination-inhibition (HAI) assays were performed by incubating serial two-fold dilutions of Day 28 hamster sera at 25° C. for 30 min with hPIV3 in V-bottom 96-well plates. Subsequently, guinea pig erythrocytes were added to each well, incubation was continued for an additional 90 min, and the presence or absence of hemagglutination in each well was recorded. Titers were expressed as the mean reciprocal log2 of the highest serum dilution that inhibited hemagglutination.

TABLE 12
Vaccination of Hamsters with b/h PIV3/RSV Induces
Serum HAI and Neutralizing Antibodies

| | Neutralizing antibody | HAI antibody |
|---|---|---|
| | response to RSV ^{b, c} | response to hPIV3 ^{c} |
| | (mean reciprocal | (mean reciprocal |
| Virus ^{a} | log₂ ± SE) | log₂ ± SE) |
| RSV | 7.9 ± 1.00 | ND |
| b/h RSV F1* N-N | 7.8 ± 0.85 | 6.6 ± 0.5 |
| b/h RSV F1 | 5.5 ± 0.53 | 5.5 ± 0.5 |
| b/h RSV G1 | 3.4 ± 0.50 | 6.6 ± 0.7 |
| b/h RSV F2 | 6.9 ± 0.65 | 6.7 ± 0.8 |
| b/h RSV G2 | 3.4 ± 0.50 | 5.2 ± 0.4 |
| b/h PIV3 | ND | 7.2 ± 0.5 |

^{a} Viruses used to immunize hamsters.
^{b} The neutralizing antibody titers were determined by a 50% plaque reduction assay.
^{c} The neutralizing antibody titers of hamster pre-serum were <1.0 and the HAI antibody titers were <4.0.

The results showed that the viruses expressing the RSV F protein in genome positions 1 or 2, displayed RSV neutralizing antibody titers of 5.5 and 6.9 log2, respectively. These titers were slightly lower than the antibody titers observed for serum obtained from animals vaccinated with wild type RSV (Table 12). In contrast, the viruses expressing the RSV G protein showed RSV neutralizing antibody titers that were reduced by ˜50% (Table 12). All of the chimeric b/h PIV3/RSV hamster sera showed levels of HAI serum antibodies that were reduced by 0.5-2.0 log2 compared to the levels observed for b/h PIV3 (Table 12). The results showed that the chimeric b/h PIV3/RSV could infect and replicate efficiently in hamsters and elicit a protective immune response to hPIV3 and RSV.

### 29. EXAMPLE 24

### Vaccination of Hamsters with Low Dose of Bovine Parainfluenza 3/Human Parainfluenza 3 Vectored Respiratory Syncytial Virus Protected Hamsters from Challenge with Respiratory Syncytial Virus A2, and Induces Serum HAI and Neutralizing Antibodies

In order to identify the best vaccine candidate, low dose virus with different constructs (see Example 2) were used to immunize hamsters. The results of the challenging experiments are summarized in Table 13.

TABLE 13
b/h PIV3/RSV-Low Dose Immunized Hamsters
are Protected From Challenge with RSV A2

| | Replication | | Challenge with RSV A2 | |
|---|---|---|---|---|
| | Mean Virus Titer on | | Mean Virus Titer on | |
| | Day 4 Post-vaccination | | Day 4 Post-challenge | |
| | (log₁₀ TCID₅₀/g tissue | | (log₁₀ pfu/g tissue | |
| | ± S.E.)^{b,c} | | ± S.E.)^{b} | |
| Immunizing | Nasal | | Nasal | |
| Virus^{a} | turbinates | Lungs | Turbinates | Lungs |
| b/h PIV3 | 4.9 ± 0.5 | 4.8 ± 1.0 | ND | ND |
| b/h RSV G1 | 3.0 ± 0.8 | 3.1 ± 0.5 | <0.9 ± 0.5 | <0.7 ± 0.4 |
| b/h RSV F1* N-N | 3.4 ± 0.1 | 3.5 ± 0.1 | <1.4 ± 0.7 | <0.5 ± 0.0 |
| b/h RSV G2 | 4.1 ± 0.6 | 3.8 ± 0.4 | <0.8 ± 0.0 | <0.5 ± 0.1 |
| b/h RSV F2 | 5.2 ± 0.6 | 3.9 ± 0.4 | <0.7 ± 0.1 | <0.5 ± 0.1 |
| RSV A2 | 2.8 ± 0.3 | 2.7 ± 0.6 | <0.8 ± 0.1 | <0.5 ± 0.0 |
| Placebo | ND^{d} | ND | 3.0 ± 0.8 | 3.2 ± 0.9 |

^{a}Virus used to immunize groups of six hamsters on day 0 with a low dose of 10⁴ PFU/ml.
^{b}On day 28, the hamsters were challenged with 10⁶ pfu of RSV A2. Four days post-challenge, the lungs and nasal turbinates of the animals were harvested.
^{c}Standard error.
^{d}not determined.

Next, the neutralizing antibody titers were determined by a 50% plaque reduction assay (neutralization assay). Neutralization assays were performed for b/h PIV3, b/h PIV3/RSV chimeric viruses or RSV using Vero cells. Serial two-fold dilutions of RSV polyclonal antiserum (Biogenesis; Poole, England), an RSV F MAb (WHO 1200 MAb) obtained from MedImmune or hPIV3 F (C191/9) and HN (68/2) MAbs, were incubated with approximately 100 PFU of either b/h PIV3, b/h PIV3/RSV chimeric viruses or RSV in 0.5 ml OptiMEM at RT for 60 min. Following the incubation, virus-serum mixtures were transferred to Vero cell monolayers, incubated at 35_C for 1 hour, overlaid with 1% methyl cellulose in EMEM/L-15 medium (JRH Biosciences; Lenexa, Kans.) and incubated at 35_C. Six days post-inoculation, the infected cell monolayers were immunostained. Neutralization titers were expressed as the reciprocal of the highest serum dilution that inhibited 50% of viral plaques. Neutralization assays were also carried out for serum obtained on Day 28 post-infection from hamsters immunized with b/h PIV3, b/h PIV3/RSV chimeric viruses, or RSV A2. The hamster sera were two-fold serially diluted, and incubated with 100 PFU of RSV A2 for one hour. Then the virus-serum mixtures were transferred to Vero cell monolayers and overlaid with methylcellulose. After 5 days of incubation at 35° C. the monolayers were immunostained using RSV polyclonal antiserum for quantification. The neutralizing antibody titers of hamster pre-serum were <1.0 and the HAI antibody titers were <4.0.

Hemagglutination-inhibition (HAI) assays were performed by incubating serial two-fold dilutions of Day 28 hamster sera at 25° C. for 30 min with hPIV3 in V-bottom 96-well plates. Subsequently, guinea pig erythrocytes were added to each well, incubation was continued for an additional 90 min, and the presence or absence of hemagglutination in each well was recorded. Table 14 summarizes the results:

TABLE 14
Vaccination of Hamsters with Lower Doses of b/h PIV3/RSV
Induces Serum HAI and Neutralizing Antibodies

| | Neutralizing antibody | HAI antibody |
|---|---|---|
| | response to RSV ^{b, c} | response to hPIV3 ^{c} |
| | (mean reciprocal | (mean reciprocal |
| Virus ^{a} | log₂ ± SE) | log₂ ± SE) |
| RSV | 6.5 ± 0.7 | ND |
| b/h RSV F1* N-N | 2.5 ± 0.7 | 5.7 ± 0.6 |
| b/h RSV G1 | 2.0 ± 0.0 | 6.0 ± 0.0 |
| b/h RSV F2 | 3.8 ± 1.5 | 6.7 ± 0.6 |
| b/h RSV G2 | 3.8 ± 1.3 | 5.5 ± 0.6 |
| b/h PIV3 | ND | 6.5 ± 0.7 |

^{a} Viruses used to immunize hamsters at a low dose of 10⁴pfu/ml.
^{b} The neutralizing antibody titers were determined by a 50% plaque reduction assay.
^{c} The neutralizing antibody titers of hamster pre-serum were <1.0 and the HAI antibody titers were <4.0.

The restricted replication phenotype of the chimeric viruses possessing RSV genes in the first position was exacerbated when the inoculation dose was reduced to 1×10⁴ PFU per animal. b/h PIV3/RSV F1 and G1 replicated in the upper respiratory tracts of hamsters to titers that were reduced by 1.0-2.0 log₁₀ compared to those of b/h PIV3 (Table 13). In contrast, b/h PIV3/RSV with the RSV genes in position 2, replicated in the upper respiratory tract to levels observed for b/h PIV3. Replication in the lungs of hamsters was also more restricted for the b/h PIV3/RSV harboring RSV genes in the first position (Table 13). In contrast, b/h PIV3/RSV F2 still replicated to high titers of 10^{5.2} and 10^{3.9} in the nasal turbinates and lungs, respectively (Table 13). The vaccinated hamsters were challenged on Day 28 with 1×10⁶ pfu of RSV A2 (Table 13). Despite the low levels of replication observed in the respiratory tracts of hamsters, the animals were protected in both the lower and upper respiratory tract from challenge with RSV (Table 13). The degree of protection was as good as was observed for animals vaccinated with wt RSV. Only the animals that received placebo medium showed high virus titers in the nasal turbinates and lungs (Table 13). Serum was collected from the immunized hamsters on Day 28, and analyzed for the presence of RSV neutralizing and PIV3 HAI serum antibodies (Table 14). An approximately 50% drop in RSV neutralizing antibody titers was observed in sera obtained from hamsters immunized with b/h PIV3/RSV as compared to the titers observed for wt RSV sera (Table 14). But the sera obtained from animals that had received b/h PIV3 harboring the RSV genes in position 2, still displayed higher RSV neutralization antibody titers than was observed for sera from b/h PIV3/RSV with the RSV genes in position 1. The PIV3 HAI serum antibody titers were also slightly reduced compared to the b/h PIV3 sera (Table 14).

### 30. EXAMPLE 25

### Bovine Parainfluenza 3/Human Parainfluenza 3 Vectored Human Metapneumovirus F Immunized Hamsters were Protected Upon Challenge with Human Parainfluenza Virus 30R Human Metapneumovirus NL/001

Five groups of Syrian Golden Hamsters (each group had six hamsters) were immunized with b/h PIV3, b/h hMPV F1, b/h hMPV F2, hMPV or placebo repectively. The five different animal groups were kept separate in micro-isolator cages. On Day 28 post-immunization, the hamsters were challenged with 1×10⁶ PFU of either hPIV3 or hMPV (NL/001 strain) to evaluate the immunogenicity induced by the b/h PIV3/hMPV F. Four days post-challenge, the animals were sacrificed. The nasal turbinates and lungs of the animals were homogenized and stored at −80° C. Virus present in the tissues was determined by TCID₅₀ assays in MDBK cells at 37° C. Virus infection was confirmed by hemabsorption with guinea pig red blood cells. Table 15 shows the replication titers of the PIV3 strain and the MPV strain in hamsters in the lungs and nasal turbinates.

TABLE 15
b/h PIV3/hMPV F-Immunized Hamsters were Protected
Upon Challenge with hPIV3 or hMPV/NL/001

| | Challenge virus: | | | |
|---|---|---|---|---|
| | hPIV3 | | hMPV | |
| | Mean virus titer on | | Mean virus titer on | |
| | day 4 post-challenge | | day 4 post-challenge | |
| | (log₁₀ TCID₅₀/g | | (log₁₀ PFU/g | |
| | tissue ± S.E)^{b} | | tissue ± S.E)^{b} | |
| Immunizing | Nasal | | Nasal | |
| virus^{a} | Turbinates | Lungs | Turbinates | Lungs |
| b/h PIV3 | <1.3 ± 0.2 | <1.1 ± 0.1 | ND | |
| b/h hMPV F1 | <1.3 ± 0.1 | <1.1 ± 0.1 | 3.5 ± 0.8 | <0.5 ± 0.2 |
| b/h hMPV F2 | <1.2 ± 0.1 | <1.2 ± 0.1 | <0.9 ± 0.4 | <0.5 ± 0.1 |
| hMPV | ND | | <0.8 ± 0.3 | <0.4 ± 0.0 |
| Placebo | 4.3 ± 0.3 | 4.5 ± 0.5 | 6.0 ± 0.3 | 4.5 ± 1.3 |

^{a}Virus used to immunize groups of six hamsters on day 0.
^{b}On day 28, the hamsters were challenged with 10⁶ pfu of hPIV3 or hMPV. Four days post-challenge, the lungs and nasal turbinates of the animals were harvested.
ND = not determined.

The results showed that animals that received the b/h PIV3/hMPV F2 (F at position two) were protected completely from hMPV as well as hPIV3 (Table 15). However, b/h PIV3/hMPV F1 (F at position one) only reduced the titers of infected hMPV in the upper respiratory tract (e.g., nasal turbinates) by 2.5 logs, while it provided complete protection in the lower respiratory tract (e.g., the lung) from both hMPV and hPIV3 infection (Table 15). The animals that were administered the placebo medium displayed high titers of challenge virus in the lower and upper respiratory tracts (Table 15).

### 31. EXAMPLE 26

### Bovine Parainfluenza 3/Human Parainfluenza 3 Vectored Human Metapneumovirus Vaccinated Hamsters Produced HMPV Neutralizing and PIV3 HAI Serum Antibodies

Serum samples were obtained from the hamsters on Day 28 prior to administering the challenge virus, and analyzed for the presence of hMPV neutralizing antibodies and HAI serum antibodies (Table 16). High levels of hMPV neutralizing antibodies, 7.36 log2, were observed for sera derived from wt hMPV-infected animals. Sera obtained from b/h PIV3/hMPV F1 or F2-vaccinated hamsters showed neutralizing antibody titers of 7.77 and 7.38 log2, respectively, that were equivalent to those observed for wild type hMPV sera (Table 16). The HAI antibody levels were also similar to those observed for b/h PIV3, the virus vector. The chimeric b/h PIV3/hMPV F1 and F2 displayed HAI titers of 5.78 and 6.33 log2, respectively, which are by 1.2 and 0.7 log2 reduced compared to the HAI titers obtained from b/h PIV3-infected hamster sera (Table 16).

TABLE 16
Vaccination of hamsters with b/h PIV3/hMPV induces
PIV3 serum HAI and hMPV neutralizing antibodies

| | Neutralizing antibody | HAI antibody |
|---|---|---|
| | response to hMPV^{b,c} | response to hPIV3^{c} |
| | (mean reciprocal | (mean reciprocal |
| Virus^{a} | log₂ ± SE) | log₂ ± SE |
| hMPV | 7.36 ± 1.5 | ND |
| b/h hMPV F1 | 7.77 ± 1.0 | 5.78 ± 0.7 |
| b/h hMPV F2 | 7.38 ± 1.0 | 6.33 ± 0.5 |
| b/h PIV3 | ND | 7.00 ± 0.8 |

^{a}Viruses used to immunize hamsters.
^{b}The neutralizing antibody titers were determined by a 50% plaque reduction assay.
^{c}The neutralizing antibody titers of hamster pre-serum were <1.0 and the HAI antibody titers were <2.0.
ND: not determined.

In summary, the results showed that b/h PIV3 expressing hMPV F protein in positions 1 or 2 of the b/h PIV3 genome can efficiently infect and replicate in Syrian Golden hamsters and induce a protective immune response and protect from challenge with hPIV3 and hMPV. The immunization of hamsters with these chimeric viruses also elicited the production of HMPV neutralizing antibodies and HAI serum antibodies to levels similar to those observed for wt hMPV or b/h PIV3, respectively.

### 32. EXAMPLE 27

### Trivalent Bovine Parainfluenza 3/Human Parainfluenza 3 Vectored Construct Replicates in Hamsters and Protects Hamsters from hMPV/NL1/00, hPIV3 and RSV A2

Five week old Syrian Golden hamsters (six animals per group) were infected intranasally with 1.0×10⁶ PFU of the b/h PIV3 virus and 1.0×10⁵ PFU of the trivalent virus b/h PIV3/RSV F1/hMPV F3 in a 0.1 ml volume, respectively. The different groups were maintained separately in micro-isolator cages. Four days post-infection, the nasal turbinates and lungs of the animals were harvested, homogenized and stored at −70° C. The titers of virus present in the tissues were determined by TCID₅₀ assay in Vero cells. Table 17 shows the replication titers of the different strains in hamsters in the lungs and nasal turbinates.

TABLE 17
Replication of Trivalent Virus in Hamsters

| | Mean virus titer on day 4 post-infection | |
|---|---|---|
| | (log₁₀ PFU/g tissue ± S.E.)^{b} | |
| Virus^{a} | Nasal turbinates | Lungs |
| b/h PIV3 | 5.4 ± 0.3 | 5.4 ± 1.2 |
| b/h PIV3/RSV F1/hMPV | 2.3 ± 0.7 | 2.7 ± 0.5 |
| F3 | | |

^{a}The RSV/hMPV animals were inoculated intranasally with 1.0 × 10⁵ PFU virus in a 0.1 ml volume, the b/h PIV3 animals received 1.0 × 10⁶ PFU virus.
^{b}Standard error.

In order to evaluate whether the levels of replication observed for b/h PIV3/RSV F1/hMPV F3 were sufficient to elicit a protective immune response in hamsters, the animals were challenged (inoculated) on day 28 intranasally with 1×10⁶ PFU of hPIV3, 1×10⁶ PFU of RSV, and 1.0×10⁵ PFU of hMPV/NL/1/00. Four days post-challenge, the nasal turbinates and lungs of the animals were isolated and assayed for challenge virus replication by plaque assays on Vero cells that were immunostained for quantification. This study showed that on Day 28 post-vaccination, animals immunized with b/h PIV3/RSV F1/hMPV F3 were protected from all three viruses, i.e., protected from hPIV3, RSV and hMPV (hMPV/NL/1/00) viruses (Table 18).

TABLE 18
Trivalent Virus Protects Hamsters From
hMPV/NL/1/00, hPIV3, and RSV A2

| | |
|---|---|
| | Mean virus titer on day 4 post-infection |
| Virus used for | (log₁₀PFU/g tissue ± S.E.) ^{b} |

| challenge ^{a} | Nasal turbinates | Lungs |
|---|---|---|
| hPIV3 | <1.2 ± 0.0 | <1.2 ± 0.2 |
| Placebo | 4.7 ± 0.1 | 5.2 ± 0.6 |
| RSV | <1.6 ± 0.3 | <0.9 ± 0.5 |
| Placebo | 3.0 ± 0.3 | 2.7 ± 0.6 |
| hMPV/NL/1/00 | <0.5 ± 0.1 | <0.5 ± 0.1 |
| Placebo | 6.0 ± 0.3 | 5.3 ± 0.3 |

^{a} All animals were inoculated intranasally with 1 × 10⁶ PFU virus in a 0.1 ml volume except the hMPV animals which received 1.0 × 10⁵ PFU virus.
^{b} Standard error.

### 33. EXAMPLE 28

### b/h PIV3 Expressing the Native or Soluble Fusion Protein of RSV Confer Complete Protection from RSV Infection in African Green monkeys

Two potential RSV vaccine candidates, b/h PIV3/RSV F2 (see Example 2) and b/h PIV3/sol RSV F2 (see Example 11), were evaluated in this study for efficacy and immunogenicity in a non-human primate model. A b/h PIV3 vector was employed to express the native and soluble forms of the RSV F protein from PIV3 genome position 2, juxtaposed between N and P. Previous analysis of b/h PIV3/RSV F2 had shown that high levels of RSV F protein were expressed by chimeric b/h PIV3 from this genome position and that hamsters vaccinated with this vaccine were protected from both RSV and hPIV3 challenge. The efficacy of two b/h PIV3 vaccines was compared that expressed either native RSV F protein capable of being inserted into the virion envelope or soluble RSV F protein which could not be incorporated into the virion. The soluble RSV F protein cannot be anchored into the virion envelope due to the absence of the transmembrane domain.

Antibodies produced in response to expression of the RSV F protein by b/h PIV3 are expected to result in cross-neutralization and cross-protection against infection by all strains of RSV, because the RSV F genes are highly conserved between subgroups A and B of RSV. Both b/h PIV3/RSV F2 and b/h PIV3/sol RSV F2 expressed the RSV F proteins efficiently from PIV3 genome position 2. These RSV vaccines were analyzed for levels of replication in the respiratory tract of African green monkeys (AGMs), and the ability to elicit a protective immune response from wildtype RSV challenge.

The studies described in this Example have shown that both RSV vaccine candidates, b/h PIV3/RSV F2 and b/h PIV3/sol RSV F2, were efficacious and protected non-human primates completely from RSV challenge. Both of the PIV3 vectored RSV chimeras represent attractive vaccines to be further evaluated in human clinical trials. So far, based on the protective immune responses produced and the RSV and hPIV3 antibody titers generated, b/h PIV3/RSV F2 and b/h PIV3/sol RSV F2 displayed equivalent responses. Additional safety evaluations for enhanced RSV disease in a cotton rat model as well as tissue tropism studies in hamsters can be performed to establish a more detailed safety profile for both PIV3/RSV vaccine candidates. The PIV3/RSV vaccine displaying the best safety profile will be further evaluated in adults and children in clinical trials to yield an efficacious yet safe RSV vaccine.

### 1. Materials and Methods

Cells and Viruses

Vero cells were maintained in Modified Eagle's Medium (MEM) (JRH Biosciences) supplemented with 2 mM L-glutamine, non-essential amino acids (NEAA), antibiotics, and 10% FBS. b/h PIV3/RSV F2, b/h PIV3/sol RSV F2, RSV A2, RSV B 9320, hMPV/NL/1/00 were propagated in Vero cells. Cells were infected with the viruses at a multiplicity of infection (MOI) of 0.1 PFU/cell. Three to five days post-infection the cells and supernatant were collected and stabilized by adding 10× SPG (10× SPG is 2.18 M Sucrose, 0.038 M KH₂PO₄, 0.072 M K₂HPO₄, 0.054 M L-Glutamate) to a final concentration of 1×. The virus stocks were stored at −70° C. The virus titers were determined by plaque assays on Vero cells. Plaques were quantified after immunoperoxidase staining using PIV3 (VMRD) or RSV goat polyclonal antisera (Biogenesis).

Primate Studies

RSV- and PIV3-seronegative African Green monkeys (Cercopithecus aethiops) (3.5 to 6.5 years old, 2.6 to 5.8 kg) were identified using an RSV F IgG ELISA (Immuno-Biological Laboratories) and a hemagglutination inhibition (HAI) assay (described below) for primate pre-sera collected on day −14 prior to the study start date. The primates were housed in individual micro-isolator cages. The monkeys were anesthetized with a ketamine-valium mixture and infected intranasally and intratracheally with b/h PIV3/RSV F2, b/h PIV3/sol RSV F2, RSV A2, and hMPV/NL/1/00. The nasal dose volume was 0.5 ml per nostril, and the intratracheal dose volume was 1 ml. On Day 1, each animal received a dose of 2 ml containing 2-3×10⁵ PFU of virus. The placebo animal group received the same dose volume of Opti-MEM. On Day 28, all animals were challenged intratracheally and intranasally with 7×10⁵ PFU of RSV A2 (1 ml at each site). Nasopharyngeal (NP) swabs were collected daily for 11 days and tracheal lavage (TL) specimens were collected on Days 1, 3, 5, 7 and 9 post-immunization and post-challenge. Blood samples obtained from the femoral vein were collected on Days 0, 7, 14, 21, 28, 35, 42, 49 and 56 for serological analysis. The animals were monitored for body temperature changes indicating a fever, signs of a cold, runny nose, sneezing, loss of appetite, and body weight. Virus present in the primate NP and TL specimens was quantitated by plaque assays using Vero cells that were immunostained with RSV goat polyclonal antiserum. Mean peak virus titers represent the mean of the peak virus titer measured for each animal on any of the 11 days following immunization or challenge.

Plaque Reduction Neutralization Assay (PRNA):

PRNAs were carried out for sera obtained on days 1, 28, and 56 post-dose from primates infected with b/h PIV3/RSV F2 and b/h PIV3/sol RSV F2, respectively. The primate sera were two-fold serially diluted, and incubated with 100 PFU of RSV A2 in the presence of guinea pig complement for one hour at 4° C. The virus-serum mixtures were transferred to Vero cell monolayers and overlaid with 1% methyl cellulose in EMEM/L-15 medium (JRH Biosciences; Lenexa, Kans.) containing 2% FBS and 1% antibiotics. After 6 days of incubation at 35° C., the monolayers were immunostained using RSV goat polyclonal antiserum for quantitation. Neutralization titers were expressed as the reciprocal log₂ of the highest serum dilution that inhibited 50% of viral plaques.

RSV F IgG Elisa:

The primate sera from days 1, 28 and 56 from the vaccinated animals were analyzed for the presence of RSV F IgG using an ELISA kit (Immuno-Biological Laboratories, Hamburg, Germany) according to the manufacturer's instructions. The secondary monkey antiserum (Rockland Inc.) was used at a 1:1000 dilution. The RSV F IgG antibody titers were expressed as log₂ IgG U/ml.

hPIV3 Microneutralization Assays:

Microneutralization assays were performed on Vero cells. Serial two-fold dilutions of primate serum, starting at 1:4, were incubated at 37° C. for 60 min with 100 TCID₅₀ of hPIV3. Then virus-serum mixtures were transferred to cell monolayers in 96-well plates and incubated at 37° C. for six days, after which all wells were observed for CPE. Neutralization titers were expressed as the reciprocal of the highest serum dilution that inhibited CPE. Neutralization antibody titers of ≦1:4 (the lowest serum dilution tested) were assigned a reciprocal log₂ titer of 2.

PIV3 Hemagglutination Inhibition (HAI) Assay:

HAI assays were performed by incubating serial two-fold dilutions of primate serum at 25° C. for 30 min with 8 HA units/0.05 ml of either bPIV3 or hPIV3. Subsequently, guinea pig red blood cells were added to each well, incubation was continued for 90 min, and each well was observed for hemagglutination. HAI titers were expressed as the reciprocal of the highest dilution of antiserum that inhibited virus-mediated agglutination of erythrocytes.

### 2. Results

b/h PIV3/RSV F2 and b/h PIV3/sol RSV F2 Replicated Efficiently in the Respiratory Tract of AGMs

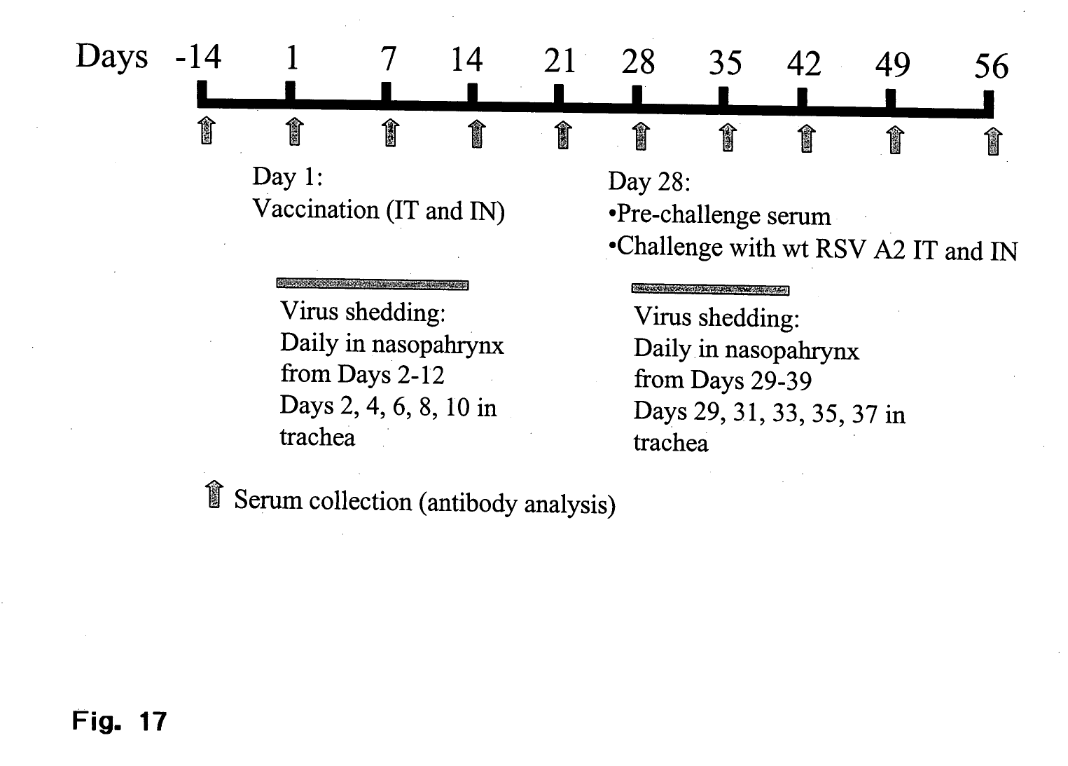

AGMs have been shown to support high levels of RSV A and RSV B replication in the lower and upper respiratory tract. To study the replication efficiency of the b/h PIV3/RSV F2 and b/h PIV3/sol RSV F2 vaccines, the experiment was designed as follows (see FIG. 17). Briefly, on Day 1, RSV and PIV3 sero-negative AGMs, four animals per group, were immunized intranasally and intratracheally with b/h PIV3/RSV F2 or b/h PIV3/sol RSV F2 with a dose of 2-3×10⁵ PFU. A positive control group was infected with wildtype RSV A2 and the negative control group was administered placebo medium. On Day 28, all animals were challenged intranasally and intratracheally with 7×10⁵ PFU of wildtype RSV A2. The animals were housed in micro-isolator cages for the duration of this study. Nasopharyngeal swabs were collected daily for 11 days post-immunization and post-challenge, and tracheal lavage samples were obtained on days 2, 4, 6, 8, and 10 post-immunization and post-challenge. Serum samples for antibody analyses were collected every seven days throughout the duration of the study (see FIG. 17).

As shown in Table 19, following vaccination with b/h PIV3/RSV F2, monkeys shed for seven days in the nasopharynx displaying a mean peak titer of 5.6 log₁₀ PFU/ml, and for nine days in the trachea with mean peak titers of 7.0 log₁₀ PFU/ml. Immunization of AGMs with vaccine virus expressing the soluble form of the RSV F protein, b/h PIV3/sol RSV F2, resulted in virus shedding for eight days in the nasopharynx showing mean peak titers of 5.6 log₁₀ PFU/ml, and for seven days in the trachea with peak titers of 6.8 log₁₀ PFU/ml Table 19. In contrast, infection of primates with wt RSV A2 resulted in six days of virus shedding in the nasopharynx achieving mean peak titers of 3.3 log₁₀ PFU/ml and eight days of virus shedding in the trachea displaying peak titers of 5.0 log₁₀ PFU/ml. The animals that were administered placebo medium did not shed virus (Table 19). Thus, immunization of non-human primates with b/h PIV3/RSV F2 or b/h PIV3/sol RSV F2, resulted in similar high levels of replication and duration of virus shedding for both vaccine candidates tested. Indeed, virus replication for the b/h PIV3/RSV vaccine candidates was 200-fold higher in the URT and 63-100-fold higher in the LRT compared to wildtype RSV A2.

TABLE 19
African Green Monkeys Immunized with b/h PIV3/RSV
F2 or b/h PIV3/sol RSV F2 Were Completely Protected
From Challenge with Wildtype RSV A2

| | | Pre-Challenge Mean | | Post-Challenge^{$} Mean | |
|---|---|---|---|---|---|
| Immunizing | No. of | Peak Titers^{#} | | Peak Titers | |
| Virus* | Animals | NP | BAL | NP | BAL |
| RSV A2 | 3 | 3.3 ± 1.5 | 5.0 ± 0.4 | <1.2 ± 0.2 | <1.0 ± 0.0 |
| b/h PIV3/RSV | 4 | 5.6 ± 1.0 | 7.0 ± 0.4 | <1.2 ± 0.4 | <1.2 ± 0.3 |
| F2 | | | | | |
| b/h PIV3/sol | 4 | 5.6 ± 0.2 | 6.8 ± 0.4 | <1.1 ± 0.2 | <1.0 ± 0.0 |
| RSV F2 | | | | | |
| hMPV | 3 | ND | ND | 4.0 ± 0.1 | 5.0 ± 0.2 |
| Placebo | 2 | 0.0 ± 0.0 | 0.0 ± 0.0 | 4.3 ± 0.3 | 5.7 ± 0.3 |

*Animals were inoculated with 2-3 × 10⁵ PFU of the indicated virus at each site intranasally and intratracheally in a one ml volumn.
^{#}Mean peak virus titer is expressed as log₁₀ PFU/ml ± standard error and is the mean of the highest titer of virus of each animal in the specific group during the course of the study.
^{$}Animals were challenged on Day 28 with 7 × 10⁵ PFU of RSV A2.
ND = not determined.

The animals were observed for 11 days post-vaccination for signs of RSV disease such as rhinorrea, runny nose, cold, or fever. No signs of disease were noted during this period of acute virus replication.

In this study, both vaccine candidates, b/h PIV3/RSV F2 and b/h PIV3/sol RSV F2, replicated to high titers of 5.6 and 7.0 log10 PFU/ml in the URT and LRT of AGMs, respectively. The replication titers observed for the two RSV vaccine candidates in the respiratory tract of AGMs were higher than those for wildtype RSV A2. The levels of replication observed for the potential RSV candidate vaccines afforded complete protection from wt RSV challenge 28 days post-dose. High replication titers of the RSV A2 challenge virus were observed only for animals administered placebo medium or animals that had been vaccinated with HMPV, a related paramyxovirus, that did not result in immunological cross-protection.

AGMs Immunized with b/h PIV3/RSV F2 or b/h PIV3/sol RSV F2 were Completely Protected From Wildtype RSV A2 Challenge

In order to evaluate immune protection from RSV infection, the vaccinated primates were challenged with a high dose of wildtype RSV A2 four weeks post-immunization. Efficacy was measured as a reduction in shed RSV challenge virus titer in the URT and LRT of the infected animals. Primates immunized with b/h PIV3/RSV F2 or b/h PIV3/sol RSV F2 were protected efficiently from wt RSV A2 challenge (Table 19). Only one animal vaccinated with b/h PIV3/RSV F2 shed low levels of challenge virus (1.8 log₁₀ PFU/ml) for one day in the nasopharynx and one day in the trachea (1.6 log₁₀ PFU/ml). The mean peak titers for this treatment group were 1.2 log₁₀ PFU/ml in the URT and 1.2 log₁₀ PFU/ml in the LRT. The animals that were administered b/h PIV3/sol RSV F2 were also completely protected from wt RSV challenge (Table 19). One animal displayed low levels of challenge virus shedding (1.3 log₁₀ PFU/ml) for three days in the nasopharynx, but this animal did not shed RSV in the trachea. The mean peak titers observed for the b/h PIV3/sol RSV F2-immunized primates were 1.1 log₁₀ PFU/ml in the nasopharynx and 1.0 log₁₀ PFU/ml in the trachea. Similar levels of immune protection were observed for the AGMs infected with wt RSV A2 (Table 19). This group showed levels of 1.2 log₁₀ PFU/ml and 1.0 log₁₀ PFU/ml of shed RSV challenge virus in the nasopharynx and trachea, respectively. One animal that was infected with RSV on Day 1 shed low levels of RSV challenge virus (1.3 log₁₀ PFU/ml) in the nasopharynx for one day. In contrast, treatment groups that had received placebo medium displayed high levels of RSV challenge virus replication, 4.3 log₁₀ PFU/ml in the nasopharynx and 5.7 log₁₀ PFU/ml in the trachea and the primates shed challenge virus for eight days in both the URT and LRT. AGMs that were administered hMPV, a related paramyxovirus, on Day 1, were not protected from RSV challenge and shed RSV challenge virus for eight days in the URT and LRT. Mean peak titers of 4.0 log₁₀ PFU/ml and 5.0 log₁₀ PFU/ml in the URT and LRT of AGMs were observed (Table 19). These results showed that vaccination with either RSV vaccine candidate could efficiently protect non-human primates from subsequent wildtype RSV infection.

AGMs Immunized with b/h PIV3/RSV F2 or b/h PIV3/sol RSV F2 Produced Protective RSV Serum Antibodies

Efficacy of the b/h PIV3 vectored RSV vaccine candidates was further evaluated by the levels of RSV neutralizing and RSV F IgG serum antibody titers produced four weeks post-immunization. The RSV neutralizing antibody titers were determined using a 50% plaque reduction neutralization assay (PRNA) (Table 20).

TABLE 20
Vaccination of African Green Monkeys with b/h PIV3/RSV
F2 and b/h PIV3/sol RSV F2 produced RSV neutralizing
and RSV F-Specific IgG Serum Antibody Titers

| | | Mean RSV | | RSV F IgG |
|---|---|---|---|---|
| | | Neutralizing | | (U/ml) |
| | | Antibody Titers* | | Geometric |
| | Day of | (50% Reciprocal | | Mean Log2 |
| Immunizing | Serum | Log₂ ± SE) | | Antibody |
| Virus | Collection* | RSV A^{#} | RSV B^{#} | Titers* |
| RSV A2 | 28 | 9.0 ± 1.0 | 4.2 ± 1.0 | 8.6 |
| | 56 | 10.7 ± 0.6 | 4.3 ± 1.3 | 9.1 |
| b/h PIV3/RSV | 28 | 4.0 ± 1.0 | 3.4 ± 1.8 | 8.2 |
| F2 | | | | |
| | 56 | 4.1 ± 2.0 | 5.0 ± 1.4 | 9.0 |
| b/h PIV3/sol | 28 | 4.1 ± 1.5 | 4.6 ± 1.4 | 8.0 |
| RSV F2 | | | | |
| | 56 | 4.3 ± 1.0 | 5.0 ± 1.1 | 9.4 |
| Placebo | 28 | <2.2 ± 0.3 | <2.0 ± 0.0 | 2.3 |
| | 56 | 9.0 ± 0.1 | 2.0 ± 0.0 | 8.1 |

*All animals displayed RSV neutralizing antibody titers of <2.4 log₂ and RSV F IgG titers of <3.6 log₂ U/ml on Day 1, serum was collected on Day 1 (prior to immunization), Day 28 (prior to RSV challenge), and Day 56 (4 weeks post-RSV challenge).
^{#}RSV A2 and RSV B 9320 were used as antigens in the neutralization assay.

AGMs infected with wildtype RSV A2 displayed high RSV neutralizing antibody titers of 9 log₂ four weeks post-infection when an RSV subgroup A was used as antigen in the PRNA. A five log₂ reduction in RSV neutralizing antibody titers was observed when RSV subgroup B was employed in the PRNA. The vaccine candidates, b/h PIV3/RSV F2 and b/h PIV3/sol RSV F2, showed RSV neutralizing antibody titers of ˜4 log₂ on Day 28 post-dose when RSV subgroup A or subgroup B were used as antigen. In contrast, serum derived from animals that were administered placebo medium did not display RSV neutralizing antibody titers for either RSV subgroup A or B. The serum obtained on Day 56, four weeks post-RSV challenge, was also tested for the presence of RSV neutralizing antibodies (Table 20). Day 56 sera derived from AGMs infected with wildtype RSV A2 showed a 1.7 log₂ increase in RSV neutralizing antibody titer when subgroup A was tested, but the RSV neutralizing antibody titer did not increase for subgroup B. A significant rise in neutralizing antibody titer for Day 56 sera originating from b/h PIV3/RSV F2- or b/h PIV3/sol RSV F2-immunized primates for either subgroup A or B antigens was not observed. Placebo animal serum samples showed a 7 log₂ increase in RSV neutralizing antibody titer on Day 56 for subgroup A RSV, but only a low level of neutralizing antibodies for subgroup B.

In order to further measure the immune responses elicited by the vectored PIV3/RSV vaccines, RSV F protein specific IgG levels were analyzed pre-dose (Day 1), four weeks post-dose (Day 28), and four weeks post-challenge (Day 56) (Table 20). The pre-dose primate sera from all treatment groups displayed values of less than 3.6 log₂ IgG U/ml indicating the absence of RSV F-specific IgG. In contrast, four weeks post-vaccination, RSV F-specific IgG levels for sera derived from b/h PIV3/RSV F2 or b/h PIV3/sol RSV F2 showed titers of 8.2 and 8.0 log₂, respectively. Similar levels of 8.6 log₂ RSV F IgG titers were observed in Day 28 sera originating from RSV A2 infected animals. Only the Day 28 sera of the placebo animals did not contain RSV F IgG. The RSV F IgG titers for Day 56 sera from RSV A2, b/h PIV3/RSV F2, and b/h PIV3/sol RSV F2-immunized animals rose by 0.5 to 1.4 log₂ in titer from the levels observed for Day 28 sera. In contrast, Day 56 sera obtained from the placebo animals challenged with wt RSV A2 showed an about 7 log₂ rise in RSV F-specific IgG titer. Non-human primates vaccinated with PIV3/RSV F vaccines clearly produced RSV specific neutralizing and IgG antibody titers sufficient to protect the animals completely from RSV challenge.

The chimeric b/h PIV3/RSV F vaccines produced RSV neutralizing antibodies specific for both RSV subgroup A and B. The high degree of conservation of the amino acid sequences between the RSV F proteins of subgroup A and B resulted in shared neutralizing epitopes. The levels of RSV neutralizing antibody titers were lower by 5 log₂ for b/h PIV3/RSV F than those observed for primate sera obtained from AGMs infected with wildtype RSV A2. In the b/h PIV3/RSV vaccines, RSV neutralizing antibodies were produced only in response to the RSV F protein rather than to the whole RSV virus particle. The levels of RSV B cross-neutralizing antibody for sera obtained from AGMs infected with wildtype RSV A2 were reduced by 5 log₂ as compared to the antibody levels observed when the homologous RSV A2 antigen was tested. In contrast, a decrease in RSV B specific-neutralizing antibody titers produced by b/h PIV3/RSV F2 and b/h PIV3/sol RSV F2 was not observed.

These results suggested that the serum neutralizing antibody levels induced by the RSV F protein were sufficient to protect primates completely from RSV challenge. Although the RSV neutralizing antibody titers were lower for b/h PIV3/RSV F primate sera, the neutralizing activity for subgroup A and B RSV strains were identical. Primate sera derived from wildtype RSV infection, displayed high RSV neutralizing titers for the homologous RSV A antigen, and lower levels for the RSV B antigen which were similar in titer to those observed for the vectored PIV3/RSV F vaccines. A significant rise (>6 log₂) in RSV F IgG antibody titers were observed for primates infected with RSV A2 or immunized with the b/h PIV3/RSV F vaccines. A further increase in either RSV neutralizing or IgG antibody titers was not observed for animals vaccinated with b/h PIV3/RSV F or b/h PIV3/sol RSV F in response to the RSV challenge. Since the RSV neutralizing antibody titers measured for PIV3/RSV F vaccines were lower than those observed for sera obtained from primates infected with wt RSV, cellular immune responses may have played a role in generating such effective protection from RSV challenge. Further studies may be done to address the contribution of the cellular immune system to the efficacy of the live attenuated PIV3/RSV vaccines.

The b/h PIV3 vector is expected to be attenuated in humans because the majority of the viral genome is derived from bPIV3 that was demonstrated to be safe in children (see Karron et al., Pediatr. Infect. Dis. J. 15: 650-654 (1996)). Skiadopoulos et al. clearly showed using a rhesus monkey attenuation model that the bPIV3 attenuation phenotype was polygenic in nature (see Skiadopoulos et al., J. Virol. 77: 1141-1148 (2003); Van Wyke Coelingh et al., J. Infect. Dis. 157: 655-662 (1988)). While the bPIV3 F and HN genes may contain some genetic determinants specifying attenuation, the greatest contribution to the attenuation phenotype was ascribed to the bPIV3 N and P proteins. Schmidt et al. evaluated a number of b/h PIV3 expressing RSV antigens from different PIV3 genome positions for replication in the respiratory tract of rhesus monkeys (see Schmidt et al., J. Virol. 76:1089-1099 (2002); Van Wyke Coelingh et al., J. Infect. Dis. 157:655-662 (1988)). All of the chimeric b/h PIV3 expressing RSV proteins replicated less efficiently than b/h PIV3 in the URT, and only slightly higher titers (˜0.5 log₁₀ TCID₅₀/ml) were observed in the LRT of rhesus monkeys compared to the vector b/h PIV3. These data further validate the expectation that b/h PIV3/RSV will be attenuated in humans.

Infants do not possess a well developed immune system and therefore multiple vaccine administrations may be necessary to develop long lasting and protective immunity to RSV. Putative vaccination schedules of 2, 4, and 6 months of age may be conceivable, ideally to be scheduled concurrently with other routine childhood vaccinations. PIV3 is highly immunogenic and the first PIV/RSV vaccination induces high levels of PIV3 antibodies. This may result in vector immunity such that subsequent immunizations with PIV/RSV may not produce a further rise in antibody titer. A recent study by Karron et al. presented data showing that multiple doses of PIV3 will not result in vector immunity provided the intervals between dose administrations are spaced far enough apart (see Karron et al., Pediatr. Infect. Dis. J. 22: 394-405 (2003)). The administration of a single dose of cp-45 PIV3 vaccine, a cold-passaged and temperature-sensitive virus, restricted the magnitude of vaccine replication after the second dose. However, the frequency of infection with a second dose of vaccine was clearly influenced by the dosing interval. Only 24% of infants shed virus when a second dose of vaccine was administered 1 month later. In contrast, 62% of infants shed virus when the second dose was administered 3 months after the first dose. These results suggested that to minimize PIV3 vector immunity effects, the interval between vaccinations should be more than one month but less than three months.

PIV3/RSV Immunization of AGMs Resulted in Production of hPIV3 Neutralizing and HAI Serum Antibodies

In order to evaluate whether the b/h PIV3/RSV vaccines could protect from RSV and hPIV3 infection, primate sera were analyzed for the presence of hPIV3 neutralization and HAI serum antibodies (Table 21).

TABLE 21
AGMs Immunized with b/h PIV3/RSV F2 or b/h PIV3/sol RSV
F2 Induced hPIV3 Neutralizing and HAI Serum Antibodies

| | | | Reciprocal | |
|---|---|---|---|---|
| | | hPIV3 | Geometric Mean | |
| | | Neutralizing | PIV3 HAI | |
| | Date of | Geometric Mean | Antibody | |
| Virus Used for | Serum | Reciprocal log₂ | Titers | |
| Immunization | Collection* | Antibody Titers | hPIV3^{#} | bPIV3^{#} |
| b/h PIV3/RSV | Day 28 | 6.1 | 128.0 | 11.3 |
| F2 | | | | |
| | Day 56 | 5.6 | 64.0 | 8.0 |
| b/h PIV3/sol | Day 28 | 5.8 | 128.0 | 16.0 |
| RSV F2 | | | | |
| | Day 56 | 5.7 | 64.0 | 8.0 |
| Placebo | Day 28 | <2.0 | <4.0 | <4.0 |
| | Day 56 | <2.0 | <4.0 | <4.0 |

*hPIV3 neutralizing antibody titers of <2.0 log₂ and PIV3 HAI titers of <4.0 were present in Day 1 pre-sera.
^{#}hPIV3/Wash/47885/57 and bPIV3/Kansas/15626/84 were used as antigens in the HAI assay.

Day 28 and 56 primate sera from animals immunized with b/h PIV3/RSV F2 and b/h PIV3/sol RSV F2 showed hPIV3 neutralizing antibody titers of ˜6 log₂. Human PIV3-specific HAI antibody titers of 128 and 64 were observed for b/h PIV3/RSV F2 and b/h PIV3/sol RSV F2 Day 28 and Day 56 sera, respectively. Lower HAI antibody titers of 11.3 and 16.0 were displayed when the bPIV3 antigen was tested using Day 28 sera. The Day 56 sera displayed even lower bPIV3 HAI titers of 8.0. Since the surface glycoproteins, F and HN, of b/h PIV3/RSV viruses were derived from human PIV3, a higher HAI serum antibody titer to the homologous antigen (hPIV3) was observed than to the heterologous bPIV3 antigen. hPIV3 neutralizing or PIV3 HAI serum antibodies were not detected in sera derived from placebo recipients. These results suggested that b/h PIV3/RSV vaccines may be also efficacious for hPIV3 infections.

This study determined whether hPIV3 serum HAI and neutralizing antibody titers were produced in response to vaccination. The levels of hPIV3 HAI and neutralizing antibodies observed for the primate sera obtained from animals immunized with both kinds of b/h PIV3/RSV F vaccines were similar to the titers displayed by rhesus monkeys vaccinated with b/h PIV3. Rhesus monkeys immunized with b/h PIV3 were protected completely from challenge with wildtype hPIV3. These results suggested that b/h PIV3 vectored RSV vaccines may be effective as bi-valent vaccines to protect infants from both RSV and hPIV3 infections and disease.

### 34. EXAMPLE 29

### Evaluating the Efficacy and Immunogenicity of b/h PIV3 Expressing an Antigenic Protein of MPV in African Green Monkeys

Potential MPV vaccine candidates, e.g., b/h PIV3 expressing an antigenic protein of MPV such as MPV F, are evaluated for efficacy and immunogenicity in a non-human primate model, such as African green monkeys.

Vero cells are maintained in Modified Eagle's Medium (MEM) (JRH Biosciences) supplemented with 2 mM L-glutamine, non-essential amino acids (NEAA), antibiotics, and 10% FBS. b/h PIV3 expressing an antigenic protein of MPV, e.g., b/h PIV3/MPV F2, and a wildtype MPV, e.g., hMPV/NL/1/00, are propagated in Vero cells. Cells are infected with the viruses at a multiplicity of infection (MOI) of 0.1 PFU/cell. Three to five days post-infection the cells and supernatant are collected and stabilized by adding 10× SPG (10× SPG is 2.18 M Sucrose, 0.038 M KH₂PO₄, 0.072 M K₂HPO₄, 0.054 M L-Glutamate) to a final concentration of 1×. The virus stocks are stored at −70° C. The virus titers are determined by plaque assays on Vero cells. Plaques are quantified after immunoperoxidase staining using PIV3 (VMRD) or MPV goat polyclonal antisera (Biogenesis).

MPV- and PIV3-seronegative African Green monkeys (Cercopithecus aethiops) (3.5 to 6.5 years old, 2.6 to 5.8 kg) are identified using an MPV F IgG ELISA (Immuno-Biological Laboratories) and a hemagglutination inhibition (HAI) assay for primate pre-sera collected on day 14 prior to the study start date. MPV F IgG ELISA is performed as follows: the primate sera from days 1, 28 and 56 from the vaccinated animals are analyzed for the presence of MPV F IgG using an ELISA kit (Immuno-Biological Laboratories, Hamburg, Germany) according to the manufacturer's instructions. The secondary monkey antiserum (Rockland Inc.) is used at a 1:1000 dilution. The MPV F IgG antibody titers are expressed as log₂ IgG U/ml. The HAI assays are performed by incubating serial two-fold dilutions of primate serum at 25° C. for 30 min with 8 HA units/0.05 ml of either bPIV3 or hPIV3. Subsequently, guinea pig red blood cells are added to each well, incubation is continued for 90 min, and each well is observed for hemagglutination. HAI titers are expressed as the reciprocal of the highest dilution of antiserum that inhibited virus-mediated agglutination of erythrocytes.

The primates are housed in individual micro-isolator cages. The monkeys are anesthetized with a ketamine-valium mixture and infected intranasally and intratracheally with a b/h PIV3 vector expressing an antigenic protein of MPV, e.g., b/h PIV3/MPV F2, and a wildtype MPV, e.g., hMPV/NL/1/00. The nasal dose volume is 0.5 ml per nostril, and the intratracheal dose volume is 1 ml. On Day 1, each animal receives a dose of 2 ml containing 2-3×10⁵ PFU of virus. The placebo animal group receives the same dose volume of Opti-MEM. On Day 28, all animals are challenged intratracheally and intranasally with 7×10⁵ PFU of hMPV/NL/100 (1 ml at each site). Nasopharyngeal (NP) swabs are collected daily for 11 days and tracheal lavage (TL) specimens are collected on Days 1, 3, 5, 7 and 9 post-immunization and post-challenge. Blood samples obtained from the femoral vein are collected on Days 0, 7, 14, 21, 28, 35, 42, 49 and 56 for serological analysis. The animals are monitored for body temperature changes indicating a fever, signs of a cold, runny nose, sneezing, loss of appetite, and body weight. Virus present in the primate NP and TL specimens is quantitated by plaque assays using Vero cells that are immunostained with MPV goat polyclonal antiserum. Mean peak virus titers represent the mean of the peak virus titer measured for each animal on any of the 11 days following immunization or challenge.

Plaque reduction neutralization assays (PRNAs) are carried out for sera obtained on days 1, 28, and 56 post-dose from primates infected with a b/h PIV3 vector expressing an antigenic protein of MPV, e.g., b/h PIV3/MPV F2. The primate sera are two-fold serially diluted, and incubated with 100 PFU of hMPV/NL/100 in the presence of guinea pig complement for one hour at 4° C. The virus-serum mixtures are transferred to Vero cell monolayers and overlaid with 1% methyl cellulose in EMEM/L-15 medium (JRH Biosciences; Lenexa, Kans.) containing 2% FBS and 1% antibiotics. After 6 days of incubation at 35° C., the monolayers are immunostained using MPV goat polyclonal antiserum for quantitation. Neutralization titers are expressed as the reciprocal log₂ of the highest serum dilution that inhibits 50% of viral plaques.

hPIV3 Microneutralization assays are performed on Vero cells. Serial two-fold dilutions of primate serum, starting at 1:4, are incubated at 37° C. for 60 min with 100 TCID₅₀ of hPIV3. Then virus-serum mixtures are transferred to cell monolayers in 96-well plates and incubated at 37° C. for six days, after which all wells are observed for CPE. Neutralization titers are expressed as the reciprocal of the highest serum dilution that inhibited CPE. Neutralization antibody titers of ≦1:4 (the lowest serum dilution tested) are assigned a reciprocal log₂ titer of 2.

To study the replication efficiency of the MPV vaccine candidates, the experiment is designed as follows. On Day 1, MPV and PIV3 sero-negative African green monkeys, four animals per group, are immunized intranasally and intratracheally with a MPV vaccine candidate, e.g., b/h PIV3/MPV F2, with a dose of 2-3×10⁵ PFU. A positive control group is infected with wildtype MPV, e.g., hMPV/NL/100, and the negative control group is administered placebo medium. On Day 28, all animals are challenged intranasally and intratracheally with 7×10⁵ PFU of wildtype MPV, e.g., hMPV/NL/100. The animals are housed in micro-isolator cages for the duration of this study. Nasopharyngeal swabs are collected daily for 11 days post-immunization and post-challenge, and tracheal lavage samples are obtained on days 2, 4, 6, 8, and 10 post-immunization and post-challenge. Serum samples for antibody analyses are collected every seven days throughout the duration of the study.

In order to evaluate immune protection from MPV infection, the vaccinated primates are challenged with a high dose of wildtype MPV, e.g., hMPV/NL/100, four weeks post-immunization. Efficacy is measured as a reduction in shed MPV challenge virus titer in the URT and LRT of the infected animals.

Efficacy of the b/h PIV3 vectored MPV vaccine candidates is further evaluated by the levels of MPV neutralizing and MPV F IgG serum antibody titers produced four weeks post-immunization. The MPV neutralizing antibody titers are determined using a 50% plaque reduction neutralization assay (PRNA). The immune responses elicited by the MPV vaccine candidates are also analyzed by measuring MPV F protein specific IgG levels at pre-dose (Day 1), four weeks post-dose (Day 28), and four weeks post-challenge (Day 56).

In order to evaluate whether the b/h PIV3 vectored MPV vaccines can protect from MPV and hPIV3 infection, primate sera are also analyzed for the presence of hPIV3 neutralization and HAI serum antibodies.

### 35. EXAMPLE 30

### Microneutralization Assay Using A b/h PIV3 Constructs Containing GFP or eGFP Gene

When viruses are inoculated into an animal, an array of antibodies against the virus are produced. Some of these antibodies can bind virus particles and neutralize the infectivity of the viruses. A microneutralization assay is used to analyze the remaining infectivity of the viruses after the viruses are incubated with dilutions of serum containing antibodies.

Microneutralization assays are performed as follows: sera are serially diluted with Opti-MEM Medium (1×). Serum and medium are mixed gently by inversing, and then place on ice. Each dilution of sera is incubated with a virus of interest, wherein the genome of the virus has been manipulated to contain one or more GFP or eGFP gene (see Section 9, Example 4). Cells are washed with phosphate buffered saline (“PBS”). The virus/sera mixture are added to cells and incubated for one hour at 35° C. All of the medium, which contain the virus, are removed, and cells are washed with PBS. Opti-MEM medium is added to the cells and the cell cultures are incubated for three days. The remaining infectivity of the viruses is measured by quantify GFP or eGFP green foci on the images captured with fluorescence microscope. Plaque reduction assay using a corresponding virus without GFP or eGFP, e.g., wildtype RSV, can also be performed for comparing the sensitivity of the microneutralization assay.

### 36. EXAMPLE 31

### Development of Arobust and High-Yielding Cell Culture for Manufacture of the Virus Vaccine Candidates

This example describes a robust and high-yielding cell culture process. This process can be used, e.g., for the manufacture of the virus vaccines described in the application. Critical process parameters were first identified, and the production process was optimized in small-scale experiments. Next, numerous studies were conducted using the optimized operating conditions to determine the scalability, robustness, and reproducibility of the production system. The process described in the Example increased infectious virus yields by over 1 log₁₀ TCID₅₀/mL.

Materials and Methods:

The virus, a bovine/human PIV-3 virus containing a RSV F gene insert construct as shown in FIG. 4, hereinafter referred as “b/h PIV3/RSV F2”) was propagated using Vero cells (ATCC) that have been adapted to grow in a serumfree medium (SFM) composed of OPTI PRO SFM (Gibco) supplemented with 4 mM Lglutamine. The anchorage-dependent Vero cells were routinely maintained by seeding at 5×10⁴ cells/ml in the SFM, refeeding the cultures 3 days post-seeding, and passaging the flask 5 days post-seeding. Virus titers were determined using a 50% tissue culture infective dose (TCID50) assay and were quantified in log₁₀TCID₅₀/mL.

Results

Process Optimization

Small-scale process optimization studies were conducted in T-75 flasks seeded with Vero cells in SFM at 1.75×10⁶ cells/flask. All pre-infection cultures were incubated at 37±1° C., 5±1% CO₂ and infected either 3 or 5 days post-seeding. For cultures infected 5 days post-seeding, complete SFM exchange was performed on the third day post-seeding. At the time of infection, the spent medium was replaced by SFM containing the b/h PIV3/RSV F2 virus. Cultures were sampled at least once daily, and assayed for infectious virus by TCID₅₀. Error bars in the Figures represent the standard deviation obtained from duplicate cultures.

Effects of Multiplicity of Infection (MOI)

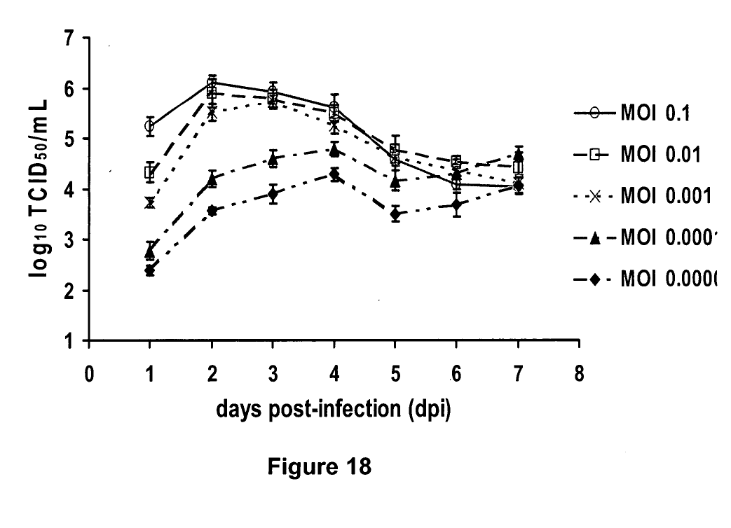

Vero cultures were infected with the b/h PIV3/RSV F2 virus at MOI of 0.1, 0.01, 0,001, 0.0001, and 0.00001 on the fifth day post-seeding. Results show that the lowest virus titers were obtained for MOI 0.0001 and 0.00001, whereas comparable virus titers were observed for MOI 0.1, 0.01, and 0.001 (FIG. 18). The experiment was repeated and the same trends were observed.

Effects of Point of Infection (POI) and Post-Infection Temperature

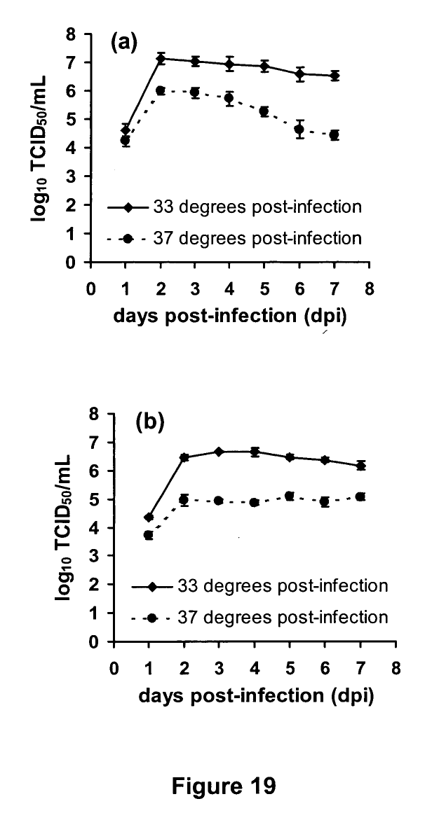

Vero cultures were infected at 2 different cell densities and incubated at either 33±1° C. or 37±1° C. post-infection. Although infectious virus titers were not enhanced by infecting at higher cell densities, they were elevated by over 1 log₁₀ TCID₅₀/mL using the lower post-infection incubation temperature (FIG. 19). The experiment was repeated and the same trends were observed.

Effects of Pre-Infection Medium Supplementation

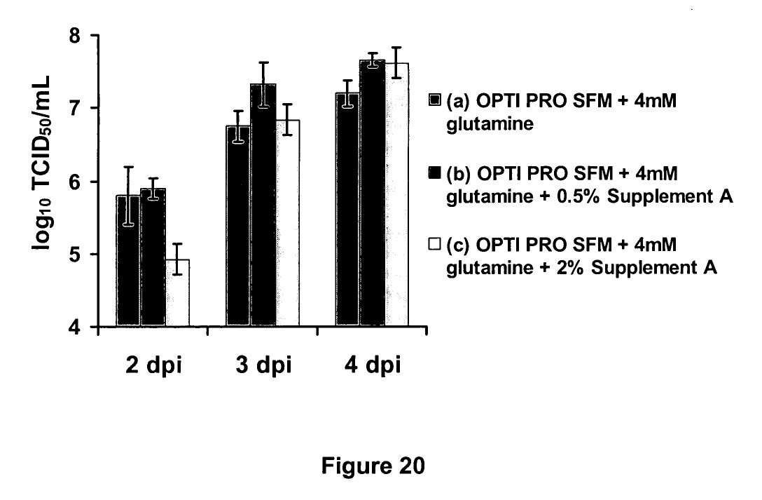

By supplementing the pre-infection medium with serum, infectious virus titers were further increased by over 1 log₁₀ TCID/ML (FIG. 20).

Expression Profile of PIV-3 HN, PIV-3 F, and RSV F Viral Proteins

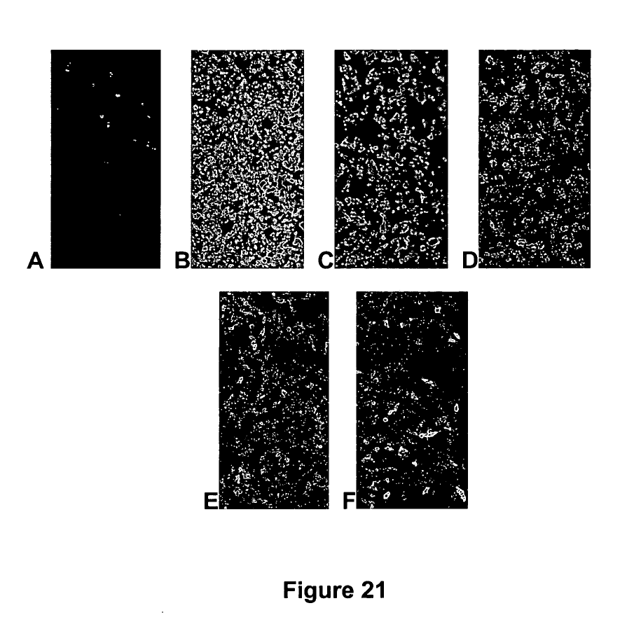

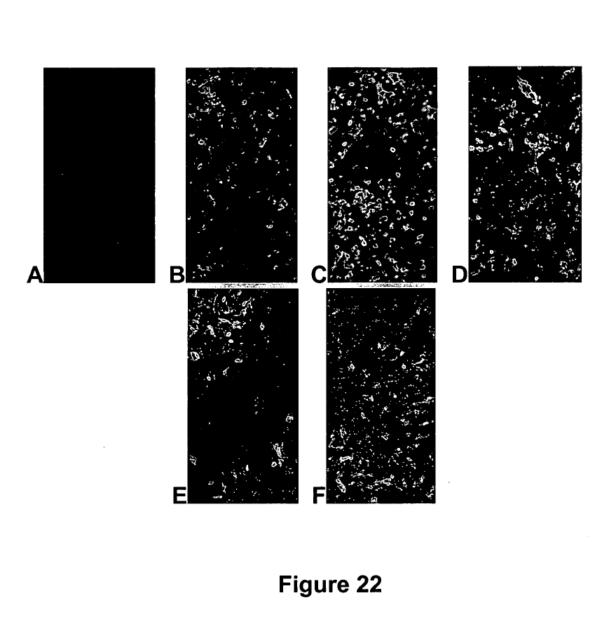

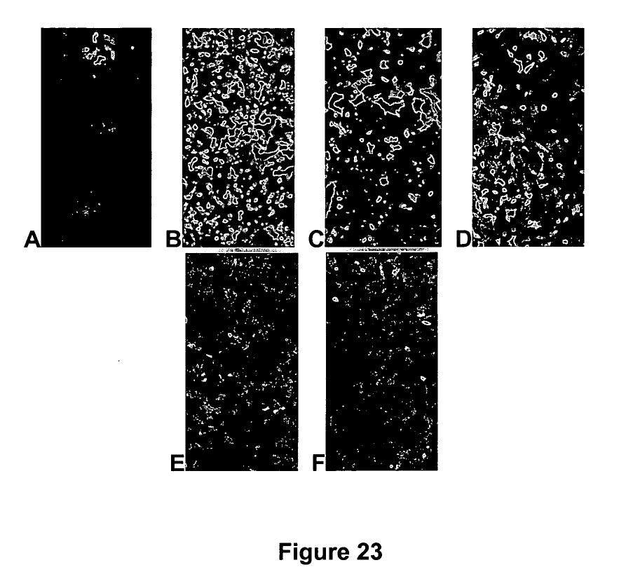

Expression of the three RSV F2 viral proteins—PIV-3 HN, PIV-3 F, and RSV F—was monitored over the course of infection in Vero cell cultures by immunofluorescence. Cells were seeded at 8×10³ cells/well in SFM into multiple 96-well plates. Four days post seeding, the plates were rinsed once with DPBS, infected with the b/h PIV3/RSV F2 virus at MOI 0.001 and incubated at 33±1° C., 5±1% CO₂. At multiple post-infection time intervals, a 96-well plate was fixed with paraformaldehyde (4%) prior to immunostaining. FIGS. 21, 22, and 23 indicate that all three viral proteins were expressed by the cultures of Vero cells in SFM. The images in these figures were captured at 5× magnification.

Process Scale-Up

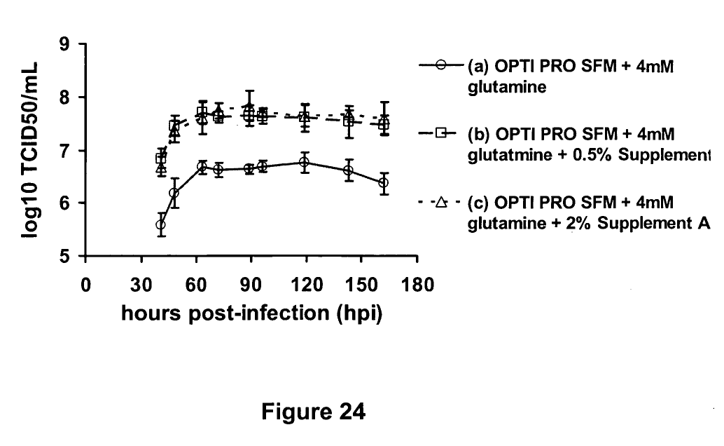

The medium supplementation experiment was repeated by seeding the Vero cells in 1700 cm² Roller Bottles (Coming) at 1.75×10⁷ cells/bottle. Infectious virus titers were noticeably higher in the cultures supplemented with serum pre-infection (FIG. 24). The experiment was repeated twice and the same trends were observed.

Summary

By identifying critical process parameters and optimizing the infection process in small-scale experiments, the RSV F2 infectious virus titers were increased by over 1 log₁₀ TCID₅₀/mL. The b/h PIV3/RSV F2 virus production process was successfully scaled-up in roller bottle experiments with consistent and reproducible results.

### 37. EXAMPLE 32

### Plasmid-Only Recovery of PIV3 in Serum Free Vero Cells by Electroporation

The process demonstrated in this example allows recovery of recombinant PIV3 using plasmids only, in the absence of helper viruses. The recovery of PIV3 was carried out using SF Vero cells, which were propagated in the absence of animal and human derived products. This process allows recovery of recombinant PIV3 with similar efficiency to previous methods using helper viruses (recombinant vaccinia or fowl-pox viruses expressing T7 polymerase). Because no helper viruses are needed in the recovery process, the vaccine viruses are free of contaminating agents, simplifying downstream vaccine production. The cells used for vaccine virus recovery were grown in media containing no animal or human derived products. This eliminates concerns about transmissible spongiform encephalopathies (e.g. BSE), for product end users.

This method enables generation of a recombinant vaccine seed that is completely free of animal or human derived components. The seed is also free of contaminating helper viruses.

Plasmid-based expression systems for rescue of viruses from cDNA are described, e.g., in RA Lerch et al., Wyeth Vaccines, Pearl River N.Y., USA (Abstract 206 from XII International Conference on Negative Strand Viruses, Jun. 14^{th}-19^{th} 2003, Pisa Italy) and G. Neumann et. al., J. Virol., 76, pp 406-410.

Methods and Results

bPIV3 N plasmids (4 μg; marker: kanamycin resistancy), bPIV3 P plasmids (4 μg; marker: kanamycin resistancy), bPIV3 L plasmids (2 μg; marker: kanamycin resistancy), cDNA encoding PIV3 antigenomic cDNA (5 μg; marker: kanamycin resistancy) and pADT7(N)DpT7 encoding T7 RNA polymerase (5 μg; marker: blasticidin) were introduced into SF Vero cells using electroporation in serum-free medium. bPIV3 N, bPIV3 P, and bPIV3 L are in pCITE vectors under the control of the T7 promoter. pADT7(N)ΔpT7 is in a modified pcDNA6/V5-His C in which the T7 promoter was deleted leaving only the CMV promoter. The T7 polymerase is expressed from the CMV promoter. Antigenomic bPIV3 is in pUC19 and transcription of the antigenome is under the T7 promoter.

The pulse for the electroporation was 220V and 950 microfarads. 5.5×10⁶ SF Vero cells were used per electroporation. The electroporated cells were allowed to recover at 33° C. in the presence of OptiC (a custom formulation from GIBCO Invitrogen Corporation) overnight. Recovered cells were washed twice with 1 mL of PBS containing calcium and magnesium and overlayed with 2 mL of OptiC. Electroporated cells were further incubated at 33° C. for 5-7 days. At the end of the incubation period, cells were scraped into the media and total cell lysate was analyzed for presence of PIV3.

Virus recovery was confirmed by immunostaining of plaque assays using RSV or hMPV specific polyclonal antibodies. The titers recovered from electroporated cells are shown in Table 22 and Table 23. Table 22 shows the titers of different viruses recovered by electroporation into SF Vero cells. The viruses are different chimeric bovine PIV3. The plasmids with the cDNAs encoding the different chimeric bovine PIV3 are PIV3 with the F gene of human RSV at position 2 (MEDI 534), the marker on the plasmid is kanamycin (position 2 is the position between the first and the second open reading frame of the native viral genome, or alternatively, the position of the second gene of the viral genome to be transcribed); bovine PIV3 with a soluble form of the F gene lacking the transmembrane and luminal domains of human RSV at position 2 (MEDI 535), the marker on the plasmid is kanamycin; bovine PIV3 with the F gene of human metapneumovirus at position 2 (MEDI 536), the marker on the plasmid is kanamycin; bovine PIV3 with the F gene of human RSV at position 2 (MEDI 534), the marker on the plasmid is ampicillin; bovine PIV3 with the F gene of human metapneumovirus at position 2 (MEDI 536), the marker on the plasmid is ampicillin.

Virus recovery by electroporation under different conditions is shown in 23 for chimeric virus MEDI 534. The Vero cells were grown in the presence of serum for titration. MEDI 534 were electroporated using (i) Opti C, (ii) Opti C containing 1× gentamicin, (iii) Opti MEM (opti C containing human transferring) to test the efficiency of virus recovery using different media. Electroporations were done under identical conditions using the same SF Vero cells. The results showed that 1× gentamicin is completely inhibitory to virus recovery and human transferrin does not play a role in the efficiency of virus recovery. The presence of bacterial RNA is also inhibitory to virus recovery. In electroporations done with plasmids prepared without RNase A treatment, no virus was recovered.

P0 and P1 in Table 22 and Table 23 refer to the viruses. P0 indicates viruses obtained from electroporated cells. P1 indicates viruses that were amplified once in vero cells grown in the presence of fetal bovine serum. Similar titers are obtained if P0 viruses were amplified in SF vero cells.

TABLE 22
Viruses (Medi 543-537) recovered by electroporation in serum-free
Vero (P17) and passaged in serum-containing Vero cells. SF Vero used
at Passage 17. P0 and P1 are passages of viruses after electroporation
and after one amplification in Vero cells respectively.

| | |
|---|---|
| P0 | P1 |

| | |
|---|---|
| Vectored Viruses* | log₁₀(pfu/ml) |

| | | |
|---|---|---|
| bh RSVF2 kan (MEDI 534) | 3.53 | 8.40 |
| bh RSVF2 sol kan (MEDI 535) | 3.20 | 8.20 |
| bh hMPVF2 kan (MEDI 536) | >4.00 | 8.18 |
| bh RSVF2 amp (MEDI 534) | <1.00 | 8.60 |
| bh hMPVF2 amp (MEDI 536) | 4.28 | 8.34 |

TABLE 23
Virus recovery by electroporation under different conditions.

| | P0² |
|---|---|
| Vectored Viruses¹ | log₁₀(pfu/ml) |
| MEDI 534 Opti C | 3.56 |
| MEDI 534 Opti C W/ Gentamicin | <0.3 |
| MEDI 534 OptiMEM | 4.00 |
| MEDI 534 RNase free³ | 2.90 |

¹Viruses recovered in serum-free Vero (P8) and titered in Vero cells grown in the presence of serum
²Passage of virus obtained from electroporated cells.
³Electroporation was performed with plasmids prepared without RNase A treatment.

The present invention is not to be limited in scope by the specific described embodiments that are intended as single illustrations of individual aspects of the invention, and any constructs, viruses or enzymes that are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

Various publications are cited herein, the disclosures of which are incorporated by reference in their entireties.

TABLE 24
LEGEND FOR SEQUENCE LISTING

| | |
|---|---|
| SEQ ID NO: 1 | Human metapneumovirus isolate 00-1 matrix protein (M) and fusion protein (F) genes |
| SEQ ID NO: 2 | Avian pneumovirus fusion protein gene, partial cds |
| SEQ ID NO: 3 | Avian pneumovirus isolate 1b fusion protein mRNA, complete cds |
| SEQ ID NO: 4 | Turkey rhinotracheitis virus gene for fusion protein (F1 and F2 subunits), complete cds |
| SEQ ID NO: 5 | Avian pneumovirus matrix protein (M) gene, partial cds and Avian pneumovirus fusion |
| | glycoprotein (F) gene, complete cds |
| SEQ ID NO: 6 | paramyxovirus F protein hRSV B |
| SEQ ID NO: 7 | paramyxovirus F protein hRSV A2 |
| SEQ ID NO: 8 | human metapneumovirus 01-71 (partial sequence) |
| SEQ ID NO: 9 | Human metapneumovirus isolate 00-1 matrix protein(M) and fusion protein (F) genes |
| SEQ ID NO: 10 | Avian pneumovirus fusion protein gene, partial cds |
| SEQ ID NO: 11 | Avian pneumovirus isolate 1b fusion protein mRNA, complete cds |
| SEQ ID NO: 12 | Turkey rhinotracheitis virus gene for fusion protein (F1 and F2 subunits), complete cds |
| SEQ ID NO: 13 | Avian pneumovirus fusion glycoprotein (F) gene, complete cds |
| SEQ ID NO: 14 | Turkey rhinotracheitis virus (strain CVL14/1)attachment protien (G) mRNA, complete cds |
| SEQ ID NO: 15 | Turkey rhinotracheitis virus (strain 6574)attachment protein (G), complete cds |
| SEQ ID NO: 16 | Turkey rhinotracheitis virus (strain CVL14/1)attachment protein (G) mRNA, complete cds |
| SEQ ID NO: 17 | Turkey rhinotracheitis virus (strain 6574)attachment protein (G), complete cds |
| SEQ ID NO: 18 | F protein sequence for HMPV isolate NL/1/00 |
| SEQ ID NO: 19 | F protein sequence for HMPV isolate NL/17/00 |
| SEQ ID NO: 20 | F protein sequence for HMPV isolate NL/1/99 |
| SEQ ID NO: 21 | F protein sequence for HMPV isolate NL/1/94 |
| SEQ ID NO: 22 | F-gene sequence for HMPV isolate NL/1/00 |
| SEQ ID NO: 23 | F-gene sequence for HMPV isolate NL/17/00 |
| SEQ ID NO: 24 | F-gene sequence for HMPV isolate NL/1/99 |
| SEQ ID NO: 25 | F-gene sequence for HMPV isolate NL/1/94 |
| SEQ ID NO: 26 | G protein sequence for HMPV isolate NL/1/00 |
| SEQ ID NO: 27 | G protein sequence for HMPV isolate NL/17/00 |
| SEQ ID NO: 28 | G protein sequence for HMPV isolate NL/1/99 |
| SEQ ID NO: 29 | G protein sequence for HMPV isolate NL/1/94 |
| SEQ ID NO: 30 | G-gene sequence for HMPV isolate NL/1/00 |
| SEQ ID NO: 31 | G-gene sequence for HMPV isolate NL/17/00 |
| SEQ ID NO: 32 | G-gene sequence for HMPV isolate NL/1/99 |
| SEQ ID NO: 33 | G-gene sequence for HMPV isolate NL/1/94 |
| SEQ ID NO: 34 | L protein sequence for HMPV isolate NL/1/00 |
| SEQ ID NO: 35 | L protein sequence for HMPV isolate NL/17/00 |
| SEQ ID NO: 36 | L protein sequence for HMPV isolate NL/1/99 |
| SEQ ID NO: 37 | L protein sequence for HMPV isolate NL/1/94 |
| SEQ ID NO: 38 | L-gene sequence for HMPV isolate NL/1/00 |
| SEQ ID NO: 39 | L-gene sequence for HMPV isolate NL/17/00 |
| SEQ ID NO: 40 | L-gene sequence for HMPV isolate NL/1/99 |
| SEQ ID NO: 41 | L-gene sequence for HMPV isolate NL/1/94 |
| SEQ ID NO: 42 | M2-1 protein sequence for HMPV isolate NL/1/00 |
| SEQ ID NO: 43 | M2-1 protein sequence for HMPV isolate NL/17/00 |
| SEQ ID NO: 44 | M2-1 protein sequence for HMPV isolate NL/1/99 |
| SEQ ID NO: 45 | M2-1 protein sequence for HMPV isolate NL/1/94 |
| SEQ ID NO: 46 | M2-1 gene sequence for HMPV isolate NL/1/00 |
| SEQ ID NO: 47 | M2-1 gene sequence for HMPV isolate NL/17/00 |
| SEQ ID NO: 48 | M2-1 gene sequence for HMPV isolate NL/1/99 |
| SEQ ID NO: 49 | M2-1 gene sequence for HMPV isolate NL/1/94 |
| SEQ ID NO: 50 | M2-2 protein sequence for HMPV isolate NL/1/00 |
| SEQ ID NO: 51 | M2-2 protein sequence for HMPV isolate NL/17/00 |
| SEQ ID NO: 52 | M2-2 protein sequence for HMPV isolate NL/1/99 |
| SEQ ID NO: 53 | M2-2 protein sequence for HMPV isolate NL/1/94 |
| SEQ ID NO: 54 | M2-2 gene sequence for HMPV isolate NL/1/00 |
| SEQ ID NO: 55 | M2-2 gene sequence for HMPV isolate NL/17/00 |
| SEQ ID NO: 56 | M2-2 gene sequence for HMPV isolate NL/1/99 |
| SEQ ID NO: 57 | M2-2 gene sequence for HMPV isolate NL/1/94 |
| SEQ ID NO: 58 | M2 gene sequence for HMPV isolate NL/1/00 |
| SEQ ID NO: 59 | M2 gene sequence for HMPV isolate NL/17/00 |
| SEQ ID NO: 60 | M2 gene sequence for HMPV isolate NL/1/99 |
| SEQ ID NO: 61 | M2 gene sequence for HMPV isolate NL/1/94 |
| SEQ ID NO: 62 | M protein sequence for HMPV isolate NL/1/00 |
| SEQ ID NO: 63 | M protein sequence for HMPV isolate NL/17/00 |
| SEQ ID NO: 64 | M protein sequence for HMPV isolate NL/1/99 |
| SEQ ID NO: 65 | M protein sequence for HMPV isolate NL/1/94 |
| SEQ ID NO: 66 | M gene sequence for HMPV isolate NL/1/00 |
| SEQ ID NO: 67 | M gene sequence for HMPV isolate NL/17/00 |
| SEQ ID NO: 68 | M gene sequence for HMPV isolate NL/1/99 |
| SEQ ID NO: 69 | M gene sequence for HMPV isolate NL/1/94 |
| SEQ ID NO: 70 | N protein sequence for HMPV isolate NL/1/00 |
| SEQ ID NO: 71 | N protein sequence for HMPV isolate NL/17/00 |
| SEQ ID NO: 72 | N protein sequence for HMPV isolate NL/1/99 |
| SEQ ID NO: 73 | N protein sequence for HMPV isolate NL/1/94 |
| SEQ ID NO: 74 | N gene sequence for HMPV isolate NL/1/00 |
| SEQ ID NO: 75 | N gene sequence for HMPV isolate NL/17/00 |
| SEQ ID NO: 76 | N gene sequence for HMPV isolate NL/1/99 |
| SEQ ID NO: 77 | N gene sequence for HMPV isolate NL/1/94 |
| SEQ ID NO: 78 | P protein sequence for HMPV isolate NL/1/00 |
| SEQ ID NO: 79 | P protein sequence for HMPV isolate NL/17/00 |
| SEQ ID NO: 80 | P protein sequence for HMPV isolate NL/1/99 |
| SEQ ID NO: 81 | P protein sequence for HMPV isolate NL/1/94 |
| SEQ ID NO: 82 | P gene sequence for HMPV isolate NL/1/00 |
| SEQ ID NO: 83 | P gene sequence for HMPV isolate NL/17/00 |
| SEQ ID NO: 84 | P gene sequence for HMPV isolate NL/1/99 |
| SEQ ID NO: 85 | P gene sequence for HMPV isolate NL/1/94 |
| SEQ ID NO: 86 | SH protein sequence for HMPV isolate NL/1/00 |
| SEQ ID NO: 87 | SH protein sequence for HMPV isolate NL/17/00 |
| SEQ ID NO: 88 | SH protein sequence for HMPV isolate NL/1/99 |
| SEQ ID NO: 89 | SH protein sequence for HMPV isolate NL/1/94 |
| SEQ ID NO: 90 | SH gene sequence for HMPV isolate NL/1/00 |
| SEQ ID NO: 91 | SH gene sequence for HMPV isolate NL/17/00 |
| SEQ ID NO: 92 | SH gene sequence for HMPV isolate NL/1/99 |
| SEQ ID NO: 93 | SH gene sequence for HMPV isolate NL/1/94 |
| SEQ ID NO: 94 | isolate NL/1/99 (99-1) HMPV (Human Metapneumovirus)cDNA sequence |
| SEQ ID NO: 95 | isolate NL/1/00 (00-1) HMPV cDNA sequence |
| SEQ ID NO: 96 | isolate NL/17/00 HMPV cDNA sequence |
| SEQ ID NO: 97 | isolate NL/1/94 HMPV cDNA sequence |
| SEQ ID NO: 98 | G-gene coding sequence for isolate NL/1/00 (A1) |
| SEQ ID NO: 99 | G-gene coding sequence for isolate BR/2/01 (A1) |
| SEQ ID NO: 100 | G-gene coding sequence for isolate FL/4/01 (A1) |
| SEQ ID NO: 101 | G-gene coding sequence for isolate FL/3/01 (A1) |
| SEQ ID NO: 102 | G-gene coding sequence for isolate FL/8/01 (A1) |
| SEQ ID NO: 103 | G-gene coding sequence for isolate FL/10/01 (A1) |
| SEQ ID NO: 104 | G-gene coding sequence for isolate NLA 10/01 (A1) |
| SEQ ID NO: 105 | G-gene coding sequence for isolate NL/2/02 (A1) |
| SEQ ID NO: 106 | G-gene coding sequence for isolate NL/17/00 (A2) |
| SEQ ID NO: 107 | G-gene coding sequence for isolate NL/1/81 (A2) |
| SEQ ID NO: 108 | G-gene coding sequence for isolate NL/1/93 (A2) |
| SEQ ID NO: 109 | G-gene coding sequence for isolate NL/2/93 (A2) |
| SEQ ID NO: 110 | G-gene coding sequence for isolate NL/3/93 (A2) |
| SEQ ID NO: 111 | G-gene coding sequence for isolate NL/1/95 (A2) |
| SEQ ID NO: 112 | G-gene coding sequence for isolate NL/2/96 (A2) |
| SEQ ID NO: 113 | G-gene coding sequence for isolate NL/3/96 (A2) |
| SEQ ID NO: 114 | G-gene coding sequence for isolate NL/22/01 (A2) |
| SEQ ID NO: 115 | G-gene coding sequence for isolate NL/24/01 (A2) |
| SEQ ID NO: 116 | G-gene coding sequence for isolate NL/23/01 (A2) |
| SEQ ID NO: 117 | G-gene coding sequence for isolate NL/29/01 (A2) |
| SEQ ID NO: 118 | G-gene coding sequence for isolate NL/3/02 (A2) |
| SEQ ID NO: 119 | G-gene coding sequence for isolate NL/1/99 (B1) |
| SEQ ID NO: 120 | G-gene coding sequence for isolate NL/11/00 (B1) |
| SEQ ID NO: 121 | G-gene coding sequence for isolate NL/12/00 (B1) |
| SEQ ID NO: 122 | G-gene coding sequence for isolate NL/5/01 (B1) |
| SEQ ID NO: 123 | G-gene coding sequence for isolate NL/9/01 (B1) |
| SEQ ID NO: 124 | G-gene coding sequence for isolate NL/21/01 (B1) |
| SEQ ID NO: 125 | G-gene coding sequence for isolate NL/1/94 (B2) |
| SEQ ID NO: 126 | G-gene coding sequence for isolate NL/1/82 (B2) |
| SEQ ID NO: 127 | G-gene coding sequence for isolate NL/1/96 (B2) |
| SEQ ID NO: 128 | G-gene coding sequence for isolate NL/6/97 (B2) |
| SEQ ID NO: 129 | G-gene coding sequence for isolate NL/9/00 (B2) |
| SEQ ID NO: 130 | G-gene coding sequence for isolate NL/3/01 (B2) |
| SEQ ID NO: 131 | G-gene coding sequence for isolate NL/4/01 (B2) |
| SEQ ID NO: 132 | G-gene coding sequence for isolate UK/5/01 (B2) |
| SEQ ID NO: 133 | G-protein sequence for isolate NL/1/00 (A1) |
| SEQ ID NO: 134 | G-protein sequence for isolate BR/2/01 (A1) |
| SEQ ID NO: 135 | G-protein sequence for isolate FL/4/01 (A1) |
| SEQ ID NO: 136 | G-protein sequence for isolate FL/3/01 (A1) |
| SEQ ID NO: 137 | G-protein sequence for isolate FL/8/01 (A1) |
| SEQ ID NO: 138 | G-protein sequence for isolate FL/10/01 (A1) |
| SEQ ID NO: 139 | G-protein sequence for isolate NL/10/01 (A1) |
| SEQ ID NO: 140 | G-protein sequence for isolate NL/2/02 (A1) |
| SEQ ID NO: 141 | G-protein sequence for isolate NL/17/00 (A2) |
| SEQ ID NO: 142 | G-protein sequence for isolate NL/1/81 (A2) |
| SEQ ID NO: 143 | G-protein sequence for isolate NL/1/93 (A2) |
| SEQ ID NO: 144 | G-protein sequence for isolate NL/2/93 (A2) |
| SEQ ID NO: 145 | G-protein sequence for isolate NL/3/93 (A2) |
| SEQ ID NO: 146 | G-protein sequence for isolate NL/1/95 (A2) |
| SEQ ID NO: 147 | G-protein sequence for isolate NL/2/96 (A2) |
| SEQ ID NO: 148 | G-protein sequence for isolate NL/3/96 (A2) |
| SEQ ID NO: 149 | G-protein sequence for isolate NL/22/01 (A2) |
| SEQ ID NO: 150 | G-protein sequence for isolate NL/24/01 (A2) |
| SEQ ID NO: 151 | G-protein sequence for isolate NL/23/01 (A2) |
| SEQ ID NO: 152 | G-protein sequence for isolate NL/29/01 (A2) |
| SEQ ID NO: 153 | G-protein sequence for isolate NL/3/02 (A2) |
| SEQ ID NO: 154 | G-protein sequence for isolate NL/1/99 (B1) |
| SEQ ID NO: 155 | G-protein sequence for isolate NL/11/00 (B1) |
| SEQ ID NO: 156 | G-protein sequence for isolate NL/12/00 (B1) |
| SEQ ID NO: 157 | G-protein sequence for isolate NL/5/01 (B1) |
| SEQ ID NO: 158 | G-protein sequence for isolate NL/9/01 (B1) |
| SEQ ID NO: 159 | G-protein sequence for isolate NL/21/01 (B1) |
| SEQ ID NO: 160 | G-protein sequence for isolate NL/1/94 (B2) |
| SEQ ID NO: 161 | G-protein sequence for isolate NL/1/82 (B2) |
| SEQ ID NO: 162 | G-protein sequence for isolate NL/1/96 (B2) |
| SEQ ID NO: 163 | G-protein sequence for isolate NL/6/97 (B2) |
| SEQ ID NO: 164 | G-protein sequence for isolate NL/9/00 (B2) |
| SEQ ID NO: 165 | G-protein sequence for isolate NL/3/01 (B2) |
| SEQ ID NO: 166 | G-protein sequence for isolate NL/4/01 (B2) |
| SEQ ID NO: 167 | G-protein sequence for isolate NL/5/01 (B2) |
| SEQ ID NO: 168 | F-gene coding sequence for isolate NL/1/00 |
| SEQ ID NO: 169 | F-gene coding sequence for isolate UK/1/00 |
| SEQ ID NO: 170 | F-gene coding sequence for isolate NL/2/00 |
| SEQ ID NO: 171 | F-gene coding sequence for isolate NL/13/00 |
| SEQ ID NO: 172 | F-gene coding sequence for isolate NL/14/00 |
| SEQ ID NO: 173 | F-gene coding sequence for isolate FL/3/01 |
| SEQ ID NO: 174 | F-gene coding sequence for isolate FL/4/01 |
| SEQ ID NO: 175 | F-gene coding sequence for isolate FL/8/01 |
| SEQ ID NO: 176 | F-gene coding sequence for isolate UK/1/01 |
| SEQ ID NO: 177 | F-gene coding sequence for isolate UK/7/01 |
| SEQ ID NO: 178 | F-gene coding sequence for isolate FL/10/01 |
| SEQ ID NO: 179 | F-gene coding sequence for isolate NL/6/01 |
| SEQ ID NO: 180 | F-gene coding sequence for isolate NL/8/01 |
| SEQ ID NO: 181 | F-gene coding sequence for isolate NL/10/01 |
| SEQ ID NO: 182 | F-gene coding sequence for isolate NL/14/01 |
| SEQ ID NO: 183 | F-gene coding sequence for isolate NL/20/01 |
| SEQ ID NO: 184 | F-gene coding sequence for isolate NL/25/01 |
| SEQ ID NO: 185 | F-gene coding sequence for isolate NL/26/01 |
| SEQ ID NO: 186 | F-gene coding sequence for isolate NL/28/01 |
| SEQ ID NO: 187 | F-gene coding sequence for isolate NL/30/01 |
| SEQ ID NO: 188 | F-gene coding sequence for isolate BR/2/01 |
| SEQ ID NO: 189 | F-gene coding sequence for isolate BR/3/01 |
| SEQ ID NO: 190 | F-gene coding sequence for isolate NL/2/02 |
| SEQ ID NO: 191 | F-gene coding sequence for isolate NL/4/02 |
| SEQ ID NO: 192 | F-gene coding sequence for isolate NL/5/02 |
| SEQ ID NO: 193 | F-gene coding sequence for isolate NL/6/02 |
| SEQ ID NO: 194 | F-gene coding sequence for isolate NL/7/02 |
| SEQ ID NO: 195 | F-gene coding sequence for isolate NL/9/02 |
| SEQ ID NO: 196 | F-gene coding sequence for isolate FL/1/02 |
| SEQ ID NO: 197 | F-gene coding sequence for isolate NL/1/81 |
| SEQ ID NO: 198 | F-gene coding sequence for isolate NL/1/93 |
| SEQ ID NO: 199 | F-gene coding sequence for isolate NL/2/93 |
| SEQ ID NO: 200 | F-gene coding sequence for isolate NL/4/93 |
| SEQ ID NO: 201 | F-gene coding sequence for isolate NL/1/95 |
| SEQ ID NO: 202 | F-gene coding sequence for isolate NL/2/96 |
| SEQ ID NO: 203 | F-gene coding sequence for isolate NL/3/96 |
| SEQ ID NO: 204 | F-gene coding sequence for isolate NL/1/98 |
| SEQ ID NO: 205 | F-gene coding sequence for isolate NL/17/00 |
| SEQ ID NO: 206 | F-gene coding sequence for isolate NL/22/01 |
| SEQ ID NO: 207 | F-gene coding sequence for isolate NL/29/01 |
| SEQ ID NO: 208 | F-gene coding sequence for isolate NL/23/01 |
| SEQ ID NO: 209 | F-gene coding sequence for isolate NL/17/01 |
| SEQ ID NO: 210 | F-gene coding sequence for isolate NL/24/01 |
| SEQ ID NO: 211 | F-gene coding sequence for isolate NL/3/02 |
| SEQ ID NO: 212 | F-gene coding sequence for isolate NL/3/98 |
| SEQ ID NO: 213 | F-gene coding sequence for isolate NL/1/99 |
| SEQ ID NO: 214 | F-gene coding sequence for isolate NL/2/99 |
| SEQ ID NO: 215 | F-gene coding sequence for isolate NL/3/99 |
| SEQ ID NO: 216 | F-gene coding sequence for isolate NL/11/00 |
| SEQ ID NO: 217 | F-gene coding sequence for isolate NL/12/00 |
| SEQ ID NO: 218 | F-gene coding sequence for isolate NL/1/01 |
| SEQ ID NO: 219 | F-gene coding sequence for isolate NL/5/01 |
| SEQ ID NO: 220 | F-gene coding sequence for isolate NL/9/01 |
| SEQ ID NO: 221 | F-gene coding sequence for isolate NL/19/01 |
| SEQ ID NO: 222 | F-gene coding sequence for isolate NL/21/01 |
| SEQ ID NO: 223 | F-gene coding sequence for isolate UK/11/01 |
| SEQ ID NO: 224 | F-gene coding sequence for isolate FL/1/01 |
| SEQ ID NO: 225 | F-gene coding sequence for isolate FL/2/01 |
| SEQ ID NO: 226 | F-gene coding sequence for isolate FL/5/01 |
| SEQ ID NO: 227 | F-gene coding sequence for isolate FL/7/01 |
| SEQ ID NO: 228 | F-gene coding sequence for isolate FL/9/01 |
| SEQ ID NO: 229 | F-gene coding sequence for isolate UK/10/01 |
| SEQ ID NO: 230 | F-gene coding sequence for isolate NL/1/02 |
| SEQ ID NO: 231 | F-gene coding sequence for isolate NL/1/94 |
| SEQ ID NO: 232 | F-gene coding sequence for isolate NL/1/96 |
| SEQ ID NO: 233 | F-gene coding sequence for isolate NL/6/97 |
| SEQ ID NO: 234 | F-gene coding sequence for isolate NL/7/00 |
| SEQ ID NO: 235 | F-gene coding sequence for isolate NL/9/00 |
| SEQ ID NO: 236 | F-gene coding sequence for isolate NL/19/00 |
| SEQ ID NO: 237 | F-gene coding sequence for isolate NL/28/00 |
| SEQ ID NO: 238 | F-gene coding sequence for isolate NL/3/01 |
| SEQ ID NO: 239 | F-gene coding sequence for isolate NL/4/01 |
| SEQ ID NO: 240 | F-gene coding sequence for isolate NL/11/01 |
| SEQ ID NO: 241 | F-gene coding sequence for isolate NL/15/01 |
| SEQ ID NO: 242 | F-gene coding sequence for isolate NL/18/01 |
| SEQ ID NO: 243 | F-gene coding sequence for isolate FL/6/01 |
| SEQ ID NO: 244 | F-gene coding sequence for isolate UK/5/01 |
| SEQ ID NO: 245 | F-gene coding sequence for isolate UK/8/01 |
| SEQ ID NO: 246 | F-gene coding sequence for isolate NL/12/02 |
| SEQ ID NO: 247 | F-gene coding sequence for isolate HK/1/02 |
| SEQ ID NO: 248 | F-protein sequence for isolate NL/1/00 |
| SEQ ID NO: 249 | F-protein sequence for isolate UK/1/00 |
| SEQ ID NO: 250 | F-protein sequence for isolate NL/2/00 |
| SEQ ID NO: 251 | F-protein sequence for isolate NL/13/00 |
| SEQ ID NO: 252 | F-protein sequence for isolate NL/14/00 |
| SEQ ID NO: 253 | F-protein sequence for isolate FL/3/01 |
| SEQ ID NO: 254 | F-protein sequence for isolate FL/4/01 |
| SEQ ID NO: 255 | F-protein sequence for isolate FL/8/01 |
| SEQ ID NO: 256 | F-protein sequence for isolate UK/1/01 |
| SEQ ID NO: 257 | F-protein sequence for isolate UK/7/01 |
| SEQ ID NO: 258 | F-protein sequence for isolate FL/10/01 |
| SEQ ID NO: 259 | F-protein sequence for isolate NL/6/01 |
| SEQ ID NO: 260 | F-protein sequence for isolate NL/8/01 |
| SEQ ID NO: 261 | F-protein sequence for isolate NL/10/01 |
| SEQ ID NO: 262 | F-protein sequence for isolate NL/14/01 |
| SEQ ID NO: 263 | F-protein sequence for isolate NL/20/01 |
| SEQ ID NO: 264 | F-protein sequence for isolate NL/25/01 |
| SEQ ID NO: 265 | F-protein sequence for isolate NL/26/01 |
| SEQ ID NO: 266 | F-protein sequence for isolate NL/28/01 |
| SEQ ID NO: 267 | F-protein sequence for isolate NL/30/01 |
| SEQ ID NO: 268 | F-protein sequence for isolate BR/2/01 |
| SEQ ID NO: 269 | F-protein sequence for isolate BR/3/01 |
| SEQ ID NO: 270 | F-protein sequence for isolate NL/2/02 |
| SEQ ID NO: 271 | F-protein sequence for isolate NL/4/02 |
| SEQ ID NO: 272 | F-protein sequence for isolate NL/5/02 |
| SEQ ID NO: 273 | F-protein sequence for isolate NL/6/02 |
| SEQ ID NO: 274 | F-protein sequence for isolate NL/7/02 |
| SEQ ID NO: 275 | F-protein sequence for isolate NL/9/02 |
| SEQ ID NO: 276 | F-protein sequence for isolate FL/1/02 |
| SEQ ID NO: 277 | F-protein sequence for isolate NL/1/81 |
| SEQ ID NO: 278 | F-protein sequence for isolate NL/1/93 |
| SEQ ID NO: 279 | F-protein sequence for isolate NL/2/93 |
| SEQ ID NO: 280 | F-protein sequence for isolate NL/4/93 |
| SEQ ID NO: 281 | F-protein sequence for isolate NL/1/95 |
| SEQ ID NO: 282 | F-protein sequence for isolate NL/2/96 |
| SEQ ID NO: 283 | F-protein sequence for isolate NL/3/96 |
| SEQ ID NO: 284 | F-protein sequence for isolate NL/1/98 |
| SEQ ID NO: 285 | F-protein sequence for isolate NL/17/00 |
| SEQ ID NO: 286 | F-protein sequence for isolate NL/22/01 |
| SEQ ID NO: 287 | F-protein sequence for isolate NL/29/01 |
| SEQ ID NO: 288 | F-protein sequence for isolate NL/23/01 |
| SEQ ID NO: 289 | F-protein sequence for isolate NL/17/01 |
| SEQ ID NO: 290 | F-protein sequence for isolate NL/24/01 |
| SEQ ID NO: 291 | F-protein sequence for isolate NL/3/02 |
| SEQ ID NO: 292 | F-protein sequence for isolate NL/3/98 |
| SEQ ID NO: 293 | F-protein sequence for isolate NL/1/99 |
| SEQ ID NO: 294 | F-protein sequence for isolate NL/2/99 |
| SEQ ID NO: 295 | F-protein sequence for isolate NL/3/99 |
| SEQ ID NO: 296 | F-protein sequence for isolate NL/11/00 |
| SEQ ID NO: 297 | F-protein sequence for isolate NL/12/00 |
| SEQ ID NO: 298 | F-protein sequence for isolate NL/1/01 |
| SEQ ID NO: 299 | F-protein sequence for isolate NL/5/01 |
| SEQ ID NO: 300 | F-protein sequence for isolate NL/9/01 |
| SEQ ID NO: 301 | F-protein sequence for isolate NL/19/01 |
| SEQ ID NO: 302 | F-protein sequence for isolate NL/21/01 |
| SEQ ID NO: 303 | F-protein sequence for isolate UK/11/01 |
| SEQ ID NO: 304 | F-protein sequence for isolate FL/1/01 |
| SEQ ID NO: 305 | F-protein sequence for isolate FL/2/01 |
| SEQ ID NO: 306 | F-protein sequence for isolate FL/5/01 |
| SEQ ID NO: 307 | F-protein sequence for isolate FL/7/01 |
| SEQ ID NO: 308 | F-protein sequence for isolate FL/9/01 |
| SEQ ID NO: 309 | F-protein sequence for isolate UK/10/01 |
| SEQ ID NO: 310 | F-protein sequence for isolate NL/1/02 |
| SEQ ID NO: 311 | F-protein sequence for isolate NL/1/94 |
| SEQ ID NO: 312 | F-protein sequence for isolate NL/1/96 |
| SEQ ID NO: 313 | F-protein sequence for isolate NL/6/97 |
| SEQ ID NO: 314 | F-protein sequence for isolate NL/7/00 |
| SEQ ID NO: 315 | F-protein sequence for isolate NL/9/00 |
| SEQ ID NO: 316 | F-protein sequence for isolate NL/19/00 |
| SEQ ID NO: 317 | F-protein sequence for isolate NL/28/00 |
| SEQ ID NO: 318 | F-protein sequence for isolate NL/3/01 |
| SEQ ID NO: 319 | F-protein sequence for isolate NL/4/01 |
| SEQ ID NO: 320 | F-protein sequence for isolate NL/11/01 |
| SEQ ID NO: 321 | F-protein sequence for isolate NL/15/01 |
| SEQ ID NO: 322 | F-protein sequence for isolate NL/18/01 |
| SEQ ID NO: 323 | F-protein sequence for isolate FL/6/01 |
| SEQ ID NO: 324 | F-protein sequence for isolate UK/5/01 |
| SEQ ID NO: 325 | F-protein sequence for isolate UK/8/01 |
| SEQ ID NO: 326 | F-protein sequence for isolate NL/12/02 |
| SEQ ID NO: 327 | F-protein sequence for isolate HK/1/02 |

1. A recombinant parainfluenza virus type 3 comprising a mammalian metapneumovirus nucleotide sequence, wherein the mammalian metapneumovirus is a negative-sense single stranded RNA virus belonging to the sub-family Pneumovirinae of the family Paramyxoviridae and wherein the mammalian metapneumovirus is phylogenetically closer related to a virus isolate deposited as I-2614 with CNCM, Paris than it is related to turkey rhinotracheitis virus (TRTV).

2. The recombinant parainfluenza virus of claim 1 wherein the mammalian metapneumovirus nucleotide sequence is substituted for a parainfluenza nucleotide sequence or inserted into the parainfluenza virus genome.

3. The recombinant parainfluenza virus of claim 1 wherein the mammalian metapneumovirus nucleotide sequence is inserted at position 1, 2, 3, 4, 5, or 6 of the parainfluenza virus genome.

4. The recombinant parainfluenza virus of claim 1 further comprising an RSV nucleotide sequence.

5. The recombinant parainfluenza virus of claim 1 wherein the parainfluenza virus is a bovine parainfluenza virus.

6. The recombinant parainfluenza virus of claim 5 further comprising one or more human parainfluenza virus nucleotide sequences.

7. The recombinant parainfluenza virus of claim 1 wherein the nucleotide sequence encodes a mammalian metapneumovirus polypeptide.

8. The recombinant parainfluenza virus of claim 7 wherein the polypeptide is the F or G protein of mammalian metapneumovirus, or a fragment thereof.

9. The recombinant parainfluenza virus of claim 7 wherein the polypeptide is at least 90% identical to SEQ ID NO: 70 or a fragment thereof; is at least 70% identical to SEQ ID NO: 78 or a fragment thereof; is at least 90% identical to SEQ ID NO: 62 or a fragment thereof; is at least 82% identical to SEQ ID NO: 18 or a fragment thereof; is at least 85% identical to SEQ ID NO: 42 or a fragment thereof; is at least 60% identical to SEQ ID NO: 50 or a fragment thereof; is at least 85% identical to SEQ ID NO: 34 or a fragment thereof; is at least 20% identical to SEQ ID NO: 26 or a fragment thereof; or is at least 30% identical to SEQ ID NO: 86 or a fragment thereof.

10. The recombinant parainfluenza virus of claim 7 wherein the polypeptide is SEQ ID NO: 78, SEQ ID NO: 62, SEQ ID NO: 18, SEQ ID NO: 42, SEQ ID NO: 50, SEQ ID NO: 34, SEQ ID NO: 26, SEQ ID NO: 86, SEQ ID NO: 70, SEQ ID NO: 28, SEQ ID NO: 72, SEQ ID NO: 80, SEQ ID NO: 64, SEQ ID NO: 20, SEQ ID NO: 44, SEQ ID NO: 52, SEQ ID NO: 88, SEQ ID NO: 36, SEQ ID NO: 29, SEQ ID NO: 73, SEQ ID NO: 81, SEQ ID NO:65, SEQ ID NO:21, SEQ ID NO:45, SEQ ID NO:53, SEQ ID NO:89, SEQ ID NO: 37, SEQ ID NO: 27, SEQ ID NO: 71, SEQ ID NO: 79, SEQ ID NO: 63, SEQ ID NO: 43, SEQ ID NO: 51, SEQ ID NO: 87, SEQ ID NO: 35, or a fragment thereof.

11. The recombinant parainfluenza virus of claim 1 wherein the nucleotide sequence is selected from the group consisting of SEQ ID NO:22-25; SEQ ID NO:30-33; SEQ ID NO:38-41; SEQ ID NO:46-49; SEQ ID NO:54-61; SEQ ID NO:66-69; SEQ ID NO:74-77; SEQ ID NO:82-85; SEQ ID NO:90-93; SEQ ID NO:98-132; SEQ ID NO:168 247; or a fragment thereof.

12. The recombinant parainfluenza virus of claims 9 or 10 wherein the fragment is at least 10, at least 15, at least 20, at least 25, at least 50, at least 75, at least 100, at least 150, at least 250, at least 500, at least 750, or at least 1000 amino acids in length.

13. The recombinant parainfluenza virus of claim 11 wherein the fragment is at least 10, at least 15, at least 20, at least 25, at least 50, at least 75, at least 100, at least 150, at least 250, at least 500, at least 750, or at least 1000 nucleotides in length.

14. The virus of claim 1 wherein the heterologous nucleotide sequence is derived from a human metapneumovirus.

15. The virus of claim 7 wherein the mammalian metapneumovirus sequence encodes an F protein, a G protein, an SH protein, an N protein, a P protein, an M2 protein, an M2-1 protein, an M2-2 protein, an L protein, or a fragment thereof.

16. A recombinant DNA or RNA molecule encoding the genome of the virus of any of claims 1-14.

17. A recombinant DNA or RNA molecule encoding the genome of the virus of claim 15.

18. A vaccine formulation comprising the recombinant virus of any of claims 1-14 and a pharmaceutically acceptable excipient.

19. A vaccine formulation comprising the recombinant virus of claim 15 and a pharmaceutically acceptable excipient.

20. A method of treating a respiratory tract infection in a mammal, said method comprising administering the vaccine of claim 18.

21. A method of treating a respiratory tract infection in a mammal, said method comprising administering the vaccine of claim 19.

22. The method of claim 20 wherein the mammal is a human.

23. The method of claim 21 wherein the mammal is a human.

24. A method for propagating the recombinant virus of any of claims 1-15, wherein the method comprises culturing cells that are infected with the virus at a temperature wherein the temperature is lower than the temperature that is optimal for growth of the cells.

25. A method for propagating the recombinant virus of any of claims 1-15, wherein the method comprises (i) culturing cells at a first temperature before infection with the virus; (ii) infecting the cells with the virus; and (iii) culturing the cells at a second temperature after infection of the cells with the virus, wherein the second temperature is lower than the first temperature.

26. A method for propagating the recombinant virus of any of claims 1-15, wherein the method comprises culturing cells that are infected with the virus in the absence of serum.

27. A method for propagating the recombinant virus of any of claims 1-15, wherein the method comprises (i) culturing cells in the presence of serum before infection with the virus; (ii) infecting the cells with the virus; and (iii) culturing the cells in the absence of serum after infection of the cells with the virus.

28. A method for propagating the recombinant virus of any of claims 1-15, wherein the method comprises culturing cells that are infected with the virus without serum at a temperature lower than the temperature that is optimal for growth of the cells.
